(19) United States
(12) Patent Application Publication
(10) Pub. No.: US 2026/0209757 A1
(43) Pub. Date: Jul. 23, 2026

(54) Compositions and Methods for Immunotherapy

(71) Applicant: Intellia Therapeutics, Inc., Cambridge, MA (US)

(72) Inventors: Amy Melissa Becker, Lexington, MA (US); Surbhi Goel, Winchester, MA (US); Sarah Beth Hesse, Arlington, MA (US); Troy Aaron Luster, Norfolk, MA (US); Birgit Schultes, Arlington, MA (US); Stephanie A. Yazinski, Lynnfield, MA (US); Pooja Vinay, St. Louis, MO (US)

(73) Assignee: Intellia Therapeutics, Inc., Cambridge, MA (US)

(21) Appl. No.: 19/357,943

(22) Filed: Oct. 14, 2025

Related U.S. Application Data
(62) Division of application No. 17/231,556, filed on Apr. 15, 2021, now Pat. No. 12,630,820.
Application No. 17/231,556 is a continuation of application No. PCT/US19/56399, filed on Oct. 15, 2019.
(60) Provisional application No. 62/746,522, filed on Oct. 16, 2018.
(60) Provisional application No. 62/747,037, filed on Oct. 17, 2018.

(51) Int. Cl. C12N 15/11; C07K 14/725; C12N 9/22

(52) U.S. Cl. CPC C12N 15/111; C07K 14/7051; C12N 9/22; C12N 2310/20; C12N 2320/30

(57) ABSTRACT

Compositions and methods for editing, e.g., altering a DNA sequence, within the TRBC1, TRBC2 and/or TRAC genes are provided. Compositions and methods for immunotherapy are provided, for example.

### CROSS-REFERENCE TO RELATED APPLICATIONS

The present application is a Divisional of U.S. application Ser. No. 17/231,556, filed Apr. 15, 2021, entitled “Compositions and Methods for Immunotherapy,” which is a Continuation of International Application No. PCT/US2019/056399, filed Oct. 15, 2019, entitled “Compositions and Methods for Immunotherapy,” which claims the benefit of priority to U.S. Provisional Application No. 62/747,037, filed Oct. 17, 2018, entitled “Compositions and Methods for Immunotherapy,” and U.S. Provisional Application No. 62/746,522, filed Oct. 16, 2018, entitled “Compositions and Methods for Immunotherapy,” all of which are incorporated herein by reference in their entirety.

### REFERENCE TO SEQUENCE LISTING

This application is being filed with a sequence listing in electronic XML format. The sequence listing is provided as a file entitled “01155-0026-01US.xml,” was created on Jan. 21, 2026, and is 713,572 bytes in size. The information in the electronic format of the sequence listing is incorporated by reference in its entirety.

CRISPRs (Clustered Regularly Interspaced Short Palindromic Repeats) evolved in bacteria as an adaptive immune system to defend against viral attack. Upon exposure to a virus, short segments of viral DNA are integrated into the CRISPR locus of the bacterial genome. RNA is transcribed from a portion of the CRISPR locus that includes the viral sequence. That RNA, which contains sequence complementary to the viral genome, mediates targeting of a Cas9 protein to the sequence in the viral genome. The Cas9 protein cleaves and thereby restricts the viral target.

Recently, the CRISPR/Cas system has been adapted for genome editing in eukaryotic cells. The introduction of site-specific single strand breaks (SSBs) or double strand breaks (DSBs) allows for target sequence alteration through, for example, non-homologous end-joining (NHEJ) or homology-directed repair (HDR).

Eyquem et al. (2017) Nature 543(7643):113-117 report that targeting a chimeric antigen receptor (CAR) to the locus of the TRAC gene with a CRISPR-Cas9 system enhances tumor rejection. In addition, such targeting to the TRAC locus averts tonic CAR signaling and establishes effective internalization and re-expression of the CAR following single or repeated exposure to antigen, thus delaying effector T-cell differentiation and exhaustion. Nonetheless, existing approaches may produce T cells with less than desired activity against the intended target (including, but not limited to, producing T cells that express a CAR from the TRAC locus and an endogenous TRBC gene product that can interact with the CAR and provide T cells with undesired reactivity). Thus, there is a need for improved compositions and methods for immunotherapy.

Accordingly, the following embodiments are provided. In some embodiments, the present invention provides compositions and methods using a guide RNA with an RNA-guided DNA binding agent such as the CRISPR/Cas system to substantially reduce or knockout expression of the TRBC and/or TRAC genes, thereby substantially reducing or eliminating the production of the native alpha and/or beta subunits of the T-cell receptor, also called TCR.

### SUMMARY

The inventions described herein relate to compositions and methods for engineered T cell therapies (e.g., immunooncology and for reducing autoimmunity), for example, cells modified at specific target sequences in their genome, including as modified by introduction of CRISPR systems that include gRNA molecules which target said target sequences, and methods of making and using therefor. For example, the present disclosure relates to and provides gRNA molecules, CRISPR systems, cells, and methods useful for genome editing of cells, e.g. T cells, e.g. T cells engineered to lack endogenous T-cell receptor expression, e.g. T cells suitable for further engineering to insert a coding sequence of interest, e.g. T cells further engineered to express a T-cell receptor, such as a modified or mutant T-cell receptor, and useful for immunotherapy.

In a first aspect, the invention provides a gRNA molecule including a tracrRNA and crRNA, wherein the crRNA includes a targeting domain that binds to a target sequence of a T cell. In an embodiment, the targeting domain is complementary with a target sequence of a T cell. The T cell may be an autologous or allogeneic T cell. The T cell may be a CD3⁺, CD4⁺, and/or CD8⁺ T cell.

The invention is as provided in the claims and further embodiments are set out below.

The invention provides a method of altering a DNA sequence within the TRBC1 and/or TRBC2 gene, comprising delivering a composition to a cell, wherein the composition comprises:
(a) a guide RNA comprising a sequence chosen from:
   i. a guide sequence selected from SEQ ID NOs: 1-89;
   ii. at least 17, 18, 19, or 20 contiguous nucleotides of a sequence selected from SEQ ID NOs: 1-89;
   iii. a guide sequence that is at least 99%, 98%, 97%, 96%, 95%, 94%, 93%, 92%, 91%, or 90% identical to a sequence selected from SEQ ID NOs: 1-89;
   iv. a guide sequence comprising any one of SEQ ID NOs: 1-24; and
   v. a guide sequence comprising any one of SEQ ID NOs: 1-6; or
(b) a nucleic acid encoding a guide RNA of (a); and optionally
(c) an RNA-guided DNA binding agent or a nucleic acid encoding an RNA-guided DNA binding agent.

The invention additionally provides a method of altering a DNA sequence, comprising delivering a composition to a cell, wherein the composition comprises:
(a) a guide RNA comprising a sequence chosen from:
   i. a sequence that comprises 15 consecutive nucleotides ±10 nucleotides of a genomic coordinate listed in any of Tables 1 and/or 3 for SEQ ID NOs: 1-89 and 179-184;
   ii. at least 17, 18, 19, or 20 contiguous nucleotides of a sequence from (i);
   iii. a guide sequence that is at least 99%, 98%, 97%, 96%, 95%, 94%, 93%, 92%, 91%, or 90% identical to a sequence selected from (i);
(b) a nucleic acid encoding a guide RNA of (a); and optionally
(c) an RNA-guided DNA binding agent or a nucleic acid encoding an RNA-guided DNA binding agent.

The invention additionally also provides a method of reducing the expression of the TRBC1 and/or TRBC2 gene comprising delivering a composition to a cell, wherein the composition comprises:
(a) a guide RNA comprising a sequence chosen from:
   i) a guide sequence selected from SEQ ID NOs: 1-89;
   ii) at least 17, 18, 19, or 20 contiguous nucleotides of a sequence selected from SEQ ID NOs: 1-89;
   iii) a guide sequence that is at least 99%, 98%, 97%, 96%, 95%, 94%, 93%, 92%, 91%, or 90% identical to a sequence selected from SEQ ID NOs: 1-89;
   iv) a guide sequence comprising any one of SEQ ID NOs: 1-24; and
   v) a guide sequence comprising any one of SEQ ID NOs: 1-6; or
(b) a nucleic acid encoding a guide RNA of (a); and optionally
(c) an RNA-guided DNA binding agent or a nucleic acid encoding an RNA-guided DNA binding agent.

The invention additionally provides a method of immunotherapy comprising administering a composition to a subject, an autologous cell thereof, and/or an allogeneic cell, wherein the composition comprises:
(a) a guide RNA comprising a sequence chosen from:
   i) a guide sequence selected from SEQ ID NOs: 1-89;
   ii) at least 17, 18, 19, or 20 contiguous nucleotides of a sequence selected from SEQ ID NOs: 1-89;
   iii) a guide sequence that is at least 99%, 98%, 97%, 96%, 95%, 94%, 93%, 92%, 91%, or 90% identical to a sequence selected from SEQ ID NOs: 1-89;
   iv) a guide sequence comprising any one of SEQ ID NOs: 1-24; and
   v) a guide sequence comprising any one of SEQ ID NOs: 1-6; or
(b) a nucleic acid encoding a guide RNA of (a); and optionally
(c) an RNA-guided DNA binding agent or nucleic acid encoding an RNA-guided DNA binding agent.

The invention additionally also provides method of altering a DNA sequence within the TRAC gene, comprising delivering a composition to a cell, wherein the composition comprises:
(a) a guide RNA comprising a sequence chosen from:
   i) a guide sequence selected from SEQ ID NOs: 90-178, 185, and 213-218;
   ii) at least 17, 18, 19, or 20 contiguous nucleotides of a sequence selected from SEQ ID NOs: 90-178, 185, and 213-218;
   iii) a guide sequence that is at least 99%, 98%, 97%, 96%, 95%, 94%, 93%, 92%, 91%, or 90% identical to a sequence selected from SEQ ID NOs: 90-178, 185, and 213-218;
   iv) a guide sequence comprising any one of SEQ ID NOs: 90-113 and 213-218; and
   v) a guide sequence comprising any one of SEQ ID NOs: 90-95; or
(b) a nucleic acid encoding a guide RNA of (a); and optionally
(c) an RNA-guided DNA binding agent or a nucleic acid encoding an RNA-guided DNA binding agent.

The invention additionally provides a method of reducing the expression of the TRAC gene comprising delivering a composition to a cell, wherein the composition comprises:
(a) a guide RNA comprising a sequence chosen from:
   i) a guide sequence selected from SEQ ID NOs: 90-178, 185, and 213-218;
   ii) at least 17, 18, 19, or 20 contiguous nucleotides of a sequence selected from SEQ ID NOs: 90-178, 185, and 213-218;
   iii) a guide sequence that is at least 99%, 98%, 97%, 96%, 95%, 94%, 93%, 92%, 91%, or 90% identical to a sequence selected from SEQ ID NOs: 90-178, 185, and 213-218;
   iv) a guide sequence comprising any one of SEQ ID NOs: 90-113 and 213-218; and
   v) a guide sequence comprising any one of SEQ ID NOs: 90-95; or
(b) a nucleic acid encoding a guide RNA of (a); and optionally
(c) an RNA-guided DNA binding agent or a nucleic acid encoding an RNA-guided DNA binding agent.

The invention additionally also provides method of immunotherapy comprising administering a composition to a subject, an autologous cell thereof, and/or an allogeneic cell, wherein the composition comprises:
(a) a guide RNA comprising a sequence chosen from:
   i) a guide sequence selected from SEQ ID NOs: 90-178, 185, and 213-218;
   ii) at least 17, 18, 19, or 20 contiguous nucleotides of a sequence selected from SEQ ID NOs: 90-178, 185, and 213-218;
   iii) a guide sequence that is at least 99%, 98%, 97%, 96%, 95%, 94%, 93%, 92%, 91%, or 90% identical to a sequence selected from SEQ ID NOs: 90-178, 185, and 213-218;
   iv) a guide sequence comprising any one of SEQ ID NOs: 90-113 and 213-218; and
   v) a guide sequence comprising any one of SEQ ID NOs: 90-95; or
(b) a nucleic acid encoding a guide RNA of (a.); and optionally
(c) an RNA-guided DNA binding agent or nucleic acid encoding an RNA-guided DNA binding agent.

The invention additionally provides a method of altering a DNA sequence, comprising delivering a composition to a cell, wherein the composition comprises:
(a) a guide RNA comprising a sequence chosen from:
   i. a sequence that comprises 15 consecutive nucleotides ±10 nucleotides of a genomic coordinate listed in any of Tables 2 and/or 3 for SEQ ID NOs: 90-218;
   ii. at least 17, 18, 19, or 20 contiguous nucleotides of a sequence from (i);
   iii. a guide sequence that is at least 99%, 98%, 97%, 96%, 95%, 94%, 93%, 92%, 91%, or 90% identical to a sequence selected from (i);
(b) a nucleic acid encoding a guide RNA of (a); and optionally
(c) an RNA-guided DNA binding agent or a nucleic acid encoding an RNA-guided DNA binding agent.

The invention additionally also provides a method of altering a DNA sequence within the TRBC1, TRBC2 and/or TRAC genes, comprising delivering to a cell a first guide RNA, a second guide RNA and optionally an RNA-guided DNA binding agent or a nucleic acid encoding an RNA-guided DNA binding agent, wherein the first guide RNA comprises a sequence chosen from:
i) a guide sequence selected from SEQ ID NOs: 1-89;
ii) at least 17, 18, 19, or 20 contiguous nucleotides of a sequence selected from SEQ ID NOs: 1-89;
iii) a guide sequence that is at least 99%, 98%, 97%, 96%, 95%, 94%, 93%, 92%, 91%, or 90% identical to a sequence selected from SEQ ID NOs: 1-89;
iv) a guide sequence comprising any one of SEQ ID NOs: 1-24; and v) a guide sequence comprising any one of SEQ ID NOs: 1-6, and
   wherein the second guide RNA comprises a sequence chosen from:
i. a guide sequence selected from SEQ ID NOs: 90-178, 185, and 213-218;
ii. at least 17, 18, 19, or 20 contiguous nucleotides of a sequence selected from SEQ ID NOs: 90-178, 185, and 213-218;
iii. a guide sequence that is at least 99%, 98%, 97%, 96%, 95%, 94%, 93%, 92%, 91%, or 90% identical to a sequence selected from SEQ ID NOs: 90-178, 185, and 213-218;
iv. a guide sequence comprising any one of SEQ ID NOs: 90-113 and 213-218; and
v. a guide sequence comprising any one of SEQ ID NOs: 90-95.

The invention additionally also provides a method of reducing the expression of the TRBC1, TRBC2 and/or TRAC genes, comprising delivering to a cell a first guide RNA, a second guide RNA and optionally an RNA-guided DNA binding agent or a nucleic acid encoding an RNA-guided DNA binding agent, wherein the first guide RNA comprises a sequence chosen from:
i) a guide sequence selected from SEQ ID NOs: 1-89;
ii) at least 17, 18, 19, or 20 contiguous nucleotides of a sequence selected from SEQ ID NOs: 1-89;
iii) a guide sequence that is at least 99%, 98%, 97%, 96%, 95%, 94%, 93%, 92%, 91%, or 90% identical to a sequence selected from SEQ ID NOs: 1-89;
iv) a guide sequence comprising any one of SEQ ID NOs: 1-24; and
v) a guide sequence comprising any one of SEQ ID NOs: 1-6, and
   wherein the second guide RNA comprises a sequence chosen from:
i) a guide sequence selected from SEQ ID NOs: 90-178, 185, and 213-218;
ii) at least 17, 18, 19, or 20 contiguous nucleotides of a sequence selected from SEQ ID NOs: 90-178, 185, and 213-218;
iii) a guide sequence that is at least 99%, 98%, 97%, 96%, 95%, 94%, 93%, 92%, 91%, or 90% identical to a sequence selected from SEQ ID NOs: 90-178, 185, and 213-218;
iv) a guide sequence comprising any one of SEQ ID NOs: 90-113 and 213-218; and
v) a guide sequence comprising any one of SEQ ID NOs: 90-95.

The invention additionally provides a method of immunotherapy comprising administering a composition to a subject, an autologous cell thereof, or an allogeneic cell, wherein the composition comprises: a first guide RNA, a second guide RNA, and optionally an RNA-guided DNA binding agent or a nucleic acid encoding an RNA-guided DNA binding agent, wherein the first guide RNA comprises a sequence chosen from:
i) a guide sequence selected from SEQ ID NOs: 1-89;
ii) at least 17, 18, 19, or 20 contiguous nucleotides of a sequence selected from SEQ ID NOs: 1-89;
iii) a guide sequence that is at least 99%, 98%, 97%, 96%, 95%, 94%, 93%, 92%, 91%, or 90% identical to a sequence selected from SEQ ID NOs: 1-89;
iv) a guide sequence comprising any one of SEQ ID NOs: 1-24; and
v) a guide sequence comprising any one of SEQ ID NOs: 1-6, and
   wherein the second guide RNA comprises a sequence chosen from:
i. a guide sequence selected from SEQ ID NOs: 90-178, 185, and 213-218;
ii. at least 17, 18, 19, or 20 contiguous nucleotides of a sequence selected from SEQ ID NOs: 90-178, 185, and 213-218;
iii. a guide sequence that is at least 99%, 98%, 97%, 96%, 95%, 94%, 93%, 92%, 91%, or 90% identical to a sequence selected from SEQ ID NOs: 90-178, 185, and 213-218;
iv. a guide sequence comprising any one of SEQ ID NOs: 90-113 and 213-218; and
v. a guide sequence comprising any one of SEQ ID NOs: 90-95.

Without wishing to be bound by theory, the use of a double TRAC/TRBC mutant, e.g., a knockout mutant is in some embodiments advantageous in the context of the present invention as a greater proportion of the engineered/introduced TCR reaches the cell surface.

In some embodiments, a TRBC knockout in combination with a TRAC knock-in of an exogenous TCR in an engineered cell is advantageous in yielding greater activity of the exogenous TCR in an engineered cell.

In some embodiments, a TRBC knockout in combination with a TRAC knock-in of an exogenous TCR in an engineered cell is advantageous in yielding greater selectivity of the exogenous TCR in an engineered cell.

In addition, in some embodiments, a TRBC knockout in combination with a TRAC knockin of an exogenous TCR in an engineered cell may be advantageous in yielding greater cell killing, e.g., as measured by a caspase assay.

In some embodiments, a TRBC knockout in combination with a TRAC knock-in of an exogenous TCR in an engineered cell is advantageous in yielding greater IFN-gamma secretion, e.g., in response to an inducing peptide (e.g. Wilms' tumor gene (WT1) antigen).

Furthermore, in some embodiments, a double TRAC/TRBC knockout with the TCR is advantageous in yielding greater IFN-gamma secretion, e.g., in CD4+ cells.

In addition, advantageously in some embodiments, the interferon response (IFNγ and/or TNFα) is increased in edited cells wherein transcription of an inserted donor construct is promoted by an endogenous promoter of the TRAC.

Furthermore, in some embodiments, a double TRAC/TRBC knockout with these particular TCR is advantageous in yielding greater IFN-7 secretion in CD4+ cells. Not wishing to be bound by theory, in some embodiments, it is rarer to observe such activity in CD4+ cells, which are not usually strongly linked with reactivity to peptides presented on HLA Class I.

In addition, in some embodiments, a double TRAC/TRBC knockout with these particular TCR is advantageous in yielding a greater response in CD4+ cells, e.g., in a degranulation assay. Again, not wishing to be bound by theory, in some embodiments, it is rarer to observe such activity in CD4+ cells, which are not usually strongly linked with reactivity to peptides presented on HLA Class I.

In addition, in some embodiments, a double TRAC/TRBC knockout mutant is advantageous in yielding a lesser degree of alloreactivity than the TRAC single knockout mutant, e.g., when tested in a mixed lymphocyte reaction.

The invention additionally provides a method of expressing a heterologous immunological receptor via in locus insertion at the TRAC locus, comprising delivering to a cell a first guide RNA, a second guide RNA and an RNA-guided DNA binding agent or a nucleic acid encoding an RNA-guided DNA binding agent, wherein the first guide RNA comprises a sequence chosen from:
i) a guide sequence selected from SEQ ID NOs: 1-89;
ii) at least 17, 18, 19, or 20 contiguous nucleotides of a sequence selected from SEQ ID NOs: 1-89;
iii) a guide sequence that is at least 99%, 98%, 97%, 96%, 95%, 94%, 93%, 92%, 91%, or 90% identical to a sequence selected from SEQ ID NOs: 1-89;
iv) a guide sequence comprising any one of SEQ ID NOs: 1, 2, 3, 5, 6; and
v) a guide sequence comprising any one of SEQ ID NOs: 2, 3, 5, 6, and
   wherein the second guide RNA comprises a sequence chosen from:
i) a guide sequence selected from SEQ ID NOs: 90, 95, 97, 98, 185, 214, 218;
ii) at least 17, 18, 19, or 20 contiguous nucleotides of a sequence selected from SEQ ID NOs: 90, 95, 97, 98, 185, 214, and 218;
iii) a guide sequence that is at least 99%, 98%, 97%, 96%, 95%, 94%, 93%, 92%, 91%, or 90% identical to a sequence selected from SEQ ID NOs: 90, 95, 97, 98, 185, 214, and 218;
iv) a guide sequence comprising any one of SEQ ID NOs: 90, 95, 97, 185, and 214;
v) a guide sequence comprising any one of SEQ ID NOs: 90, 95, and 185; and
vi) a guide sequence comprising SEQ ID NO: 90 or 214.

A single cleavage and/or editing event may allow the “knock out” of the endogenous TRAC gene. As a further advantage, if a template is introduced or supplied during the editing process an exogenous polypeptide of interest may be inserted into the target locus of the editing event. This advantage flows particularly from the use of guide RNAs with the following SEQ ID NOs: 90, 95, 97, 185, 203, 204, 205, 206, 210, 211, and/or 214. These guide RNAs may also be introduced in concert with a gRNA targeting the locus or loci of the TRBC genes.

The first guide RNA may comprise the sequence of SEQ ID NO: 2 and the second guide RNA comprises the sequence of SEQ ID NO: 90.

The first guide RNA may comprise the sequence of SEQ ID NO: 180 and the second guide RNA may comprise the sequence of SEQ ID NO: 186.

The first guide RNA may comprise the sequence of any one of SEQ ID NOs: 1, 2, 3, 5, 6, and the second guide RNA may comprise the sequence of SEQ ID NO: 90.

The first guide RNA may comprise the sequence of any one of SEQ ID NOs: 1, 2, 3, 5, 6, and the second guide RNA may comprise the sequence of SEQ ID NO: 214.

The first guide RNA, the second guide RNA and the RNA-guided DNA binding agent or the nucleic acid encoding an RNA-guided DNA binding agent may be introduced or administered at substantially the same time.

The DNA sequences within the TRBC1, TRBC2 and/or TRAC genes may be altered simultaneously.

An RNA-guided DNA binding agent or nucleic acid encoding an RNA-guided DNA binding agent may be introduced or administered as a part of methods of modification.

The methods may further comprise:
(a) inducing a double-stranded break (DSB) within the TRBC1, TRBC2 and/or TRAC genes in a cell and/or subject; or
(b) inducing a single-stranded break (SSB) within the TRBC1, TRBC2 and/or TRAC genes in a cell and/or a subject; or
(c) reducing the expression of the TRBC1, TRBC2 and/or TRAC genes in a cell and/or subject.

The methods may further comprise introducing a nucleic acid sequence encoding a polypeptide of interest, optionally wherein:
(a) the one or more polypeptides of interest comprise a receptor;
(b) the one or more polypeptides of interest comprise an immunological receptor;
(c) the one or more polypeptides of interest comprise a T-cell receptor, further optionally wherein the T-cell receptor recognizes a cancer antigen;
(d) the one or more polypeptides of interest comprise a WT1-specific T-cell receptor, wherein the T-cell receptor recognizes WT1 or a fragment thereof;
(e) the one or more polypeptides of interest comprise a chimeric antigen receptor, further optionally wherein the chimeric antigen receptor recognizes a cancer antigen; or
(f) the one or more polypeptides of interest comprise a WT1-specific chimeric antigen receptor, wherein the chimeric antigen receptor recognizes WT1 or a fragment thereof.

The methods may further comprise:
a. introducing a TCR α chain and a TCR β chain;
b. introducing one or more nucleic acid sequences that encode a TCR α chain and a TCR β chain;
c. introducing a WT1-specific TCR α chain and β chain;
d. introducing one or more nucleic acid sequences that encode a WT1-specific TCR α chain and β chain;
e. introducing a first TCR sequence selected from: (i) SEQ ID NO: 501 or 504; (ii) an amino acid sequence that is at least 99%, 95%, 90%, 85%, 80%, 70%, of 60% identical to SEQ ID NO: 501 or 504; and (iii) a contiguous subsequence of at least 20, 30, 40, 50, 60, 70, 80, 90, 100, 150, 200, or 250 amino acids of SEQ ID NO: 501 or 504, and
   introducing a second TCR sequence selected from: (i) SEQ ID NO: 502 or 505; (ii) an amino acid sequence that is at least 99%, 95%, 90%, 85%, 80%, 70%, of 60% identical to SEQ ID NO: 502 or 505; and (iii) a contiguous subsequence of at least 20, 30, 40, 50, 60, 70, 80, 90, 100, 150, 200, 250, or 300 amino acids of SEQ ID NO: 502 or 505,
f. introducing a first TCR sequence selected from: (i) SEQ ID NO:501 or 513; (ii) an amino acid sequence that is at least 99%, 95%, 90%, 85%, 80%, 70%, of 60% identical to SEQ ID NO:510 or 513; and (iii) a contiguous subsequence of at least 20, 30, 40, 50, 60, 70, 80, 90, 100, 150, 200, or 250 amino acids of SEQ ID NO:510 or 513, and
   introducing a second TCR sequence selected from: (i) SEQ ID NO:511 or 514; (ii) an amino acid sequence that is at least 99%, 95%, 90%, 85%, 80%, 70%, of 60% identical to SEQ ID NO:511 or 514; and (iii) a contiguous subsequence of at least 20, 30, 40, 50, 60, 70, 80, 90, 100, 150, 200, 250, or 300 amino acids of SEQ ID NO:511 or 514,
g. introducing a nucleic acid sequence comprising a sequence that encodes a first TCR sequence of (e) or (f);
h. introducing a nucleic acid sequence comprising a sequence that encodes a second TCR sequence of (e)-(f);
i. introducing a nucleic acid sequence comprising the nucleic acid sequence of (g) and (h);
j. introducing a polypeptide selected from SEQ ID NO: 500, 503, 506, 509, 512, 515, 518, or 521 or an amino acid sequence that is at least 99%, 95%, 90% identical thereto, optionally by introducing a nucleic acid sequence encoding the same; or
k. introducing a TCR α chain and a TCR β chain polypeptides selected from (i)-(viii) below, or an amino acid sequence that is at least 99%, 95%, 90% identical thereto:
   i) SEQ ID NO: 501 and SEQ ID NO: 502;
   ii) SEQ ID NO: 504 and SEQ ID NO:505;
   iii) SEQ ID NO: 507 and SEQ ID NO:508;
   iv) SEQ ID NO: 510 and SEQ ID NO: 511;
   v) SEQ ID NO: 513 and SEQ ID NO: 514;
   vi) SEQ ID NO: 516 and SEQ ID NO: 517;
   vii) SEQ ID NO: 519 and SEQ ID NO:520;
l. introducing a nucleic acid sequence encoding a TCR α chain and a TCR β chain polypeptide of (k).

The first nucleic acid sequence may be flanked by sequences homologous to a first target locus. The second nucleic acid sequence may be flanked by sequences homologous to a second target locus. The flanking sequences may be of at least 17, 18, 19, 20, 21, 22, 23, 24, 25, 30, 35, or 40 nucleotides in length.

The first target locus may be the TRAC, TRBC1 or TRBC2 gene, e.g., the TRAC gene. The second target locus may be the TRAC, TRBC1 or TRBC2 gene, e.g., either the TRBC1 or TRBC2 genes.

The introduced nucleic acid sequence, or the first nucleic acid sequence and the second nucleic acid sequence may be constituted without a promoter region, i.e. the nucleic acid may be “promoterless”.

The introduced nucleic acid sequence, or the first nucleic acid sequence and the second nucleic acid sequence, may be operably linked to a promoter, optionally wherein the promoter is an EF-1α promoter (SEQ ID NO: 603).

The introduced nucleic acid sequence, or the first nucleic acid sequence and the second nucleic acid sequence, may be introduced via a vector, via transfection, via a lipid nanoparticle, or via microinjection.

The vector may be a viral vector, further optionally wherein the viral vector is an adeno-associated virus vector.

The invention also provides a method of in locus insertion of a TCR (such as a WT1-specific TCR), comprising delivering to a cell a first guide RNA for inserting the TCR that comprises a guide sequence selected from: SEQ ID NOs: 90, 95, 97, 98, 185, 214, and 218, and optionally (i) an RNA-guided DNA binding agent or a nucleic acid encoding an RNA-guided DNA binding agent; and/or (ii) a donor nucleic acid molecule that encodes a TCR(such as a WT1-specific TCR). The method may further comprise delivering a second guide RNA comprising a sequence selected from SEQ ID NOs: 1-89. The method may further comprise delivering a second guide RNA comprising a sequence selected from SEQ ID NOs: 179-184.

The TCR may be a WT1-specific TCR that comprises:
i) a polypeptide selected from SEQ ID NO: 500, 503, 506, 509, 512, 515, 518, or 521 or an amino acid sequence that is at least 99%, 95%, 90% identical thereto; or
ii) TCR α chain and a TCR β chain polypeptides selected from (i)-(viii) below, or an amino acid sequence that is at least 99%, 95%, 90% identical thereto:
   i) SEQ ID NO: 502 and SEQ ID NO:503;
   ii) SEQ ID NO: 504 and SEQ ID NO:505;
   iii) SEQ ID NO: 507 and SEQ ID NO:508;
   iv) SEQ ID NO: 510 and SEQ ID NO: 511;
   v) SEQ ID NO: 513 and SEQ ID NO: 514;
   vi) SEQ ID NO: 516 and SEQ ID NO: 517;
   vii) SEQ ID NO: 519 and SEQ ID NO: 520;
   viii) SEQ ID NO: 522 and SEQ ID NO: 523.

The invention also provides a composition comprising:
a. a guide RNA comprising
   i. a guide sequence selected from SEQ ID NOs: 1-89; or
   ii. at least 17, 18, 19, or 20 contiguous nucleotides of a sequence selected from SEQ ID NOs: 1-89; or
   iii. a guide sequence that is at least 99%, 98%, 97%, 96%, 95%, 94%, 93%, 92%, 91%, or 90% identical to a sequence selected from SEQ ID NOs: 1-89; or
   iv. a guide sequence comprising any one of SEQ ID NOs: 1-24; or
   v. a guide sequence comprising any one of SEQ ID NOs: 1-6; and optionally
b. an RNA-guided DNA binding agent or nucleic acid encoding an RNA-guided DNA binding agent.

The composition may be for use in altering a DNA sequence within the TRBC1 and/or TRBC2 genes in a cell. The composition may be for use in reducing the expression of the TRBC1 and/or TRBC2 genes in a cell. The guide RNA in the composition may comprise a sequence selected from any of SEQ ID NOs 196-200.

The invention also provides a composition comprising:
a. a guide RNA comprising
   i. a guide sequence selected from SEQ ID NOs: 90-178, 185, and 213-218; or
   ii. at least 17, 18, 19, or 20 contiguous nucleotides of a sequence selected from SEQ ID NOs: 90-178, 185, and 213-218; or
   iii. a guide sequence that is at least 99%, 98%, 97%, 96%, 95%, 94%, 93%, 92%, 91%, or 90% identical to a sequence selected from SEQ ID NOs: 90-178, 185, and 213-218; or
   iv. a guide sequence comprising any one of SEQ ID NOs: 90-113 and 213-218; or
   v. a guide sequence comprising any one of SEQ ID NOs: 90-95; and optionally
b. an RNA-guided DNA binding agent or nucleic acid encoding an RNA-guided DNA binding agent.

The composition may be for use in altering a DNA sequence within the TRAC gene in a cell. The composition may be for use in reducing the expression of the TRAC gene in a cell. The guide RNA may be selected from any of SEQ ID NOs 185-192 and 201-212.

The invention also provides a cell, altered by the methods described herein. The cell may be altered ex vivo.

The cell may be a T cell. The cell may be a CD3⁺, CD4⁺ and/or CD8⁺ T cell. The cell may be a mammalian, primate, or human cell.

The cell may lack an endogenous T-cell receptor. The cell may be suitable for preparation of a T cell expressing a non-endogenous T-cell receptor. The cell may thus be used for preparation of a T cell expressing a CAR.

The cell altered as described herein may be a CD3-cell. The cell may be a CD3⁺ cell before being altered.

The cell may additionally comprise one or more nucleic acid sequences encoding a polypeptide of interest, optionally wherein:
(a) the one or more polypeptides of interest comprise a receptor;
(b) the one or more polypeptides of interest comprise an immunological receptor;
(c) the one or more polypeptides of interest comprise a T-cell receptor, further optionally wherein the T-cell receptor is specific for WT1; or
(d) the one or more polypeptides of interest comprise a chimeric antigen receptor, further optionally wherein the chimeric antigen receptor is specific for WT1.

The cell may additionally comprise one or more nucleic acid sequences encoding α and β chains of an exogenous T-cell receptor. The cell may additionally comprise one or more nucleic acid sequences encoding γ and δ chains of an exogenous T-cell receptor.

One or more nucleic acid sequences encoding the α and β chains of the exogenous T-cell receptor of the cells described herein may be in the TRAC locus of the genome. One or more nucleic acid sequences encoding the γ and δ chains of the exogenous T-cell receptor of the cells described herein may be in the TRAC locus of the genome.

The sequence of the TCR α chain may be selected from: (i) SEQ ID NO: 501 or 504; (ii) a sequence that is at least 99%, 95%, 90%, 85%, 80%, 70%, of 60% identical to SEQ ID NO: 501 or 504; and a contiguous subsequence of at least 20, 30, 40, 50, 60, 70, 80, 90, 100, 150, 200, or 250 amino acids of SEQ ID NO: 501 or 504; and the sequence of the TCR β chain may be selected from: (i) SEQ ID NO: 502 or 505; (ii) a sequence that is at least 99%, 95%, 90%, 85%, 80%, 70%, of 60% identical to SEQ ID NO: 502 or 505; and a contiguous subsequence of at least 20, 30, 40, 50, 60, 70, 80, 90, 100, 150, 200, 250, or 300 amino acids of SEQ ID NO: 502 or 505.

The TCR α chain of the cell may be encoded by a nucleic acid sequence according to any of SEQ ID NOs: 500, 501, 503, and 504, and the β TCR chain is encoded according to any of SEQ ID NOs: 500, 502, 503, and 505.

The sequence of the TCR α chain may be selected from: (i) SEQ ID NO:513; (ii) a sequence that is at least 99%, 95%, 90%, 85%, 80%, 70%, of 60% identical to SEQ ID NO:513; and a contiguous subsequence of at least 20, 30, 40, 50, 60, 70, 80, 90, 100, 150, 200, 250, 300, 350, 400, 500, 600, 700, or 800 amino acids of SEQ ID NO: 513, and wherein the sequence of the TCR β chain is selected from: (i) SEQ ID NO: 514; (ii) a sequence that is at least 99%, 95%, 90%, 85%, 80%, 70%, of 60% identical to SEQ ID NO: 514; and a contiguous subsequence of at least 20, 30, 40, 50, 60, 70, 80, 90, 100, 150, 200, 250, 300, 350, 400, 500, 600, 700, or 800 amino acids of SEQ ID NO:514.

The α TCR chain may be SEQ ID NO:513 and the β TCR chain may be SEQ ID NO: 514.

The one or more genes of the cells disclosed herein may be expressed from an endogenous promoter.

The one or more genes of the cells disclosed herein may be expressed from a heterologous promoter, optionally wherein the heterologous promoter is an EF-1α promoter.

The altered cell may comprise genes encoding α and β chains of an exogenous T-cell receptor and/or genes encoding γ and δ chains of an exogenous T-cell receptor. Suitable α, β, γ, and δ chains are known in the art. See, e.g., WO2018/197492.

The α and β chains of the exogenous T-cell receptor may be in the TRAC locus of the genome. The α and β chains of the exogenous T-cell receptor provided within a transcript, separated by a P2A or other cleavage sequence.

The invention also provides a population of cells comprising disclosed herein, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the altered population are CD3− cells.

The invention also provides a population of cells comprising disclosed herein, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the population lacks an endogenous T-cell receptor.

The invention also provides a population of cells comprising disclosed herein, wherein the expression of the TRBC1, TRBC2 and/or TRAC genes in the population has been reduced relative to an unaltered population of the same cell by at least about 50%, at least about 55%, at least about 60%, at least about 65%, at least about 70%, at least about 75%, at least about 80%, at least about 85%, at least about 90%, at least about 95%, at least about 98%, or at least about 99%.

The reduction in expression may be of the TRBC1 gene. The reduction in expression may be of the TRBC2 gene. The reduction in expression may be of the TRAC gene.

Between 10 and 100% of the population of cells, e.g. between 30 and 99% of the population, may have an indel in the TRBC1, TRBC2 and/or TRAC genes.

Furthermore, between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the population may have an indel in the TRBC1, TRBC2 and/or TRAC genes.

The indel or insertion may be in the TRBC1 gene. The indel or insertion may be in the TRBC2 gene. The indel or insertion may be in the TRAC gene.

The method of, or composition for use of, described herein may result in editing of the TRBC1 and/or TRBC2 genes. The method of, or composition for use of, described herein may result in editing of the TRAC gene. The method of, or composition for use of, described herein may result in editing of a TRBC gene and the TRAC gene.

The editing may be calculated as a percentage of the population that is edited (percent editing or percent indels). The percent editing may be between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the population.

The composition described herein may comprise a sgRNA comprising:
(e) any one of SEQ ID NOs: 179-184 and 196-200; or
(f) a guide sequence selected from any one of SEQ ID NOs: 1-89; or
(g) a guide sequence selected from SEQ ID NOs: 1-24; or
(h) a guide sequence selected from SEQ ID NOs: 1-6.

The composition described herein may comprise a sgRNA comprising:
(i) any one of SEQ ID NOs: 186-192 and 201-212; or
(j) a guide sequence selected from any one of SEQ ID NOs: 90-178, 185, and 213-218; or
(k) a guide sequence selected from SEQ ID NOs: 90-113 and 213-218; or
(l) a guide sequence selected from SEQ ID NOs: 90-95.

The target sequence may be in exon 1, 2, 3, or 4 of the TRBC1, TRBC2 and/or TRAC genes. The target sequence may be in in the human TRBC1, TRBC2 and/or TRAC genes.

The target sequence may be in in exon 1 of the TRBC1, TRBC2 and/or TRAC genes. The target sequence may be in in exon 2 of the TRBC1, TRBC2 and/or TRAC genes. The target sequence may be in in exon 3 of the TRBC1, TRBC2 and/or TRAC genes. The target sequence may be in in exon 4 of the TRBC1, TRBC2 and/or TRAC genes.

The guide sequence may be complementary to a target sequence in the positive strand of TRBC1, TRBC2 and/or TRAC genes. The guide sequence may be complementary to a target sequence in the negative strand of TRBC1, TRBC2 and/or TRAC genes.

The first guide sequence may be complementary to a first target sequence in the positive strand of the TRBC1, TRBC2 and/or TRAC genes, and wherein the composition further comprises a second guide sequence that is complementary to a second target sequence in the negative strand of the TRBC1, TRBC2 and/or TRAC genes.

The guide RNA may comprise a guide sequence selected from any one of SEQ ID NOs 1-178 and further comprises a nucleotide sequence of SEQ ID NO: 400, wherein the nucleotides of SEQ ID NO: 400 follow the guide sequence at its 3′ end.

The guide RNA may comprise a guide sequence selected from any one of SEQ ID NOs 1-178 and further comprises a nucleotide sequence of SEQ ID NO: 401, wherein the nucleotides of SEQ ID NO: 401 follow the guide sequence at its 3′ end.

The guide RNA may be modified according to the pattern of SEQ ID NO: 300, wherein the N's are collectively any one of the guide sequences of SEQ ID NOs 1-89. Each N in SEQ ID NO: 300 may be any natural or non-natural nucleotide, wherein the N's form the guide sequence, and the guide sequence targets Cas9 to the TRBC1, TRBC2 and/or TRAC genes.

The first guide RNA may comprise the sequence of SEQ ID NO: 2 and the second guide RNA may comprise the sequence of SEQ ID NO: 90.

Any of the foregoing methods may further comprise:
(a) introducing a first nucleic acid sequence comprising a nucleic acid sequence selected from: (i) SEQ ID NO: 250; (ii) a nucleic acid sequence that is at least 99%, 95%, 90%, 85%, 80%, 70%, of 60% identical to SEQ ID NO: 250; and (iii) a contiguous subsequence of at least 20, 30, 40, 50, 60, 70, 80, 90, 100, 150, 200, 250, 300, 350, 400, 500, 600, 700, or 800 nucleotides of SEQ ID NO: 250, and
   introducing a second nucleic acid sequence comprising a nucleic acid sequence selected from: (i) SEQ ID NO: 252; (ii) a nucleic acid sequence that is at least 99%, 95%, 90%, 85%, 80%, 70%, of 60% identical to SEQ ID NO: 252; and (iii) a contiguous subsequence of at least 20, 30, 40, 50, 60, 70, 80, 90, 100, 150, 200, 250, 300, 350, 400, 500, 600, 700, 800, or 900 nucleotides of SEQ ID NO: 252,
(b) introducing a first nucleic acid sequence comprising a nucleic acid sequence selected from: (i) SEQ ID NO:513; (ii) a nucleic acid sequence that is at least 99%, 95%, 90%, 85%, 80%, 70%, of 60% identical to SEQ ID NO: 513; and (iii) a contiguous subsequence of at least 20, 30, 40, 50, 60, 70, 80, 90, 100, 150, 200, 250, 300, 350, 400, 500, 600, 700, or 800 nucleotides of SEQ ID NO: 513, and
   introducing a second nucleic acid sequence comprising a nucleic acid sequence selected from: (i) SEQ ID NO:514; (ii) a nucleic acid sequence that is at least 99%, 95%, 90%, 85%, 80%, 70%, of 60% identical to SEQ ID NO:514; and (iii) a contiguous subsequence of at least 20, 30, 40, 50, 60, 70, 80, 90, 100, 150, 200, 250, 300, 350, 400, 500, 600, 700, 800, or 900 nucleotides of SEQ ID NO:514,
(c) introducing a nucleic acid sequence comprising a first nucleic acid sequence of (a) and a second nucleic acid sequence of (a); or
(d) introducing a nucleic acid sequence comprising a first nucleic acid sequence of (b) and a second nucleic acid sequence of (b).

The sgRNA may comprise a guide sequence that is at least 99%, 98%, 97%, 96%, 95%, 94%, 93%, 92%, 91%, or 90% identical to a sequence selected from SEQ ID NOs: 1-89.

The guide RNA may be chemically synthesized. The guide RNA may be comprised in an RNP. The template nucleic acid may be comprised in a viral vector. The template nucleic acid may be comprised in a nonviral delivery construct. Suitable viral vectors are known in the art, e.g., retroviruses, adenovirus, lentivirus, adeno-associated virus, and hybrids thereof. The viral vector may be a lentiviral (LV) vector or an adeno-associated virus (AAV) vector.

The guide RNA may optionally comprise one or more of the following modifications:
i) a 2′-O-methyl (2′-O-Me) modified nucleotide.
ii) a phosphorothioate (PS) bond between nucleotides.
iii) a 2′-fluoro (2′-F) modified nucleotide.
iv) a modification at one or more of the first five nucleotides at the 5′ end of the guide RNA.
v) a modification at one or more of the last five nucleotides at the 3′ end of the guide RNA.
vi) a PS bond between the first four nucleotides at the 5′ end of the guide RNA.
vii) a PS bond between the last four nucleotides at the 3′ end of the guide RNA.
viii) a 2′-O-Me modified nucleotide at the first three nucleotides at the 5′ end of the guide RNA.
ix) a 2′-O-Me modified nucleotide at the last three nucleotides at the 3′ end of the guide RNA.

The guide RNA may comprise the nucleotides of SEQ ID NO: 300 with modifications as set out above.

The compositions may further comprise a pharmaceutically acceptable excipient.

The LNP may comprises a biodegradable, ionizable lipid, e.g., the ionizable lipid is be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

The LNP may comprise a neutral lipid, e.g., the neutral lipid is DSPC.

The LNP may comprise a helper lipid, e.g., the helper lipid is cholesterol.

The LNP may comprise a stealth lipid, e.g., the stealth lipid is PEG2k-DMG.

The composition may further comprise an RNA-guided DNA binding agent. The guide RNA and RNA-guided DNA binding agent composition may be comprised in a ribonucleoprotein (RNP).

The composition may comprise an RNA-guided DNA binding agent, e.g. Cas9, or an mRNA that encodes an RNA-guided DNA binding agent, e.g., the RNA-guided DNA binding agent may be encoded by a Cas9 gene or be a Cas9 protein.

The composition may be a pharmaceutical formulation and further comprise a pharmaceutically acceptable carrier.

The invention also provides for the use of a composition, formulation, population, or cell described herein in the preparation of a medicament.

The invention also provides for the use of a composition, formulation, population, or cell described herein in the treatment of cancer.

The invention also provides for the use of a composition, formulation, population, or cell described herein in immunotherapy of a subject.

The invention also provides for the use of a composition, formulation, population, or cell described herein in the treatment of tumors that overexpress Wilms' tumor antigen (WT1).

The invention also provides a composition, formulation, population, or cell described herein for use in the treatment of a disease or disorder.

The invention also provides a composition, formulation, population, or cell described herein for use in immunotherapy.

The invention also provides a composition, formulation, population, or cell described herein for use in the treatment of cancer.

The invention also provides a composition, formulation, population, or cell described herein for use in the treatment of tumors that overexpress Wilms' tumor antigen (WT1).

The invention also provides a composition, formulation, population, or cell described herein in a method of treatment of a human or animal comprising administration of a composition, formulation, population, or cell as described herein.

The invention also provides a composition, formulation, population, or cell described herein in a method of treatment of cancer in a human or animal comprising administration of a composition, formulation, population, or cell as described herein.

The invention also provides a composition, formulation, population, or cell described herein in a method of immunotherapy of a human or animal comprising administration of a composition, formulation, population, or cell as described herein.

The invention also provides a composition, formulation, population, or cell described herein in a method of treatment of tumors that overexpress Wilms' tumor antigen (WT1) in a human or animal comprising administration of a composition, formulation, population, or cell as described herein.

The guide RNA may have a sequence selected from any of SEQ ID NOs: 1-89.

The guide RNA may have a sequence selected from any of SEQ ID NOs: 90-178, 185, and 213-218.

Also disclosed are embodiments wherein a guide sequence is selected from a group of guide sequences yielding a frequency of indels in the altered products of, respectively, 20% or greater, 30% or greater, 40% or greater, or 50% or greater.

Also disclosed are embodiments wherein a guide sequence is selected from a group of guide sequences yielding a frequency of insertion of donor nucleic acid molecules in the altered products of, respectively, 20% or greater, 30% or greater, 40% or greater, or 50% or greater.

Also disclosed is the use of a composition, formulation, population, or cell of any of the foregoing embodiments for the preparation of a medicament for treating a subject. The subject may be human or animal (e.g. human; cynomolgus monkey). In some embodiments, the subject is human.

Also disclosed are any of the foregoing compositions or formulations for use in treating or for use in modifying (e.g., forming an indel in, or forming a frameshift or nonsense mutation in) any one or more of the TRBC1, TRBC2 and TRAC genes.

In any of the aforementioned cell aspects and embodiments, a gene including a target sequence complementary to the targeting domain of the first gRNA molecule, and, optionally, a gene including a target sequence complementary to the targeting domain of the second gRNA molecule and/or a gene including a target sequence complementary to the targeting domain of the third gRNA molecule, has been altered such that expression of a functional product of the gene including a target sequence complementary to the targeting domain of the first gRNA molecule, and, optionally, the gene including a target sequence complementary to the targeting domain of the second gRNA molecule and/or a functional product of a gene including a target sequence complementary to the targeting domain of the third gRNA molecule, has been reduced or eliminated.

In another aspect, the invention provides a method of providing an immunotherapy in a subject, the method including administering to the subject an effective amount of a cell as described herein, for example, a cell of any of the aforementioned cell aspects and embodiments.

In embodiments of the methods, the method includes administering a lymphodepleting agent or immunosuppressant prior to administering to the subject an effective amount of the cell as described herein, for example, a cell of any of the aforementioned cell aspects and embodiments. In another aspect, the invention provides a method of preparing cells (e.g., a population of cells).

Immunotherapy is the treatment of disease by activating or suppressing the immune system. Immunotherapies designed to elicit or amplify an immune response are classified as activation immunotherapies. Cell-based immunotherapies have been demonstrated to be effective in the treatment of some cancers. Immune effector cells such as lymphocytes, macrophages, dendritic cells, natural killer cells (NK Cell), and/or cytotoxic T lymphocytes (CTL) can be programmed to act in response to abnormal antigens expressed on the surface of tumor cells. Thus, cancer immunotherapy allows components of the immune system to destroy tumors or other cancerous cells.

In another aspect, the invention provides a method of preparing cells (e.g., a population of cells) for immunotherapy, the method including: (a) modifying cells by reducing or eliminating expression of one or more or all components of a T-cell receptor (TCR), for example, by introducing into said cells a gRNA molecule (as described herein), or more than one gRNA molecule, as disclosed herein; and (b) expanding said cells. Cells of the invention are suitable for further engineering, e.g. by introduction of a heterologous sequence coding for a polypeptide that mediates TCR/CD3 zeta chain signaling. In some embodiments, the polypeptide is a wild-type or variant TCR. Cells of the invention may also be suitable for further engineering by introduction of a heterologous sequence coding for an alternative antigen binding moiety, e.g. by introduction of a heterologous sequence coding for an alternative (non-endogenous) T-cell receptor, e.g. a chimeric antigen receptors (CAR) engineered to target a specific protein. CAR are also known as chimeric immunoreceptors, chimeric T-cell receptors or artificial T-cell receptors).

In another aspect, the invention provides a method of treating a subject in need thereof that includes administering cells (e.g., a population of cells) prepared by a method of preparing cells described herein, for example, a method of any of the aforementioned aspects and embodiments of methods of preparing cells.

### BRIEF DESCRIPTION OF THE DRAWINGS

FIG. 1 shows the degree of TRAC editing in HEK-Cas9 cells.

FIG. 2 shows the degree of TRAC editing in human CD3⁺ T cells.

FIG. 3 shows the amount of TCR expression following TRAC editing in human CD3⁺ T cells.

FIGS. 4A-B show the degree of TRBC editing in HEK-Cas9 cells. FIG. 4A shows editing in TRBC1 and FIG. 4B shows editing in TRBC2.

FIG. 5 shows the degree of TRBC editing in human CD3⁺ T cells.

FIG. 6 shows the amount of TCR expression following TRBC editing in human CD3⁺ T cells.

FIGS. 7A-B show biochemical off target analysis(measurement of potential off-target editing sites) for TRBC, TRAC and control guides (SEQ ID NOs: 193 and 194).

FIGS. 8A-D demonstrate knockout of the T-cell receptor and insertion of a TCR construct to produce cells with TCR edited T cells. FIG. 8A shows the efficiency of T-cell receptor knockout. FIG. 8B shows the efficiency of lentiviral transduction of the TCR construct. FIG. 8C shows expression of the TCR insert in edited T cells. FIG. 8D shows the phenotype of TCR edited T cells.

FIGS. 9A-D show the ability of T cells with a TCR insertion to kill primary AML blasts. FIGS. 9A to 9C show results obtained with primary AML blasts obtained from 3 different patients harboring the HLA-A*02:01 allele. FIG. 9D shows the results from using the control sample: primary blasts not harboring the specific HLA allele.

FIG. 10 demonstrates the editing frequency obtained by simultaneous editing of TRAC and TRBC loci using multiple dual guide RNAs.

FIGS. 11A-D show the results of using TRAC and TRBC targeting crRNAs for combined editing. FIG. 11A shows the efficiency of T-cell receptor knockout. FIG. 11B shows lentiviral transduction of the HD1-TCR construct in the cells. FIG. 11C shows expression of the TCR insert in edited T cells. FIG. 11D shows the phenotype of TCR edited T cells.

FIGS. 12A-B demonstrate the editing frequency obtained by simultaneous editing of TRAC and TRBC loci using multiple single guide RNAs. FIG. 12A shows phenotypic percentage of CD3⁻ T cells following editing of the TRAC and TRBC loci. FIG. 11B shows genotypic percentage of Indel formation in the TRAC and TRBC loci in human CD3⁻ T cells.

FIGS. 13A-B show degree of GFP insertion at the TRAC locus employing dual RNA guides and an AAV vector (AV9) with a gapped insertion template (FIG. 13A) and degree of TRAC knock out employing these dual RNA guides and AAV vector that are CD3− (FIG. 13B). A gapped insertion template (or simply gapped template) comprises two sequences that flank a “gap,” i.e., a region in the corresponding target sequence not present in the gapped insertion template. The gapped insertion template is compatible with guides that target a site within the gap for cleavage and is useful for quantitatively comparing such guides regardless of whether they target the exact same cleavage site, provided that they both target a cleavage site within the gap.

FIGS. 14A-14B show degree of GFP insertion at TRAC locus with sgRNA and gapped template AV9 (FIG. 14A), and percentage of cells engineered with these sgRNA and gapped template AV9 that are phenotypically CD3− (FIG. 14B).

FIGS. 15A-B compare different promoter and inverted terminal repeat (ITR) lengths for TCR insertion as measured by positive tetramer stain (FIG. 15A) and mean fluorescence intensity (MFI) (FIG. 15B) during FACS analysis.

FIGS. 16A-C show the amount of GFP expression driven from the endogenous TRAC promoter in cells engineered by an insertion protocol with AAV insertion of a promoterless GFP construct at the TRAC locus. FIG. 16A shows the mock transfection control, with FIG. 16B showing RNP and FIG. 16C showing RNP+AV10-EGFP. X-axis: GFP expression; Y-axis: CD3e expression; as measured by FACS.

FIGS. 17A-C show insertion efficiency of TCR construct templates with and without promoter sequences (PL—promoterless; EF1a—promoter EF1alpha or EF-1a) at the TRAC locus.

FIG. 18A-B measure degranulation in insertion transformants with & without promoters comprised in the transforming constructs using two different TCRs.

FIG. 19A-B measure interferon gamma expression in insertion transformants with & without promoters comprised in the transforming constructs using two different TCRs.

FIGS. 20A-B show surface presence of two TCR constructs, one with and one without Cys modifications.

FIGS. 21A-D measure the degree of mispairing between TCR chains of the inserted constructs and endogenous TCR chains as measured by flow cytometry. The FACS data of FIG. 21A and FIG. 21B depict the fraction of engineered cells expressing the transgenic or mispaired TCRs, and the intensity of TCR expression in the engineered cells of FIG. 21A and FIG. 21B are graphed as MFI in FIG. 21C and FIG. 21D, respectively.

FIG. 22 measures the degree of mispairing between TCR chains of inserted constructs and endogenous TCR chains in CD8+ or CD4+ cells as measured by flow cytometry.

FIG. 23 shows VLD (Wilms' tumor antigen) tetramer staining for four different versions of the TCR with and without knockout of TRBC, indicating the degree of mispairing for the WT1-TCR engineered CD8+ cells.

FIG. 24 shows VLD (Wilms' tumor antigen) tetramer staining for four different TCRs with and without knockout of TRBC, indicating the degree of mispairing for the WT1-TCR engineered CD4+ cells.

FIG. 25 show the intensity of TCR expression in CD8⁺ cells with four TCR constructs inserted in the TRAC locus along with knockout of TRBC as measured by the MFI of the tetramer stain.

FIG. 26 show the intensity of TCR expression in CD4⁺ cells with four TCR constructs inserted in the TRAC locus along with knockout of TRBC as measured by the MFI of the tetramer stain.

FIGS. 27A-C show the cytotoxicity of T cells containing inserted transgenic TCR (AV11-TCR-A, AV13-TCR-B, AV12-TCR-C) that are also TRAC/TRBC double knockouts or TRAC single knockouts. Cytotoxicity was measured by measuring the florescence from apoptotic cells in response to Caspase 3/7 apoptotic cells after 6 hours.

FIGS. 28A-D show the cytotoxicity of T cells containing inserted transgenic TCR (AV11-TCR-A, AV14-TCR-F, AV15-TCR-G, AV16-TCR-H) that are also TRAC/TRBC double knockouts or TRAC single knockouts. Cytotoxicity was measured by measuring the florescence from apoptotic cells in response to Caspase 3/7 apoptotic cells after 6 hours.

FIGS. 29A-G show the level of peptide-specific IL-2 secretion from T cells containing inserted transgenic TCR (AV 11-TCR-A, AV13-TCR-B, AV12-TCR-C, AV14-TCR-F, AV15-TCR-G, AV16-TCR-H) that are also TRAC/TRBC double knockouts or TRAC single knockouts; as measured by ELISA.

FIGS. 30A-D show the level of peptide-specific IFN-γ secretion from T-cells containing inserted transgenic TCR (AV 11-TCR-A, AV14-TCR-F, AV15-TCR-G, AV16-TCR-H) that are also TRAC/TRBC double knockouts or TRAC single knockouts; as measured by ELISA.

FIGS. 31A-B show the level of peptide-specific IFN-γ staining in CD4⁺ and CD8⁺ T-cells containing inserted transgenic (AV 11-TCR-A, AV14-TCR-F, AV15-TCR-G, AV16-TCR-H) that are also TRAC/TRBC double knockouts or TRAC single knockouts.

FIGS. 32A-B show the results of a CD107a degranulation assay demonstrating the extent of alloreactivity of CD4⁺ and CD8⁺ cells with a transgenic TCR in the TRAC locus wherein the native TRAC is knocked out and in the presence or absence of TRBC knockout. (AV11-TCR-A, AV14-TCR-F, AV15-TCR-G, AV16-TCR-H). FIG. 32A shows percentage of CD107a+ staining cells in CD8⁺ T-cells. FIG. 31B shows percentage of CD107a+ staining cells in CD4⁺ T-cells.

FIGS. 33A-D show the results of a mixed lymphocyte reaction demonstrating the extent of alloreactivity of CD4⁺ and CD8⁺ cells with a transgenic TCR in the TRAC locus wherein the native TRAC is knocked out and in the presence or absence of TRBC knockout. CTV, CellTrace Violet (ThermoFisher).

### DETAILED DESCRIPTION

Reference will now be made in detail to certain embodiments of the invention, examples of which are illustrated in the accompanying drawings. While the present teachings are described in conjunction with various embodiments, it is not intended to limit the present teachings to those embodiments. On the contrary, the present teaching encompass various alternatives, modifications, and equivalents, as will be appreciated by those of skill in the art.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a conjugate” includes a plurality of conjugates and reference to “a cell” includes a plurality of cells and the like.

Numeric ranges are inclusive of the numbers defining the range. Measured and measureable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims). The term “or” is used in an inclusive sense in the specification, i.e., equivalent to “and/or,” unless the context clearly indicates otherwise. The term “about”, when used before a list, modifies each member of the list.

The section headings used herein are for organizational purposes only and are not to be construed as limiting the desired subject matter in any way. In the event that any material incorporated by reference contradicts any term defined in this specification or any other express content of this specification, this specification controls.

### I. Definitions

Unless stated otherwise, the following terms and phrases as used herein are intended to have the following meanings:

“Polynucleotide” and “nucleic acid” are used herein to refer to a multimeric compound comprising nucleosides or nucleoside analogs which have nitrogenous heterocyclic bases or base analogs linked together along a backbone, including conventional RNA, DNA, mixed RNA-DNA, and polymers that are analogs thereof. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see The Biochemistry of the Nucleic Acids 5-36, Adams et al., ed., 11^{th} ed., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional nucleosides with 2′ methoxy substituents, or polymers containing both conventional nucleosides and one or more nucleoside analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester and Wengel, 2004, Biochemistry 43(42):13233-41). RNA and DNA have different sugar moieties and can differ by the presence of uracil or analogs thereof in RNA and thymine or analogs thereof in DNA.

“Guide RNA”, “gRNA”, and simply “guide” are used herein interchangeably to refer to either a crRNA (also known as CRISPR RNA), or the combination of a crRNA and a trRNA (also known as tracrRNA). The crRNA and trRNA may be associated as a single RNA molecule (single guide RNA, sgRNA) or in two separate RNA molecules (dual guide RNA, dgRNA). “Guide RNA” or “gRNA” refers to each type. The trRNA may be a naturally-occurring sequence, or a trRNA sequence with modifications or variations compared to naturally-occurring sequences.

As used herein, a “guide sequence” refers to a sequence within a guide RNA that is complementary to a target sequence and functions to direct a guide RNA to a target sequence for binding or modification (e.g., cleavage) by an RNA-guided DNA binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. For example, in some embodiments, the guide sequence comprises at least 17, 18, 19, or 20 contiguous nucleotides of a sequence selected from SEQ ID NOs: 1-178. In some embodiments, the target sequence is in a gene or on a chromosome, for example, and is complementary to the guide sequence. In some embodiments, the degree of complementarity or identity between a guide sequence and its corresponding target sequence may be about 75%, 80%, 85%, 90%, 95%, 96%, 97%, 98%, 99%, or 100%. For example, in some embodiments, the guide sequence comprises a sequence with about 75%, 80%, 85%, 90%, 95%, 96%, 97%, 98%, 99%, or 100% identity to at least 17, 18, 19, or 20 contiguous nucleotides of a sequence selected from SEQ ID NOs: 1-178. In some embodiments, the guide sequence and the target region may be 100% complementary or identical. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

Target sequences for RNA-guided DNA binding agents include both the positive and negative strands of genomic DNA (i.e., the sequence given and the sequence's reverse compliment), as a nucleic acid substrate for an RNA-guided DNA binding agent is a double stranded nucleic acid. Accordingly, where a guide sequence is said to be “complementary to a target sequence”, it is to be understood that the guide sequence may direct a guide RNA to bind to the sense or antisense strand (e.g. reverse complement) of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

As used herein, an “RNA-guided DNA binding agent” means a polypeptide or complex of polypeptides having RNA and DNA binding activity, or a DNA-binding subunit of such a complex, wherein the DNA binding activity is sequence-specific and depends on the sequence of the RNA. Exemplary RNA-guided DNA binding agents include Cas cleavases/nickases and inactivated forms thereof (“dCas DNA binding agents”). “Cas nuclease”, as used herein, encompasses Cas cleavases, Cas nickases, and dCas DNA binding agents. In some embodiments the Cas cleavase or Cas nickase encompasses a dCas DNA binding agent modified to permit DNA cleavage, e.g. via fusion with a FokI domain. Cas cleavases/nickases and dCas DNA binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA binding activity. Class 2 Cas nucleases include Class 2 Cas cleavases/nickases (e.g., H840A, D10A, or N863A variants), which further have RNA-guided DNA cleavases or nickase activity, and Class 2 dCas DNA binding agents, in which cleavase/nickase activity is inactivated. Class 2 Cas nucleases include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g., K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables S1 and S3. See, e.g., Makarova et al., Nat Rev Microbiol, 13(11): 722-36 (2015); Shmakov et al., Molecular Cell, 60:385-397 (2015).

As used herein, “ribonucleoprotein” (RNP) or “RNP complex” refers to a guide RNA together with an RNA-guided DNA binding agent, such as a Cas nuclease, e.g., a Cas cleavase, Cas nickase, or dCas DNA binding agent (e.g., Cas9). In some embodiments, the guide RNA guides the RNA-guided DNA binding agent such as Cas9 to a target sequence, and the guide RNA hybridizes with and the agent binds to the target sequence; in cases where the agent is a cleavase or nickase, binding can be followed by cleaving or nicking.

As used herein, a first sequence is considered to “comprise a sequence with at least X % identity to” a second sequence if an alignment of the first sequence to the second sequence shows that X % or more of the positions of the second sequence in its entirety are matched by the first sequence. For example, the sequence AAGA comprises a sequence with 100% identity to the sequence AAG because an alignment would give 100% identity in that there are matches to all three positions of the second sequence. The differences between RNA and DNA (generally the exchange of uridine for thymidine or vice versa) and the presence of nucleoside analogs such as modified uridines do not contribute to differences in identity or complementarity among polynucleotides as long as the relevant nucleotides (such as thymidine, uridine, or modified uridine) have the same complement (e.g., adenosine for all of thymidine, uridine, or modified uridine; another example is cytosine and 5-methylcytosine, both of which have guanosine or modified guanosine as a complement). Thus, for example, the sequence 5′-AXG where X is any modified uridine, such as pseudouridine, N1-methyl pseudouridine, or 5-methoxyuridine, is considered 100% identical to AUG in that both are perfectly complementary to the same sequence (5′-CAU). Exemplary alignment algorithms are the Smith-Waterman and Needleman-Wunsch algorithms, which are well-known in the art. One skilled in the art will understand what choice of algorithm and parameter settings are appropriate for a given pair of sequences to be aligned; for sequences of generally similar length and expected identity >50% for amino acids or >75% for nucleotides, the Needleman-Wunsch algorithm with default settings of the Needleman-Wunsch algorithm interface provided by the EBI at the www.ebi.ac.uk web server is generally appropriate. [00170]“mRNA” is used herein to refer to a polynucleotide that comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof.

Exemplary guide sequences useful in the guide RNA compositions and methods described herein are shown in Tables 1, 2 and 3 and throughout the application.

As used herein, “indels” refer to insertion/deletion mutations consisting of a number of nucleotides that are either inserted or deleted at the site of double-stranded breaks (DSBs) in a target nucleic acid.

As used herein, “knockdown” refers to a decrease in expression of a particular gene product (e.g., protein, mRNA, or both). Knockdown of a protein can be measured by detecting total cellular amount of the protein from a tissue or cell population of interest. Methods for measuring knockdown of mRNA are known and include sequencing of mRNA isolated from a tissue or cell population of interest. Flow cytometry analysis is a known method for measuring knockdown of protein expression. In some embodiments, “knockdown” may refer to some loss of expression of a particular gene product, for example a decrease in the amount of mRNA transcribed or a decrease in the amount of protein expressed by a population of cells. In some embodiments, “knockdown” may refer to some loss of expression of a particular gene product, for example a TRAC or TRBC gene product at the cell surface.

As used herein, “knockout” refers to a loss of expression of a particular protein in a cell. Knockout can be measured either by detecting total cellular amount of a protein in a cell, a tissue or a population of cells. In some embodiments, the methods of the invention “knockout” TRBC1, TRBC2 and/or TRAC in one or more cells (e.g., in a population of cells. In some embodiments, a knockout is the complete loss of expression of a protein component of the T-cell receptor (e.g. TRBC1, TRBC2 and/or TRAC) in a cell, rather than the formation of a mutant T-cell receptor protein.

As used herein, “TRBC1” and “TRBC2” refer to two homologous genes encoding the T-cell receptor β-chain, which are the gene products of the TRBC1 or TRBC2 genes.

“TRBC” is used herein to refer to TRBC1 and TRBC2.

The human wild-type TRBC1 sequence is available at NCBI Gene ID: 28639; Ensembl: ENSG00000211751. T-cell receptor Beta Constant, V_segment Translation Product, BV05S1J2.2, TCRBC1, and TCRB are gene synonyms for TRBC1.

The human wild-type TRBC2 sequence is available at NCBI Gene ID: 28638; Ensembl: ENSG00000211772. T-cell receptor Beta Constant, V_segment Translation Product, and TCRBC2 are gene synonyms for TRBC2.

The human wild-type TRAC sequence is available at NCBI Gene ID: 28755; Ensembl: ENSG00000277734. T-cell receptor Alpha Constant, TCRA, IMD7, TRCA and TRA are gene synonyms for TRAC.

Wilms' tumor protein (also sometimes referred to as Wilms' tumor antigen) is a protein that is encoded by the WT1 gene. The human wild-type WT1 sequence is available at NCBI Gene ID: 7490; Ensembl: ENSG00000184937. GUD, AWT1, WAGR, WT33, NPHS4, and WIT-2 are gene synonyms for WT1. The Wilms' tumor protein has been reported to be expressed in various cancers including solid tumors (including but not limited to Wilms' tumor) and hematological cancers, and also in tumor vascular endothelial cells and hematopoietic progenitor cells.

As used herein, a “target sequence” refers to a sequence of nucleic acid in a target gene that has complementarity to the guide sequence of the gRNA. The interaction of the target sequence and the guide sequence directs an RNA-guided DNA binding agent to bind, and potentially nick or cleave (depending on the activity of the agent), within the target sequence.

As used herein, “treatment” refers to any administration or application of a therapeutic for disease or disorder in a subject, and includes inhibiting the disease, arresting its development, relieving one or more symptoms of the disease, curing the disease, or preventing reoccurrence of one or more symptoms of the disease.

The term “about” or “approximately” means an acceptable error for a particular value as determined by one of ordinary skill in the art, which depends in part on how the value is measured or determined.

### II. Compositions

A. Compositions Comprising Guide RNA (gRNAs)

Provided herein are compositions useful for altering a DNA sequence, e.g. inducing a single-stranded (SSB) or double-stranded break (DSB), within the TRBC and/or TRAC genes, e.g., using a guide RNA with an RNA-guided DNA binding agent (e.g., a CRISPR/Cas system). In some embodiments, the compositions are useful for altering a TRAC and/or TRBC sequence and further inserting a TCR gene.

In some embodiments, altering a TRAC and/or TRBC sequence and further inserting a TCR gene advantageously reduces mispairing of TRAC and/or TRBC as compared to a control, e.g., a control in which TRAC and/or TRBC is not altered. For example, altering a TRBC sequence (e.g., knocking out TRBC) and further inserting a TCR gene may advantageously reduce mispairing between the inserted TCR and native TRBC. Or altering a TRAC sequence (e.g., knocking out TRAC) and further inserting a TCR gene may advantageously reduce mispairing between the inserted TCR and native TRAC. Guide sequences targeting the TRBC gene are shown in Table 1 at SEQ ID NOs: 1-89. Guide sequences targeting the TRAC gene are shown in Table 2 at SEQ ID NOs: 90-178, 185, and 213-218.

In some embodiments, the guide sequences are complementary to the corresponding genomic region shown in the tables below, according to coordinates from human reference genome hg38. Guide sequences of further embodiments may be complementary to sequences in the close vicinity of the genomic coordinate listed in any of Tables, 1, 2 and/or 3. For example, guide sequences of further embodiments may be complementary to sequences that comprise 15 consecutive nucleotides ±10 nucleotides of a genomic coordinate listed in any of Tables, 1, 2 and/or 3.

Each of the guide sequences shown in Table 1 and Table 2 at SEQ ID NOs: 1-89, 90-178, 185, and 213-218 may further comprise additional nucleotides to form a crRNA, e.g., with the following exemplary nucleotide sequence following the guide sequence at its 3′ end: GUUJUUAGAGCUAUGCUGUUUUG (SEQ ID NO: 400) in 5′ to 3′ orientation. In the case of a sgRNA, the guide sequences may further comprise additional nucleotides to form a sgRNA, e.g., with the following exemplary nucleotide sequence following the 3′ end of the guide sequence:

(SEQ ID NO: 401)
GUUUUAGAGCUAGAAAUAGCAAGUUAAAAUAAGGCUAGUCCGUUAUCAAC
UUGAAAAAGUGGCACCGAGUCGGUGCUUUU
in 5′ to 3′ orientation.

The guide sequences may further comprise additional nucleotides to form a sgRNA, e.g., comprising the following exemplary nucleotide sequence following the 3′ end of the guide sequence:

(SEQ ID NO: 402)
GUUUUAGAGCUAGAAAUAGCAAGUUAAAAUAAGGCUAGUCCGUUAUCAAC
UUGAAAAAGUGGCACCGAGUCGGUGC
in 5′ to 3′ orientation.

TABLE 1
TRBC targeted and control guide sequences and
chromosomal coordinates

| SEQ | | |
|---|---|---|
| ID | | Genomic Coordinates |
| NO: | Guide Sequence | (hg38) |
| 1 | GGCUCUCGGAGAAUGACGAG | chr7: 142791996- |
| | | 142792016 |
| 2 | GGCCUCGGCGCUGACGAUCU | chr7: 142792047- |
| | | 142792067 |
| 3 | AUGACGAGUGGACCCAGGAU | chr7: 142792008- |
| | | 142792028 |
| 4 | AGAAGGUGGCCGAGACCCUC | chr7: 142791931- |
| | | 142791951 |
| 5 | UGAGGGUCUCGGCCACCUUC | chr7: 142791930- |
| | | 142791950 |
| 6 | AGAGAUCUCCCACACCCAAA | chr7: 142791748- |
| | | 142791768 |
| 7 | UGGCUCAAACACAGCGACCU | chr7: 142791720- |
| | | 142791740 |
| 8 | GGCGCUGACGAUCUGGGUGA | chr7: 142792041- |
| | | 142792061 |
| 9 | UGGCAGACAGGACCCCUUGC | chr7: 142802114- |
| | | 142802134 |
| 10 | UGACGAGUGGACCCAGGAUA | chr7: 142792009- |
| | | 142792029 |
| 11† | AGACAGGACCCCUUGCUGGU | chr7: 142792697- |
| | | 142792717 |
| 12 | UUGACAGCGGAAGUGGUUGC | chr7: 142791963- |
| | | 142791983 |
| 13 | CGUAGAACUGGACUUGACAG | chr7: 142791976- |
| | | 142791996 |
| 14 | CGCUGUCAAGUCCAGUUCUA | chr7: 142791974- |
| | | 142791994 |
| 15 | ACUGGACUUGACAGCGGAAG | chr7: 142791970- |
| | | 142791990 |
| 16 | GUUGCGGGGGUUCUGCCAGA | chr7: 142791948- |
| | | 142791968 |
| 17 | CUGCCUGAGCAGCCGCCUGA | chr7: 142791913- |
| | | 142791933 |
| 18 | GACAGCGGAAGUGGUUGCGG | chr7: 142791961- |
| | | 142791981 |
| 19 | CCACUCACCUGCUCUACCCC | chr7: 142792068- |
| | | 142792088 |
| 20 | GCUGUCAAGUCCAGUUCUAC | chr7: 142791975- |
| | | 142791995 |
| 21 | ACACUGGUGUGCCUGGCCAC | chr7: 142791773- |
| | | 142791793 |
| 22 | AGACCCUCAGGCGGCUGCUC | chr7: 142791919- |
| | | 142791939 |
| 23 | AUGGGAAGGAGGUGCACAGU | chr7: 142791834- |
| | | 142791854 |
| 24 | AGGGCGGGCUGCUCCUUGAG | chr7: 142791878- |
| | | 142791898 |
| 25 | UCCCUAGCAAGAUCUCAUAG | chr7: 142802141- |
| | | 142802161 |
| 26† | GGUGCACAGUGGGGUCAGCA | chr7: 142791844- |
| | | 142791864 |
| 27 | CCAGCUCAGCUCCACGUGGU | chr7: 142801154- |
| | | 142801174 |
| 28 | CCGCAACCACUUCCGCUGUC | chr7: 142791961- |
| | | 142791981 |
| 29 | GUCCACUCGUCAUUCUCCGA | chr7: 142792001- |
| | | 142792021 |
| 30 | GCCCGUAGAACUGGACUUGA | chr7: 142791979- |
| | | 142791999 |
| 31 | UCACCCAGAUCGUCAGCGCC | chr7: 142792041- |
| | | 142792061 |
| 32 | GGGUCCACUCGUCAUUCUCC | chr7: 142792003- |
| | | 142792023 |
| 33 | UCCAGUUCUACGGGCUCUCG | chr7: 142791984- |
| | | 142792004 |
| 34 | CGGAGAAUGACGAGUGGACC | chr7: 142792002- |
| | | 142792022 |
| 35 | ACCACUUCCGCUGUCAAGUC | chr7: 142791966- |
| | | 142791986 |
| 36 | AAUGACGAGUGGACCCAGGA | chr7: 142792007- |
| | | 142792027 |
| 37 | ACGGGCUCUCGGAGAAUGAC | chr7: 142791993- |
| | | 142792013 |
| 38 | GACUCCAGAUACUGCCUGAG | chr7: 142791902- |
| | | 142791922 |
| 39 | CGCUGUGUUUGAGCCAUCAG | chr7: 142791724- |
| | | 142791744 |
| 40 | AGAACUGGACUUGACAGCGG | chr7: 142791973- |
| | | 142791993 |
| 41 | GAGACCCUCAGGCGGCUGCU | chr7: 142791920- |
| | | 142791940 |
| 42 | CGUCAUUCUCCGAGAGCCCG | chr7: 142791994- |
| | | 142792014 |
| 43 | CAGCCCGCCCUCAAUGACUC | chr7: 142791887- |
| | | 142791907 |
| 44 | GGCUGCUCAGGCAGUAUCUG | chr7: 142791907- |
| | | 142791927 |
| 45 | AGUGGUUGCGGGGGUUCUGC | chr7: 142791952- |
| | | 142791972 |
| 46 | GGUCGCUGUGUUUGAGCCAU | chr7: 142791721- |
| | | 142791741 |
| 47 | GAUCUCAUAGAGGAUGGUGG | chr7: 142792718- |
| | | 142792738 |
| 48 | UGUUUGAGCCAUCAGAAGCA | chr7: 142791729- |
| | | 142791749 |
| 49 | UACUGCCUGAGCAGCCGCCU | chr7: 142791911- |
| | | 142791931 |
| 50 | ACCCGCAGCCCCUCAAGGAG | chr7: 142791867- |
| | | 142791887 |
| 51 | AGGCAGUAUCUGGAGUCAUU | chr7: 142791899- |
| | | 142791919 |
| 52 | UGUGUUUGAGCCAUCAGAAG | chr7: 142791727- |
| | | 142791747 |
| 53 | GGUUGCGGGGGUUCUGCCAG | chr7: 142791949- |
| | | 142791969 |
| 54 | GGGUCUCGGCCACCUUCUGG | chr7: 142791933- |
| | | 142791953 |
| 55 | CAGAAGGUGGCCGAGACCCU | chr7: 142791932- |
| | | 142791952 |
| 56 | CGCCGAGGCCUGGGGUAGAG | chr7: 142792057- |
| | | 142792077 |
| 57 | GGUUCUGCCAGAAGGUGGCC | chr7: 142791940- |
| | | 142791960 |
| 58 | CAGAGAUCUCCCACACCCAA | chr7: 142791747- |
| | | 142791767 |
| 59 | UUGAGGGCGGGCUGCUCCUU | chr7: 142791881- |
| | | 142791901 |
| 60 | AAGCCUGUGGCCAGGCACAC | chr7: 142791779- |
| | | 142791799 |
| 61 | CAGCGCCGAGGCCUGGGGUA | chr7: 142792054- |
| | | 142792074 |
| 62 | CCCACUCACCUGCUCUACCC | chr7: 142792069- |
| | | 142792089 |
| 63 | UGUCUGCCACCAUCCUCUAU | chr7: 142792712- |
| | | 142792732 |
| 64 | UGCUUCUGAUGGCUCAAACA | chr7: 142791729- |
| | | 142791749 |
| 65 | UUCCCAUUCACCCACCAGCU | chr7: 142791821- |
| | | 142791841 |
| 66 | GUCAGCGCCGAGGCCUGGGG | chr7: 142792052- |
| | | 142792072 |
| 67 | CCCUCAGGCGGCUGCUCAGG | chr7: 142791916- |
| | | 142791936 |
| 68 | AAUGACUCCAGAUACUGCCU | chr7: 142791899- |
| | | 142791919 |
| 69 | CACACUGGUGUGCCUGGCCA | chr7: 142791772- |
| | | 142791792 |
| 70 | UCAUAGAGGAUGGUGGCAGA | chr7: 142792714- |
| | | 142792734 |
| 71 | CACCCAGAUCGUCAGCGCCG | chr7: 142792042- |
| | | 142792062 |
| 72 | UGACAGCGGAAGUGGUUGCG | chr7: 142791962- |
| | | 142791982 |
| 73 | UCUCCGAGAGCCCGUAGAAC | chr7: 142791988- |
| | | 142792008 |
| 74 | AGUCCAGUUCUACGGGCUCU | chr7: 142791982- |
| | | 142792002 |
| 75 | AUCGUCAGCGCCGAGGCCUG | chr7: 142792049- |
| | | 142792069 |
| 76 | AAGGAGGUGCACAGUGGGGU | chr7: 142791839- |
| | | 142791859 |
| 77 | UAUCUGGAGUCAUUGAGGGC | chr7: 142791893- |
| | | 142791913 |
| 78 | GCGGGGGUUCUGCCAGAAGG | chr7: 142791945- |
| | | 142791965 |
| 79 | CUUGACAGCGGAAGUGGUUG | chr7: 142791964- |
| | | 142791984 |
| 80 | GUGUGGCCUUUUGGGUGUGG | chr7: 142791757- |
| | | 142791777 |
| 81 | GAUCGUCAGCGCCGAGGCCU | chr7: 142792048- |
| | | 142792068 |
| 82 | UGUGGCCAGGCACACCAGUG | chr7: 142791774- |
| | | 142791794 |
| 83 | AGGCCUCGGCGCUGACGAUC | chr7: 142792048- |
| | | 142792068 |
| 84 | GUGAAUGGGAAGGAGGUGCA | chr7: 142791830- |
| | | 142791850 |
| 85 | GCGGCUGCUCAGGCAGUAUC | chr7: 142791909- |
| | | 142791929 |
| 86 | ACUGCCUGAGCAGCCGCCUG | chr7: 142791912- |
| | | 142791932 |
| 87 | AAAGGCCACACUGGUGUGCC | chr7: 142791766- |
| | | 142791786 |
| 88 | UGAGGGCGGGCUGCUCCUUG | chr7: 142791880- |
| | | 142791900 |
| 89 | GAGCAGCCGCCUGAGGGUCU | chr7: 142791919- |
| | | 142791939 |

Each of these guide sequences generally targets both TRBC1 and TRBC2 genes with the following exceptions, which are marked as follows: † SEQ ID NOs: 11 and 26 target TRBC1 specifically.

Each of these guide sequences generally targets both TRBC1 and TRBC2 genes with the following exceptions, which are marked as follows:
† SEQ ID NOs: 11 and 26 target TRBC1 specifically.

TABLE 2
TRAC targeted and control guide sequences and
chromosomal coordinates

| SEQ | | |
|---|---|---|
| ID | | Genomic Coordinates |
| NO: | Guide Sequence | (hg38) |
| 90 | CUCUCAGCUGGUACACGGCA | chr14: 22547524-22547544 |
| 91 | UUCGGAACCCAAUCACUGAC | chr14: 22550581-22550601 |
| 92 | UAAACCCGGCCACUUUCAGG | chr14: 22550608-22550628 |
| 93 | GAUUAAACCCGGCCACUUUC | chr14: 22550611-22550631 |
| 94 | CGUCAUGAGCAGAUUAAACC | chr14: 22550622-22550642 |
| 95 | AGAGUCUCUCAGCUGGUACA | chr14: 22547529-22547549 |
| 96 | ACACGGCAGGGUCAGGGUUC | chr14: 22547512-22547532 |
| 97 | UCUCUCAGCUGGUACACGGC | chr14: 22547525-22547545 |
| 98 | UGGAUUUAGAGUCUCUCAGC | chr14: 22547536-22547556 |
| 99 | GAGAAUCAAAAUCGGUGAAU | chr14: 22547575-22547595 |
| 100 | ACAAAACUGUGCUAGACAUG | chr14: 22547640-22547660 |
| 101 | UGUGCUAGACAUGAGGUCUA | chr14: 22547647-22547667 |
| 102 | GCACCAAAGCUGCCCUUACC | chr14: 22547777-22547797 |
| 103 | AAGUUCCUGUGAUGUCAAGC | chr14: 22549638-22549658 |
| 104 | CUCGACCAGCUUGACAUCAC | chr14: 22549646-22549666 |
| 105 | AUCCUCCUCCUGAAAGUGGC | chr14: 22550600-22550620 |
| 106 | ACCCGGCCACUUUCAGGAGG | chr14: 22550605-22550625 |
| 107 | UUAAUCUGCUCAUGACGCUG | chr14: 22550625-22550645 |
| 108 | ACACGGAUGAACAAUAAGGC | chr14: 22539116-22539136 |
| 109 | ACUUACACGGAUGAACAAUA | chr14: 22539120-22539140 |
| 110 | GCUGGUACACGGCAGGGUCA | chr14: 22547518-22547538 |
| 111 | GGAAGCUACAUACCUACAUU | chr14: 22539082-22539102 |
| 112 | UCCUCACUGUGUGCAUCAGG | chr14: 22539061-22539081 |
| 113 | CUGGUUCCUCUUCCAAAUGU | chr14: 22539097-22539117 |
| 114 | AAAGUCAGAUUUGUUGCUCC | chr14: 22547697-22547717 |
| 115 | UUCAAAACCUGUCAGUGAUU | chr14: 22550571-22550591 |
| 116 | UGCUCAUGACGCUGCGGCUG | chr14: 22550631-22550651 |
| 117 | UCAAGGCCCCUCACCUCAGC | chr14: 22550658-22550678 |
| 118 | GGCGUUUGCACAUGCAAAGU | chr14: 22547712-22547732 |
| 119 | GACCACAGCCGCAGCGUCAU | chr14: 22550636-22550656 |
| 120 | AUGACGCUGCGGCUGUGGUC | chr14: 22550636-22550656 |
| 121 | AUUCGGAACCCAAUCACUGA | chr14: 22550582-22550602 |
| 122 | AACCCGGCCACUUUCAGGAG | chr14: 22550606-22550626 |
| 123 | UUAAACCCGGCCACUUUCAG | chr14: 22550609-22550629 |
| 124 | AGAUUUGUUGCUCCAGGCCA | chr14: 22547691-22547711 |
| 125 | UGAGAAUCAAAAUCGGUGAA | chr14: 22547576-22547596 |
| 126 | GAUGUCAAGCUGGUCGAGAA | chr14: 22549648-22549668 |
| 127 | GUUUCAAAGCUUUUCUCGAC | chr14: 22549660-22549680 |
| 128 | UGAAGGCGUUUGCACAUGCA | chr14: 22547716-22547736 |
| 129 | ACCCUGACCCUGCCGUGUAC | chr14: 22547514-22547534 |
| 130 | AGCUUCAAGGCCCCUCACCU | chr14: 22550662-22550682 |
| 131 | GUUCCGAAUCCUCCUCCUGA | chr14: 22550593-22550613 |
| 132 | AGAUUAAACCCGGCCACUUU | chr14: 22550612-22550632 |
| 133 | CCCUGCCGUGUACCAGCUGA | chr14: 22547521-22547541 |
| 134 | UCACUGGAUUUAGAGUCUCU | chr14: 22547540-22547560 |
| 135 | UACUUACACGGAUGAACAAU | chr14: 22539121-22539141 |
| 136 | UAUCACAGACAAAACUGUGC | chr14: 22547632-22547652 |
| 137 | CCACAGCACUGUUGCUCUUG | chr14: 22547674-22547694 |
| 138 | CCUGUGAUGUCAAGCUGGUC | chr14: 22549643-22549663 |
| 139 | ACAUGAGGUCUAUGGACUUC | chr14: 22547655-22547675 |
| 140 | ACUGUUGCUCUUGAAGUCCA | chr14: 22547667-22547687 |
| 141 | AGCUACAUACCUACAUUUGG | chr14: 22539085-22539105 |
| 142 | UAGAAAGUUCCUGUGAUGUC | chr14: 22549634-22549654 |
| 143 | UCACUGUGUGCAUCAGGAGG | chr14: 22539064-22539084 |
| 144 | GACAAAACUGUGCUAGACAU | chr14: 22547639-22547659 |
| 145 | CUUCAACAACAGCAUUAUUC | chr14: 22547731-22547751 |
| 146 | CAACAACAGCAUUAUUCCAG | chr14: 22547734-22547754 |
| 147 | UCUCAAACAAAUGUGUCACA | chr14: 22547591-22547611 |
| 148 | AUGAGGUCUAUGGACUUCAA | chr14: 22547657-22547677 |
| 149 | GACCCUGCCGUGUACCAGCU | chr14: 22547519-22547539 |
| 150 | CCCCUGUCUUACCUGUUUCA | chr14: 22549674-22549694 |
| 151 | AGCAACAGUGCUGUGGCCUG | chr14: 22547678-22547698 |
| 152 | CUACAUACCUACAUUUGGAA | chr14: 22539087-22539107 |
| 153 | ACUUUGUGACACAUUUGUUU | chr14: 22547595-22547615 |
| 154 | AUCACAGACAAAACUGUGCU | chr14: 22547633-22547653 |
| 155 | UUCAACAACAGCAUUAUUCC | chr14: 22547732-22547752 |
| 156 | CAUGAGGUCUAUGGACUUCA | chr14: 22547656-22547676 |
| 157 | GCUACAUACCUACAUUUGGA | chr14: 22539086-22539106 |
| 158 | CUCUUGUCCCACAGAUAUCC | chr14: 22547491-22547511 |
| 159 | GAUUCUGAUGUGUAUAUCAC | chr14: 22547618-22547638 |
| 160 | CUGUGAUGUCAAGCUGGUCG | chr14: 22549644-22549664 |
| 161 | CCUGCCGUGUACCAGCUGAG | chr14: 22547522-22547542 |
| 162 | ACAUACCUACAUUUGGAAGA | chr14: 22539089-22539109 |
| 163 | CCUCACUGUGUGCAUCAGGA | chr14: 22539062-22539082 |
| 164 | ACAAAUGUGUCACAAAGUAA | chr14: 22547597-22547617 |
| 165 | GAGCAACAGUGCUGUGGCCU | chr14: 22547677-22547697 |
| 166 | UCGACCAGCUUGACAUCACA | chr14: 22549645-22549665 |
| 167 | AUUAAACCCGGCCACUUUCA | chr14: 22550610-22550630 |
| 168 | CACGGCAGGGUCAGGGUUCU | chr14: 22547511-22547531 |
| 169 | AAACCCGGCCACUUUCAGGA | chr14: 22550607-22550627 |
| 170 | CAAGGCCCCUCACCUCAGCU | chr14: 22550657-22550677 |
| 171 | CCCGGCCACUUUCAGGAGGA | chr14: 22550604-22550624 |
| 172 | CAUUUCUAUAAUACUUACAC | chr14: 22539132-22539152 |
| 173 | ACAGCCGCAGCGUCAUGAGC | chr14: 22550632-22550652 |
| 174 | AUCAAAAUCGGUGAAUAGGC | chr14: 22547571-22547591 |
| 175 | GCGUUUGCACAUGCAAAGUC | chr14: 22547711-22547731 |
| 176 | CUGUUGCUCUUGAAGUCCAU | chr14: 22547666-22547686 |
| 177 | AAAUCGGUGAAUAGGCAGAC | chr14: 22547567-22547587 |
| 178 | UUGUCUGUGAUAUACACAUC | chr14: 22547624-22547644 |
| 185 | UCAGGGUUCUGGAUAUCUGU | chr14: 22547501-22547521 |
| 213 | AGCUGGUACACGGCAGGGUC | chr14: 22547519-22547539 |
| 214 | UAGGCAGACAGACUUGUCAC | chr14: 22547556-22547576 |
| 215 | UCUGUGGGACAAGAGGAUCA | chr14: 22547486-22547506 |
| 216 | AUCUGUGGGACAAGAGGAUC | chr14: 22547487-22547507 |
| 217 | CUGGAUAUCUGUGGGACAAG | chr14: 22547493-22547513 |
| 218 | GUCAGGGUUCUGGAUAUCUG | chr14: 22547502-22547522 |

TABLE 3
sgRNA sequences, nomenclature, and component
subsequences

| SEQ | | Genomic |
|---|---|---|
| ID | | Coordinates |
| NO: | sgRNA Sequence | (hg38) |
| 179 | GGCUCUCGGAGAAUGACGAGGUUUUAGA | chr7: 142791996- |
| | GCUAGAAAUAGCAAGUUAAAAUAAGGCU | 142792016 |
| | AGUCCGUUAUCAACUUGAAAAAGUGGCA | |
| | CCGAGUCGGUGCUUUU | |
| 180 | GGCCUCGGCGCUGACGAUCUGUUUUAGA | chr7: 142792047- |
| | GCUAGAAAUAGCAAGUUAAAAUAAGGCU | 142792067 |
| | AGUCCGUUAUCAACUUGAAAAAGUGGCA | |
| | CCGAGUCGGUGCUUUU | |
| 181 | AUGACGAGUGGACCCAGGAUGUUUUAGA | chr7: 142792008- |
| | GCUAGAAAUAGCAAGUUAAAAUAAGGCU | 142792028 |
| | AGUCCGUUAUCAACUUGAAAAAGUGGCA | |
| | CCGAGUCGGUGCUUUU | |
| 182 | AGAAGGUGGCCGAGACCCUCGUUUUAGA | chr7: 142791931- |
| | GCUAGAAAUAGCAAGUUAAAAUAAGGCU | 142791951 |
| | AGUCCGUUAUCAACUUGAAAAAGUGGCA | |
| | CCGAGUCGGUGCUUUU | |
| 183 | UGAGGGUCUCGGCCACCUUCGUUUUAGA | chr7: 142791930- |
| | GCUAGAAAUAGCAAGUUAAAAUAAGGCU | 142791950 |
| | AGUCCGUUAUCAACUUGAAAAAGUGGCA | |
| | CCGAGUCGGUGCUUUU | |
| 184 | AGAGAUCUCCCACACCCAAAGUUUUAGA | chr7: 142791748- |
| | GCUAGAAAUAGCAAGUUAAAAUAAGGCU | 142791768 |
| | AGUCCGUUAUCAACUUGAAAAAGUGGCA | |
| | CCGAGUCGGUGCUUUU | |
| 186 | CUCUCAGCUGGUACACGGCAGUUUUAGA | chr14: 22547529- |
| | GCUAGAAAUAGCAAGUUAAAAUAAGGCU | 22547549 |
| | AGUCCGUUAUCAACUUGAAAAAGUGGCA | |
| | CCGAGUCGGUGCUUUU | |
| 187 | UUCGGAACCCAAUCACUGACGUUUUAGA | chr14: 22550611- |
| | GCUAGAAAUAGCAAGUUAAAAUAAGGCU | 22550631 |
| | AGUCCGUUAUCAACUUGAAAAAGUGGCA | |
| | CCGAGUCGGUGCUUUU | |
| 188 | UAAACCCGGCCACUUUCAGGGUUUUAGA | chr14: 22547501- |
| | GCUAGAAAUAGCAAGUUAAAAUAAGGCU | 22547521 |
| | AGUCCGUUAUCAACUUGAAAAAGUGGCA | |
| | CCGAGUCGGUGCUUUU | |
| 189 | GAUUAAACCCGGCCACUUUCGUUUUAGA | chr14: 22550608- |
| | GCUAGAAAUAGCAAGUUAAAAUAAGGCU | 22550628 |
| | AGUCCGUUAUCAACUUGAAAAAGUGGCA | |
| | CCGAGUCGGUGCUUUU | |
| 190 | CGUCAUGAGCAGAUUAAACCGUUUUAGA | chr14: 22547529- |
| | GCUAGAAAUAGCAAGUUAAAAUAAGGCU | 22547549 |
| | AGUCCGUUAUCAACUUGAAAAAGUGGCA | |
| | CCGAGUCGGUGCUUUU | |
| 191 | AGAGUCUCUCAGCUGGUACAGUUUUAGA | chr14: 22547524- |
| | GCUAGAAAUAGCAAGUUAAAAUAAGGCU | 22547544 |
| | AGUCCGUUAUCAACUUGAAAAAGUGGCA | |
| | CCGAGUCGGUGCUUUU | |
| 192 | UCAGGGUUCUGGAUAUCUGUGUUUUAGA | chr14: 22550622- |
| | GCUAGAAAUAGCAAGUUAAAAUAAGGCU | 22550642 |
| | AGUCCGUUAUCAACUUGAAAAAGUGGCA | |
| | CCGAGUCGGUGCUUUU | |
| 193 | mG*mA*mG*UCCGAGCAGAAGAAGAAGUU | chr2: 72933852- |
| | UUAGAmGmCmUmAmGmAmAmAmUmAmG | 72933872 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 194 | mG*mA*mC*CCCCUCCACCCCGCCUCGUU | chr6: 43770821- |
| | UUAGAmGmCmUmAmGmAmAmAmUmAmG | 43770841 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 195 | AACAGCAUAGCAAGUUAAAAUAAGGCUA | chr14: 22550581- |
| | GUCCGUUAUCAACUUGAAAAAGUGGCAC | 22550601 |
| | CGAGUCGGUGCUUUUUUU | |
| 196 | GGCUCUCGGAGAAUGACGAGGUUUUAGA | |
| | GCUAGAAAUAGCAAGUUAAAAUAAGGCU | chr7: 142791996- |
| | AGUCCGUUAUCAACUUGAAAAAGUGGCA | 142792016 |
| | CCGAGUCGGUGCUUUU | |
| 197 | GGCCUCGGCGCUGACGAUCUGUUUUAGA | chr7: 142792047- |
| | GCUAGAAAUAGCAAGUUAAAAUAAGGCU | 142792067 |
| | AGUCCGUUAUCAACUUGAAAAAGUGGCA | |
| | CCGAGUCGGUGCUUUU | |
| 198 | AUGACGAGUGGACCCAGGAUGUUUUAGA | chr7: 142792008- |
| | GCUAGAAAUAGCAAGUUAAAAUAAGGCU | 142792028 |
| | AGUCCGUUAUCAACUUGAAAAAGUGGCA | |
| | CCGAGUCGGUGCUUUU | |
| 199 | UGAGGGUCUCGGCCACCUUCGUUUUAGA | chr7: 142791930- |
| | GCUAGAAAUAGCAAGUUAAAAUAAGGCU | 142791950 |
| | AGUCCGUUAUCAACUUGAAAAAGUGGCA | |
| | CCGAGUCGGUGCUUUU | |
| 200 | AGAGAUCUCCCACACCCAAAGUUUUAGA | chr7: 142791748- |
| | GCUAGAAAUAGCAAGUUAAAAUAAGGCU | 142791768 |
| | AGUCCGUUAUCAACUUGAAAAAGUGGCA | |
| | CCGAGUCGGUGCUUUU | |
| 201 | mA*mC*mA*CGGCAGGGUCAGGGUUCGUU | chr14: 22547512- |
| | UUAGAmGmCmUmAmGmAmAmAmUmAmG | 22547532 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 202 | mA*mG*mC*UGGUACACGGCAGGGUCGUU | chr14: 22547519- |
| | UUAGAmGmCmUmAmGmAmAmAmUmAmG | 22547539 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 203 | mC*mU*mC*UCAGCUGGUACACGGCAGUU | chr14: 22547524- |
| | UUAGAmGmCmUmAmGmAmAmAmUmAmG | 22547544 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 204 | mU*mC*mU*CUCAGCUGGUACACGGCGUU | chr14: 22547525- |
| | UUAGAmGmCmUmAmGmAmAmAmUmAmG | 22547545 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 205 | mU*mG*mG*AUUUAGAGUCUCUCAGCGUU | chr14: 22547536- |
| | UUAGAmGmCmUmAmGmAmAmAmUmAmG | 22547556 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 206 | mU*mA*mG*GCAGACAGACUUGUCACGUU | chr14: 22547556- |
| | UUAGAmGmCmUmAmGmAmAmAmUmAmG | 22547576 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 207 | mU*mC*mU*GUGGGACAAGAGGAUCAGUU | chr14: 22547486- |
| | UUAGAmGmCmUmAmGmAmAmAmUmAmG | 22547506 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 208 | mA*mU*mC*UGUGGGACAAGAGGAUCGUU | chr14: 22547487- |
| | UUAGAmGmCmUmAmGmAmAmAmUmAmG | 22547507 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 209 | mC*mU*mG*GAUAUCUGUGGGACAAGGUU | chr14: 22547493- |
| | UUAGAmGmCmUmAmGmAmAmAmUmAmG | 22547513 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 210 | mG*mU*mC*AGGGUUCUGGAUAUCUGGUU | chr14: 22547502- |
| | UUAGAmGmCmUmAmGmAmAmAmUmAmG | 22547522 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 211 | mU*mC*mA*GGGUUCUGGAUAUCUGUGUU | chr14: 22547501- |
| | UUAGAmGmCmUmAmGmAmAmAmUmAmG | 22547521 |
| | mCAAGUUAAAAUAAGGCUAGUCCGUUAU | |
| | CAmAmCmUmUmGmAmAmAmAmAmGmUm | |
| | GmGmCmAmCmCmGmAmGmUmCmGmGmU | |
| | mGmCmU*mU*mU*mU | |
| 212 | UAGGCAGACAGACUUGUCACGUUUUAGA | chr14: 22547556- |
| | GCUAGAAAUAGCAAGUUAAAAUAAGGCU | 22547576 |
| | AGUCCGUUAUCAACUUGAAAAAGUGGCA | |
| | CCGAGUCGGUGCUUUU | |

* = PS linkage; m = 2′-O-Me nucleotide; N = any natural or non-natural nucleotide

In some embodiments, the invention provides a composition comprising one or more guide RNA (gRNA) comprising guide sequences that direct an RNA-guided DNA binding agent, which can be a nuclease (e.g., a Cas nuclease such as Cas9), to a target DNA sequence in TRBC1, TRBC2 and/or TRAC. The gRNA may comprise a guide sequence shown in Tables 1 or 2. In some embodiments, the gRNA comprises the following sequence: (N)ₓGUUUUAGAGCUAGAAAUAGCAAGUUAAAAUAAGGCUAGUCCGUUAUCAAC UUGAAAAAGUGGCAC CGAGUCGGUGC (SEQ ID NO: 403), where “N” may be any natural or non-natural nucleotide, and wherein the totality of N's comprise a TRBC or TRAC guide sequence as described herein. The gRNA may comprise a crRNA comprising a guide sequence shown in Tables 1-3. The gRNA may comprise a crRNA comprising 17, 18, 19, or 20 contiguous nucleotides of a guide sequence shown in Tables 1 and 2. In some embodiments, the gRNA comprises a crRNA comprising a sequence with about 75%, 80%, 85%, 90%, 95%, 96%, 97%, 98%, 99%, or 100% identity to at least 17, 18, 19, or 20 contiguous nucleotides of a guide sequence shown in Tables 1 and 2. In some embodiments, the gRNA comprises a crRNA comprising a sequence with about 75%, 80%, 85%, 90%, 95%, 96%, 97%, 98%, 99%, or 100% identity to a guide sequence shown in Tables 1 and 2. The gRNA may further comprise a trRNA. In each embodiment described herein, the crRNA and trRNA may be associated as a single RNA (sgRNA) or may be on separate RNAs (dgRNA). In the context of sgRNAs, the crRNA and trRNA components may be covalently linked, e.g., via a phosphodiester bond or other covalent bond.

In each embodiment described herein, the guide RNA may comprise two RNA molecules as a “dual guide RNA” or “dgRNA”. The dgRNA comprises a first RNA molecule comprising a crRNA comprising, e.g., a guide sequence shown in Tables 1 and 2, and a second RNA molecule comprising a trRNA. The first and second RNA molecules may not be covalently linked, but may form a RNA duplex via the base pairing between portions of the crRNA and the trRNA.

In each embodiment described herein, the guide RNA may comprise a single RNA molecule as a “single guide RNA” or “sgRNA”. The sgRNA may comprise a crRNA (or a portion thereof) comprising a guide sequence shown in Tables 1 and 2 covalently linked to a trRNA. The sgRNA may comprise 17, 18, 19, or 20 contiguous nucleotides of a guide sequence shown in Tables 1 and 2. In some embodiments, the crRNA and the trRNA are covalently linked via a linker. In some embodiments, the sgRNA forms a stem-loop structure via the base pairing between portions of the crRNA and the trRNA. In some embodiments, the crRNA and the trRNA are covalently linked via one or more bonds that are not a phosphodiester bond.

In some embodiments, the trRNA may comprise all or a portion of a trRNA sequence derived from a naturally-occurring CRISPR/Cas system. In some embodiments, the trRNA comprises a truncated or modified wild type trRNA. The length of the trRNA depends on the CRISPR/Cas system used. In some embodiments, the trRNA comprises or consists of 5, 6, 7, 8, 9, 10, 11, 12, 13, 14, 15, 16, 17, 18, 19, 20, 25, 30, 40, 50, 60, 70, 80, 90, 100, or more than 100 nucleotides. In some embodiments, the trRNA may comprise certain secondary structures, such as, for example, one or more hairpin or stem-loop structures, or one or more bulge structures.

In some embodiments, the invention provides a composition comprising one or more guide RNAs comprising a guide sequence of any one of SEQ ID NOs: 1-89 and 90-178.

In some embodiments, the invention provides a composition comprising one or more sgRNAs comprising any one of SEQ ID NOs: 179-184.

In one aspect, the invention provides a composition comprising a gRNA that comprises a guide sequence that is at least 99%, 98%, 97%, 96%, 95%, 94%, 93%, 92%, 91%, or 90% identical to any of the nucleic acids of SEQ ID NOs: 1-89, 90-178, 185, and 213-218.

In other embodiments, the composition comprises at least one, e.g., at least two gRNA's comprising guide sequences selected from any two or more of the guide sequences of SEQ ID NOs: 1-89, 90-178, 185, and 213-218. In some embodiments, the composition comprises at least two gRNA's that each comprise a guide sequence at least 99%, 98%, 97%, 96%, 95%, 94%, 93%, 92%, 91%, or 90% identical to any of the nucleic acids of SEQ ID NOs: 1-89, 90-178, 185, and 213-218.

The guide RNA compositions of the present invention are designed to recognize (e.g., hybridize to) a target sequence in the TRBC1, TRBC2 and/or TRAC genes. For example, the TRBC1, TRBC2 and/or TRAC target sequence may be recognized and cleaved by a provided Cas cleavase comprising a guide RNA. In some embodiments, an RNA-guided DNA binding agent, such as a Cas cleavase, may be directed by a guide RNA to a target sequence of the TRBC1, TRBC2 and/or TRAC genes, where the guide sequence of the guide RNA hybridizes with the target sequence and the RNA-guided DNA binding agent, such as a Cas cleavase, cleaves the target sequence.

In some embodiments, the selection of the one or more guide RNAs is determined based on target sequences within the TRBC1, TRBC2 and/or TRAC genes.

Without being bound by any particular theory, mutations (e.g., frameshift mutations resulting from indels occurring as a result of a nuclease-mediated DSB) in certain regions of the gene may be less tolerable than mutations in other regions of the gene, thus the location of a DSB is an important factor in the amount or type of protein knockdown that may result. In some embodiments, a gRNA complementary or having complementarity to a target sequence within TRBC1, TRBC2 and/or TRAC is used to direct the RNA-guided DNA binding agent to a particular location in the appropriate TRBC1, TRBC2 and/or TRAC gene. In some embodiments, gRNAs are designed to have guide sequences that are complementary or have complementarity to target sequences in exon 1, exon 2, exon 3, or exon 4 of TRBC1 and/or TRBC2, and/or target sequences in exon 1, exon 2, exon 3, or exon 4 of TRAC.

In some embodiments, the guide sequence is at least 99%, 98%, 97%, 96%, 95%, 94%, 93%, 92%, 91%, or 90% identical to a target sequence present in the human TRBC1, TRBC2 and/or TRAC genes. In some embodiments, the target sequence may be complementary to the guide sequence of the guide RNA. In some embodiments, the degree of complementarity or identity between a guide sequence of a guide RNA and its corresponding target sequence may be at least 80%, 85%, 90%, 95%, 96%, 97%, 98%, 99%, or 100%. In some embodiments, the target sequence and the guide sequence of the gRNA may be 100% complementary or identical. In other embodiments, the target sequence and the guide sequence of the gRNA may contain at least one mismatch. For example, the target sequence and the guide sequence of the gRNA may contain 1, 2, 3, or 4 mismatches, where the total length of the guide sequence is 20. In some embodiments, the target sequence and the guide sequence of the gRNA may contain 1-4 mismatches where the guide sequence is 20 nucleotides.

In some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered.

B. Modified gRNAs and mRNAs

In some embodiments, the gRNA is chemically modified. In some embodiments, a gRNA comprises one or more modified nucleosides or nucleotides and can becalled a “modified” gRNA or “chemically modified” gRNA, to describe the presence of one or more non-naturally and/or naturally occurring components or configurations that are used instead of or in addition to the canonical A, G, C, and U residues. In some embodiments, a modified gRNA is synthesized with a non-canonical nucleoside or nucleotide, is here called “modified.” Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the oligonucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification).

Chemical modifications such as those listed above can be combined to provide modified gRNAs and/or mRNAs comprising nucleosides and nucleotides (collectively “residues”) that can have two, three, four, or more modifications. For example, a modified residue can have a modified sugar and a modified nucleobase. In some embodiments, every base of a gRNA is modified, e.g., all bases have a modified phosphate group, such as a phosphorothioate group. In certain embodiments, all, or substantially all, of the phosphate groups of a gRNA molecule are replaced with phosphorothioate groups. In some embodiments, modified gRNAs comprise at least one modified residue at or near the 5′ end of the RNA. In some embodiments, modified gRNAs comprise at least one modified residue at or near the 3′ end of the RNA.

In some embodiments, the gRNA comprises one, two, three or more modified residues. In some embodiments, at least 5% (e.g., at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%) of the positions in a modified gRNA are modified nucleosides or nucleotides.

Unmodified nucleic acids can be prone to degradation by, e.g., intracellular nucleases or those found in serum. For example, nucleases can hydrolyze nucleic acid phosphodiester bonds. Accordingly, in one aspect the gRNAs described herein can contain one or more modified nucleosides or nucleotides, e.g., to introduce stability toward intracellular or serum-based nucleases. In some embodiments, the modified gRNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group, i.e. at sugar modification. For example, the 2′ hydroxyl group (OH) can be modified, e.g. replaced with a number of different “oxy” or “deoxy” substituents. In some embodiments, modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). In some embodiments, the 2′ hydroxyl group modification can be 2′-O-Me. In some embodiments, the 2′ hydroxyl group modification can be a 2′-fluoro modification, which replaces the 2′ hydroxyl group with a fluoride. In some embodiments, the 2′ hydroxyl group modification can include “locked” nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, wherein amino can be, e.g., NH₂; alkylamino, dialkylamino, heterocyclyl, arylamino, diarylamino, heteroarylamino, or diheteroarylamino, ethylenediamine, or polyamino). In some embodiments, the 2′ hydroxyl group modification can include “unlocked” nucleic acids (UNA) in which the ribose ring lacks the C2′-C3′ bond. In some embodiments, the 2′ hydroxyl group modification can include the methoxyethyl group (MOE), (OCH₂CH₂OCH₃, e.g., a PEG derivative).

“Deoxy” 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form, e.g. L-nucleosides.

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In embodiments employing a dual guide RNA, each of the crRNA and the tracr RNA can contain modifications. Such modifications may be at one or both ends of the crRNA and/or tracr RNA. In embodiments comprising an sgRNA, one or more residues at one or both ends of the sgRNA may be chemically modified, and/or internal nucleosides may be modified, and/or the entire sgRNA may be chemically modified. Certain embodiments comprise a 5′ end modification. Certain embodiments comprise a 3′ end modification. Certain embodiments comprise a 5′ end modification and a 3′ end modification.

In some embodiments, the guide RNAs disclosed herein comprise one of the modification patterns disclosed in WO2018/107028 A1, filed Dec. 8, 2017, titled “Chemically Modified Guide RNAs,” the contents of which are hereby incorporated by reference in their entirety. In some embodiments, the guide RNAs disclosed herein comprise one of the structures/modification patterns disclosed in US20170114334, the contents of which are hereby incorporated by reference in their entirety. In some embodiments, the guide RNAs disclosed herein comprise one of the structures/modification patterns disclosed in WO2017/136794, the contents of which are hereby incorporated by reference in their entirety.

In some embodiments, the sgRNA comprises any of the modification patterns shown herein, where N is any natural or non-natural nucleotide, and wherein the totality of the N's comprise a TRBC1, TRBC2 and/or TRAC guide sequence as described herein in Tables 1-3. In some embodiments, the modified sgRNA comprises the following sequence: mN*mN*mN*NNNNNNNNNNNNNNNNNGUUUUAGAmGmCmUmAmGmAmAmAmU mAmGmCAAGUUAAAAUAAGGCUAGUCCGUUAUCAmAmCmUmUmGmAmAmAm AmAmGmUmGmGmCmAmCmCmGmAmGmUmCmGmGmUmGmCmU*mU*mU*mU (SEQ ID NO: 300), where “N” may be any natural or non-natural nucleotide, and wherein the totality of N's comprise an TRBC1, TRBC2 and/or TRAC guide sequence as described in Tables 1 and 2 or as described herein. For example, encompassed herein is SEQ ID NO: 300, where the N's are replaced with any of the guide sequences disclosed herein in Tables 1 and 2 (SEQ ID NOs: 1-89, 90-178, 185, and 213-218).

Any of the modifications described below may be present in the gRNAs and mRNAs described herein.

The terms “mA,” “mC,” “mU,” or “mG” may be used to denote a nucleotide that has been modified with 2′-O-Me.

Modification of 2′-O-methyl can be depicted as follows:

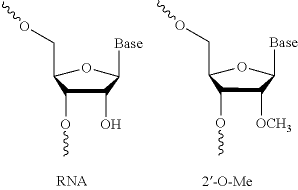

Another chemical modification that has been shown to influence nucleotide sugar rings is halogen substitution. For example, 2′-fluoro (2′-F) substitution on nucleotide sugar rings can increase oligonucleotide binding affinity and nuclease stability.

In this application, the terms “fA,” “fC,” “fU,” or “fG” may be used to denote a nucleotide that has been substituted with 2′-F.

Substitution of 2′-F can be depicted as follows:

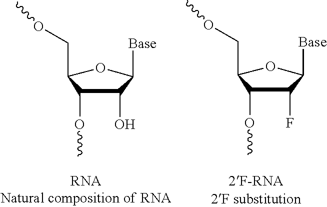

Phosphorothioate (PS) linkage or bond refers to a bond where a sulfur is substituted for one non-bridging phosphate oxygen in a phosphodiester linkage, for example in the bonds between nucleotides bases. When phosphorothioates are used to generate oligonucleotides, the modified oligonucleotides may also be referred to as S-oligos.

A “*” may be used to depict a PS modification. In this application, the terms A*, C*, U*, or G* may be used to denote a nucleotide that is linked to the next (e.g., 3′) nucleotide with a PS bond.

In this application, the terms “mA*,” “mC*,” “mU*,” or “mG*” may be used to denote a nucleotide that has been substituted with 2′-O-Me and that is linked to the next (e.g., 3′) nucleotide with a PS bond.

The diagram below shows the substitution of S— into a non-bridging phosphate oxygen, generating a PS bond in lieu of a phosphodiester bond:

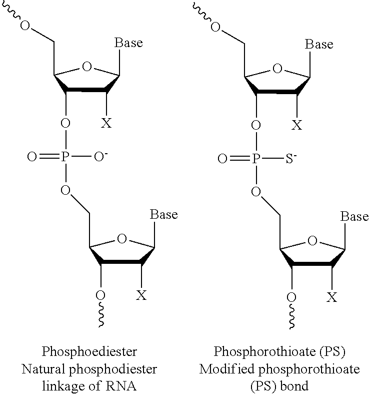

Abasic nucleotides refer to those which lack nitrogenous bases. The figure below depicts an oligonucleotide with an abasic (also known as apurinic) site that lacks a base:

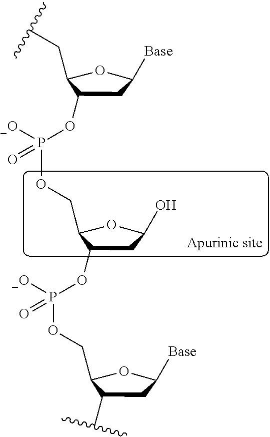

Inverted bases refer to those with linkages that are inverted from the normal 5′ to 3′ linkage (i.e., either a 5′ to 5′ linkage or a 3′ to 3′ linkage). For example:

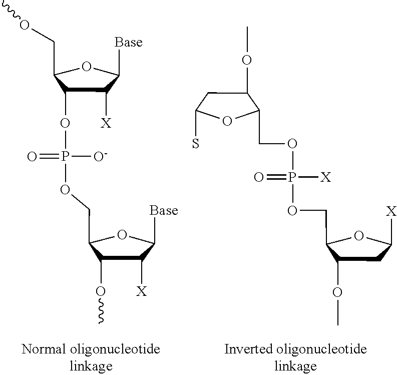

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In some embodiments, one or more of the first three, four, or five nucleotides at the 5′ terminus, and one or more of the last three, four, or five nucleotides at the 3′ terminus are modified. In some embodiments, the modification is a 2′-O-Me, 2′-F, inverted abasic nucleotide, PS bond, or other nucleotide modification well-known in the art to increase stability and/or performance.

In some embodiments, the first four nucleotides at the 5′ terminus, and the last four nucleotides at the 3′ terminus are linked with phosphorothioate (PS) bonds.

In some embodiments, the first three nucleotides at the 5′ terminus, and the last three nucleotides at the 3′ terminus comprise a 2′-O-methyl (2′-O-Me) modified nucleotide. In some embodiments, the first three nucleotides at the 5′ terminus, and the last three nucleotides at the 3′ terminus comprise a 2′-fluoro (2′-F) modified nucleotide. In some embodiments, the first three nucleotides at the 5′ terminus, and the last three nucleotides at the 3′ terminus comprise an inverted abasic nucleotide.

In some embodiments, the guide RNA comprises a modified sgRNA. In some embodiments, the sgRNA comprises the modification pattern shown in SEQ ID NO: 401, where N is any natural or non-natural nucleotide, and where the totality of the N's comprise a guide sequence that directs a nuclease to a target sequence in TRBC1, TRBC2 and/or TRAC, e.g., as shown in Tables 1 and 2.

In some embodiments, the guide RNA comprises a sgRNA shown in any one of SEQ ID NOs: 179-184. In some embodiments, the guide RNA comprises a sgRNA comprising any one of the guide sequences of SEQ ID NOs: 1-178 and the nucleotides of SEQ ID NO: 401, wherein the nucleotides of SEQ ID NO: 401 are on the 3′ end of the guide sequence, and wherein the sgRNA may be modified as shown herein or SEQ ID NO: 300.

As noted above, in some embodiments, a composition or formulation disclosed herein comprises an mRNA comprising an open reading frame (ORF) encoding an RNA-guided DNA binding agent, such as a Cas nuclease as described herein. In some embodiments, an mRNA comprising an ORF encoding an RNA-guided DNA binding agent, such as a Cas nuclease, is provided, used, or administered. In some embodiments, the ORF encoding an RNA-guided DNA nuclease is a “modified RNA-guided DNA binding agent ORF” or simply a “modified ORF,” which is used as shorthand to indicate that the ORF is modified.

In some embodiments, the modified ORF may comprise a modified uridine at least at one, a plurality of, or all uridine positions. In some embodiments, the modified uridine is a uridine modified at the 5 position, e.g., with a halogen, methyl, or ethyl. In some embodiments, the modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine.

In some embodiments, an mRNA disclosed herein comprises a 5′ cap, such as a Cap0, Cap1, or Cap2. A 5′ cap is generally a 7-methylguanine ribonucleotide (which may be further modified, as discussed below e.g. with respect to ARCA) linked through a 5′-triphosphate to the 5′ position of the first nucleotide of the 5′-to-3′ chain of the mRNA, i.e., the first cap-proximal nucleotide. In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc Natl Acad Sci USA 111(33):12025-30; Abbas et al. (2017) Proc Natl Acad Sci USA 114(11):E2106-E2115. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as “non-self” by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al., (2001) “Synthesis and properties of mRNAs containing the novel ‘anti-reverse’ cap analogs 7-methyl(3′-O-methyl)GpppG and 7-methyl(3′deoxy)GpppG,” RNA 7: 1486-1495. The ARCA structure is shown below.

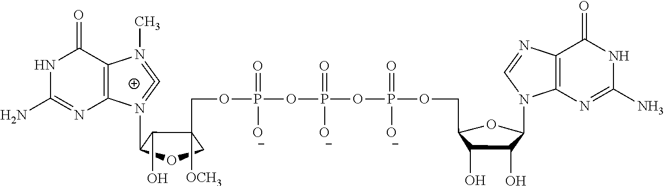

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively. The CleanCap™ AG structure is shown below.

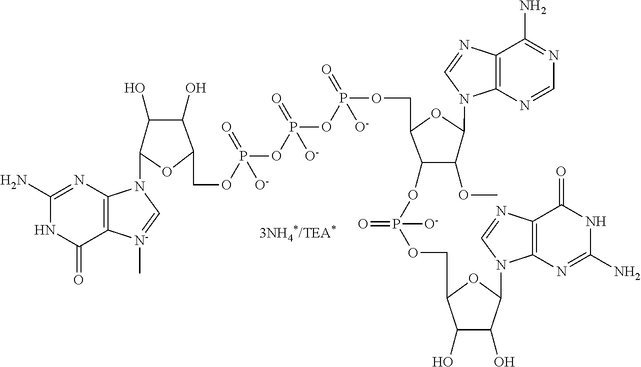

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo, P. and Moss, B. (1990) Proc. Natl. Acad. Sci. USA 87, 4023-4027; Mao, X. and Shuman, S. (1994) J. Biol. Chem. 269, 24472-24479.

In some embodiments, the mRNA further comprises a poly-adenylated (poly-A) tail. In some embodiments, the poly-A tail comprises at least 20, 30, 40, 50, 60, 70, 80, 90, or 100 adenines, optionally up to 300 adenines. In some embodiments, the poly-A tail comprises 95, 96, 97, 98, 99, or 100 adenine nucleotides.

### C. Ribonucleoprotein Complex

In some embodiments, a composition is encompassed comprising one or more gRNAs comprising one or more guide sequences from Tables 1 and 2 or one or more sgRNAs from Table 3 and an RNA-guided DNA binding agent, e.g., a nuclease, such as a Cas nuclease, such as Cas9. In some embodiments, the RNA-guided DNA-binding agent. e.g. Cas9, has cleavase activity, which can also be referred to as double-strand endonuclease activity. In some embodiments, the RNA-guided DNA-binding agent comprises a Cas9 nuclease. Examples of Cas9 nucleases include those of the type II CRISPR systems of S. pyogenes, S. aureus, and other prokaryotes (see, e.g., the list in the next paragraph), and modified (e.g., engineered or mutant) versions thereof. See, e.g., US2016/0312198 A1; US 2016/0312199 A1. Other examples of Cas nucleases include a Csm or Cmr complex of a type III CRISPR system or the Cas10, Csm1, or Cmr2 subunit thereof, and a Cascade complex of a type I CRISPR system, or the Cas3 subunit thereof. In some embodiments, the Cas nuclease may be from a Type-IIA, Type-JIB, or Type-IIC system. For discussion of various CRISPR systems and Cas nucleases see, e.g., Makarova et al., NAT. REV. MICROBIOL. 9:467-477 (2011); Makarova et al., NAT. REV. MICROBIOL, 13: 722-36 (2015); Shmakov et al., MOLECULAR CELL, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gamma proteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In some embodiments, the Cas nuclease is the Cas9 nuclease from Streptococcus thermophilus. In some embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In some embodiments, the Cas nuclease is the Cpf1 nuclease from Acidaminococcus sp. In some embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In certain embodiments, the Cas nuclease is a Cpf1 nuclease from an Acidaminococcus or Lachnospiraceae.

In some embodiments, the gRNA together with an RNA-guided DNA binding agent is called a ribonucleoprotein complex (RNP). In some embodiments, the RNA-guided DNA binding agent is a Cas nuclease. In some embodiments, the gRNA together with a Cas nuclease is called a Cas RNP. In some embodiments, the RNP comprises Type-I, Type-II, or Type-III components. In some embodiments, the Cas nuclease is the Cas9 protein from the Type-II CRISPR/Cas system. In some embodiment, the gRNA together with Cas9 is called a Cas9 RNP.

Wild type Cas9 has two nuclease domains: RuvC and HNH. The RuvC domain cleaves the non-target DNA strand, and the HNH domain cleaves the target strand of DNA. In some embodiments, the Cas9 protein comprises more than one RuvC domain and/or more than one HNH domain. In some embodiments, the Cas9 protein is a wild type Cas9. In each of the composition, use, and method embodiments, the Cas induces a double strand break in target DNA.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain.

In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS). In some embodiments, the RNA-guided DNA-binding agent may be fused with 1-10 NLS(s). In some embodiments, the RNA-guided DNA-binding agent may be fused with 1-5 NLS(s). In some embodiments, the RNA-guided DNA-binding agent may be fused with one NLS. Where one NLS is used, the NLS may be linked at the N-terminus or the C-terminus of the RNA-guided DNA-binding agent sequence. It may also be inserted within the RNA-guided DNA binding agent sequence. In other embodiments, the RNA-guided DNA-binding agent may be fused with more than one NLS. In some embodiments, the RNA-guided DNA-binding agent may be fused with 2, 3, 4, or 5 NLSs. In some embodiments, the RNA-guided DNA-binding agent may be fused with two NLSs. In certain circumstances, the two NLSs may be the same (e.g., two SV40 NLSs) or different. In some embodiments, the RNA-guided DNA-binding agent is fused to two SV40 NLS sequences linked at the carboxy terminus. In some embodiments, the RNA-guided DNA-binding agent may be fused with two NLSs, one linked at the N-terminus and one at the C-terminus. In some embodiments, the RNA-guided DNA-binding agent may be fused with 3 NLSs. In some embodiments, the RNA-guided DNA-binding agent may be fused with no NLS. In some embodiments, the NLS may be a monopartite sequence, such as, e.g., the SV40 NLS, PKKKRKV (SEQ ID NO: 600) or PKKKRRV (SEQ ID NO: 601). In some embodiments, the NLS may be a bipartite sequence, such as the NLS of nucleoplasmin, KRPAATKKAGQAKKKK (SEQ ID NO: 602). In a specific embodiment, a single PKKKRKV (SEQ ID NO: 600) NLS may be linked at the C-terminus of the RNA-guided DNA-binding agent. One or more linkers are optionally included at the fusion site.

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin-like protein (UBL). Non-limiting examples of ubiquitin-like proteins include small ubiquitin-like modifier (SUMO), ubiquitin cross-reactive protein (UCRP, also known as interferon-stimulated gene-15 (ISG15)), ubiquitin-related modifier-1 (URM1), neuronal-precursor-cell-expressed developmentally downregulated protein-8 (NEDD8, also called RubI in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain. When the RNA-guided DNA-binding agent is directed to its target sequence, e.g., when a Cas nuclease is directed to a target sequence by a gRNA, the effector domain may modify or affect the target sequence. In some embodiments, the effector domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA.

D. Determination of Efficacy of gRNAs

In some embodiments, the efficacy of a gRNA is determined when delivered or expressed together with other components forming an RNP. In some embodiments, the gRNA is expressed together with an RNA-guided DNA binding agent, such as a Cas protein, e.g. Cas9. In some embodiments, the gRNA is delivered to or expressed in a cell line that already stably expresses an RNA-guided DNA nuclease, such as a Cas nuclease or nickase, e.g. Cas9 nuclease or nickase. In some embodiments the gRNA is delivered to a cell as part of a RNP. In some embodiments, the gRNA is delivered to a cell along with a mRNA encoding an RNA-guided DNA nuclease, such as a Cas nuclease or nickase, e.g. Cas9 nuclease or nickase.

As described herein, use of an RNA-guided DNA nuclease and a guide RNA disclosed herein can lead to double-stranded breaks in the DNA which can produce errors in the form of insertion/deletion (indel) mutations upon repair by cellular machinery. Many mutations due to indels alter the reading frame or introduce premature stop codons and, therefore, produce a non-functional protein.

In some embodiments, the efficacy of particular gRNAs is determined based on in vitro models. In some embodiments, the in vitro model is HEK293 cells stably expressing Cas9 (HEK293_Cas9). In some embodiments the in vitro model is a peripheral blood mononuclear cell (PBMC). In some embodiments, the in vitro model is a T cell, such as primary human T cells. With respect to using primary cells, commercially available primary cells can be used to provide greater consistency between experiments. In some embodiments, the number of off-target sites at which a deletion or insertion occurs in an in vitro model (e.g., in T cell) is determined, e.g., by analyzing genomic DNA from transfected cells in vitro with Cas9 mRNA and the guide RNA. In some embodiments, such a determination comprises analyzing genomic DNA from the cells transfected in vitro with Cas9 mRNA, the guide RNA, and a donor oligonucleotide. Exemplary procedures for such determinations are provided in the working examples in which HEK293 cells, PBMCs, and human CD3⁺ T cells are used.

In some embodiments, the efficacy of particular gRNAs is determined across multiple in vitro cell models for a gRNA selection process. In some embodiments, a cell line comparison of data with selected gRNAs is performed. In some embodiments, cross screening in multiple cell models is performed.

In some embodiments, the efficacy of a guide RNA is measured by percent indels of TRBC1, TRBC2 and/or TRAC. In some embodiments, the efficacy of a guide RNA is measured by percent indels of TRBC and/or TRAC. In some embodiments, the efficacy of a guide RNA is measured by percent indels of TRBC or TRAC. In some embodiments, the percent editing of TRBC1, TRBC2 and/or TRAC is compared to the percent indels necessary to achieve knockdown of the TRBC1, TRBC2 and/or TRAC protein products

In some embodiments, the efficacy of a guide RNA is measured by reduced or eliminated expression of a component of the T-cell receptor (TCR). In embodiments, the reduced or eliminated expression of a component of the T-cell receptor (TCR) includes reduced or eliminated expression of TRAC, TRBC1, TRBC2, CD3E, CD3G, and/or CD3D. In embodiments, said reduced or eliminated expression of said component of the TCR is the result of introduction of one or more, e.g., one or two, e.g., one gRNA molecule described herein to said component of the TCR into said cell. In embodiments, said reduced or eliminated expression of a component of the TCR is as measured by flow cytometry, e.g., as described herein.

In some embodiments, the efficacy of a guide RNA is measured by the number and/or frequency of indels at off-target sequences within the genome of the target cell type, such as a T cell. In some embodiments, efficacious guide RNAs are provided which produce indels at off target sites at very low frequencies (e.g., <5%) in a cell population and/or relative to the frequency of indel creation at the target site. Thus, the disclosure provides for guide RNAs which do not exhibit off-target indel formation in the target cell type (e.g., a T cell), or which produce a frequency of off-target indel formation of <5% in a cell population and/or relative to the frequency of indel creation at the target site. In some embodiments, the disclosure provides guide RNAs which do not exhibit any off target indel formation in the target cell type (e.g., T cell). In some embodiments, guide RNAs are provided which produce indels at less than 5 off-target sites, e.g., as evaluated by one or more methods described herein. In some embodiments, guide RNAs are provided which produce indels at less than or equal to 4, 3, 2, or 1 off-target site(s) e.g., as evaluated by one or more methods described herein. In some embodiments, the off-target site(s) does not occur in a protein coding region in the target cell (e.g., hepatocyte) genome.

In some embodiments, detecting gene editing events, such as the formation of insertion/deletion (“indel”) mutations and homology directed repair (HDR) events in target DNA utilize linear amplification with a tagged primer and isolating the tagged amplification products (herein after referred to as “LAM-PCR,” or “Linear Amplification (LA)” method).

In some embodiments, the efficacy of a guide RNA is measured by the levels of functional protein complexes comprising the expressed protein product of the gene. In some embodiments, the efficacy of a guide RNA is measured by flow cytometric analysis of TCR expression by which the live population of edited cells is analyzed for loss of the TCR.

### III. Methods Including Therapeutic Methods and Methods of Preparing Engineered Cells or Immunotherapy Reagents

The gRNAs and associated methods and compositions disclosed herein are useful in immunotherapies and for making immunotherapy reagents, such as engineered cells.

In some embodiments, the gRNAs comprising the guide sequences of Tables 1 and 2 together with an RNA-guided DNA nuclease such as a Cas nuclease induce DSBs, and non-homologous ending joining (NHEJ) during repair leads to a mutation in the TRBC1, TRBC2 and/or TRAC genes. In some embodiments, NHEJ leads to a deletion or insertion of a nucleotide(s), which induces a frame shift or nonsense mutation in the TRBC1, TRBC2 and/or TRAC genes.

In some embodiments, the subject is mammalian. In some embodiments, the subject is human. In some embodiments, the subject is cow, pig, monkey, sheep, dog, cat, fish, or poultry.

In some embodiments, the use of a guide RNAs comprising any one or more of the guide sequences in Tables 1 and 2 or one or more sgRNAs from Table 3 (e.g., in a composition provided herein) is provided for the preparation of a medicament for treating a human subject having need for immunotherapy.

In some embodiments, the guide RNAs, compositions, and formulations are used to produce ex vivo a T cell with mutated TRBC1, TRBC2 and/or TRAC genes. The modified T cell may be a natural killer (NK) T cell. The modified T cell may express a T-cell receptor, such as a universal TCR or a modified TCR. The T cell may express a CAR or a CAR construct with a zeta chain signaling motif.

In some embodiments, a single administration of a composition comprising a guide RNA provided herein is sufficient to knock down expression of TRBC1, TRBC2 or TRAC. In other embodiments, more than one administration of a composition comprising a guide RNA provided herein may be beneficial to increase therapeutic effects.

Delivery of gRNA Compositions

Lipid nanoparticles (LNPs) are a useful means for delivery of nucleotide and protein cargo, and may be used for delivery of the guide RNAs, compositions, or pharmaceutical formulations disclosed herein. In some embodiments, the LNPs deliver nucleic acid, protein, or nucleic acid together with protein.

The guide RNA or other nucleic acid (e.g. encoding a polypeptide) may be associated with a lipid nanoparticle (LNP). Suitable LNPs are known in the art. See, e.g., lipids of PCT/US2018/053559, WO/2017/173054, WO2015/095340, and WO2014/136086, as well as references provided therein.

In some embodiments, the invention comprises a method for delivering any one of the gRNAs disclosed herein to a subject, wherein the gRNA is delivered via an LNP. In some embodiments, the gRNA/LNP is also associated with a Cas9 or an mRNA encoding Cas9.

In some embodiments, the invention comprises a composition comprising any one of the gRNAs disclosed and an LNP. In some embodiments, the composition further comprises a Cas9 or an mRNA encoding Cas9.

In some embodiments, the LNPs comprise an ionizable lipid. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., lipids of PCT/US2018/053559 (filed Sep. 28, 2018), WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, LNPs associated with the gRNAs disclosed herein are for use in preparing a medicament for treating a disease or disorder.

Electroporation is another useful means for delivery of cargo, and any electroporation methodology may be used for delivery of any one of the gRNAs disclosed herein. In some embodiments, electroporation may be used to deliver any one of the gRNAs disclosed herein and Cas9 or an mRNA encoding Cas9.

In some embodiments, the invention comprises a method for delivering any one of the gRNAs disclosed herein to an ex vivo cell, wherein the gRNA is associated with an LNP or not associated with an LNP. In some embodiments, the gRNA/LNP or gRNA is also associated with a Cas9 or an mRNA encoding Cas9.

In some embodiments, the guide RNA compositions described herein, alone or encoded on one or more vectors, are formulated in or administered via a lipid nanoparticle; see e.g., WO/2017/173054, the contents of which are hereby incorporated by reference in their entirety.

In certain embodiments, the invention comprises DNA or RNA vectors encoding any of the guide RNAs comprising any one or more of the guide sequences described herein. In some embodiments, in addition to guide RNA sequences, the vectors further comprise nucleic acids that do not encode guide RNAs. Nucleic acids that do not encode guide RNA include, but are not limited to, promoters, enhancers, regulatory sequences, and nucleic acids encoding an RNA-guided DNA nuclease, which can be a nuclease such as Cas9. In some embodiments, the vector comprises one or more nucleotide sequence(s) encoding a crRNA, a trRNA, or a crRNA and trRNA. In some embodiments, the vector comprises one or more nucleotide sequence(s) encoding a sgRNA and an mRNA encoding an RNA-guided DNA nuclease, which can be a Cas nuclease, such as Cas9 or Cpf1. In some embodiments, the vector comprises one or more nucleotide sequence(s) encoding a crRNA, a trRNA, and an mRNA encoding an RNA-guided DNA nuclease, which can be a Cas protein, such as, Cas9. In one embodiment, the Cas9 is from Streptococcus pyogenes (i.e., Spy Cas9). In some embodiments, the nucleotide sequence encoding the crRNA, trRNA, or crRNA and trRNA (which may be a sgRNA) comprises or consists of a guide sequence flanked by all or a portion of a repeat sequence from a naturally-occurring CRISPR/Cas system. The nucleic acid comprising or consisting of the crRNA, trRNA, or crRNA and trRNA may further comprise a vector sequence wherein the vector sequence comprises or consists of nucleic acids that are not naturally found together with the crRNA, trRNA, or crRNA and trRNA.

In some embodiments, the components can be introduced as naked nucleic acid, as nucleic acid complexed with an agent such as a liposome or poloxamer, or they can be introduced as nucleic acid (e.g. gRNA) and protein as a ribonucleoprotein complex (RNP). In some embodiments, the components may be delivered by vectors, such as viral vectors (e.g., adenovirus, AAV, herpesvirus, retrovirus, lentivirus). Methods and compositions for non-viral delivery of nucleic acids include electroporation, lipofection, microinjection, biolistics, virosomes, liposomes, immunoliposomes, LNPs, polycation or lipid:nucleic acid conjugates, naked nucleic acid (e.g., naked DNA/RNA), artificial virions, and agent-enhanced uptake of DNA. Sonoporation using, e.g., the Sonitron 2000 system (Rich-Mar) can also be used for delivery of nucleic acids.

A polypeptide can be introduced to a cell by delivering a nucleic acid, such as a nucleic acid template that encodes the polypeptide of interest to the cell. A nucleic acid template may encode a polypeptide of interest, optionally as part of an expression cassette, wherein the template further comprises flanking sequences. Such flanking sequences are optionally homology arms, designed to facilitate homology-directed repair and integration of the template nucleic acid in the cell. A nucleic acid template may include an open reading frame (ORF) for a polypeptide of interest flanked by sequences homologous to a first target locus to facilitate homologous recombination at the target locus. A template may include homology arms flanking a gRNA cleavage site, e.g. wherein the homology arms comprise flanking sequences. In certain embodiments, the flanking sequences are designed to omit a region of the target locus at or around the cleavage site to yield a deletion of this region that thus prevents cutting of the site after repair and also prevents the gRNA/Cas9 from cutting the template nucleic acid. The omitted region or “gap” that includes the gRNA cleavage site may be 5, 10, 20, 30, 40, 50, 60, 70, 80, 90, 100, 150, or more nucleotides in length. The flanking sequences may be designed to delete a 10-100 nucleotide gap, a 10-50 nucleotide gap, a 25-100, nucleotide gap, or a 50-100 nucleotide gap, for example.

A nucleic acid encoding a polypeptide of interest may be comprised in a viral template or a nonviral template. A viral vector may be any suitable virus, such as a retrovirus, adenovirus, lentivirus, adeno-associated virus, or hybrid thereof. In some embodiments, the viral vector may be an adeno-associated virus (AAV) vector. The nucleic acid template may comprise an exogenous promoter sequence upstream of an ORF. The nucleic acid template may be promoterless, in that the endogenous TRAC promoter may drive expression of the polypeptide of interest after it is inserted.

In addition to a sequence encoding a polypeptide of interest, a nucleic acid template may comprise one or more of a promoter sequence, a Kozak sequence, an IRES sequence, a splice acceptor sequence, a polyA sequence, and a sequence that encodes a cleavable peptide such as P2A, T2A, E2A, and the like.

In some embodiments, methods described herein further comprise delivering one or more sequences comprising one or more genes encoding one or more polypeptides of interest to a cell. In some embodiments, the polypeptide of interest is involved in regulation of immune tolerance. In some embodiments, the polypeptide of interest is involved in regulation of a cancer phenotype.

In some embodiments, the one or more polypeptides comprise a receptor, e.g., an immunological receptor. An immunological receptor means a receptor that can recognize an antigen. Exemplary types of immunological receptors are T-cell receptors (TCR) and chimeric antigen receptors (CAR). In some embodiments, the immunological receptor recognizes a cancer antigen, such as WT1. In certain embodiments, the polypeptide of interest is a WT1 TCR. The WT1 TCR may recognize the VLDFAPPGA (VLD) or RMVFPNAPYL (RMVIF) WT1 peptide. In certain embodiments the WT1 TCR recognizes the VLD peptide. The polypeptide of interest may comprise a WT1 TCR α chain. The polypeptide of interest may comprise a WT1 TCR β chain. In certain embodiments, the polypeptide of interest may comprise a WT1 TCR α chain and a WT1 TCR β chain. Exemplary WT1-specific TCR sequences are known in the art and include:

TABLE 4

| | | SEQ |
|---|---|---|
| | | ID |
| TCR ID | Amino Acid Sequence | NO: |
| TCR-A | MGSWTLCCVSLCILVAKHTDAGVIQSPRHEVTEMGQEVT | 500 |
| (β-linker-α | LRCKPISGHDYLFWYRQTMMRGLELLIYFNNNVPIDDSG | |
| configuration) | MPEDRFSAKMPNASFSTLKIQPSEPRDSAVYFCASRKTGG | |
| | YSNQPQHFGDGTRLSILEDLKNVFPPEVAVFEPSEAEISHT | |
| | QKATLVCLATGFYPDHVELSWWVNGKEVHSGVSTDPQPL | |
| | KEQPALNDSRYCLSSRLRVSATFWQNPRNHFRCQVQFYG | |
| | LSENDEWTQDRAKPVTQIVSAEAWGRADCGFTSESYQQG | |
| | VLSATILYEILLGKATLYAVLVSALVLMAMVKRKDSRGGS | |
| | GATNFSLLKQAGDVEENPGPMETLLKVLSGTLLWQLTWV | |
| | RSQQPVQSPQAVILREGEDAVINCSSSKALYSVHWYRQKH | |
| | GEAPVFLMILLKGGEQKGHEKISASFNEKKQQSSLYLTAS | |
| | QLSYSGTYFCGTAWINDYKLSFGAGTTVTVRANIQNPDP | |
| | AVYQLRDSKSSDKSVCLFTDFDSQTNVSQSKDSDVYITDK | |
| | TVLDMRSMDFKSNSAVAWSNKSDFACANAFNNSIIPEDTF | |
| | FPSPESSCDVKLVEKSFETDTNLNFQNLSVIGFRILLLKVAG | |
| | FNLLMTLRLWSS | |
| TCR-A, α | METLLKVLSGTLLWQLTWVRSQQPVQSPQAVILREGEDA | 501 |
| chain | VINCSSSKALYSVHWYRQKHGEAPVFLMILLKGGEQKG | |
| | HEKISASFNEKKQQSSLYLTASQLSYSGTYFCGTAWINDY | |
| | KLSFGAGTTVTVRANIQNPDPAVYQLRDSKSSDKSVCLFT | |
| | DFDSQTNVSQSKDSDVYITDKTVLDMRSMDFKSNSAVAW | |
| | SNKSDFACANAFNNSIIPEDTFFPSPESSCDVKLVEKSFETD | |
| | TNLNFQNLSVIGFRILLLKVAGFNLLMTLRLWSS | |
| TCR-A, β | MGSWTLCCVSLCILVAKHTDAGVIQSPRHEVTEMGQEVT | 502 |
| chain | LRCKPISGHDYLFWYRQTMMRGLELLIYFNNNVPIDDSG | |
| | MPEDRFSAKMPNASFSTLKIQPSEPRDSAVYFCASRKTGG | |
| | YSNQPQHFGDGTRLSILEDLKNVFPPEVAVFEPSEAEISHT | |
| | QKATLVCLATGFYPDHVELSWWVNGKEVHSGVSTDPQPL | |
| | KEQPALNDSRYCLSSRLRVSATFWQNPRNHFRCQVQFYG | |
| | LSENDEWTQDRAKPVTQIVSAEAWGRADCGFTSESYQQG | |
| | VLSATILYEILLGKATLYAVLVSALVLMAMVKRKDSRG | |
| TCR-B | MGSWTLCCVSLCILVAKHTDAGVIQSPRHEVTEMGQEVT | 503 |
| (β-linker-α | LRCKPISGHDYLFWYRQTMMRGLELLIYFNNNVPIDDSG | |
| configuration) | MPEDRFSAKMPNASFSTLKIQPSEPRDSAVYFCASRKTGG | |
| | YSNQPQHFGDGTRLSILEDLKNVFPPEVAVFEPSEAEISHT | |
| | QKATLVCLATGFYPDHVELSWWVNGKEVHSGVSTDPQPL | |
| | KEQPALNDSRYCLSSRLRVSATFWQNPRNHFRCQVQFYG | |
| | LSENDEWTQDRAKPVTQIVSAEAWGRADCGFTSESYQQG | |
| | VLSATILYEILLGKATLYAVLVSALVLMAMVKRKDSRGGS | |
| | GATNFSLLKQAGDVEENPGPMETLLKVLSGTLLWQLTWV | |
| | RSQQPVQSPQAVILREGEDAVINCSSSKALYSVHWYRQKH | |
| | GEAPVFLMILLKGGEQKGHEKISASFNEKKQQSSLYLTAS | |
| | QLSYSGTYFCGTAWINDYKLSFGAGTTVTVRANIQNPDP | |
| | AVYQLRDSKSSDKSVCLFTDFDSQTNVSQSKDSDVYITDK | |
| | TVLDMRSMDFKSNSAVAWSNKSDFACANAFNNSIIPEDTF | |
| | FPSPESSCDVKLVEKSFETDTNLNFQNLSVIGFRILLLKVAG | |
| | FNLLMTLRLWSS | |
| TCR-B, α | METLLKVLSGTLLWQLTWVRSQQPVQSPQAVILREGEDA | 504 |
| chain | VINCSSSKALYSVHWYRQKHGEAPVFLMILLKGGEQKG | |
| | HEKISASFNEKKQQSSLYLTASQLSYSGTYFCGTAWINDY | |
| | KLSFGAGTTVTVRANIQNPDPAVYQLRDSKSSDKSVCLFT | |
| | DFDSQTNVSQSKDSDVYITDKCVLDMRSMDFKSNSAVAW | |
| | SNKSDFACANAFNNSIIPEDTFFPSPESSCDVKLVEKSFETD | |
| | TNLNFQNLSVIGFRILLLKVAGFNLLMTLRLWSS | |
| TCR-B, β | MGSWTLCCVSLCILVAKHTDAGVIQSPRHEVTEMGQEVT | 505 |
| chain | LRCKPISGHDYLFWYRQTMMRGLELLIYFNNNVPIDDSG | |
| | MPEDRFSAKMPNASFSTLKIQPSEPRDSAVYFCASRKTGG | |
| | YSNQPQHFGDGTRLSILEDLKNVFPPEVAVFEPSEAEISHT | |
| | QKATLVCLATGFYPDHVELSWWVNGKEVHSGVSTDPQPL | |
| | KEQPALNDSRYCLSSRLRVSATFWQNPRNHFRCQVQFYG | |
| | LSENDEWTQDRAKPVTQIVSAEAWGRADCGFTSESYQQG | |
| | VLSATILYEILLGKATLYAVLVSALVLMAMVKRKDSRG | |
| TCR-C | MSNQVLCCVVLCFLGANTVDGGITQSPKYLFRKEGQNVT | 506 |
| (β-linker-α | LSCEQNLNHDAMYWYRQDPGQGLRLIYYSQIVNDFQKGD | |
| configuration) | IAEGYSVSREKKESFPLTVTSAQKNPTAFYLCASSPGALYE | |
| | QYFGPGTRLTVTEDLKNVFPPEVAVFEPSEAEISHTQKATL | |
| | VCLATGFYPDHVELSWWVNGKEVHSGVCTDPQPLKEQPA | |
| | LNDSRYCLSSRLRVSATFWQNPRNHFRCQVQFYGLSENDE | |
| | WTQDRAKPVTQIVSAEAWGRADCGFTSESYQQGVLSATI | |
| | LYEILLGKATLYAVLVSALVLMAMVKRKDSRGGSGATNF | |
| | SLLKQAGDVEENPGPMTSIRAVFIFLWLQLDLVNGENVEQ | |
| | HPSTLSVQEGDSAVIKCTYSDSASNYFPWYKQELGKRPQL | |
| | IIDIRSNVGEKKDQRIAVTLNKTAKHFSLHITETQPEDSAVY | |
| | FCAATEDLTLIWGAGTKLIIKPDIQNPDPAVYQLRDSKSSD | |
| | KSVCLFTDFDSQTNVSQSKDSDVYITDKCVLDMRSMDFKS | |
| | NSAVAWSNKSDFACANAFNNSIIPEDTFFPSPESSCDVKLV | |
| | EKSFETDTNLNFQNLSVIGFRILLLKVAGFNLLMTLRLWSS | |
| TCR-C, α | MTSIRAVFIFLWLQLDLVNGENVEQHPSTLSVQEGDSAVI | 507 |
| chain | KCTYSDSASNYFPWYKQELGKRPQLIIDIRSNVGEKKDQRI | |
| | AVTLNKTAKHFSLHITETQPEDSAVYFCAATEDLTLIWGA | |
| | GTKLIIKPDIQNPDPAVYQLRDSKSSDKSVCLFTDFDSQTN | |
| | VSQSKDSDVYITDKCVLDMRSMDFKSNSAVAWSNKSDFA | |
| | CANAFNNSIIPEDTFFPSPESSCDVKLVEKSFETDTNLNFQN | |
| | LSVIGFRILLLKVAGFNLLMTLRLWSS | |
| TCR-C, β | MSNQVLCCVVLCFLGANTVDGGITQSPKYLFRKEGQNVT | 508 |
| chain | LSCEQNLNHDAMYWYRQDPGQGLRLIYYSQIVNDFQKGD | |
| | IAEGYSVSREKKESFPLTVTSAQKNPTAFYLCASSPGALYE | |
| | QYFGPGTRLTVTEDLKNVFPPEVAVFEPSEAEISHTQKATL | |
| | VCLATGFYPDHVELSWWVNGKEVHSGVCTDPQPLKEQPA | |
| | LNDSRYCLSSRLRVSATFWQNPRNHFRCQVQFYGLSENDE | |
| | WTQDRAKPVTQIVSAEAWGRADCGFTSESYQQGVLSATI | |
| | LYEILLGKATLYAVLVSALVLMAMVKRKDSRG | |
| TCR-D | MGCRLLCCAVLCLLGAGELVPMETGVTQTPRHLVMGMT | 509 |
| (β-linker-α | NKKSLKCEQHLGHNAMYWYKQSAKKPLELMFVYSLEER | |
| configuration) | VENNSVPSRFSPECPNSSHLFLHLHTLQPEDSALYLCASSQ | |
| | DYLVSNEKLFFGSGTQLSVLEDLKNVFPPEVAVFEPSEAEI | |
| | SHTQKATLVCLATGFYPDHVELSWWVNGKEVHSGVSTDP | |
| | QPLKEQPALNDSRYCLSSRLRVSATFWQNPRNHFRCQVQF | |
| | YGLSENDEWTQDRAKPVTQIVSAEAWGRADCGFTSESYQ | |
| | QGVLSATILYEILLGKATLYAVLVSALVLMAMVKRKDSR | |
| | GGSGATNFSLLKQAGDVEENPGPMISLRVLLVILWLQLSW | |
| | VWSQRKEVEQDPGPFNVPEGATVAFNCTYSNSASQSFFW | |
| | YRQDCRKEPKLLMSVYSSGNEDGRFTAQLNRASQYISLLI | |
| | RDSKLSDSATYLCVVNLLSNQGGKLIFGQGTELSVKPNIQ | |
| | NPDPAVYQLRDSKSSDKSVCLFTDFDSQTNVSQSKDSDVY | |
| | ITDKTVLDMRSMDFKSNSAVAWSNKSDFACANAFNNSIIP | |
| | EDTFFPSPESSCDVKLVEKSFETDTNLNFQNLSVIGFRILLL | |
| | KVAGFNLLMTLRLWSS | |
| TCR-D, α | MISLRVLLVILWLQLSWVWSQRKEVEQDPGPFNVPEGAT | 510 |
| chain | VAFNCTYSNSASQSFFWYRQDCRKEPKLLMSVYSSGNED | |
| | GRFTAQLNRASQYISLLIRDSKLSDSATYLCVVNLLSNQGG | |
| | KLIFGQGTELSVKPNIQNPDPAVYQLRDSKSSDKSVCLFTD | |
| | FDSQTNVSQSKDSDVYITDKTVLDMRSMDFKSNSAVAWS | |
| | NKSDFACANAFNNSIIPEDTFFPSPESSCDVKLVEKSFETDT | |
| | NLNFQNLSVIGFRILLLKVAGFNLLMTLRLWSS | |
| TCR-D, β | MGCRLLCCAVLCLLGAGELVPMETGVTQTPRHLVMGMT | 511 |
| chain | NKKSLKCEQHLGHNAMYWYKQSAKKPLELMFVYSLEER | |
| | VENNSVPSRFSPECPNSSHLFLHLHTLQPEDSALYLCASSQ | |
| | DYLVSNEKLFFGSGTQLSVLEDLKNVFPPEVAVFEPSEAEI | |
| | SHTQKATLVCLATGFYPDHVELSWWVNGKEVHSGVSTDP | |
| | QPLKEQPALNDSRYCLSSRLRVSATFWQNPRNHFRCQVQF | |
| | YGLSENDEWTQDRAKPVTQIVSAEAWGRADCGFTSESYQ | |
| | QGVLSATILYEILLGKATLYAVLVSALVLMAMVKRKDSR | |
| | G | |
| TCR-E | MGCRLLCCAVLCLLGAGELVPMETGVTQTPRHLVMGMT | 512 |
| (β-linker-α | NKKSLKCEQHLGHNAMYWYKQSAKKPLELMFVYSLEER | |
| configuration) | VENNSVPSRFSPECPNSSHLFLHLHTLQPEDSALYLCASSQ | |
| | DYLVSNEKLFFGSGTQLSVLEDLKNVFPPEVAVFEPSEAEI | |
| | SHTQKATLVCLATGFYPDHVELSWWVNGKEVHSGVSTDP | |
| | QPLKEQPALNDSRYCLSSRLRVSATFWQNPRNHFRCQVQF | |
| | YGLSENDEWTQDRAKPVTQIVSAEAWGRADCGFTSESYQ | |
| | QGVLSATILYEILLGKATLYAVLVSALVLMAMVKRKDSR | |
| | GGSGATNFSLLKQAGDVEENPGPMISLRVLLVILWLQLSW | |
| | VWSQRKEVEQDPGPFNVPEGATVAFNCTYSNSASQSFFW | |
| | YRQDCRKEPKLLMSVYSSGNEDGRFTAQLNRASQYISLLI | |
| | RDSKLSDSATYLCVVNLLSNQGGKLIFGQGTELSVKPNIQ | |
| | NPDPAVYQLRDSKSSDKSVCLFTDFDSQTNVSQSKDSDVY | |
| | ITDKTVLDMRSMDFKSNSAVAWSNKSDFACANAFNNSIIP | |
| | EDTFFPSPESSCDVKLVEKSFETDTNLNFQNLSVIGFRILLL | |
| | KVAGFNLLMTLRLWSS | |
| TCR-E, α | MISLRVLLVILWLQLSWVWSQRKEVEQDPGPFNVPEGAT | 513 |
| chain | VAFNCTYSNSASQSFFWYRQDCRKEPKLLMSVYSSGNED | |
| | GRFTAQLNRASQYISLLIRDSKLSDSATYLCVVNLLSNQGG | |
| | KLIFGQGTELSVKPNIQNPDPAVYQLRDSKSSDKSVCLFTD | |
| | FDSQTNVSQSKDSDVYITDKTVLDMRSMDFKSNSAVAWS | |
| | NKSDFACANAFNNSIIPEDTFFPSPESSCDVKLVEKSFETDT | |
| | NLNFQNLSVIGFRILLLKVAGFNLLMTLRLWSS | |
| TCR-E, β | MGCRLLCCAVLCLLGAGELVPMETGVTQTPRHLVMGMT | 514 |
| chain | NKKSLKCEQHLGHNAMYWYKQSAKKPLELMFVYSLEER | |
| | VENNSVPSRFSPECPNSSHLFLHLHTLQPEDSALYLCASSQ | |
| | DYLVSNEKLFFGSGTQLSVLEDLKNVFPPEVAVFEPSEAEI | |
| | SHTQKATLVCLATGFYPDHVELSWWVNGKEVHSGVSTDP | |
| | QPLKEQPALNDSRYCLSSRLRVSATFWQNPRNHFRCQVQF | |
| | YGLSENDEWTQDRAKPVTQIVSAEAWGRADCGFTSESYQ | |
| | QGVLSATILYEILLGKATLYAVLVSALVLMAMVKRKDSR | |
| | G | |
| TCR-F | MLSPDLPDSAWNTRLLCHVMLCLLGAVSVAAGVIQSPRH | 515 |
| (β-linker-α | LIKEKRETATLKCYPIPRHDTVYWYQQGPGQDPQFLISFYE | |
| configuration) | KMQSDKGSIPDRFSAQQFSDYHSELNMSSLELGDSALYFC | |
| | ASSLRGGLEKLFFGSGTQLSVLEDLNKVFPPEVAVFEPSEA | |
| | EISHTQKATLVCLATGFFPDHVELSWWVNGKEVHSGVCT | |
| | DPQPLKEQPALNDSRYCLSSRLRVSATFWQNPRNHFRCQV | |
| | QFYGLSENDEWTQDRAKPVTQIVSAEAWGRADCGFTSVS | |
| | YQQGVLSATILYEILLGKATLYAVLVSALVLMAMVKRKD | |
| | FGSGATNFSLLKQAGDVEENPGPMSLSSLLKVVTASLWLG | |
| | PGIAQKITQTQPGMFVQEKEAVTLDCTYDTSDPSYGLFWY | |
| | KQPSSGEMIFLIYQGSYDQQNATEGRYSLNFQKARKSANL | |
| | VISASQLGDSAMYFCAISGNTPLVFGKGTRLSVIANIQNPD | |
| | PAVYQLRDSKSSDKSVCLFTDFDSQTNVSQSKDSDVYITD | |
| | KCVLDMRSMDFKSNSAVAWSNKSDFACANAFNNSIIPED | |
| | TFFPSPESSCDVKLVEKSFETDTNLNFQNLSVIGFRILLLKV | |
| | AGFNLLMTLRLWSS | |
| TCR-F, α | MSLSSLLKVVTASLWLGPGIAQKITQTQPGMFVQEKEAVT | 516 |
| chain | LDCTYDTSDPSYGLFWYKQPSSGEMIFLIYQGSYDQQNAT | |
| | EGRYSLNFQKARKSANLVISASQLGDSAMYFCAISGNTPL | |
| | VFGKGTRLSVIANIQNPDPAVYQLRDSKSSDKSVCLFTDFD | |
| | SQTNVSQSKDSDVYITDKCVLDMRSMDFKSNSAVAWSNK | |
| | SDFACANAFNNSIIPEDTFFPSPESSCDVKLVEKSFETDTNL | |
| | NFQNLSVIGFRILLLKVAGFNLLMTLRLWSS | |
| TCR-F, β | MLSPDLPDSAWNTRLLCHVMLCLLGAVSVAAGVIQSPRH | 517 |
| chain | LIKEKRETATLKCYPIPRHDTVYWYQQGPGQDPQFLISFYE | |
| | KMQSDKGSIPDRFSAQQFSDYHSELNMSSLELGDSALYFC | |
| | ASSLRGGLEKLFFGSGTQLSVLEDLNKVFPPEVAVFEPSEA | |
| | EISHTQKATLVCLATGFFPDHVELSWWVNGKEVHSGVCT | |
| | DPQPLKEQPALNDSRYCLSSRLRVSATFWQNPRNHFRCQV | |
| | QFYGLSENDEWTQDRAKPVTQIVSAEAWGRADCGFTSVS | |
| | YQQGVLSATILYEILLGKATLYAVLVSALVLMAMVKRKD | |
| | F | |
| TCR-G | MGTRLLCWAALCLLGAELTEAGVAQSPRYKIIEKRQSVAF | 518 |
| (β-linker-α | WCNPISGHATLYWYQQILGQGPKLLIQFQNNGVVDDSQLP | |
| configuration) | KDRFSAERLKGVDSTLKIQPAKLEDSAVYLCASSLYRGEQ | |
| | YFGPGTRLTVTEDLKNVFPPEVAVFEPSEAEISHTQKATLV | |
| | CLATGFYPDHVELSWWVNGKEVHSGVCTDPQPLKEQPAL | |
| | NDSRYCLSSRLRVSATFWQNPRNHFRCQVQFYGLSENDE | |
| | WTQDRAKPVTQIVSAEAWGRADCGFTSESYQQGVLSATI | |
| | LYEILLGKATLYAVLVSALVLMAMVKRKDSRGGSGATNF | |
| | SLLKQAGDVEENPGPMAMLLGASVLILWLQPDWVNSQQ | |
| | KNDDQQVKQNSPSLSVQEGRISILNCDYTNSMFDYFLWY | |
| | KKYPAEGPTFLISISSIKDKNEDGRFTVFLNKSAKHLSLHIV | |
| | PSQPGDSAVYFCAARGQGNLIFGKGTKLSVKPNIQNPDPA | |
| | VYQLRDSKSSDKSVCLFTDFDSQTNVSQSKDSDVYITDKC | |
| | VLDMRSMDFKSNSAVAWSNKSDFACANAFNNSIIPEDTFF | |
| | PSPESSCDVKLVEKSFETDTNLNFQNLSVIGFRILLLKVAGF | |
| | NLLMTLRLWSS | |
| TCR-G, α | MAMLLGASVLILWLQPDWVNSQQKNDDQQVKQNSPSLS | 519 |
| chain (amino | VQEGRISILNCDYTNSMFDYFLWYKKYPAEGPTFLISISSIK | |
| acid | DKNEDGRFTVFLNKSAKHLSLHIVPSQPGDSAVYFCAARG | |
| sequence) | QGNLIFGKGTKLSVKPNIQNPDPAVYQLRDSKSSDKSVCL | |
| | FTDFDSQTNVSQSKDSDVYITDKCVLDMRSMDFKSNSAV | |
| | AWSNKSDFACANAFNNSIIPEDTFFPSPESSCDVKLVEKSF | |
| | ETDTNLNFQNLSVIGFRILLLKVAGFNLLMTLRLWSS | |
| TCR-G, β | MGTRLLCWAALCLLGAELTEAGVAQSPRYKIIEKRQSVAF | 520 |
| chain (amino | WCNPISGHATLYWYQQILGQGPKLLIQFQNNGVVDDSQLP | |
| acid | KDRFSAERLKGVDSTLKIQPAKLEDSAVYLCASSLYRGEQ | |
| sequence) | YFGPGTRLTVTEDLKNVFPPEVAVFEPSEAEISHTQKATLV | |
| | CLATGFYPDHVELSWWVNGKEVHSGVCTDPQPLKEQPAL | |
| | NDSRYCLSSRLRVSATFWQNPRNHFRCQVQFYGLSENDE | |
| | WTQDRAKPVTQIVSAEAWGRADCGFTSESYQQGVLSATI | |
| | LYEILLGKATLYAVLVSALVLMAMVKRKDSRG | |
| TCR-H | MSLGLLCCGAFSLLWAGPVNAGVTQTPKFRVLKTGQSMT | 521 |
| (β-linker-α | LLCAQDMNHEYMYWYRQDPGMGLRLIHYSVGEGTTAKG | |
| configuration) | EVPDGYNVSRLKKQNFLLGLESAAPSQTSVYFCASRGYHR | |
| | LNNEQFFGPGTRLTVLEDLKNVFPPEVAVFEPSEAEISHTQ | |
| | KATLVCLATGFYPDHVELSWWVNGKEVHSGVCTDPQPL | |
| | KEQPALNDSRYCLSSRLRVSATFWQNPRNHFRCQVQFYG | |
| | LSENDEWTQDRAKPVTQIVSAEAWGRADCGFTSESYQQG | |
| | VLSATILYEILLGKATLYAVLVSALVLMAMVKRKDSRGGS | |
| | GATNFSLLKQAGDVEENPGPMISLRVLLVILWLQLSWVW | |
| | SGGGSWSHPQFEKGGGSGGGSGGSAWSHPQFEKQRKEVE | |
| | QDPGPFNVPEGATVAFNCTYSNSASQSFFWYRQDCRKEPK | |
| | LLMSVYSSGNEDGRFTAQLNRASQYISLLIRDSKLSDSATY | |
| | LCVVKPDPGAGSYQLTFGKGTKLSVIPNIQNPDPAVYQLR | |
| | DSKSSDKSVCLFTDFDSQTNVSQSKDSDVYITDKCVLDMR | |
| | SMDFKSNSAVAWSNKSDFACANAFNNSIIPEDTFFPSPESS | |
| | CDVKLVEKSFETDTNLNFQNLSVIGFRILLLKVAGFNLLMT | |
| | LRLWSS | |
| TCR-H, α | MISLRVLLVILWLQLSWVWSGGGSWSHPQFEKGGGSGGG | 522 |
| chain (amino | SGGSAWSHPQFEKQRKEVEQDPGPFNVPEGATVAFNCTY | |
| acid | SNSASQSFFWYRQDCRKEPKLLMSVYSSGNEDGRFTAQL | |
| sequence) | NRASQYISLLIRDSKLSDSATYLCVVKPDPGAGSYQLTFGK | |
| | GTKLSVIPNIQNPDPAVYQLRDSKSSDKSVCLFTDFDSQTN | |
| | VSQSKDSDVYITDKCVLDMRSMDFKSNSAVAWSNKSDFA | |
| | CANAFNNSIIPEDTFFPSPESSCDVKLVEKSFETDTNLNFQN | |
| | LSVIGFRILLLKVAGFNLLMTLRLWSS | |
| TCR-H, β | MSLGLLCCGAFSLLWAGPVNAGVTQTPKFRVLKTGQSMT | 523 |
| chain (amino | LLCAQDMNHEYMYWYRQDPGMGLRLIHYSVGEGTTAKG | |
| acid | EVPDGYNVSRLKKQNFLLGLESAAPSQTSVYFCASRGYHR | |
| sequence) | LNNEQFFGPGTRLTVLEDLKNVFPPEVAVFEPSEAEISHTQ | |
| | KATLVCLATGFYPDHVELSWWVNGKEVHSGVCTDPQPL | |
| | KEQPALNDSRYCLSSRLRVSATFWQNPRNHFRCQVQFYG | |
| | LSENDEWTQDRAKPVTQIVSAEAWGRADCGFTSESYQQG | |
| | VLSATILYEILLGKATLYAVLVSALVLMAMVKRKDSRG | |

Complementarity determining regions (CDRs) exist within variable domains, e.g., of TCRs and antibodies. There are three CDRs in each of the variable domains of TCR alpha and TCR beta, which are designated CDR1, CDR2 and CDR3 for each of the variable domains. The exact boundaries of these CDRs may be defined according to known methods, see, e.g., Kabat et al., “Sequences of Proteins of Immunological Interest” and Dunbar et al., “ANARCI: antigen receptor numbering and receptor classification,” which provide a residue numbering system applicable to a variable domain as well as residue boundaries defining the three CDRs. Other CDR definitions methods may produce CDRs that overlap with the Kabat-based CDRs and are shortened or lengthened in light of prediction or experimental findings that particular residues or groups of residues or even entire CDRs do not significantly impact antigen binding. The CDRs herein may be defined according to any of these methods.

The one or more sequences may insert, e.g., undergo integration, into a locus such as TRAC, TRBC1, and/or TRBC2. In some embodiments, the one or more genes insert, e.g., undergo integration, into the TRAC locus. The one or more genes may be provided in a form operably linked to a heterologous promoter, such as an EF-1α promoter. Alternatively, the one or more genes may be provided such that they are configured to be expressed from an endogenous promoter upon integration, e.g., the TRAC, TRBC1, or TRBC2 promoter. The one or more genes may comprise a translational fusion of two or more polypeptides linked by a self-cleaving sequence. This approach can be helpful where coding sequences for a multi-chain receptor (e.g., T-cell receptor) are provided and it is desired to use a single promoter (whether endogenous or heterologous) to drive expression of the receptor. As such, the activity of the promoter results in an mRNA comprising a continuous coding sequence including the amino acids of a plurality of receptor chains that is translated into a polypeptide that undergoes cleavage to provide the individual receptor chains.

The one or more sequences may be delivered by any appropriate means, e.g., transfection, lipid nanoparticle, electroporation, microinjection, or viral vector. In some embodiments, a viral vector such as an adeno-associated virus vector is used.

### IV. Additional Exemplary Embodiments

The following additional exemplary embodiments are provided.

Embodiment 001. A method of altering a DNA sequence within the TRBC1 and/or TRBC2 gene, comprising delivering a composition to a cell, wherein the composition comprises:
a. a guide RNA comprising a sequence chosen from:
   i. a guide sequence selected from SEQ ID NOs: 1-89;
   ii. at least 17, 18, 19, or 20 contiguous nucleotides of a sequence selected from SEQ ID NOs: 1-89;
   iii. a guide sequence that is at least 99%, 98%, 97%, 96%, 95%, 94%, 93%, 92%, 91%, or 90% identical to a sequence selected from SEQ ID NOs: 1-89;
   iv. a guide sequence comprising any one of SEQ ID Nos: 1-24; and
   v. a guide sequence comprising any one of SEQ ID Nos: 1-6; or
b. a nucleic acid encoding a guide RNA of (a.); and optionally
c. an RNA-guided DNA binding agent or a nucleic acid encoding an RNA-guided DNA binding agent.

Embodiment 002. A method of reducing the expression of the TRBC1 and/or TRBC2 gene comprising delivering a composition to a cell, wherein the composition comprises:
a. a guide RNA comprising a sequence chosen from:
   i. a guide sequence selected from SEQ ID NOs: 1-89;
   ii. at least 17, 18, 19, or 20 contiguous nucleotides of a sequence selected from SEQ ID NOs: 1-89;
   iii. a guide sequence that is at least 99%, 98%, 97%, 96%, 95%, 94%, 93%, 92%, 91%, or 90% identical to a sequence selected from SEQ ID NOs: 1-89;
   iv. a guide sequence comprising any one of SEQ ID Nos: 1-24; and
   v. a guide sequence comprising any one of SEQ ID Nos: 1-6; or
b. a nucleic acid encoding a guide RNA of (a.); and optionally
c. an RNA-guided DNA binding agent or a nucleic acid encoding an RNA-guided DNA binding agent.

Embodiment 003. A method of immunotherapy comprising administering a composition to a subject, an autologous cell thereof, and/or an allogeneic cell, wherein the composition comprises:
a. a guide RNA comprising a sequence chosen from:
   i. a guide sequence selected from SEQ ID NOs: 1-89;
   ii. at least 17, 18, 19, or 20 contiguous nucleotides of a sequence selected from SEQ ID NOs: 1-89;
   iii. a guide sequence that is at least 99%, 98%, 97%, 96%, 95%, 94%, 93%, 92%, 91%, or 90% identical to a sequence selected from SEQ ID NOs: 1-89;
   iv. a guide sequence comprising any one of SEQ ID Nos: 1-24; and
   v. a guide sequence comprising any one of SEQ ID Nos: 1-6; or
b. a nucleic acid encoding a guide RNA of (a.); and optionally
c. an RNA-guided DNA binding agent or nucleic acid encoding an RNA-guided DNA binding agent.

Embodiment 004. A method of altering a DNA sequence within the TRAC gene, comprising delivering a composition to a cell, wherein the composition comprises:
a. a guide RNA comprising a sequence chosen from:
   i. a guide sequence selected from SEQ ID NOs: 90-178;
   ii. at least 17, 18, 19, or 20 contiguous nucleotides of a sequence selected from SEQ ID NOs: 90-178;
   iii. a guide sequence that is at least 99%, 98%, 97%, 96%, 95%, 94%, 93%, 92%, 91%, or 90% identical to a sequence selected from SEQ ID NOs: 90-178;
   iv. a guide sequence comprising any one of SEQ ID Nos: 90-113; and
   v. a guide sequence comprising any one of SEQ ID Nos: 90-95; or
b. a nucleic acid encoding a guide RNA of (a.); and optionally
c. an RNA-guided DNA binding agent or a nucleic acid encoding an RNA-guided DNA binding agent.

Embodiment 005. A method of reducing the expression of the TRAC gene comprising delivering a composition to a cell, wherein the composition comprises:
a. a guide RNA comprising a sequence chosen from:
   i. a guide sequence selected from SEQ ID NOs: 90-178;
   ii. at least 17, 18, 19, or 20 contiguous nucleotides of a sequence selected from SEQ ID NOs: 90-178;
   iii. a guide sequence that is at least 99%, 98%, 97%, 96%, 95%, 94%, 93%, 92%, 91%, or 90% identical to a sequence selected from SEQ ID NOs: 90-178;
   iv. a guide sequence comprising any one of SEQ ID Nos: 90-113; and
   v. a guide sequence comprising any one of SEQ ID Nos: 90-95; or
b. a nucleic acid encoding a guide RNA of (a.); and optionally
c. an RNA-guided DNA binding agent or a nucleic acid encoding an RNA-guided DNA binding agent.

Embodiment 006. A method of immunotherapy comprising administering a composition to a subject, an autologous cell thereof, and/or an allogeneic cell, wherein the composition comprises:
a. a guide RNA comprising a sequence chosen from:
   i. a guide sequence selected from SEQ ID NOs: 90-178;
   ii. at least 17, 18, 19, or 20 contiguous nucleotides of a sequence selected from SEQ ID NOs: 90-178;
   iii. a guide sequence that is at least 99%, 98%, 97%, 96%, 95%, 94%, 93%, 92%, 91%, or 90% identical to a sequence selected from SEQ ID NOs: 90-178;
   iv. a guide sequence comprising any one of SEQ ID Nos: 90-113; and
   v. a guide sequence comprising any one of SEQ ID Nos: 90-95; or
b. a nucleic acid encoding a guide RNA of (a.); and optionally
c. an RNA-guided DNA binding agent or nucleic acid encoding an RNA-guided DNA binding agent.

Embodiment 007. A method of altering a DNA sequence within the TRBC1, TRBC2 and/or TRAC genes, comprising delivering to a cell a first guide RNA, a second guide RNA and optionally an RNA-guided DNA binding agent or a nucleic acid encoding an RNA-guided DNA binding agent, wherein the first guide RNA comprises a sequence chosen from:
i. a guide sequence selected from SEQ ID NOs: 1-89;
ii. at least 17, 18, 19, or 20 contiguous nucleotides of a sequence selected from SEQ ID NOs: 1-89;
iii. a guide sequence that is at least 99%, 98%, 97%, 96%, 95%, 94%, 93%, 92%, 91%, or 90% identical to a sequence selected from SEQ ID NOs: 1-89;
iv. a guide sequence comprising any one of SEQ ID Nos: 1-24; and
v. a guide sequence comprising any one of SEQ ID Nos: 1-6, and
   wherein the second guide RNA comprises a sequence chosen from:
i. a guide sequence selected from SEQ ID NOs: 90-178;
ii. at least 17, 18, 19, or 20 contiguous nucleotides of a sequence selected from SEQ ID NOs: 90-178;
iii. a guide sequence that is at least 99%, 98%, 97%, 96%, 95%, 94%, 93%, 92%, 91%, or 90% identical to a sequence selected from SEQ ID NOs: 90-178;
iv. a guide sequence comprising any one of SEQ ID Nos: 90-113; and
v. a guide sequence comprising any one of SEQ ID Nos: 90-95.

Embodiment 008. A method of reducing the expression of the TRBC1, TRBC2 and/or TRAC genes, comprising delivering to a cell a first guide RNA, a second guide RNA and optionally an RNA-guided DNA binding agent or a nucleic acid encoding an RNA-guided DNA binding agent, wherein the first guide RNA comprises a sequence chosen from:
i. a guide sequence selected from SEQ ID NOs: 1-89;
ii. at least 17, 18, 19, or 20 contiguous nucleotides of a sequence selected from SEQ ID NOs: 1-89;
iii. a guide sequence that is at least 99%, 98%, 97%, 96%, 95%, 94%, 93%, 92%, 91%, or 90% identical to a sequence selected from SEQ ID NOs: 1-89;
iv. a guide sequence comprising any one of SEQ ID Nos: 1-24; and
v. a guide sequence comprising any one of SEQ ID Nos: 1-6, and
   wherein the second guide RNA comprises a sequence chosen from:
i. a guide sequence selected from SEQ ID NOs: 90-178;
ii. at least 17, 18, 19, or 20 contiguous nucleotides of a sequence selected from SEQ ID NOs: 90-178;
iii. a guide sequence that is at least 99%, 98%, 97%, 96%, 95%, 94%, 93%, 92%, 91%, or 90% identical to a sequence selected from SEQ ID NOs: 90-178;
iv. a guide sequence comprising any one of SEQ ID Nos: 90-113; and
v. a guide sequence comprising any one of SEQ ID Nos: 90-95.

Embodiment 009. A method of immunotherapy comprising administering a composition to a subject, an autologous cell thereof, or an allogeneic cell, wherein the composition comprises: a first guide RNA, a second guide RNA, and optionally an RNA-guided DNA binding agent or a nucleic acid encoding an RNA-guided DNA binding agent, wherein the first guide RNA comprises a sequence chosen from:
i. a guide sequence selected from SEQ ID NOs: 1-89;
ii. at least 17, 18, 19, or 20 contiguous nucleotides of a sequence selected from SEQ ID NOs: 1-89;
iii. a guide sequence that is at least 99%, 98%, 97%, 96%, 95%, 94%, 93%, 92%, 91%, or 90% identical to a sequence selected from SEQ ID NOs: 1-89;
iv. a guide sequence comprising any one of SEQ ID Nos: 1-24; and
v. a guide sequence comprising any one of SEQ ID Nos: 1-6, and
   wherein the second guide RNA comprises a sequence chosen from:
i. a guide sequence selected from SEQ ID NOs: 90-178;
ii. at least 17, 18, 19, or 20 contiguous nucleotides of a sequence selected from SEQ ID NOs: 90-178;
iii. a guide sequence that is at least 99%, 98%, 97%, 96%, 95%, 94%, 93%, 92%, 91%, or 90% identical to a sequence selected from SEQ ID NOs: 90-178;
iv. a guide sequence comprising any one of SEQ ID Nos: 90-113; and
v. a guide sequence comprising any one of SEQ ID Nos: 90-95.

Embodiment 010. The method of any one of the preceding Embodiments, wherein an RNA-guided DNA binding agent or nucleic acid encoding an RNA-guided DNA binding agent is administered.

Embodiment 011. A composition comprising:
a. a guide RNA comprising
   i. a guide sequence selected from SEQ ID NOs: 1-89; or
   ii. at least 17, 18, 19, or 20 contiguous nucleotides of a sequence selected from SEQ ID NOs: 1-89; or
   iii. a guide sequence that is at least 99%, 98%, 97%, 96%, 95%, 94%, 93%, 92%, 91%, or 90% identical to a sequence selected from SEQ ID NOs: 1-89; or
   iv. a guide sequence comprising any one of SEQ ID Nos: 1-24; or
   v. a guide sequence comprising any one of SEQ ID Nos: 1-6; and optionally
b. an RNA-guided DNA binding agent or nucleic acid encoding an RNA-guided DNA binding agent.

Embodiment 012. The composition of Embodiment 11, for use in altering a DNA sequence within the TRBC1 and/or TRBC2 genes in a cell.

Embodiment 013. The composition of Embodiment 11, for use in reducing the expression of the TRBC1 and/or TRBC2 genes in a cell.

Embodiment 014. A composition comprising:
a. a guide RNA comprising:
   i. a guide sequence selected from SEQ ID NOs: 90-178; or
   ii. at least 17, 18, 19, or 20 contiguous nucleotides of a sequence selected from SEQ ID NOs: 90-178; or
   iii. a guide sequence that is at least 99%, 98%, 97%, 96%, 95%, 94%, 93%, 92%, 91%, or 90% identical to a sequence selected from SEQ ID NOs: 90-178; or
   iv. a guide sequence comprising any one of SEQ ID Nos: 90-113; or
   v. a guide sequence comprising any one of SEQ ID Nos: 90-95; and optionally
b. an RNA-guided DNA binding agent or nucleic acid encoding an RNA-guided DNA binding agent.

Embodiment 015. The composition of Embodiment 14, for use in altering a DNA sequence within the TRAC gene in a cell.

Embodiment 016. The composition of Embodiment 14, for use in reducing the expression of the TRAC gene in a cell.

Embodiment 017. The composition of any of Embodiments 11-17, for use in immunotherapy of a subject.

Embodiment 018. A cell, altered by the method of any of Embodiments 1-10.

Embodiment 019. The cell according to Embodiment 18, wherein the cell is altered ex vivo.

Embodiment 020. The cell according to Embodiment 18 or Embodiment 19, wherein the cell is a T cell.

Embodiment 021. The cell according to any of Embodiments 18-20, wherein the cell is a CD3⁺, CD4⁺, and/or CD8⁺ T cell.

Embodiment 022. The cell according to any of Embodiments 18-21, wherein the cell is a mammalian, primate, or human cell.

Embodiment 023. The cell according to any of Embodiments 18-21, for use in immunotherapy of a subject.

Embodiment 024. The method of any of Embodiments 1-10, further comprising:
a. inducing a double-stranded break (DSB) within the TRBC1, TRBC2 and/or TRAC genes in a cell and/or subject; or
b. inducing a single-stranded break (SSB) within the TRBC1, TRBC2 and/or TRAC genes in a cell and/or a subject; or
c. reducing the expression of the TRBC1, TRBC2 and/or TRAC genes in a cell and/or subject.

Embodiment 025. The method or composition for use in any one of Embodiments 1-3, 7-13, wherein the composition results in editing of the TRBC1 and/or TRBC2 genes.

Embodiment 026. The method or composition for use in any one of Embodiments 4-10 and 14-17, wherein the composition results in editing of the TRAC gene.

Embodiment 027. The method or composition for use in any one of Embodiments 7-10, wherein the composition results in editing of a TRBC gene and the TRAC gene.

Embodiment 028. The method or composition for use in any of Embodiments 25-27, wherein the editing is calculated as a percentage of the population that is edited (percent editing or percent indels).

Embodiment 029. The method or composition for use in any of Embodiments 25-28, wherein the percent editing is between 10 and 100% of the population, e.g. between 30 and 99% of the population.

Embodiment 030. The method or composition for use in any of Embodiments 25-29, wherein the percent editing is between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the population.

Embodiment 031. The method or composition of any one of Embodiments 1-3, 7-13, 17, and 24-30, wherein the composition comprises a sgRNA comprising
a. any one of SEQ ID NOs: 179-184; or
b. a guide sequence selected from any one of SEQ ID NOs: 1-89; or
c. a guide sequence selected from SEQ ID Nos: 1-24; or
d. a guide sequence selected from SEQ ID Nos: 1-6.

Embodiment 032. The method or composition of any one of Embodiments 4-10, 14-17, and 24-30, wherein the composition comprises a sgRNA comprising
a. a guide sequence selected from any one of SEQ ID NOs: 90-178; or
b. a guide sequence selected from SEQ ID Nos: 90-113; or
c. a guide sequence selected from SEQ ID Nos: 90-95.

Embodiment 033. The method or composition of any one of Embodiments 1-17 and 24-32, wherein the target sequence is in exon 1, 2, 3, or 4 of the TRBC1, TRBC2 and/or TRAC genes.

Embodiment 034. The method or composition of Embodiment 33, wherein the target sequence is in exon 1 of the TRBC1, TRBC2 and/or TRAC genes.

Embodiment 035. The method or composition of Embodiment 33, wherein the target sequence is in exon 2 of the TRBC1, TRBC2 and/or TRAC genes.

Embodiment 036. The method or composition of Embodiment 33, wherein the target sequence is in exon 3 of the TRBC1, TRBC2 and/or TRAC genes.

Embodiment 037. The method or composition of Embodiment 33, wherein the target sequence is in exon 4 of the TRBC1, TRBC2 and/or TRAC genes.

Embodiment 038. The method or composition of any of Embodiments 33-37, wherein the target sequence is in the human TRBC1, TRBC2 and/or TRAC genes.

Embodiment 039. The method or composition of any one of Embodiments 1-17 or 24-38, wherein the guide sequence is complementary to a target sequence in the positive strand of TRBC1, TRBC2 and/or TRAC.

Embodiment 040. The method or composition of any one of Embodiments 1-17 or 24-38, wherein the guide sequence is complementary to a target sequence in the negative strand of the TRBC1, TRBC2 and/or TRAC genes.

Embodiment 041. The method or composition of any one of Embodiments 1-17 or 24-40, wherein the first guide sequence is complementary to a first target sequence in the positive strand of the TRBC1, TRBC2 and/or TRAC genes, and wherein the composition further comprises a second guide sequence that is complementary to a second target sequence in the negative strand of the TRBC1, TRBC2 and/or TRAC genes.

Embodiment 042. The method or composition of any one of any one of Embodiments 1-17 or 24-41, wherein the guide RNA comprises a guide sequence selected from any one of SEQ ID Nos 1-178 and further comprises a nucleotide sequence of SEQ ID NO: 200, wherein the nucleotides of SEQ ID NO: 200 follow the guide sequence at its 3′ end.

Embodiment 043. The method or composition of any one of any one of Embodiments 1-17 or 24-41, wherein the guide RNA comprises a guide sequence selected from any one of SEQ ID Nos 1-178 and further comprises a nucleotide sequence of SEQ ID NO: 201, wherein the nucleotides of SEQ ID NO: 201 follow the guide sequence at its 3′ end.

Embodiment 044. The method or composition of any one of any one of Embodiments 1-17 or 24-43, wherein the guide RNA is modified according to the pattern of SEQ ID NO: 300, wherein the N's are collectively any one of the guide sequences of Table 1 (SEQ ID Nos 1-89).

Embodiment 045. The method or composition of Embodiment 44, wherein each N in SEQ ID NO: 300 is any natural or non-natural nucleotide, wherein the N's form the guide sequence, and the guide sequence targets Cas9 to the TRBC1, TRBC2 and/or TRAC genes.

Embodiment 046. The method or composition of any one of Embodiments 42-44, wherein the sgRNA comprises a guide sequence that is at least 99%, 98%, 97%, 96%, 95%, 94%, 93%, 92%, 91%, or 90% identical to a sequence selected from SEQ ID Nos: 1-89.

Embodiment 047. The method or composition any one of any one of Embodiments 1-17 or 24-46, wherein the guide RNA comprises at least one modification.

Embodiment 048. The method or composition of Embodiment 47, wherein the at least one modification includes a 2′-O-methyl (2′-O-Me) modified nucleotide.

Embodiment 049. The method or composition of Embodiment 47 or 48, comprising a phosphorothioate (PS) bond between nucleotides.

Embodiment 050. The method or composition of any one of Embodiments 47-49, comprising a 2′-fluoro (2′-F) modified nucleotide.

Embodiment 051. The method or composition of any one of Embodiments 47-50, comprising a modification at one or more of the first five nucleotides at the 5′ end of the guide RNA.

Embodiment 052. The method or composition of any one of Embodiments 47-51, comprising a modification at one or more of the last five nucleotides at the 3′ end of the guide RNA.

Embodiment 053. The method or composition of any one of Embodiments 47-52, comprising a PS bond between the first four nucleotides of the guide RNA.

Embodiment 054. The method or composition of any one of Embodiments 47-53, comprising a PS bond between the last four nucleotides of the guide RNA.

Embodiment 055. The method or composition of any one of Embodiments 47-54, comprising a 2′-O-Me modified nucleotide at the first three nucleotides at the 5′ end of the guide RNA.

Embodiment 056. The method or composition of any one of Embodiments 47-55, comprising a 2′-O-Me modified nucleotide at the last three nucleotides at the 3′ end of the guide RNA.

Embodiment 057. The method or composition of any one of Embodiments 47-56, wherein the guide RNA comprises the modified nucleotides of SEQ ID NO: 300.

Embodiment 058. The method or composition any one of any one of Embodiments 1-17 or 24-57, wherein the composition further comprises a pharmaceutically acceptable excipient.

Embodiment 059. The method or composition of any one of Embodiments 1-58, wherein the guide RNA is associated with a lipid nanoparticle (LNP).

Embodiment 060. The method or composition of Embodiment 59, wherein the LNP comprises a biodegradable, ionizable lipid.

Embodiment 061. The method or composition of Embodiment 60, wherein the ionizable lipid is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate.

Embodiment 062. The method or composition of any one of Embodiments 59-61, wherein the LNP comprises a neutral lipid.

Embodiment 063. The method or composition of Embodiment 62, wherein the neutral lipid is DSPC.

Embodiment 064. The method or composition of any one of Embodiments 59-63, wherein the LNP comprises a helper lipid.

Embodiment 065. The method or composition of Embodiment 64, wherein the helper lipid is cholesterol.

Embodiment 066. The method or composition of any one of Embodiments 59-65, wherein the LNP comprises a stealth lipid.

Embodiment 067. The method or composition of Embodiment 66, wherein the stealth lipid is PEG2k-DMG.

Embodiment 068. The method or composition any one of any one of Embodiments 1-17 or 24-66, wherein the composition further comprises an RNA-guided DNA binding agent.

Embodiment 069. The method or composition any one of any one of Embodiments 1-17 or 24-68, wherein the composition further comprises an mRNA that encodes an RNA-guided DNA binding agent.

Embodiment 070. The method or composition of Embodiment 68 or 69, wherein the RNA-guided DNA binding agent is Cas9.

Embodiment 071. The method or composition any one of any one of Embodiments 1-17 or 24-70, wherein the composition is a pharmaceutical formulation and further comprises a pharmaceutically acceptable carrier.

Embodiment 072. The method, composition or cell of any one of Embodiments 1-3, 7-13, 17, 24, 25, 27-31, or 33-71, wherein the sequence selected from SEQ ID NOs: 1-89 is SEQ ID NO: 1.

Embodiment 073. The method or composition of any one of Embodiments 1-3, 7-13, 17, 24, 25, 27-31, or 33-71, wherein the sequence selected from SEQ ID NOs: 1-89 is SEQ ID NO: 2.

Embodiment 074. The method or composition of any one of Embodiments 1-3, 7-13, 17, 24, 25, 27-31, or 33-71, wherein the sequence selected from SEQ ID NOs: 1-89 is SEQ ID NO: 3.

Embodiment 075. The method or composition of any one of Embodiments 1-3, 7-13, 17, 24, 25, 27-31, or 33-71, wherein the sequence selected from SEQ ID NOs: 1-89 is SEQ ID NO: 4.

Embodiment 076. The method or composition of any one of Embodiments 1-3, 7-13, 17, 24, 25, 27-31, or 33-71, wherein the sequence selected from SEQ ID NOs: 1-89 is SEQ ID NO: 5.

Embodiment 077. The method or composition of any one of Embodiments 1-3, 7-13, 17, 24, 25, 27-31, or 33-71, wherein the sequence selected from SEQ ID NOs: 1-89 is SEQ ID NO: 6.

Embodiment 078. The method or composition of any one of Embodiments 1-3, 7-13, 17, 24, 25, 27-31, or 33-71, wherein the sequence selected from SEQ ID NOs: 1-89 is SEQ ID NO: 7.

Embodiment 079. The method or composition of any one of Embodiments 1-3, 7-13, 17, 24, 25, 27-31, or 33-71, wherein the sequence selected from SEQ ID NOs: 1-89 is SEQ ID NO: 8.

Embodiment 080. The method or composition of any one of Embodiments 1-3, 7-13, 17, 24, 25, 27-31, or 33-71, wherein the sequence selected from SEQ ID NOs: 1-89 is SEQ ID NO: 9.

Embodiment 081. The method or composition of any one of Embodiments 1-3, 7-13, 17, 24, 25, 27-31, or 33-71, wherein the sequence selected from SEQ ID NOs: 1-89 is SEQ ID NO: 10.

Embodiment 082. The method or composition of any one of Embodiments 1-3, 7-13, 17, 24, 25, 27-31, or 33-71, wherein the sequence selected from SEQ ID NOs: 1-89 is SEQ ID NO: 11.

Embodiment 083. The method or composition of any one of Embodiments 1-3, 7-13, 17, 24, 25, 27-31, or 33-71, wherein the sequence selected from SEQ ID NOs: 1-89 is SEQ ID NO: 12.

Embodiment 084. The method or composition of any one of Embodiments 1-3, 7-13, 17, 24, 25, 27-31, or 33-71, wherein the sequence selected from SEQ ID NOs: 1-89 is SEQ ID NO: 13.

Embodiment 085. The method or composition of any one of Embodiments 1-3, 7-13, 17, 24, 25, 27-31, or 33-71, wherein the sequence selected from SEQ ID NOs: 1-89 is SEQ ID NO: 14.

Embodiment 086. The method or composition of any one of Embodiments 1-3, 7-13, 17, 24, 25, 27-31, or 33-71, wherein the sequence selected from SEQ ID NOs: 1-89 is SEQ ID NO: 15.

Embodiment 087. The method or composition of any one of Embodiments 1-3, 7-13, 17, 24, 25, 27-31, or 33-71, wherein the sequence selected from SEQ ID NOs: 1-89 is SEQ ID NO: 16.

Embodiment 088. The method or composition of any one of Embodiments 1-3, 7-13, 17, 24, 25, 27-31, or 33-71, wherein the sequence selected from SEQ ID NOs: 1-89 is SEQ ID NO: 17.

Embodiment 089. The method or composition of any one of Embodiments 1-3, 7-13, 17, 24, 25, 27-31, or 33-71, wherein the sequence selected from SEQ ID NOs: 1-89 is SEQ ID NO: 18.

Embodiment 090. The method or composition of any one of Embodiments 1-3, 7-13, 17, 24, 25, 27-31, or 33-71, wherein the sequence selected from SEQ ID NOs: 1-89 is SEQ ID NO: 19.

Embodiment 091. The method or composition of any one of Embodiments 1-3, 7-13, 17, 24, 25, 27-31, or 33-71, wherein the sequence selected from SEQ ID NOs: 1-89 is SEQ ID NO: 20.

Embodiment 092. The method or composition of any one of Embodiments 1-3, 7-13, 17, 24, 25, 27-31, or 33-71, wherein the sequence selected from SEQ ID NOs: 1-89 is SEQ ID NO: 21.

Embodiment 093. The method or composition of any one of Embodiments 1-3, 7-13, 17, 24, 25, 27-31, or 33-71, wherein the sequence selected from SEQ ID NOs: 1-89 is SEQ ID NO: 22.

Embodiment 094. The method or composition of any one of Embodiments 1-3, 7-13, 17, 24, 25, 27-31, or 33-71, wherein the sequence selected from SEQ ID NOs: 1-89 is SEQ ID NO: 23.

Embodiment 095. The method or composition of any one of Embodiments 1-3, 7-13, 17, 24, 25, 27-31, or 33-71, wherein the sequence selected from SEQ ID NOs: 1-89 is SEQ ID NO: 24.

Embodiment 096. The method or composition of any one of Embodiments 1-3, 7-13, 17, 24, 25, 27-31, or 33-71, wherein the sequence selected from SEQ ID NOs: 1-89 is SEQ ID NO: 25.

Embodiment 097. The method or composition of any one of Embodiments 1-3, 7-13, 17, 24, 25, 27-31, or 33-71, wherein the sequence selected from SEQ ID NOs: 1-89 is SEQ ID NO: 26.

Embodiment 098. The method or composition of any one of Embodiments 1-3, 7-13, 17, 24, 25, 27-31, or 33-71, wherein the sequence selected from SEQ ID NOs: 1-89 is SEQ ID NO: 27.

Embodiment 099. The method or composition of any one of Embodiments 1-3, 7-13, 17, 24, 25, 27-31, or 33-71, wherein the sequence selected from SEQ ID NOs: 1-89 is SEQ ID NO: 28.

Embodiment 100. The method or composition of any one of Embodiments 1-3, 7-13, 17, 24, 25, 27-31, or 33-71, wherein the sequence selected from SEQ ID NOs: 1-89 is SEQ ID NO: 29.

Embodiment 101. The method or composition of any one of Embodiments 1-3, 7-13, 17, 24, 25, 27-31, or 33-71, wherein the sequence selected from SEQ ID NOs: 1-89 is SEQ ID NO: 30.

Embodiment 102. The method or composition of any one of Embodiments 1-3, 7-13, 17, 24, 25, 27-31, or 33-71, wherein the sequence selected from SEQ ID NOs: 1-89 is SEQ ID NO: 31.

Embodiment 103. The method or composition of any one of Embodiments 1-3, 7-13, 17, 24, 25, 27-31, or 33-71, wherein the sequence selected from SEQ ID NOs: 1-89 is SEQ ID NO: 32.

Embodiment 104. The method or composition of any one of Embodiments 1-3, 7-13, 17, 24, 25, 27-31, or 33-71, wherein the sequence selected from SEQ ID NOs: 1-89 is SEQ ID NO: 33.

Embodiment 105. The method or composition of any one of Embodiments 1-3, 7-13, 17, 24, 25, 27-31, or 33-71, wherein the sequence selected from SEQ ID NOs: 1-89 is SEQ ID NO: 34.

Embodiment 106. The method or composition of any one of Embodiments 1-3, 7-13, 17, 24, 25, 27-31, or 33-71, wherein the sequence selected from SEQ ID NOs: 1-89 is SEQ ID NO: 35.

Embodiment 107. The method or composition of any one of Embodiments 1-3, 7-13, 17, 24, 25, 27-31, or 33-71, wherein the sequence selected from SEQ ID NOs: 1-89 is SEQ ID NO: 36.

Embodiment 108. The method or composition of any one of Embodiments 1-3, 7-13, 17, 24, 25, 27-31, or 33-71, wherein the sequence selected from SEQ ID NOs: 1-89 is SEQ ID NO: 37.

Embodiment 109. The method or composition of any one of Embodiments 1-3, 7-13, 17, 24, 25, 27-31, or 33-71, wherein the sequence selected from SEQ ID NOs: 1-89 is SEQ ID NO: 38.

Embodiment 110. The method or composition of any one of Embodiments 1-3, 7-13, 17, 24, 25, 27-31, or 33-71, wherein the sequence selected from SEQ ID NOs: 1-89 is SEQ ID NO: 39.

Embodiment 111. The method or composition of any one of Embodiments 1-3, 7-13, 17, 24, 25, 27-31, or 33-71, wherein the sequence selected from SEQ ID NOs: 1-89 is SEQ ID NO: 40.

Embodiment 112. The method or composition of any one of Embodiments 1-3, 7-13, 17, 24, 25, 27-31, or 33-71, wherein the sequence selected from SEQ ID NOs: 1-89 is SEQ ID NO: 41.

Embodiment 113. The method or composition of any one of Embodiments 1-3, 7-13, 17, 24, 25, 27-31, or 33-71, wherein the sequence selected from SEQ ID NOs: 1-89 is SEQ ID NO: 42.

Embodiment 114. The method or composition of any one of Embodiments 1-3, 7-13, 17, 24, 25, 27-31, or 33-71, wherein the sequence selected from SEQ ID NOs: 1-89 is SEQ ID NO: 43.

Embodiment 115. The method or composition of any one of Embodiments 1-3, 7-13, 17, 24, 25, 27-31, or 33-71, wherein the sequence selected from SEQ ID NOs: 1-89 is SEQ ID NO: 44.

Embodiment 116. The method or composition of any one of Embodiments 1-3, 7-13, 17, 24, 25, 27-31, or 33-71, wherein the sequence selected from SEQ ID NOs: 1-89 is SEQ ID NO: 45.

Embodiment 117. The method or composition of any one of Embodiments 1-3, 7-13, 17, 24, 25, 27-31, or 33-71, wherein the sequence selected from SEQ ID NOs: 1-89 is SEQ ID NO: 46.

Embodiment 118. The method or composition of any one of Embodiments 1-3, 7-13, 17, 24, 25, 27-31, or 33-71, wherein the sequence selected from SEQ ID NOs: 1-89 is SEQ ID NO: 47.

Embodiment 119. The method or composition of any one of Embodiments 1-3, 7-13, 17, 24, 25, 27-31, or 33-71, wherein the sequence selected from SEQ ID NOs: 1-89 is SEQ ID NO: 48.

Embodiment 120. The method or composition of any one of Embodiments 1-3, 7-13, 17, 24, 25, 27-31, or 33-71, wherein the sequence selected from SEQ ID NOs: 1-89 is SEQ ID NO: 49.

Embodiment 121. The method or composition of any one of Embodiments 1-3, 7-13, 17, 24, 25, 27-31, or 33-71, wherein the sequence selected from SEQ ID NOs: 1-89 is SEQ ID NO: 50.

Embodiment 122. The method or composition of any one of Embodiments 1-3, 7-13, 17, 24, 25, 27-31, or 33-71, wherein the sequence selected from SEQ ID NOs: 1-89 is SEQ ID NO: 51.

Embodiment 123. The method or composition of any one of Embodiments 1-3, 7-13, 17, 24, 25, 27-31, or 33-71, wherein the sequence selected from SEQ ID NOs: 1-89 is SEQ ID NO: 52.

Embodiment 124. The method or composition of any one of Embodiments 1-3, 7-13, 17, 24, 25, 27-31, or 33-71, wherein the sequence selected from SEQ ID NOs: 1-89 is SEQ ID NO: 53.

Embodiment 125. The method or composition of any one of Embodiments 1-3, 7-13, 17, 24, 25, 27-31, or 33-71, wherein the sequence selected from SEQ ID NOs: 1-89 is SEQ ID NO: 54.

Embodiment 126. The method or composition of any one of Embodiments 1-3, 7-13, 17, 24, 25, 27-31, or 33-71, wherein the sequence selected from SEQ ID NOs: 1-89 is SEQ ID NO: 55.

Embodiment 127. The method or composition of any one of Embodiments 1-3, 7-13, 17, 24, 25, 27-31, or 33-71, wherein the sequence selected from SEQ ID NOs: 1-89 is SEQ ID NO: 56.

Embodiment 128. The method or composition of any one of Embodiments 1-3, 7-13, 17, 24, 25, 27-31, or 33-71, wherein the sequence selected from SEQ ID NOs: 1-89 is SEQ ID NO: 57.

Embodiment 129. The method or composition of any one of Embodiments 1-3, 7-13, 17, 24, 25, 27-31, or 33-71, wherein the sequence selected from SEQ ID NOs: 1-89 is SEQ ID NO: 58.

Embodiment 130. The method or composition of any one of Embodiments 1-3, 7-13, 17, 24, 25, 27-31, or 33-71, wherein the sequence selected from SEQ ID NOs: 1-89 is SEQ ID NO: 59.

Embodiment 131. The method or composition of any one of Embodiments 1-3, 7-13, 17, 24, 25, 27-31, or 33-71, wherein the sequence selected from SEQ ID NOs: 1-89 is SEQ ID NO: 60.

Embodiment 132. The method or composition of any one of Embodiments 1-3, 7-13, 17, 24, 25, 27-31, or 33-71, wherein the sequence selected from SEQ ID NOs: 1-89 is SEQ ID NO: 61.

Embodiment 133. The method or composition of any one of Embodiments 1-3, 7-13, 17, 24, 25, 27-31, or 33-71, wherein the sequence selected from SEQ ID NOs: 1-89 is SEQ ID NO: 62.

Embodiment 134. The method or composition of any one of Embodiments 1-3, 7-13, 17, 24, 25, 27-31, or 33-71, wherein the sequence selected from SEQ ID NOs: 1-89 is SEQ ID NO: 63.

Embodiment 135. The method or composition of any one of Embodiments 1-3, 7-13, 17, 24, 25, 27-31, or 33-71, wherein the sequence selected from SEQ ID NOs: 1-89 is SEQ ID NO: 64.

Embodiment 136. The method or composition of any one of Embodiments 1-3, 7-13, 17, 24, 25, 27-31, or 33-71, wherein the sequence selected from SEQ ID NOs: 1-89 is SEQ ID NO: 65.

Embodiment 137. The method or composition of any one of Embodiments 1-3, 7-13, 17, 24, 25, 27-31, or 33-71, wherein the sequence selected from SEQ ID NOs: 1-89 is SEQ ID NO: 66.

Embodiment 138. The method or composition of any one of Embodiments 1-3, 7-13, 17, 24, 25, 27-31, or 33-71, wherein the sequence selected from SEQ ID NOs: 1-89 is SEQ ID NO: 67.

Embodiment 139. The method or composition of any one of Embodiments 1-3, 7-13, 17, 24, 25, 27-31, or 33-71, wherein the sequence selected from SEQ ID NOs: 1-89 is SEQ ID NO: 68.

Embodiment 140. The method or composition of any one of Embodiments 1-3, 7-13, 17, 24, 25, 27-31, or 33-71, wherein the sequence selected from SEQ ID NOs: 1-89 is SEQ ID NO: 69.

Embodiment 141. The method or composition of any one of Embodiments 1-3, 7-13, 17, 24, 25, 27-31, or 33-71, wherein the sequence selected from SEQ ID NOs: 1-89 is SEQ ID NO: 70.

Embodiment 142. The method or composition of any one of Embodiments 1-3, 7-13, 17, 24, 25, 27-31, or 33-71, wherein the sequence selected from SEQ ID NOs: 1-89 is SEQ ID NO: 71.

Embodiment 143. The method or composition of any one of Embodiments 1-3, 7-13, 17, 24, 25, 27-31, or 33-71, wherein the sequence selected from SEQ ID NOs: 1-89 is SEQ ID NO: 72.

Embodiment 144. The method or composition of any one of Embodiments 1-3, 7-13, 17, 24, 25, 27-31, or 33-71, wherein the sequence selected from SEQ ID NOs: 1-89 is SEQ ID NO: 73.

Embodiment 145. The method or composition of any one of Embodiments 1-3, 7-13, 17, 24, 25, 27-31, or 33-71, wherein the sequence selected from SEQ ID NOs: 1-89 is SEQ ID NO: 74.

Embodiment 146. The method or composition of any one of Embodiments 1-3, 7-13, 17, 24, 25, 27-31, or 33-71, wherein the sequence selected from SEQ ID NOs: 1-89 is SEQ ID NO: 75.

Embodiment 147. The method or composition of any one of Embodiments 1-3, 7-13, 17, 24, 25, 27-31, or 33-71, wherein the sequence selected from SEQ ID NOs: 1-89 is SEQ ID NO: 76.

Embodiment 148. The method or composition of any one of Embodiments 1-3, 7-13, 17, 24, 25, 27-31, or 33-71, wherein the sequence selected from SEQ ID NOs: 1-89 is SEQ ID NO: 77.

Embodiment 149. The method or composition of any one of Embodiments 1-3, 7-13, 17, 24, 25, 27-31, or 33-71, wherein the sequence selected from SEQ ID NOs: 1-89 is SEQ ID NO: 78.

Embodiment 150. The method or composition of any one of Embodiments 1-3, 7-13, 17, 24, 25, 27-31, or 33-71, wherein the sequence selected from SEQ ID NOs: 1-89 is SEQ ID NO: 79.

Embodiment 151. The method or composition of any one of Embodiments 1-3, 7-13, 17, 24, 25, 27-31, or 33-71, wherein the sequence selected from SEQ ID NOs: 1-89 is SEQ ID NO: 80.

Embodiment 152. The method or composition of any one of Embodiments 1-3, 7-13, 17, 24, 25, 27-31, or 33-71, wherein the sequence selected from SEQ ID NOs: 1-89 is SEQ ID NO: 81.

Embodiment 153. The method or composition of any one of Embodiments 1-3, 7-13, 17, 24, 25, 27-31, or 33-71, wherein the sequence selected from SEQ ID NOs: 1-89 is SEQ ID NO: 82.

Embodiment 154. The method or composition of any one of Embodiments 1-3, 7-13, 17, 24, 25, 27-31, or 33-71, wherein the sequence selected from SEQ ID NOs: 1-89 is SEQ ID NO: 83.

Embodiment 155. The method or composition of any one of Embodiments 1-3, 7-13, 17, 24, 25, 27-31, or 33-71, wherein the sequence selected from SEQ ID NOs: 1-89 is SEQ ID NO: 84.

Embodiment 156. The method or composition of any one of Embodiments 1-3, 7-13, 17, 24, 25, 27-31, or 33-71, wherein the sequence selected from SEQ ID NOs: 1-89 is SEQ ID NO: 85.

Embodiment 157. The method or composition of any one of Embodiments 1-3, 7-13, 17, 24, 25, 27-31, or 33-71, wherein the sequence selected from SEQ ID NOs: 1-89 is SEQ ID NO: 86.

Embodiment 158. The method or composition of any one of Embodiments 1-3, 7-13, 17, 24, 25, 27-31, or 33-71, wherein the sequence selected from SEQ ID NOs: 1-89 is SEQ ID NO: 87.

Embodiment 159. The method or composition of any one of Embodiments 1-3, 7-13, 17, 24, 25, 27-31, or 33-71, wherein the sequence selected from SEQ ID NOs: 1-89 is SEQ ID NO: 88.

Embodiment 160. The method or composition of any one of Embodiments 1-3, 7-13, 17, 24, 25, 27-31, or 33-71, wherein the sequence selected from SEQ ID NOs: 1-89 is SEQ ID NO: 89.

Embodiment 161. The method or composition of any one of Embodiments 4-10, 14-17, 24, 26-30, or 32-71, wherein the sequence selected from SEQ ID NOs: 90-178 is SEQ ID NO: 90.

Embodiment 162. The method or composition of any one of Embodiments 4-10, 14-17, 24, 26-30, or 32-71, wherein the sequence selected from SEQ ID NOs: 90-178 is SEQ ID NO: 91.

Embodiment 163. The method or composition of any one of Embodiments 4-10, 14-17, 24, 26-30, or 32-71, wherein the sequence selected from SEQ ID NOs: 90-178 is SEQ ID NO: 92.

Embodiment 164. The method or composition of any one of Embodiments 4-10, 14-17, 24, 26-30, or 32-71, wherein the sequence selected from SEQ ID NOs: 90-178 is SEQ ID NO: 93.

Embodiment 165. The method or composition of any one of Embodiments 4-10, 14-17, 24, 26-30, or 32-71, wherein the sequence selected from SEQ ID NOs: 90-178 is SEQ ID NO: 94.

Embodiment 166. The method or composition of any one of Embodiments 4-10, 14-17, 24, 26-30, or 32-71, wherein the sequence selected from SEQ ID NOs: 90-178 is SEQ ID NO: 95.

Embodiment 167. The method or composition of any one of Embodiments 4-10, 14-17, 24, 26-30, or 32-71, wherein the sequence selected from SEQ ID NOs: 90-178 is SEQ ID NO: 96.

Embodiment 168. The method or composition of any one of Embodiments 4-10, 14-17, 24, 26-30, or 32-71, wherein the sequence selected from SEQ ID NOs: 90-178 is SEQ ID NO: 97.

Embodiment 169. The method or composition of any one of Embodiments 4-10, 14-17, 24, 26-30, or 32-71, wherein the sequence selected from SEQ ID NOs: 90-178 is SEQ ID NO: 98.

Embodiment 170. The method or composition of any one of Embodiments 4-10, 14-17, 24, 26-30, or 32-71, wherein the sequence selected from SEQ ID NOs: 90-178 is SEQ ID NO: 99.

Embodiment 171. The method or composition of any one of Embodiments 4-10, 14-17, 24, 26-30, or 32-71, wherein the sequence selected from SEQ ID NOs: 90-178 is SEQ ID NO: 100.

Embodiment 172. The method or composition of any one of Embodiments 4-10, 14-17, 24, 26-30, or 32-71, wherein the sequence selected from SEQ ID NOs: 90-178 is SEQ ID NO: 101.

Embodiment 173. The method or composition of any one of Embodiments 4-10, 14-17, 24, 26-30, or 32-71, wherein the sequence selected from SEQ ID NOs: 90-178 is SEQ ID NO: 102.

Embodiment 174. The method or composition of any one of Embodiments 4-10, 14-17, 24, 26-30, or 32-71, wherein the sequence selected from SEQ ID NOs: 90-178 is SEQ ID NO: 103.

Embodiment 175. The method or composition of any one of Embodiments 4-10, 14-17, 24, 26-30, or 32-71, wherein the sequence selected from SEQ ID NOs: 90-178 is SEQ ID NO: 104.

Embodiment 176. The method or composition of any one of Embodiments 4-10, 14-17, 24, 26-30, or 32-71, wherein the sequence selected from SEQ ID NOs: 90-178 is SEQ ID NO: 105.

Embodiment 177. The method or composition of any one of Embodiments 4-10, 14-17, 24, 26-30, or 32-71, wherein the sequence selected from SEQ ID NOs: 90-178 is SEQ ID NO: 106.

Embodiment 178. The method or composition of any one of Embodiments 4-10, 14-17, 24, 26-30, or 32-71, wherein the sequence selected from SEQ ID NOs: 90-178 is SEQ ID NO: 107.

Embodiment 179. The method or composition of any one of Embodiments 4-10, 14-17, 24, 26-30, or 32-71, wherein the sequence selected from SEQ ID NOs: 90-178 is SEQ ID NO: 108.

Embodiment 180. The method or composition of any one of Embodiments 4-10, 14-17, 24, 26-30, or 32-71, wherein the sequence selected from SEQ ID NOs: 90-178 is SEQ ID NO: 109.

Embodiment 181. The method or composition of any one of Embodiments 4-10, 14-17, 24, 26-30, or 32-71, wherein the sequence selected from SEQ ID NOs: 90-178 is SEQ ID NO: 110.

Embodiment 182. The method or composition of any one of Embodiments 4-10, 14-17, 24, 26-30, or 32-71, wherein the sequence selected from SEQ ID NOs: 90-178 is SEQ ID NO: 111.

Embodiment 183. The method or composition of any one of Embodiments 4-10, 14-17, 24, 26-30, or 32-71, wherein the sequence selected from SEQ ID NOs: 90-178 is SEQ ID NO: 112.

Embodiment 184. The method or composition of any one of Embodiments 4-10, 14-17, 24, 26-30, or 32-71, wherein the sequence selected from SEQ ID NOs: 90-178 is SEQ ID NO: 113.

Embodiment 185. The method or composition of any one of Embodiments 4-10, 14-17, 24, 26-30, or 32-71, wherein the sequence selected from SEQ ID NOs: 90-178 is SEQ ID NO: 114.

Embodiment 186. The method or composition of any one of Embodiments 4-10, 14-17, 24, 26-30, or 32-71, wherein the sequence selected from SEQ ID NOs: 90-178 is SEQ ID NO: 115.

Embodiment 187. The method or composition of any one of Embodiments 4-10, 14-17, 24, 26-30, or 32-71, wherein the sequence selected from SEQ ID NOs: 90-178 is SEQ ID NO: 116.

Embodiment 188. The method or composition of any one of Embodiments 4-10, 14-17, 24, 26-30, or 32-71, wherein the sequence selected from SEQ ID NOs: 90-178 is SEQ ID NO: 117.

Embodiment 189. The method or composition of any one of Embodiments 4-10, 14-17, 24, 26-30, or 32-71, wherein the sequence selected from SEQ ID NOs: 90-178 is SEQ ID NO: 118.

Embodiment 190. The method or composition of any one of Embodiments 4-10, 14-17, 24, 26-30, or 32-71, wherein the sequence selected from SEQ ID NOs: 90-178 is SEQ ID NO: 119.

Embodiment 191. The method or composition of any one of Embodiments 4-10, 14-17, 24, 26-30, or 32-71, wherein the sequence selected from SEQ ID NOs: 90-178 is SEQ ID NO: 120.

Embodiment 192. The method or composition of any one of Embodiments 4-10, 14-17, 24, 26-30, or 32-71, wherein the sequence selected from SEQ ID NOs: 90-178 is SEQ ID NO: 121.

Embodiment 193. The method or composition of any one of Embodiments 4-10, 14-17, 24, 26-30, or 32-71, wherein the sequence selected from SEQ ID NOs: 90-178 is SEQ ID NO: 122.

Embodiment 194. The method or composition of any one of Embodiments 4-10, 14-17, 24, 26-30, or 32-71, wherein the sequence selected from SEQ ID NOs: 90-178 is SEQ ID NO: 123.

Embodiment 195. The method or composition of any one of Embodiments 4-10, 14-17, 24, 26-30, or 32-71, wherein the sequence selected from SEQ ID NOs: 90-178 is SEQ ID NO: 124.

Embodiment 196. The method or composition of any one of Embodiments 4-10, 14-17, 24, 26-30, or 32-71, wherein the sequence selected from SEQ ID NOs: 90-178 is SEQ ID NO: 125.

Embodiment 197. The method or composition of any one of Embodiments 4-10, 14-17, 24, 26-30, or 32-71, wherein the sequence selected from SEQ ID NOs: 90-178 is SEQ ID NO: 126.

Embodiment 198. The method or composition of any one of Embodiments 4-10, 14-17, 24, 26-30, or 32-71, wherein the sequence selected from SEQ ID NOs: 90-178 is SEQ ID NO: 127.

Embodiment 199. The method or composition of any one of Embodiments 4-10, 14-17, 24, 26-30, or 32-71, wherein the sequence selected from SEQ ID NOs: 90-178 is SEQ ID NO: 128.

Embodiment 200. The method or composition of any one of Embodiments 4-10, 14-17, 24, 26-30, or 32-71, wherein the sequence selected from SEQ ID NOs: 90-178 is SEQ ID NO: 129.

Embodiment 201. The method or composition of any one of Embodiments 4-10, 14-17, 24, 26-30, or 32-71, wherein the sequence selected from SEQ ID NOs: 90-178 is SEQ ID NO: 130.

Embodiment 202. The method or composition of any one of Embodiments 4-10, 14-17, 24, 26-30, or 32-71, wherein the sequence selected from SEQ ID NOs: 90-178 is SEQ ID NO: 131.

Embodiment 203. The method or composition of any one of Embodiments 4-10, 14-17, 24, 26-30, or 32-71, wherein the sequence selected from SEQ ID NOs: 90-178 is SEQ ID NO: 132.

Embodiment 204. The method or composition of any one of Embodiments 4-10, 14-17, 24, 26-30, or 32-71, wherein the sequence selected from SEQ ID NOs: 90-178 is SEQ ID NO: 133.

Embodiment 205. The method or composition of any one of Embodiments 4-10, 14-17, 24, 26-30, or 32-71, wherein the sequence selected from SEQ ID NOs: 90-178 is SEQ ID NO: 134.

Embodiment 206. The method or composition of any one of Embodiments 4-10, 14-17, 24, 26-30, or 32-71, wherein the sequence selected from SEQ ID NOs: 90-178 is SEQ ID NO: 135.

Embodiment 207. The method or composition of any one of Embodiments 4-10, 14-17, 24, 26-30, or 32-71, wherein the sequence selected from SEQ ID NOs: 90-178 is SEQ ID NO: 136.

Embodiment 208. The method or composition of any one of Embodiments 4-10, 14-17, 24, 26-30, or 32-71, wherein the sequence selected from SEQ ID NOs: 90-178 is SEQ ID NO: 137.

Embodiment 209. The method or composition of any one of Embodiments 4-10, 14-17, 24, 26-30, or 32-71, wherein the sequence selected from SEQ ID NOs: 90-178 is SEQ ID NO: 138.

Embodiment 210. The method or composition of any one of Embodiments 4-10, 14-17, 24, 26-30, or 32-71, wherein the sequence selected from SEQ ID NOs: 90-178 is SEQ ID NO: 139.

Embodiment 211. The method or composition of any one of Embodiments 4-10, 14-17, 24, 26-30, or 32-71, wherein the sequence selected from SEQ ID NOs: 90-178 is SEQ ID NO: 140.

Embodiment 212. The method or composition of any one of Embodiments 4-10, 14-17, 24, 26-30, or 32-71, wherein the sequence selected from SEQ ID NOs: 90-178 is SEQ ID NO: 141.

Embodiment 213. The method or composition of any one of Embodiments 4-10, 14-17, 24, 26-30, or 32-71, wherein the sequence selected from SEQ ID NOs: 90-178 is SEQ ID NO: 142.

Embodiment 214. The method or composition of any one of Embodiments 4-10, 14-17, 24, 26-30, or 32-71, wherein the sequence selected from SEQ ID NOs: 90-178 is SEQ ID NO: 143.

Embodiment 215. The method or composition of any one of Embodiments 4-10, 14-17, 24, 26-30, or 32-71, wherein the sequence selected from SEQ ID NOs: 90-178 is SEQ ID NO: 144.

Embodiment 216. The method or composition of any one of Embodiments 4-10, 14-17, 24, 26-30, or 32-71, wherein the sequence selected from SEQ ID NOs: 90-178 is SEQ ID NO: 145.

Embodiment 217. The method or composition of any one of Embodiments 4-10, 14-17, 24, 26-30, or 32-71, wherein the sequence selected from SEQ ID NOs: 90-178 is SEQ ID NO: 146.

Embodiment 218. The method or composition of any one of Embodiments 4-10, 14-17, 24, 26-30, or 32-71, wherein the sequence selected from SEQ ID NOs: 90-178 is SEQ ID NO: 147.

Embodiment 219. The method or composition of any one of Embodiments 4-10, 14-17, 24, 26-30, or 32-71, wherein the sequence selected from SEQ ID NOs: 90-178 is SEQ ID NO: 148.

Embodiment 220. The method or composition of any one of Embodiments 4-10, 14-17, 24, 26-30, or 32-71, wherein the sequence selected from SEQ ID NOs: 90-178 is SEQ ID NO: 149.

Embodiment 221. The method or composition of any one of Embodiments 4-10, 14-17, 24, 26-30, or 32-71, wherein the sequence selected from SEQ ID NOs: 90-178 is SEQ ID NO: 150.

Embodiment 222. The method or composition of any one of Embodiments 4-10, 14-17, 24, 26-30, or 32-71, wherein the sequence selected from SEQ ID NOs: 90-178 is SEQ ID NO: 151.

Embodiment 223. The method or composition of any one of Embodiments 4-10, 14-17, 24, 26-30, or 32-71, wherein the sequence selected from SEQ ID NOs: 90-178 is SEQ ID NO: 152.

Embodiment 224. The method or composition of any one of Embodiments 4-10, 14-17, 24, 26-30, or 32-71, wherein the sequence selected from SEQ ID NOs: 90-178 is SEQ ID NO: 153.

Embodiment 225. The method or composition of any one of Embodiments 4-10, 14-17, 24, 26-30, or 32-71, wherein the sequence selected from SEQ ID NOs: 90-178 is SEQ ID NO: 154.

Embodiment 226. The method or composition of any one of Embodiments 4-10, 14-17, 24, 26-30, or 32-71, wherein the sequence selected from SEQ ID NOs: 90-178 is SEQ ID NO: 155.

Embodiment 227. The method or composition of any one of Embodiments 4-10, 14-17, 24, 26-30, or 32-71, wherein the sequence selected from SEQ ID NOs: 90-178 is SEQ ID NO: 156.

Embodiment 228. The method or composition of any one of Embodiments 4-10, 14-17, 24, 26-30, or 32-71, wherein the sequence selected from SEQ ID NOs: 90-178 is SEQ ID NO: 157.

Embodiment 229. The method or composition of any one of Embodiments 4-10, 14-17, 24, 26-30, or 32-71, wherein the sequence selected from SEQ ID NOs: 90-178 is SEQ ID NO: 158.

Embodiment 230. The method or composition of any one of Embodiments 4-10, 14-17, 24, 26-30, or 32-71, wherein the sequence selected from SEQ ID NOs: 90-178 is SEQ ID NO: 159.

Embodiment 231. The method or composition of any one of Embodiments 4-10, 14-17, 24, 26-30, or 32-71, wherein the sequence selected from SEQ ID NOs: 90-178 is SEQ ID NO: 160.

Embodiment 232. The method or composition of any one of Embodiments 4-10, 14-17, 24, 26-30, or 32-71, wherein the sequence selected from SEQ ID NOs: 90-178 is SEQ ID NO: 161.

Embodiment 233. The method or composition of any one of Embodiments 4-10, 14-17, 24, 26-30, or 32-71, wherein the sequence selected from SEQ ID NOs: 90-178 is SEQ ID NO: 162.

Embodiment 234. The method or composition of any one of Embodiments 4-10, 14-17, 24, 26-30, or 32-71, wherein the sequence selected from SEQ ID NOs: 90-178 is SEQ ID NO: 163.

Embodiment 235. The method or composition of any one of Embodiments 4-10, 14-17, 24, 26-30, or 32-71, wherein the sequence selected from SEQ ID NOs: 90-178 is SEQ ID NO: 164.

Embodiment 236. The method or composition of any one of Embodiments 4-10, 14-17, 24, 26-30, or 32-71, wherein the sequence selected from SEQ ID NOs: 90-178 is SEQ ID NO: 165.

Embodiment 237. The method or composition of any one of Embodiments 4-10, 14-17, 24, 26-30, or 32-71, wherein the sequence selected from SEQ ID NOs: 90-178 is SEQ ID NO: 166.

Embodiment 238. The method or composition of any one of Embodiments 4-10, 14-17, 24, 26-30, or 32-71, wherein the sequence selected from SEQ ID NOs: 90-178 is SEQ ID NO: 167.

Embodiment 239. The method or composition of any one of Embodiments 4-10, 14-17, 24, 26-30, or 32-71, wherein the sequence selected from SEQ ID NOs: 90-178 is SEQ ID NO: 168.

Embodiment 240. The method or composition of any one of Embodiments 4-10, 14-17, 24, 26-30, or 32-71, wherein the sequence selected from SEQ ID NOs: 90-178 is SEQ ID NO: 169.

Embodiment 241. The method or composition of any one of Embodiments 4-10, 14-17, 24, 26-30, or 32-71, wherein the sequence selected from SEQ ID NOs: 90-178 is SEQ ID NO: 170.

Embodiment 242. The method or composition of any one of Embodiments 4-10, 14-17, 24, 26-30, or 32-71, wherein the sequence selected from SEQ ID NOs: 90-178 is SEQ ID NO: 171.

Embodiment 243. The method or composition of any one of Embodiments 4-10, 14-17, 24, 26-30, or 32-71, wherein the sequence selected from SEQ ID NOs: 90-178 is SEQ ID NO: 172.

Embodiment 244. The method or composition of any one of Embodiments 4-10, 14-17, 24, 26-30, or 32-71, wherein the sequence selected from SEQ ID NOs: 90-178 is SEQ ID NO: 173.

Embodiment 245. The method or composition of any one of Embodiments 4-10, 14-17, 24, 26-30, or 32-71, wherein the sequence selected from SEQ ID NOs: 90-178 is SEQ ID NO: 174.

Embodiment 246. The method or composition of any one of Embodiments 4-10, 14-17, 24, 26-30, or 32-71, wherein the sequence selected from SEQ ID NOs: 90-178 is SEQ ID NO: 175.

Embodiment 247. The method or composition of any one of Embodiments 4-10, 14-17, 24, 26-30, or 32-71, wherein the sequence selected from SEQ ID NOs: 90-178 is SEQ ID NO: 176.

Embodiment 248. The method or composition of any one of Embodiments 4-10, 14-17, 24, 26-30, or 32-71, wherein the sequence selected from SEQ ID NOs: 90-178 is SEQ ID NO: 177.

Embodiment 249. The method or composition of any one of Embodiments 4-10, 14-17, 24, 26-30, or 32-71, wherein the sequence selected from SEQ ID NOs: 90-178 is SEQ ID NO: 178.

Embodiment 250. Use of a composition, formulation or cell of any preceding Embodiment for the preparation of a medicament.

Embodiment 251. A composition, formulation or cell of any preceding Embodiment for use as a medicament.

Embodiment 252. The cell of any of Embodiments 18-23, lacking an endogenous T cell receptor, for preparation of a T cell expressing a non-endogenous T cell receptor.

Embodiment 253. The cell of any of Embodiments 18-23, lacking an endogenous T cell receptor, for preparation of a T cell expressing a CAR.

Embodiment 254. The cell of any of Embodiments 18-23, wherein the altered cell is a CD3− cell.

Embodiment 255. The cell of any of Embodiments 18-23, wherein the cell is a CD3⁺ cell before being altered and the altered cell is a CD3− cell.

Embodiment 256. A population of cells comprising cells of any one of embodiments 18-23, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the altered population are CD3− cells.

Embodiment 257. The population of embodiment 256, wherein greater than about 90% of the population is CD3−.

Embodiment 258. The population of embodiment 256, wherein greater than about 95% of the population is CD3−.

Embodiment 259. The population of embodiment 256, wherein greater than about 99% of the population is CD3−.

Embodiment 260. A population of cells comprising cells of any one of embodiments 18-23, wherein greater than about 50%, greater than about 55%, greater than about 60%, greater than about 65%, greater than about 70%, greater than about 75%, greater than about 80%, greater than about 85%, greater than about 90%, greater than about 95%, greater than about 98%, or greater than about 99% of the population lacks an endogenous T cell receptor.

Embodiment 261. The population of embodiment 260, wherein greater than about 90% of the population lacks an endogenous T cell receptor.

Embodiment 262. The population of embodiment 260, wherein greater than about 95% of the population lacks an endogenous T cell receptor.

Embodiment 263. The population of embodiment 260, wherein greater than about 99% of the population lacks an endogenous T cell receptor.

Embodiment 264. A population of cells comprising cells of any one of embodiments 18-23, wherein the expression of the TRBC1, TRBC2 and/or TRAC genes in the population has been reduced relative to an unaltered population of the same cell by at least about 50%, at least about 55%, at least about 60%, at least about 65%, at least about 70%, at least about 75%, at least about 80%, at least about 85%, at least about 90%, at least about 95%, at least about 98%, or at least about 99%.

Embodiment 265. The population of embodiment 264, wherein the reduction in the expression of the TRBC1, TRBC2 and/or TRAC genes is at least about 90%.

Embodiment 266. The population of embodiment 264, wherein the reduction in the expression of the TRBC1, TRBC2 and/or TRAC genes is at least about 95%.

Embodiment 267. The population of embodiment 264, wherein the reduction in the expression of the TRBC1, TRBC2 and/or TRAC genes is at least about 99%.

Embodiment 268. The population of any of embodiments 264-267, wherein the reduction is in the expression of the TRBC1 gene.

Embodiment 269. The population of any of embodiments 264-267, wherein the reduction is in the expression of the TRBC2 gene.

Embodiment 270. The population of any of embodiments 264-267, wherein the reduction is in the expression of the TRAC gene.

Embodiment 271. A population of cells comprising cells of any one of embodiments 18-23, wherein between 10 and 100% of the population, e.g. between 30 and 99% of the population, has an indel in the TRBC1, TRBC2 and/or TRAC genes.

Embodiment 272. The population of embodiment 271, wherein between 30 and 35%, 35 and 40%, 40 and 45%, 45 and 50%, 50 and 55%, 55 and 60%, 60 and 65%, 65 and 70%, 70 and 75%, 75 and 80%, 80 and 85%, 85 and 90%, 90 and 95%, or 95 and 99% of the population has an indel in the TRBC1, TRBC2 and/or TRAC genes.

Embodiment 273. The population of embodiments 271 or 272, wherein the indel is in the TRBC1 gene.

Embodiment 274. The population of embodiments 271 or 272, wherein the indel is in the TRBC2 gene.

Embodiment 275. The population of embodiments 271 or 272, wherein the indel is in the TRAC gene.

This description and exemplary embodiments should not be taken as limiting. For the purposes of this specification and appended claims, unless otherwise indicated, all numbers expressing quantities, percentages, or proportions, and other numerical values used in the specification and claims, are to be understood as being modified in all instances by the term “about,” to the extent they are not already so modified. Accordingly, unless indicated to the contrary, the numerical parameters set forth in the following specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

### EXAMPLES

The following examples are provided to illustrate certain disclosed embodiments and are not to be construed as limiting the scope of this disclosure in any way.

### Example 1: Materials and Methods

### Genomic DNA Isolation

HEK293_Cas9 transfected cells were harvested post-transfection at 24 hours. The gDNA was extracted from each well of a 96-well plate using 50 μL/well QuickExtract™ DNA Extraction Solution (Lucigen, Cat. QE09050) according to manufacturer's protocol. DNA samples were subjected to PCR and subsequent NGS analysis, as described herein.

### Next-Generation Sequencing (“NGS”) and Analysis for On-Target Cleavage Efficiency

To quantitatively determine the efficiency of editing at the target location in the genome, deep sequencing was utilized to identify the presence of insertions and deletions introduced by gene editing. PCR primers were designed around the target site within the gene of interest (e.g. TRAC), and the genomic area of interest was amplified. Primer sequence design was done as is standard in the field.

Additional PCR was performed according to the manufacturer's protocols (Illumina) to add chemistry for sequencing. The amplicons were sequenced on an Illumina MiSeq instrument. The reads were aligned to the human reference genome (e.g., hg38) after eliminating those having low quality scores. Reads that overlapped the target region of interest were selected and the number of wild type reads versus the number of reads which contain an insertion or deletion (“indel”) was calculated.

The editing percentage (e.g., the “editing efficiency” or “indel percent”) is defined as the total number of sequence reads with insertions or deletions (“indels”) over the total number of sequence reads, including wild type.

### Example 2: TRAC Guide Design and Screening in HEK Cells

### Human TRAC Guide Design

Initial guide selection was performed in silico using a human reference genome (e.g., hg38) and user defined genomic regions of interest (e.g., TRAC protein coding exons), for identifying PAMs in the regions of interest. For each identified PAM, analyses were performed and statistics reported. gRNA molecules were further selected and rank-ordered based on a number of criteria known in the art (e.g., GC content, predicted on-target activity, and potential off-target activity).

A total of 88 guide RNAs were designed toward TRAC (ENSG00000277734) targeting the protein exonic coding regions and intron 1. Guide sequences and corresponding genomic coordinates are provided (Table 2).

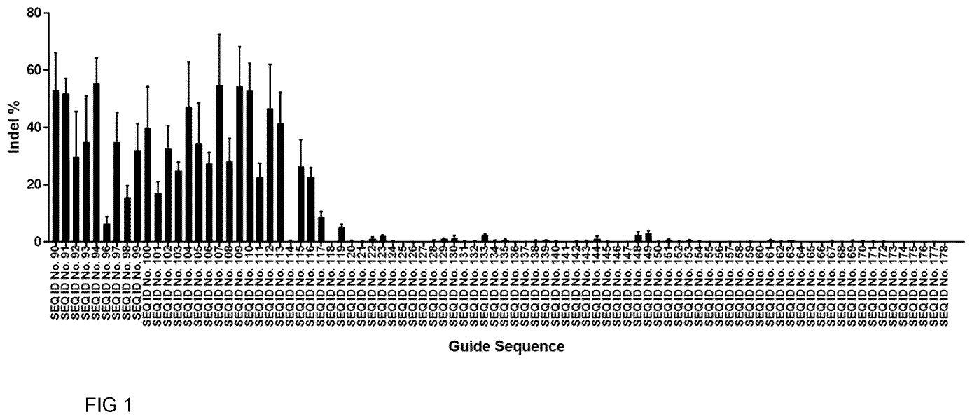

Guides were screened for editing efficiency in HEK293_Cas9 cells. The human embryonic kidney adenocarcinoma cell line HEK293 constitutively expressing Spy Cas9 (“HEK293_Cas9”) was cultured in DMEM media supplemented with 10% fetal bovine serum. Cells were plated at a density of 10,000 cells/well in a 96-well plate about 24 hours prior to transfection (~70% confluent at time of transfection). Cells were transfected in duplicate with Lipofectamine RNAiMAX (ThermoFisher, Cat. 13778150) according to the manufacturer's protocol. Cells were transfected with a lipoplex containing individual guide (25 nM), trRNA (25 nM), Lipofectamine RNAiMAX (0.3 L/well) and OptiMem. DNA isolation and NGS analysis were performed as described in Example 1. FIG. 1 and Table 5 show indel % at the TRAC locus by these guides in HEK293_Cas9 cells.

TABLE 5
TRAC guide editing in HEK293_Cas9 cells

| SEQ | % Indels | |
|---|---|---|
| ID NO: | (Mean) | SD |
| 90 | 53.1 | 13 |
| 91 | 51.9 | 5.2 |
| 92 | 29.8 | 15.8 |
| 93 | 35.1 | 16.1 |
| 94 | 55.4 | 9 |
| 96 | 6.6 | 2.3 |
| 97 | 35.3 | 9.8 |
| 98 | 15.6 | 4.2 |
| 99 | 32 | 9.5 |
| 100 | 40 | 14.3 |
| 101 | 17 | 4.1 |
| 102 | 32.9 | 7.8 |
| 103 | 24.8 | 3.1 |
| 104 | 47.2 | 15.7 |
| 105 | 34.7 | 13.9 |
| 106 | 27.4 | 3.8 |
| 107 | 54.9 | 17.8 |
| 108 | 28.2 | 7.9 |
| 109 | 54.4 | 14 |
| 110 | 52.9 | 9.5 |
| 111 | 22.6 | 5 |
| 112 | 46.8 | 15.3 |
| 113 | 41.6 | 10.8 |
| 114 | 0.4 | 0.1 |
| 115 | 26.4 | 9.4 |
| 116 | 22.8 | 3.3 |
| 117 | 8.9 | 1.8 |
| 118 | 0 | 0 |
| 119 | 5.3 | 1.1 |
| 120 | 0.4 | 0.1 |
| 121 | 0.2 | 0.1 |
| 122 | 1.2 | 0.6 |
| 123 | 2.2 | 0.4 |
| 124 | 0.3 | 0.1 |
| 125 | 0.1 | 0 |
| 126 | 0.1 | 0.1 |
| 127 | 0 | 0 |
| 128 | 0.4 | 0.4 |
| 129 | 1.1 | 0.3 |
| 130 | 1.6 | 0.7 |
| 131 | 0.2 | 0.1 |
| 132 | 0.4 | 0.1 |
| 133 | 2.5 | 0.5 |
| 134 | 0.5 | 0.2 |
| 135 | 0.7 | 0.3 |
| 136 | 0.1 | 0.1 |
| 137 | 0.1 | 0.1 |
| 138 | 0.5 | 0.2 |
| 139 | 0.5 | 0.1 |
| 140 | 0.3 | 0.1 |
| 141 | 0.1 | 0 |
| 142 | 0.4 | 0.1 |
| 143 | 0.4 | 0.1 |
| 144 | 1.3 | 0.8 |
| 145 | 0.2 | 0.1 |
| 146 | 0.1 | 0 |
| 147 | 0.1 | 0.1 |
| 148 | 2.5 | 1.2 |
| 149 | 3 | 1 |
| 150 | 0.2 | 0.1 |
| 151 | 0.7 | 0.4 |
| 152 | 0.2 | 0.1 |
| 153 | 0.7 | 0.1 |
| 154 | 0.3 | 0.1 |
| 155 | 0.1 | 0.1 |
| 156 | 0.1 | 0 |
| 157 | 0.1 | 0.1 |
| 158 | 0.1 | 0.1 |
| 159 | 0.2 | 0.1 |
| 160 | 0.1 | 0 |
| 161 | 0.7 | 0.2 |
| 162 | 0.1 | 0.1 |
| 163 | 0.7 | 0 |
| 164 | 0.1 | 0 |
| 165 | 0 | 0 |
| 166 | 0.3 | 0 |
| 167 | 0.3 | 0.3 |
| 168 | 0.1 | 0.1 |
| 169 | 0.5 | 0.2 |
| 170 | 0.2 | 0.1 |
| 171 | 0.2 | 0.1 |
| 172 | 0.2 | 0.1 |
| 173 | 0 | 0 |
| 174 | 0.1 | 0 |
| 175 | 0.1 | 0.1 |
| 176 | 0.1 | 0 |
| 177 | 0.1 | 0.1 |
| 178 | 0.1 | 0 |

### Example 3: TRAC Guide Screening in Human CD3⁺ T Cells

The 24 guides with the highest indel percent editing in HEK293_Cas9 cells from Example 2 were screened for editing efficiency and T-cell receptor (TCR) knockdown in human CD3⁺ T cells. CD3⁺ T cells are comprised of multiple T cell populations including CD4⁺ T helper cells and CD8⁺ cytotoxic T cells. These cells can be isolated from whole blood or from leuokopheresis samples. T cells can be modified to specifically target cancerous cells and to be less immunogenic, by engineering patient T cells using Cas9-mediated editing. This example describes a basic method used to deliver Cas9 RNP, for example, Cas9 RNP targeting TRAC, in T cells. Only the targeting crRNA in the RNP would need to be changed to adapt this protocol to a different T cell target (e.g., any of those provided herein).

### Delivery of RNPs to T Cells

T cells were either obtained commercially (e.g. Human Peripheral Blood CD4⁺CD45RA⁺ T Cells, Frozen, Stem Cell Technology, Cat. 70029) or prepared internally from a leukopak. For internal preparation, T cells were first enriched from a leukopak using a commercially available kit (e.g., EasySep™ Human T Cell Isolation Kit, Stem Cell Technology). Enriched T cells were aliquoted and frozen down (at 5×10⁶/vial) for future use. Vials were subsequently thawed as needed, and activated by addition of 3:1 ratio of CD3/CD28 beads (Dynabeads, Life Technologies) in T cell media (RPMI 1640, FBS, L-glutamine, non-essential amino acids, sodium pyruvate, HEPES buffer, 2-mercaptoethanol and optionally IL2). RNP was generated by pre-annealing individual crRNA and trRNA by mixing equivalent amounts of reagent and incubating at 95° C. for 2 min and cooling to room temperature. The dual guide (dgRNA) consisting of pre-annealed crRNA and trRNA, was incubated with Spy Cas9 protein to form a ribonucleoprotein (RNP) complex. CD3 T cells were transfected in triplicate with an RNP containing Spy Cas9 ((nnM), individual guide (10 nM) and tracer RNA (10 nM) nucleofected using the P3 Primary Cell 96-well Nucleofector™ Kit (Lonza, Cat. V4SP-3960) using the manufacturer's Amaxa™ 96-well Shuttle™ Protocol for Stimulated Human T Cells. T cell media was added to cells immediately post-nucleofection and cultured for 2 days or more.

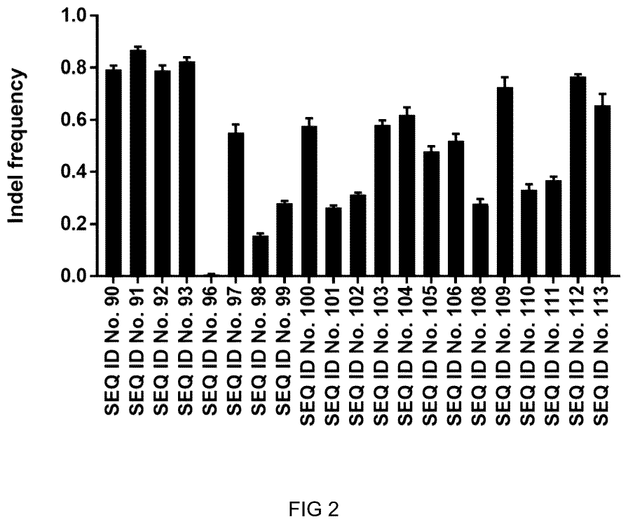

Two days post nucleofection, genomic DNA was prepared as described in Example 1 and NGS analysis performed. Table 6 and FIG. 2 show results for indel frequency following TRAC editing with various guides in CD3⁺ T cells.

TABLE 6
Indel frequency for TRAC editing in human CD3⁺ T cells

| SEQ | Indel | Standard | Sample |
|---|---|---|---|
| ID NO: | Frequency | Deviation | Number (n) |
| 90 | 79.3% | 1.5% | 3 |
| 91 | 86.9% | 1.2% | 3 |
| 92 | 79.0% | 1.9% | 3 |
| 93 | 82.6% | 1.4% | 3 |
| 96 | 0.7% | 0.1% | 3 |
| 97 | 55.2% | 3.1% | 3 |
| 98 | 15.5% | 1.0% | 3 |
| 99 | 27.9% | 0.9% | 3 |
| 100 | 57.7% | 3.0% | 3 |
| 101 | 26.2% | 0.8% | 3 |
| 102 | 31.2% | 0.8% | 3 |
| 103 | 58.1% | 1.8% | 3 |
| 104 | 62.0% | 2.8% | 3 |
| 105 | 47.8% | 2.0% | 3 |
| 106 | 52.0% | 2.7% | 3 |
| 108 | 27.7% | 1.9% | 3 |
| 109 | 72.7% | 3.7% | 3 |
| 110 | 33.0% | 2.2% | 3 |
| 111 | 36.9% | 1.3% | 3 |
| 112 | 76.7% | 1.0% | 3 |
| 113 | 65.7% | 4.3% | 3 |

### Flow Cytometric Analysis of TCR Expression

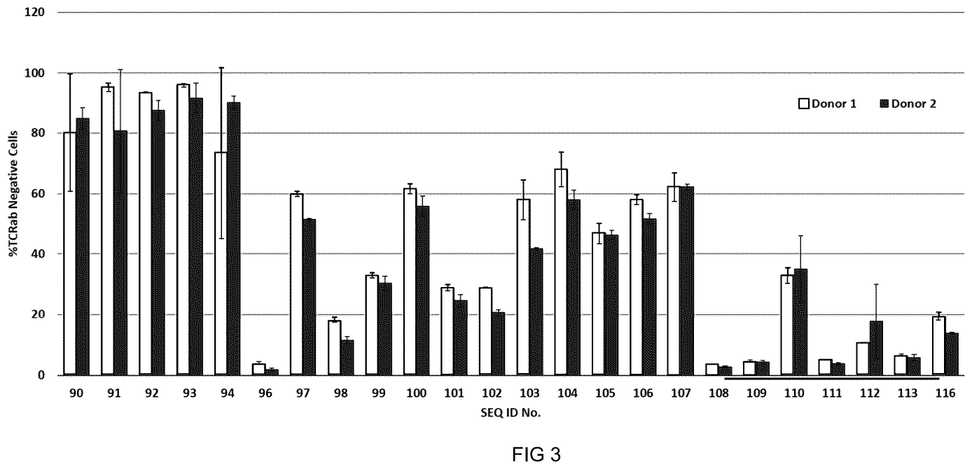

Expression of the TCR was measured 4 days post nucleofection. Cells were stained with fixable live dead dye (Thermo fisher L34975) and the TCR was detected using Alexa Fluor® 647 anti-human TCR α/β Antibody (Biolegend, Cat. 306714) Cells were incubated with 2 ul of antibody for at least 20 minutes one ice, analyzed by flow cytometery using, for example, Beckman Coulter CytoflexS). Data was analyzed using Flow Jo software. Live cell population was analyzed for TCR loss. The results are shown in Table 7 and FIG. 3.

TABLE 7
TCR expression after TRAC editing in CD3⁺ T cells

| | Donor 1- | Donor 1- | Donor 2- | Donor 2- |
|---|---|---|---|---|
| SEQ | % TCR | Standard | % TCR | Standard |
| ID NO: | Negative | Deviation | Negative | Deviation |
| 90 | 80.2 | 19.5 | 84.9 | 3.6 |
| 91 | 95.3 | 1.4 | 80.7 | 20.5 |
| 92 | 93.5 | 0.3 | 87.6 | 3.4 |
| 93 | 95.9 | 0.6 | 91.7 | 5 |
| 94 | 73.5 | 28.3 | 90.2 | 2.1 |
| 96 | 3.6 | 0.8 | 1.8 | 0.4 |
| 97 | 59.9 | 0.9 | 51.3 | 0.4 |
| 98 | 17.9 | 1.1 | 11.5 | 1.2 |
| 99 | 32.9 | 1.1 | 30.4 | 2.3 |
| 100 | 61.4 | 1.7 | 55.8 | 3.4 |
| 101 | 28.8 | 1.3 | 24.6 | 2 |
| 102 | 28.7 | 0.5 | 20.7 | 1 |
| 103 | 58.2 | 6.4 | 41.6 | 0.4 |
| 104 | 68 | 5.7 | 58 | 3.2 |
| 105 | 46.8 | 3.3 | 46.2 | 1.5 |
| 106 | 58 | 1.6 | 51.6 | 1.7 |
| 107 | 62.3 | 4.6 | 62.2 | 0.9 |
| 108 | 3.3 | 0.3 | 2.6 | 0.4 |
| 109 | 4.5 | 0.5 | 4.2 | 0.5 |
| 110 | 33.1 | 2.5 | 35.1 | 10.9 |
| 111 | 5.1 | 0.1 | 3.7 | 0.3 |
| 112 | 10.5 | 0.2 | 17.7 | 12.3 |
| 113 | 6.3 | 0.7 | 5.7 | 1 |
| 116 | 19.3 | 1.5 | 13.7 | 0.4 |

### Example 4: TRBC Guide Design and Screening in HEK Cells

### Human TRBC Guide Design

Initial guide selection for editing human TRBC1 (ENSG00000211751) and TRBC2 (ENSG00000211772) was as described in Example 2. A total of 89 guide RNAs were designed: 2 targeting TRBC1 only (SEQ ID NO: 11 and SEQ ID NO: 26), 2 targeting TRBC2 only (SEQ ID NO: 8) and 86 targeting both TRBC1 and TRBC2. Guides were designed to target the protein exonic coding regions. Guides and corresponding genomic coordinates are provided above (Table 1).

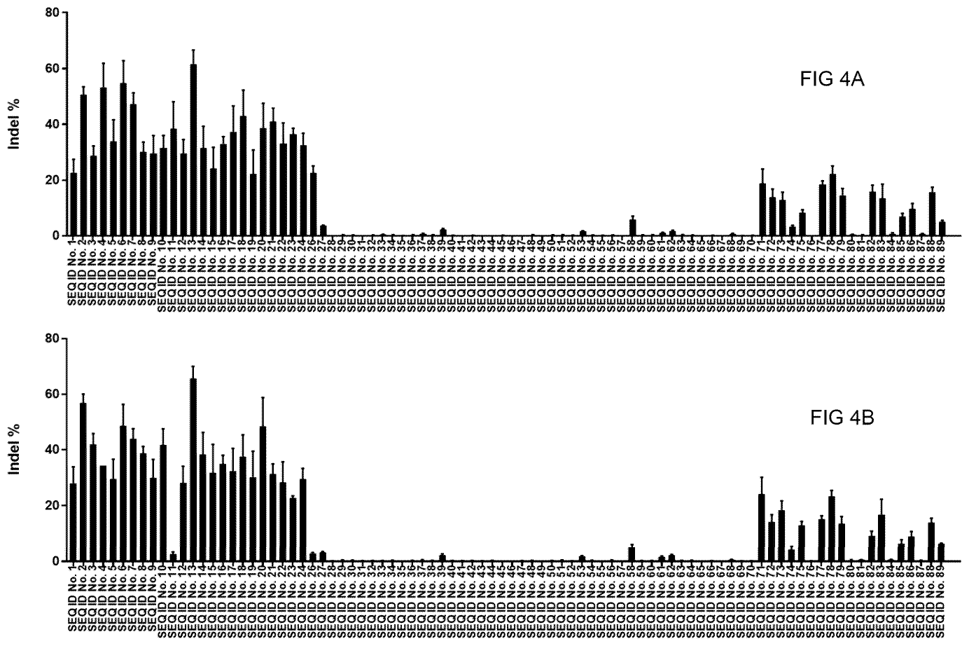

TRBC guides were screened for editing efficiency in HEK293_Cas9 cells. Transfection was performed as described in Example 2. DNA isolation and NGS analysis were performed as described in Example 1. FIGS. 4A and 4B and Table 8 show indel 00 at the TRBC1 and TRBC2 loci by these guides in HEK293 Cas9 cells.

TABLE 8
TRBC guide editing in HEK293_Cas9 cells

| | TRBC1 | | Number | TRBC2 | | Number |
|---|---|---|---|---|---|---|
| SEQ | Mean | TRBC1 | of values | Mean | TRBC2 | of values |
| ID NO: | Indel % | SD | (n) | Indel % | SD | (n) |
| 1 | 22.7 | 4.8 | 3 | 28.0 | 5.9 | 3 |
| 2 | 50.6 | 2.9 | 3 | 56.9 | 3.1 | 3 |
| 3 | 28.7 | 3.5 | 3 | 41.9 | 4 | 3 |
| 4 | 53.4 | 8.6 | 3 | 34.4 | NA | 1 |
| 5 | 33.9 | 7.7 | 3 | 29.5 | 7.1 | 3 |
| 6 | 54.8 | 8 | 3 | 48.7 | 7.7 | 3 |
| 7 | 47.3 | 4 | 3 | 43.9 | 3.8 | 3 |
| 8 | 30.2 | 3.5 | 3 | 38.6 | 2.5 | 3 |
| 9 | 29.5 | 6.4 | 3 | 30 | 6.5 | 3 |
| 10 | 31.5 | 4.6 | 3 | 41.7 | 5.9 | 3 |
| 11 | 38.4 | 9.7 | 3 | 2.6 | 0.8 | 3 |
| 12 | 29.6 | 5 | 3 | 28.1 | 6 | 3 |
| 13 | 61.7 | 4.9 | 3 | 65.7 | 4.4 | 3 |
| 14 | 31.5 | 7.8 | 3 | 38.3 | 8 | 3 |
| 15 | 24.2 | 7.5 | 3 | 31.7 | 10.3 | 3 |
| 16 | 33 | 2.6 | 3 | 34.9 | 3.2 | 3 |
| 17 | 37.4 | 9.3 | 3 | 32.3 | 8.2 | 3 |
| 18 | 43 | 9.4 | 3 | 37.6 | 7.9 | 3 |
| 19 | 22.2 | 8.6 | 3 | 30.2 | 9.3 | 3 |
| 20 | 38.7 | 8.9 | 3 | 48.5 | 10.4 | 3 |
| 21 | 40.9 | 5 | 3 | 31.3 | 3.6 | 3 |
| 22 | 33.1 | 7.5 | 3 | 28.3 | 7.4 | 3 |
| 23 | 36.4 | 2.2 | 3 | 22.7 | 0.8 | 3 |
| 24 | 32.6 | 4.2 | 3 | 29.5 | 3.8 | 3 |
| 26 | 22.5 | 2.6 | 3 | 2.9 | 0.3 | 3 |
| 27 | 3.7 | 0.2 | 3 | 3.2 | 0.3 | 3 |
| 28 | 0.1 | 0 | 3 | 0.1 | 0.1 | 3 |
| 29 | 0.3 | 0.1 | 3 | 0.4 | 0.2 | 3 |
| 30 | 0.2 | 0.1 | 3 | 0.3 | 0.2 | 3 |
| 31 | 0.1 | 0 | 3 | 0.2 | 0.1 | 3 |
| 32 | 0.2 | 0.1 | 3 | 0.2 | 0.1 | 3 |
| 33 | 0.4 | 0.1 | 3 | 0.2 | 0.1 | 3 |
| 34 | 0.4 | 0.1 | 3 | 0.3 | 0.1 | 3 |
| 35 | 0 | 0.1 | 3 | 0.1 | 0.1 | 3 |
| 36 | 0.3 | 0.1 | 3 | 0.3 | 0.1 | 3 |
| 37 | 0.8 | 0.1 | 3 | 0.5 | 0.2 | 3 |
| 38 | 0.3 | 0.1 | 3 | 0.3 | 0.1 | 3 |
| 39 | 2.2 | 0.4 | 3 | 2.2 | 0.4 | 3 |
| 40 | 0.1 | 0.1 | 3 | 0.2 | 0.1 | 3 |
| 41 | 0.1 | 0 | 3 | 0.2 | 0.1 | 3 |
| 42 | 0.1 | 0.1 | 3 | 0.2 | 0.1 | 3 |
| 43 | 0.2 | 0 | 3 | 0.1 | 0.1 | 3 |
| 44 | 0.2 | 0 | 3 | 0.3 | 0.1 | 3 |
| 45 | 0.1 | 0.1 | 3 | 0.1 | 0 | 3 |
| 46 | 0.1 | 0 | 3 | 0.1 | 0 | 3 |
| 47 | 0.1 | 0 | 3 | 0.2 | 0.1 | 3 |
| 48 | 0.2 | 0.2 | 3 | 0.3 | 0.1 | 3 |
| 49 | 0.1 | 0 | 3 | 0.1 | 0 | 3 |
| 50 | 0.3 | 0.1 | 3 | 0.2 | 0.1 | 3 |
| 51 | 0.4 | 0.1 | 3 | 0.4 | 0.1 | 3 |
| 52 | 0.2 | 0.1 | 3 | 0.1 | 0.1 | 3 |
| 53 | 1.6 | 0.2 | 3 | 1.8 | 0.2 | 3 |
| 54 | 0.2 | 0.1 | 3 | 0.3 | 0.1 | 3 |
| 55 | 0.1 | 0.1 | 3 | 0 | NA | 1 |
| 56 | 0.2 | 0.1 | 3 | 0.4 | 0.1 | 3 |
| 57 | 0.1 | 0 | 3 | 0.1 | 0 | 3 |
| 58 | 5.8 | 1.3 | 3 | 5 | 1.1 | 3 |
| 59 | 0.3 | 0.1 | 3 | 0.2 | 0.1 | 3 |
| 60 | 0.3 | 0.1 | 3 | 0.2 | 0.1 | 3 |
| 61 | 1 | 0.2 | 3 | 1.6 | 0.3 | 3 |
| 62 | 1.7 | 0.3 | 3 | 2.2 | 0.2 | 3 |
| 63 | 0.3 | 0.1 | 3 | 0.3 | 0.1 | 3 |
| 64 | 0.2 | 0.1 | 3 | 0.3 | 0.1 | 3 |
| 65 | 0.2 | 0 | 3 | 0.2 | 0 | 3 |
| 66 | 0.1 | 0.1 | 3 | 0.2 | 0.1 | 3 |
| 67 | 0.1 | 0 | 3 | 0.1 | 0.1 | 3 |
| 68 | 0.7 | 0.2 | 3 | 0.6 | 0.1 | 3 |
| 69 | 0 | 0.1 | 3 | 0.1 | 0.1 | 3 |
| 70 | 0.1 | 0.1 | 3 | 0.1 | 0.1 | 3 |
| 71 | 18.9 | 5.1 | 3 | 24.2 | 6 | 3 |
| 72 | 13.8 | 3 | 3 | 14.2 | 2.6 | 3 |
| 73 | 12.9 | 2.9 | 3 | 18.4 | 3.3 | 3 |
| 74 | 3.3 | 0.4 | 3 | 4.2 | 1.2 | 3 |
| 75 | 8.3 | 1.1 | 3 | 13 | 1.4 | 3 |
| 76 | 0.3 | 0 | 3 | 0.1 | 0.1 | 3 |
| 77 | 18.5 | 1.3 | 3 | 15.1 | 1.3 | 3 |
| 78 | 22.2 | 2.8 | 3 | 23.3 | 2.2 | 3 |
| 79 | 14.6 | 2.5 | 3 | 13.6 | 2.6 | 3 |
| 80 | 0.4 | 0.1 | 3 | 0.5 | 0.2 | 3 |
| 81 | 0.3 | 0.1 | 3 | 0.5 | 0.1 | 3 |
| 82 | 15.9 | 2.3 | 3 | 9.1 | 1.8 | 3 |
| 83 | 13.6 | 5 | 3 | 16.8 | 5.5 | 3 |
| 84 | 0.8 | 0.3 | 3 | 0.7 | 0.1 | 3 |
| 85 | 6.9 | 1.2 | 3 | 6.3 | 1.5 | 3 |
| 86 | 9.7 | 2 | 3 | 9 | 1.8 | 3 |
| 87 | 0.7 | 0.1 | 3 | 0.2 | 0.2 | 3 |
| 88 | 15.8 | 1.7 | 3 | 13.9 | 1.6 | 3 |
| 89 | 5 | 0.6 | 3 | 6.3 | 0.3 | 3 |

### Example 5: TRBC Guide Screening in Human CD3⁺ T Cells

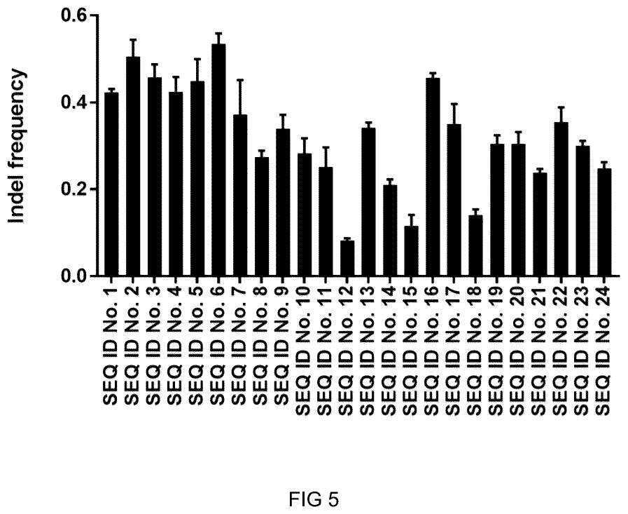

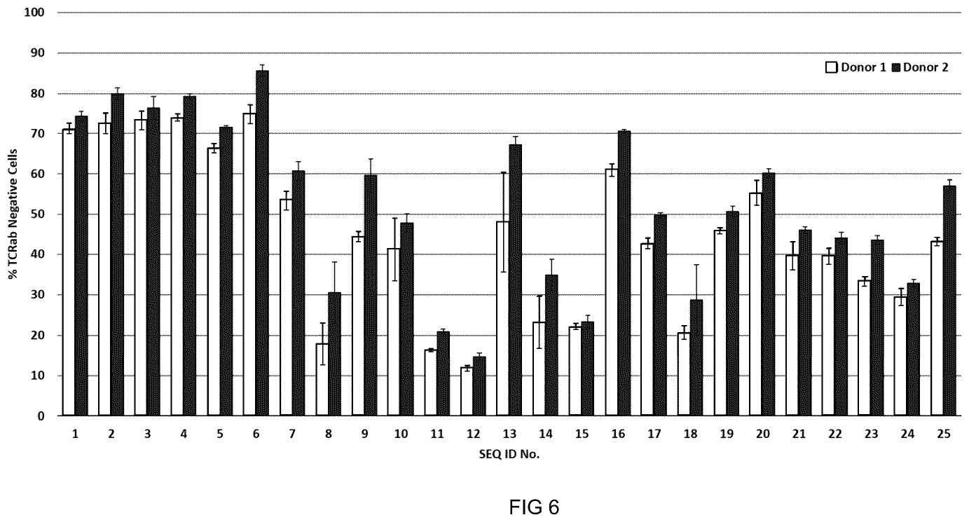

TRBC guides with high indel percent editing in HEK293_Cas9 cells from Example 4 were screened for editing efficiency and T-cell receptor (TCR) expression in human CD3⁺ T cells. Nucleofection, editing analysis and TCR expression analysis were performed as in Example 3. Table 9 and FIG. 5 show results for indel frequency following TRBC editing with various guides in CD3⁺ T cells. Table 10 and FIG. 6 show TCR expression measurements (as % Oo TCR negative cells) following TRBC editing in CD3⁺ T cells.

TABLE 9
Indel Frequency at TRBC1 in CD3⁺ T cells

| SEQ | Mean Indel | Standard | Sample |
|---|---|---|---|
| ID NO: | frequency | Deviation | Number (n) |
| 1 | 42.40% | 0.80% | 3 |
| 2 | 50.50% | 3.90% | 3 |
| 3 | 45.80% | 3.00% | 3 |
| 4 | 42.50% | 3.40% | 3 |
| 5 | 45.00% | 5.00% | 3 |
| 6 | 53.50% | 2.40% | 3 |
| 7 | 37.30% | 7.90% | 3 |
| 8 | 27.40% | 1.50% | 3 |
| 9 | 34.00% | 3.20% | 3 |
| 10 | 28.20% | 3.50% | 3 |
| 11 | 25.10% | 4.60% | 3 |
| 12 | 8.30% | 0.40% | 3 |
| 13 | 34.20% | 1.20% | 3 |
| 14 | 21.00% | 1.30% | 3 |
| 15 | 11.60% | 2.50% | 3 |
| 16 | 45.70% | 1.00% | 3 |
| 17 | 35.10% | 4.60% | 3 |
| 18 | 14.10% | 1.30% | 3 |
| 19 | 30.40% | 2.10% | 3 |
| 20 | 30.50% | 2.70% | 3 |
| 21 | 23.80% | 0.90% | 3 |
| 22 | 35.50% | 3.40% | 3 |
| 23 | 30.00% | 1.10% | 3 |
| 24 | 24.80% | 1.40% | 3 |

TABLE 10
TCR expression after TRBC editing in CD3⁺ T cells

| | Donor 1- | Donor 1- | Donor 2- | Donor 2- |
|---|---|---|---|---|
| SEQ | % TCR | Standard | % TCR | Standard |
| ID NO: | Negative | Deviation | negative | Deviation |
| 1 | 71.2 | 1.4 | 74.4 | 1.3 |
| 2 | 72.4 | 2.6 | 79.9 | 1.5 |
| 3 | 73.3 | 2.3 | 76.4 | 2.8 |
| 4 | 74 | 0.9 | 79.2 | 0.7 |
| 5 | 66.4 | 1.1 | 71.5 | 0.5 |
| 6 | 75 | 2.2 | 85.7 | 1.4 |
| 7 | 53.5 | 2.2 | 60.7 | 2.3 |
| 8 | 18 | 5.1 | 30.6 | 7.6 |
| 9 | 44.4 | 1.3 | 59.6 | 4.1 |
| 10 | 41.2 | 7.9 | 47.9 | 2.3 |
| 11 | 16.3 | 0.3 | 20.9 | 0.7 |
| 12 | 11.8 | 0.7 | 14.6 | 1 |
| 13 | 48.2 | 12.3 | 67.2 | 2 |
| 14 | 23.3 | 6.5 | 34.8 | 3.9 |
| 15 | 22.1 | 0.8 | 23.3 | 1.6 |
| 16 | 60.8 | 1.7 | 70.5 | 0.5 |
| 17 | 42.8 | 1.4 | 49.7 | 0.7 |
| 18 | 20.6 | 1.8 | 28.7 | 8.7 |
| 19 | 45.9 | 0.8 | 50.7 | 1.4 |
| 20 | 55.3 | 3.2 | 60.2 | 1.1 |
| 21 | 39.7 | 3.4 | 46.1 | 0.8 |
| 22 | 39.6 | 2.1 | 44.1 | 1.4 |
| 23 | 33.5 | 1 | 43.6 | 1.1 |
| 24 | 29.5 | 2.1 | 32.8 | 1 |
| 25 | 43.2 | 1 | 57 | 1.5 |

### Example 6: Off-Target Analysis of TRBC and TRAC Guides

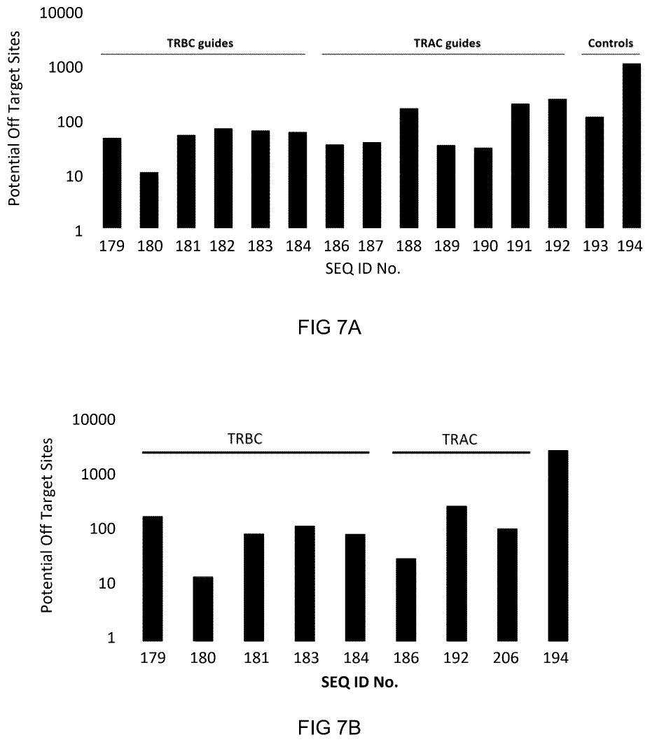

A biochemical method (See, e.g., Cameron et al., Nature Methods. 6, 600-606; 2017) was used to determine potential off-target genomic sites cleaved by Cas9 targeting TRAC, TRBC1 or TRBC2. Guides showing the most CD3 negative cells in Example 3 and Example 5 were tested for potential off-target genomic cleavage sites with this assay. In this experiment, 7 sgRNA targeting human TRAC, 6 sgRNA targeting TRBC and two control guides with known off-target profiles were screened using isolated HEK293_Cas genomic DNA. The number of potential off-target sites detected using an RNP concentration of 16 nM in the biochemical assay were plotted in FIG. 7A and shown in Table 11. The assay identified potential off-target sites for the sgRNAs tested.

TABLE 11
Biochemical Off-Target Analysis

| SEQ | | Number |
|---|---|---|
| ID NO: | Target | of Sites |
| 179 | TRBC1/2 | 48 |
| 180 | TRBC1/2 | 11 |
| 181 | TRBC1/2 | 54 |
| 182 | TRBC1/2 | 72 |
| 183 | TRBC1/2 | 65 |
| 184 | TRBC1/2 | 61 |
| 186 | TRAC | 36 |
| 187 | TRAC | 40 |
| 188 | TRAC | 168 |
| 189 | TRAC | 35 |
| 190 | TRAC | 31 |
| 191 | TRAC | 207 |
| 192 | TRAC | 253 |
| 193 | EMX1 | 118 |
| 194 | VEGFA | 1113 |

### Targeted Sequencing for Validating Potential Off-Target Sites

In known off-target detection assays such as the biochemical method used above, a large number of potential off-target sites are typically recovered, by design, so as to “cast a wide net” for potential sites that can be validated in other contexts, e.g., in a primary cell of interest. For example, the biochemical method typically overrepresents the number of potential off-target sites as the assay utilizes purified high molecular weight genomic DNA free of the cell environment and is dependent on the dose of Cas9 RNP used. Accordingly, potential off-target sites identified by these methods may be validated using targeted sequencing of the identified potential off-target sites.

In one approach, Cas9 and a sgRNA of interest (e.g., a sgRNA having potential off-target sites for evaluation) are introduced to primary T cells. The T cells are then lysed and primers flanking the potential off-target site(s) are used to generate an amplicon for NGS analysis. Identification of indels at a certain level may validate potential off-target site, whereas the lack of indels found at the potential off-target site may indicate a false positive in the off-target assay that was utilized.

Guides showing on target indel activity were tested for potential off-target genomic cleavage sites with this assay. In this experiment, 5 sgRNAs targeting human TRBC1 or TRBC2 and 3 guides targeting human TRAC were screened using genomic DNA purified from pooled male human peripheral blood mononuclear cells (PBMCs) alongside a control guide, SEQ ID NO: 194 (VEGFA) with known off-target profiles. The number of potential off-target sites detected using a guide concentration of 64 nM in the biochemical assay are shown in FIG. 7B and Table 12.

TABLE 12
64 nM Off Target Analysis

| SEQ ID NO: | | Guide Sequence | Off-target |
|---|---|---|---|
| of sgRNA | Target | (SEQ ID NO:) | Site Count |
| 179 | TRBC1/2 | GGCUCUCGGAGAAUGACGAG | 190 |
| | | (SEQ ID NO: 1) | |
| 180 | TRBC1/2 | GGCCUCGGCGCUGACGAUCU | 15 |
| | | (SEQ ID NO: 2) | |
| 181 | TRBC1/2 | AUGACGAGUGGACCCAGGAU | 92 |
| | | (SEQ ID NO: 3) | |
| 183 | TRBC1/2 | UGAGGGUCUCGGCCACCUUC | 127 |
| | | (SEQ ID NO: 5) | |
| 184 | TRBC1/2 | AGAGAUCUCCCACACCCAAA | 90 |
| | | (SEQ ID NO: 6) | |
| 186 | TRAC | CUCUCAGCUGGUACACGGCA | 32 |
| | | (SEQ ID NO: 90) | |
| 192 | TRAC | UCAGGGUUCUGGAUAUCUGU | 293 |
| | | (SEQ ID NO: 185) | |
| 206 | TRAC | UAGGCAGACAGACUUGUCAC | 114 |
| | | (SEQ ID NO: 214) | |
| 194 | VEGFA | See Table 3 | 2945 |

### Example 7: Using TRAC and TRBC Guides for Multiple Edits

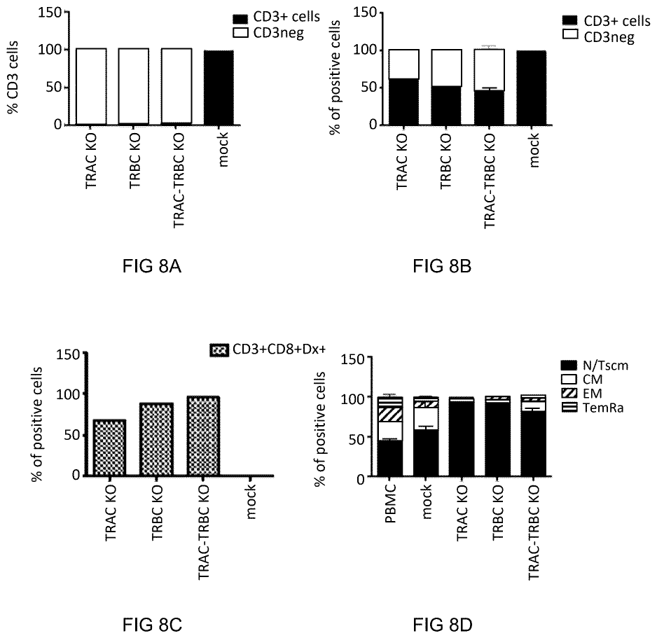

Guides were also tested for simultaneous editing of TRAC and TRBC loci. Peripheral blood mononuclear cells (PBMCs) from 3 different healthy donors were isolated from buffy coat using Lymphoprep™ (Stem Cell Technology) for density gradient centrifugation. These cells were activated and sorted using Dynabeads™ ClinExVivo™ CD3/CD28 (Invitrogen) following the manufacturer instructions. T cells were seeded at a concentration of 106 cells/ml in X-VIVO™ 15 Hematopoietic Serum-Free Culture Media (Lonza™) supplemented with 5% FBS and with IL7 and IL15 (5 ng/ml each). Two days after stimulation, T cells were electroporated as described in Example 3 simultaneously with a crRNA targeting TRAC and a crRNA targeting TRBC. Using cells from one donor, TRAC and TRBC guides were electroporated in isolation. At day 6 post stimulation, beads were detached and cells were seeded at a concentration of 1 million/ml in X-VIVO™ 15 supplemented with 5% FBS and with IL7 and IL15 (5 ng/ml each). TRAC and TRBC knockout efficiency at day 7 was assessed by evaluating the percentage of T cells devoid of the CD3 molecule by flow cytometry as shown in FIG. 8A and Table 13.

TABLE 13
Efficiency of TCR knockdown

| SEQ | CD3+ | | CD3neg | | |
|---|---|---|---|---|---|
| ID NO: | Mean % | SD | Mean % | SD | Samples (n) |
| 95 | 1 | N/A | 98 | N/A | 1 |
| 6 | 1 | N/A | 99 | N/A | 1 |
| 6 and 95 | 2 | 1 | 98 | 1 | 3 |
| mock | 97 | 1 | 2 | 1 | 3 |

Simultaneous TRAC and TRBC Editing with Lentiviral Insertion of a TCR Gene.

Three days post stimulation, edited T cells were transduced with a lentiviral vector (LV) encoding a WT1-specific TCR derived from a healthy donor (HD1-TCR) randomly inserting the LV. Briefly, the TCR α and β chain genes were isolated, codon-optimized, cysteine-modified, and cloned in a LV under a bidirectional promoter. The DNA and amino acid sequences of the HD1 TCR α and β chain genes are given below. The alpha chain was cloned in antisense orientation under the minimal human CMV promoter and the beta chain in sense orientation under the PGK promoter. LVs were packaged by an integrase-competent third-generation construct and pseudotyped by the vescicular stomatitis virus (VSV) envelope. T lymphocytes were infected with the LV for 24 h. Afterwards, T cells were cultured at 10⁶ cells/ml and expanded. Two days after transduction, transduction efficiency was evaluated by determining the percentage of T cells expressing the specific Vβ gene (TRBV12-3/TRBV12-4) as well as the specific dextramer (epitope: VLDFAPPGA). At day 15 post stimulation, T cell phenotype was evaluated by cytofluorimetric analysis. FIG. 8B and Table 14 show that more than 45% of T cells expressed the CD3 molecule (mean±SEM=45,3±5,7). Percentage of WT1-specific CD8⁺ T cells evaluated by measuring Dextramer positivity was greater than 95% (mean±SEM=95,3±0,7) Percentage of WT1-specific CD8⁺ T cells evaluated by measuring Dextramer positivity was greater than 95% when SEQ ID NOs 6 and 95 were used (mean±SEM=95,3±0,7) (FIG. 8C, Table 14). T cell phenotype of the edited T cells was evaluated at day15 as shown in FIG. 8D and Table 15.

SEQ ID NO: 250: HD1 TCR α chain - DNA sequence
ATGGAAACCCTGCTGAAGGTGCTGAGCGGCACACTGCTGTGGCAGCTG
ACATGGGTCCGATCTCAGCAGCCTGTGCAGTCTCCTCAGGCCGTGATTCTGAGAG
AAGGCGAGGACGCCGTGATCAACTGCAGCAGCTCTAAGGCCCTGTACAGCGTGC
ACTGGTACAGACAGAAGCACGGCGAGGCCCCTGTGTTCCTGATGATCCTGCTGA
AAGGCGGCGAGCAGAAGGGCCACGAGAAGATCAGCGCCAGCTTCAACGAGAAG
AAGCAGCAGTCCAGCCTGTACCTGACAGCCAGCCAGCTGAGCTACAGCGGCACC
TACTTTTGTGGCACCGCCTGGATCAACGACTACAAGCTGTCTTTCGGAGCCGGCA
CCACAGTGACAGTGCGGGCCAATATTCAGAACCCCGATCCTGCCGTGTACCAGCT
GAGAGACAGCAAGAGCAGCGACAAGAGCGTGTGCCTGTTCACCGACTTCGACAG
CCAGACCAACGTGTCCCAGAGCAAGGACAGCGACGTGTACATCACCGATAAGTG
CGTGCTGGACATGCGGAGCATGGACTTCAAGAGCAACAGCGCCGTGGCCTGGTC
CAACAAGAGCGATTTCGCCTGCGCCAACGCCTTCAACAACAGCATTATCCCCGA
GGACACATTCTTCCCAAGTCCTGAGAGCAGCTGCGACGTGAAGCTGGTGGAAAA
GAGCTTCGAGACAGACACCAACCTGAACTTCCAGAACCTGAGCGTGATCGGCTT
CAGAATCCTGCTGCTCAAGGTGGCCGGCTTCAACCTGCTGATGACCCTGAGACTG
TGGTCCAGCTGA
SEQ ID NO: 251: HD1 TCR α chain - amino acid sequence
METLLKVLSGTLLWQLTWVRSQQPVQSPQAVILREGEDAVINCSSSKALYS
VHWYRQKHGEAPVFLMILLKGGEQKGHEKISASFNEKKQQSSLYLTASQLSYSGTYF
CGTAWINDYKLSFGAGTTVTVRANIQNPDPAVYQLRDSKSSDKSVCLFTDFDSQTNV
SQSKDSDVYITDKCVLDMRSMDFKSNSAVAWSNKSDFACANAFNNSIIPEDTFFPSPE
SSCDVKLVEKSFETDTNLNFQNLSVIGFRILLLKVAGFNLLMTLRLWSS*
SEQ ID NO: 252: HD1 TCR β chain - DNA sequence
ATGGGATCTTGGACACTGTGTTGCGTGTCCCTGTGCATCCTGGTGGCCA
AGCACACAGATGCCGGCGTGATCCAGTCTCCTAGACACGAAGTGACCGAGATGG
GCCAAGAAGTGACCCTGCGCTGCAAGCCTATCAGCGGCCACGATTACCTGTTCTG
GTACAGACAGACCATGATGAGAGGCCTGGAACTGCTGATCTACTTCAACAACAA
CGTGCCCATCGACGACAGCGGCATGCCCGAGGATAGATTCAGCGCCAAGATGCC
CAACGCCAGCTTCAGCACCCTGAAGATCCAGCCTAGCGAGCCCAGAGATAGCGC
CGTGTACTTCTGCGCCAGCAGAAAGACAGGCGGCTACAGCAATCAGCCCCAGCA
CTTTGGAGATGGCACCCGGCTGAGCATCCTGGAAGATCTGAAGAACGTGTTCCCA
CCTGAGGTGGCCGTGTTCGAGCCTTCTGAGGCCGAGATCAGCCACACACAGAAA
GCCACACTCGTGTGTCTGGCCACCGGCTTCTATCCCGATCACGTGGAACTGTCTT
GGTGGGTCAACGGCAAAGAGGTGCACAGCGGCGTCTGTACCGATCCTCAGCCTC
TGAAAGAGCAGCCCGCTCTGAACGACAGCAGATACTGCCTGAGCAGCAGACTGA
GAGTGTCCGCCACCTTCTGGCAGAACCCCAGAAACCACTTCAGATGCCAGGTGC
AGTTCTACGGCCTGAGCGAGAACGATGAGTGGACCCAGGATAGAGCCAAGCCTG
TGACACAGATCGTGTCTGCCGAAGCCTGGGGCAGAGCCGATTGTGGCTTTACCAG
CGAGAGCTACCAGCAGGGCGTGCTGTCTGCCACAATCCTGTACGAGATCCTGCTG
GGCAAAGCCACTCTGTACGCCGTGCTGGTGTCTGCCCTGGTGCTGATGGCCATGG
TCAAGCGGAAGGATAGCAGGGGCTGA
SEQ ID NO: 253: HD1 TCR β chain - amino acid sequence
MGSWTLCCVSLCILVAKHTDAGVIQSPRHEVTEMGQEVTLRCKPISGHDY
LFWYRQTMMRGLELLIYFNNNVPIDDSGMPEDRFSAKMPNASFSTLKIQPSEPRDSA
VYFCASRKTGGYSNQPQHFGDGTRLSILEDLKNVFPPEVAVFEPSEAEISHTQKATLV
CLATGFYPDHVELSWWVNGKEVHSGVCTDPQPLKEQPALNDSRYCLSSRLRVSATF
WQNPRNHFRCQVQFYGLSENDEWTQDRAKPVTQIVSAEAWGRADCGFTSESYQQG
VLSATILYEILLGKATLYAVLVSALVLMAMVKRKDSRG*

TABLE 14
Efficiency of TCR insertion in edited T cells

| | CD3+ | | CD3 neg | | CD3+CD8+Dx+ | | |
|---|---|---|---|---|---|---|---|
| SEQ | Mean | | Mean | | Mean | | Samples |
| ID NO: | % | SD | % | SD | % | SD | (n) |
| 95 | 62 | N/A | 39 | N/A | 68 | N/A | 1 |
| 6 | 51 | N/A | 49 | N/A | 88 | N/A | 1 |
| 6 and 95 | 45 | 10 | 55 | 10 | 95 | 1 | 3 |
| mock | 98 | 1 | 2 | 1 | 0 | 0 | 3 |

TABLE 15
Phenotype of TCR edited T cells

| | | | | | | | | | Sam- |
|---|---|---|---|---|---|---|---|---|---|
| SEQ | N/Tscm | | CM | | EM | | TemRa | | ples |
| ID NO | Mean | SD | Mean | SD | Mean | SD | Mean | SD | (n) |
| PBMC | 44 | 4 | 25 | 5 | 19 | 3 | 12 | 5 | 3 |
| mock | 56 | 12 | 31 | 13 | 9 | 2 | 4 | 2 | 3 |
| 95 | 93 | N/A | 4 | N/A | 0 | N/A | 2 | N/A | 1 |
| 6 | 94 | N/A | 4 | N/A | 1 | N/A | 2 | N/A | 1 |
| 6 and | 82 | 8 | 13 | 6 | 3 | 2 | 2 | 0 | 3 |
| 95 | | | | | | | | | |

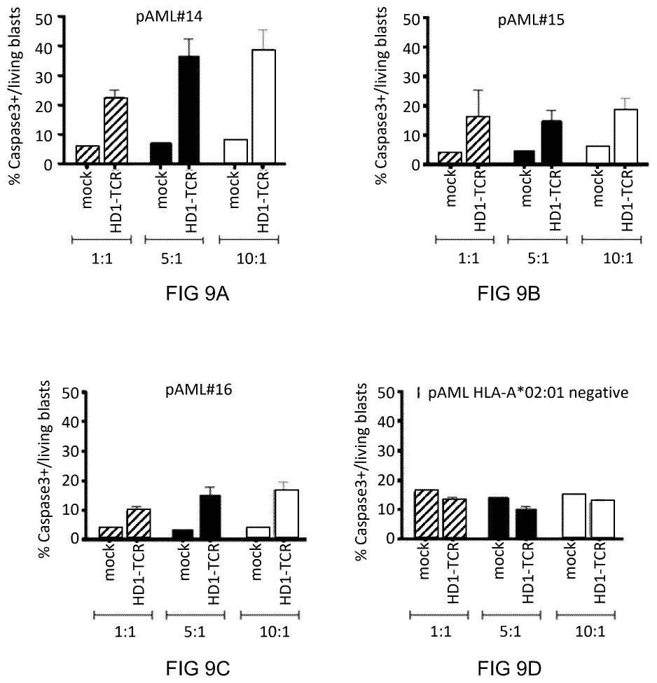

T cells with TRAC and TRBC edits and an LV HD1-TCR insertion were tested for ability to kill primary AML blasts. Edited T cells were magnetically sorted in order to enrich for WT1-CD8⁺ specific T cells using dextramer staining and magnetic sorting. Twenty-one days after the transduction, WT1-CD8⁺ T cells were co-cultured with primary AML blasts obtained from 3 different patients harboring the HLA-A*02:01 allele and, as control, with primary blasts not harboring the specific HLA allele. Co-cultures were seeded at different effector to target ratios (1:1; 5:1; 10:1). Upon 6 hours co-culture, expression of Caspase 3 in living target cells was used as read out. Results are shown in FIGS. 9A-D and Table 16. Induction of apoptosis in up to 43% of leukemic blasts expressing WT1 and harboring the HLA-A*02:01 (mean±SEM=31±6,6) was observed. No recognition was detectable against AML blasts not expressing the HLA-A*02:01 allele.

TABLE 16
AML killing

| | | | HD1-TCR+ | |
|---|---|---|---|---|
| Condition | E:F ratio | mock | Mean % | SD |
| pAML#14 | 1:1 | 5.9 | 22.1 | 4.9 |
| | 5:1 | 6.7 | 36.1 | 10.9 |
| | 10:1 | 7.5 | 38.6 | 11.6 |
| pAML#15 | 1:1 | 3.6 | 15.7 | 16.9 |
| | 5:1 | 4.4 | 14.5 | 6.7 |
| | 10:1 | 5.3 | 18.1 | 8.1 |
| pAML#16 | 1:1 | 3.1 | 9.4 | 3.1 |
| | 5:1 | 3.1 | 14.7 | 5.5 |
| | 10:1 | 3.4 | 16.4 | 5.6 |
| pAML HLA-A*02:01 | 1:1 | 16.2 | 13.2 | 1.8 |
| | 5:1 | 13.8 | 9.8 | 2.2 |
| | 10:1 | 15.1 | 12.1 | 2.5 |

### Example 8: Additional TRAC and TRBC Guides for Multiple Edits

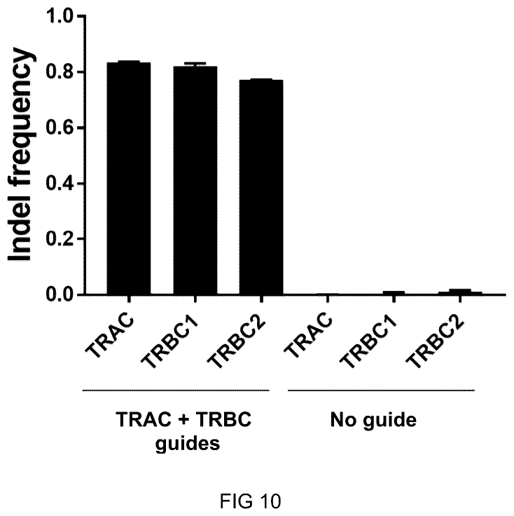

Additional crRNAs were also tested for simultaneous editing of TRAC and TRBC loci. T cells were nucleofected with RNP containing SEQ ID NO: 185, RNP containing SEQ ID NO: 6, or a mixture of both RNP species. Procedures were carried out as in Example 3 except using BioWhittaker™ X-VIVO™ 15 Hematopoietic Serum-Free Culture Media (Lonza™, Cat. 04-418Q) supplemented with 5% FBS, 50 uM beta-mercaptoethanol, 10 mM N-Acetyl L-Cystine, 1× Penicillin-Streptomycin Solution (Corning, Cat. 30-002-CI) and optionally with IL7 (5 ng/ml), IL15 (5 ng/ml), and IL2 (200-500 U/ml). Seventeen days after nucleofection, T cells were harvested and analyzed by NGS. FIG. 10 and Table 17 show editing at each locus (as indel frequency) following the simultaneous nucleofection. Flow cytometry can also be used to test editing efficiency by measuring the portion of T cells expressing TCR proteins on their surface.

TABLE 17
Editing with multiple guides

| | | Mean Indel | Std. |
|---|---|---|---|
| Condition | Amplicon | Frequency | Deviation |
| SEQ ID NO: | TRAC | 83.4% | 0.3% |
| 6 and SEQ | TRBC1 | 82.2% | 0.9% |
| ID NO: 185 | TRBC2 | 77.1% | 0.2% |
| No guide | TRAC | 0.1% | 0.1% |
| | TRBC1 | 0.4% | 0.6% |
| | TRBC2 | 1.0% | 0.7% |

### Example 9

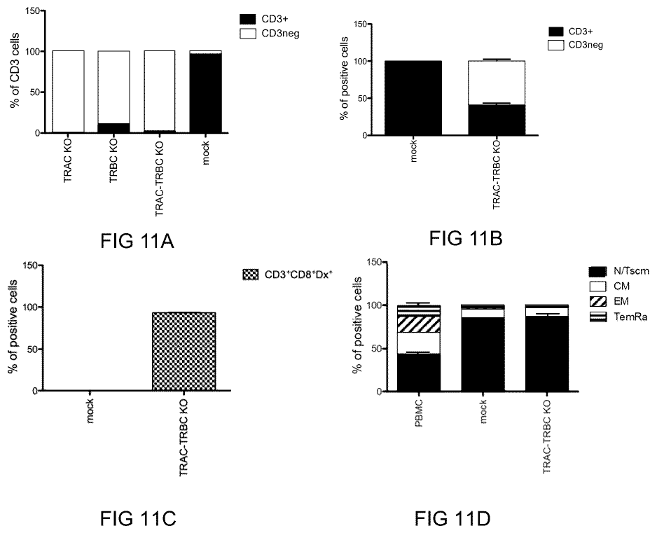

An additional pair of TRAC and TRBC targeting crRNAs were tested for combined editing as described in Example 7. T cells were nucleofected with RNP containing SEQ ID NO: 95, RNP containing SEQ ID NO: 2, or a mixture of both RNP species. TRAC and TRBC knockout efficiency was assessed by evaluating the percentage of T cells devoid of the CD3 molecule by flow cytometry as shown in FIG. 11A and Table 18.

TABLE 18
Efficiency of TCR knockdown

| SEQ | CD3+ | | CD3neg | | |
|---|---|---|---|---|---|
| ID NO: | Mean % | SD | Mean % | SD | Samples (n) |
| 95 | 0.5 | N/A | 99.5 | N/A | 1 |
| 2 | 10.2 | N/A | 89.3 | N/A | 1 |
| 2 and 95 | 1.8 | 1.0 | 98.1 | 1.0 | 3 |
| mock | 96.8 | N/A | 2.4 | N/A | 1 |

Following lentiviral transduction, T cell phenotype was evaluated by cytofluorimetric analysis as shown in FIG. 11B and Table 19. Percentage of WT1-specific CD8⁺ T cells was evaluated by measuring Dextramer positivity (FIG. 11C, Table 19). T cell phenotype of the edited T cells is shown in FIG. 11D and Table 20.

TABLE 19
Efficiency of TCR transduction in edited T cells

| | CD3+ | | CD3 neg | | CD3+CD8+Dx+ | | |
|---|---|---|---|---|---|---|---|
| SEQ | Mean | | Mean | | Mean | | Samples |
| ID NO: | % | SD | % | SD | % | SD | (n) |
| mock | 99.6 | N/A | 0.4 | N/A | 0 | N/A | 1 |
| 2 and 95 | 40.1 | 5.4 | 59.9 | 5.4 | 93 | 1 | 3 |

TABLE 20
Phenotype of TCR edited T cells

| SEQ | N/Tscm | | CM | | EM | | TemRa | | |
|---|---|---|---|---|---|---|---|---|---|
| ID NO | Mean | SD | Mean | SD | Mean | SD | Mean | SD | Samples (n) |
| PBMC | 44 | 4 | 25 | 5 | 19 | 3 | 12 | 5 | 3 |
| mock | 86 | N/A | 10 | N/A | 1 | N/A | 3 | N/A | 1 |
| 2 and 95 | 89 | 4 | 8 | 3 | 1 | 0 | 3 | 1 | 3 |

The engineered cells of examples 7 and 9 combine a TRAC gRNA and a TRBC gRNA with a lentiviral vector that encodes a WT1-specific TCR having a TCR α chain of SEQ ID NO: 251 and a TCR β chain of SEQ ID NO: 253. The engineered cell can be altered by delivering a first and a second composition to the cell, and wherein the first composition comprises: (a) a guide RNA comprising a sequence chosen from: (i) a guide sequence selected from SEQ ID NOs: 1-89; (ii) at least 17, 18, 19, or 20 contiguous nucleotides of a sequence selected from SEQ ID NOs: 1-89; (iii) a guide sequence that is at least 99%, 98%, 97%, 96%, 95%, 94%, 93%, 92%, 91%, or 90% identical to a sequence selected from SEQ ID NOs: 1-89; (iv) a guide sequence comprising any one of SEQ ID NOs: 1-24; and (iv) a guide sequence comprising any one of SEQ ID NOs: 1-6; or (b) a nucleic acid encoding a guide RNA of (a.); and optionally (c) an RNA-guided DNA binding agent or a nucleic acid encoding an RNA-guided DNA binding agent; and wherein the second composition comprises a lentiviral vector that encodes a WT1-specific TCR having a TCR α chain of SEQ ID NO: 251 and a TCR β chain of SEQ ID NO: 253.

In some embodiments, the engineered cell can be altered by delivering a composition further comprising: (a) a guide RNA comprising a sequence chosen from: (i) a guide sequence selected from SEQ ID NOs: 90-178, 185, and 213-218; (ii) at least 17, 18, 19, or 20 contiguous nucleotides of a sequence selected from SEQ ID NOs: 90-178, 185, and 213-218; (iii) a guide sequence that is at least 99%, 98%, 97%, 96%, 95%, 94%, 93%, 92%, 91%, or 90% identical to a sequence selected from SEQ ID NOs: 90-178, 185, and 213-218; (iv) a guide sequence comprising any one of SEQ ID NOs: 90-113 and 213-218; and (iv) a guide sequence comprising any one of SEQ ID NOs: 90-95; or (b) a nucleic acid encoding a guide RNA of (a.).

### Example 10. Editing with Single Guides

Examples below were performed by the following method unless noted otherwise in the examples.

### Genomic DNA Isolation

T cells were harvested >48 hours post-nucleotransfection. DNA isolation was performed as described in Example 1. DNA samples were subjected to PCR and subsequent NGS analysis as described in Example 1.

### Delivery of RNPs to T Cells

Healthy donor PBMCs or leukopaks were obtained commercially and T cells isolated by positive selection using the CD4/CD8 straight from Microbeads kit (Miltenyi Biotec) or by negative selection using the EasySep Human T-cell Isolation Kit (Stem Cell Technology, Catalog #17951) following the manufacturers protocol. T cells were cryopreserved in Cryostor CS10 freezing media (Catalog #07930) for future use. Upon thaw, T cells were rested overnight in base media (XVIVO15 supplemented with 2-mercaptoethanol, N-acetyl-cysteine and 5% human AB serum or FBS) supplemented with 200 U/mL IL2 (Peprotech) and 5 ng/mL each of IL7 and IL15. T cells were subsequently activated with T Cell TransAct (Miltenyi Biotec, 130-111-160) as recommended by manufacturer and cultured for 48-72 hours prior to electroporation.

The ribonucleoprotein (RNP) complex comprising a Cas9 protein and ggRNA was generated by first pre-annealing individual crRNA and trRNA by mixing equivalent amounts of reagent and incubating at 95° C. for 2 min and cooling to room temperature. Guide(s) in the RNP utilized crRNA and trRNA associated as a single RNA molecule (single guide RNA, sgRNA) or in two separate RNA molecules (dual guide RNA, dgRNA) that were previously annealed by incubating at 95° C. for 2 min followed by cooling to ambient temperature. RNPs targeting the TRAC or TRBC locus were complexed by mixing either SEQ ID NO: 186 (TRAC) or SEQ ID NO: 180 (TRBC1/2) with SpyCas9 at a 2:1 molar ratio to a final concentration of 20 uM Cas9-RNP. For electroporation, T cells were suspended at 5-20×10⁶ cells/100 uL with TRAC and TRBC RNPs added as indicated to a final concentration of 2 μM RNP. T cells were nucleofected with RNP using the P3 Primary Cell 96-well Nucleofector™ Kit (Lonza, Cat. V4SP-3960) or Lonza 4D-Nucleofector X Unit (Catalog #: AAF-1002X) using Buffer P3 and manufacturer's pulse codes. Base media was added to cells immediately post electroporation and cells allowed to rest for at least 4 hours.

For select assays, cells were expanded using the G-Rex protocol. Briefly, after electroporation, transfer 1×10\hat{ }6 cells in 1 ml of cytokine media in 1 well of a 24 well G-Rex and virus equivalent to MOI 3×10\hat{ }5 to the well. After 24 h, make up the volume to 7 ml using cytokine media. Every 2-3 days, remove half the media (3.5 ml) without disturbing the cells and add 2× cytokine media. Harvest after on Day 11-13.

### Cell Staining for Flow Cytometry

Four to 12 days after RNP nucleofection, edited T cells (200 ul/200,000 cells) were collected in a 96 well round bottom plate and spun down at 500 g for 5 minutes. Cells were then resuspended in an antibody mixture containing a combination of antibodies targeting CD3, CD4, CD8, CD45RO, CD45RA, CD27, CCR7, CD62L, and/or the TCR specific pMHC tetramer or TCR specific Vbeta chain antibody. The mixture was incubated for 45 minutes at room temperature in the dark. Samples were spun down at 500 g for 5 minutes, cell pellets collected, and resuspended in 1:10,000 DAPI (Biolegend—#422801) in FACS buffer. Flow cytometry was then performed using the cytoflex according to the manufacturer's instructions.

Single guides were also tested for individual and simultaneous editing at TRAC and TRBC loci in stimulated T cells. T cells were nucleofected with RNP containing guide SEQ ID NO: 180 targeting the TRBC1 and TRBC2 loci, RNP containing guide SEQ ID NO: 186 targeting TRAC, or a mixture of both RNP species at a 2:1 ratio of guide to Cas9. Two days post electroporation, T cells were passaged and lysates collected.

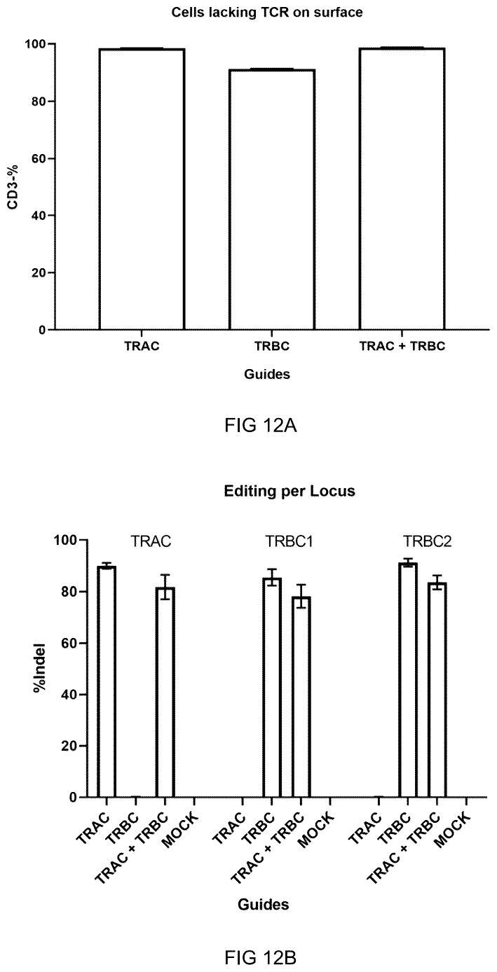

Genomic DNA was prepared and NGS analysis performed as described in Example 1. Test samples were performed in triplicate, with a single mock sample. The knockdown efficiency of TCR surface expression for individual and simultaneous editing of TRAC and TRBC guides was assessed by flow cytometry targeting the presence or absence of CD3. The percentages of T cells devoid of the CD3 molecule are shown in Table 21 and FIG. 12A.

TABLE 21
Edited CD3− cells in population

| | Mean % | | |
|---|---|---|---|
| Sample | CD3− | SD | n |
| TRAC RNP | 98.47 | 0.06 | 3 |
| TRBC RNP | 91.20 | 0.17 | 3 |
| TRAC/TRBC RNP | 95.83 | 5.05 | 3 |

The indel frequency was assessed by NGS analysis following the individual and combinatorial editing of TRAC and TRBC in CD3− T cells. The percentage of guide editing in TRAC and TRBC are shown in Table 22 and FIG. 12B.

TABLE 22
TRAC and TRBC guide editing in T cells

| | TRAC | | TRBC1 | | TRBC2 | | |
|---|---|---|---|---|---|---|---|
| SEQ | Mean | TRAC | Mean | TRBC1 | Mean | TRBC2 | |
| ID NO: | Indel % | SD | Indel % | SD | Indel % | SD | N |
| TRAC KO | 90 | 1.2 | 0.4 | 0 | 0.2 | 0.1 | 3 |
| TRBC KO | 0.2 | 0.1 | 85.4 | 3.2 | 91.2 | 1.5 | 3 |
| TRAC | 81.8 | 4.7 | 78.2 | 4.4 | 83.6 | 2.7 | 3 |
| KO + | | | | | | | |
| TRBC KO | | | | | | | |
| Mock | 0.2 | — | 0.2 | — | 0.1 | — | 1 |

### Example 11. TRAC Guide Insertion Screen

Insertion of AAV Template into T Cells

The location of 12 guides targeting the TRAC locus was used to design an adenovirus-associated virus (AAV) template that would delete an 80 bp region of the TRAC locus to prevent cutting of the site after repair and preventing the guide/Cas9 from cutting the template. This would create a locus in which a TCR can be inserted into the TRAC locus. Using the homology arms with the 80 bp deletion, a construct was designed to insert the model TCR and the second to insert GFP with both driven by the EF1a promoter that were then synthesized by GenScript USA Inc. and inserted into a pUC19 vector with BglII restriction sites for subcloning into an AAV vector. AAV templates were designed with homology arms flanking TRAC guide cut sites to insert genes encoding TCRs or reporter molecules (ie. GFP) site-specifically in T cells.

T cell isolation and RNP nucleotransfection procedures were performed as described in Example 10. Ten minutes post electroporation, T cells were added to cytokine media at 1×10⁶ cells/mL with AAV template added at a MOI of 3×10⁵. Twenty-four hours post electroporation, cells were split and expanded for an additional 4-12 days in static culture or G-REX multiwell plates (Wilson Wolf Manufacturing) prior to use in functional assays or cryopreserved. Flow cytometry was performed.

### TRAC Guide Insertion Screen

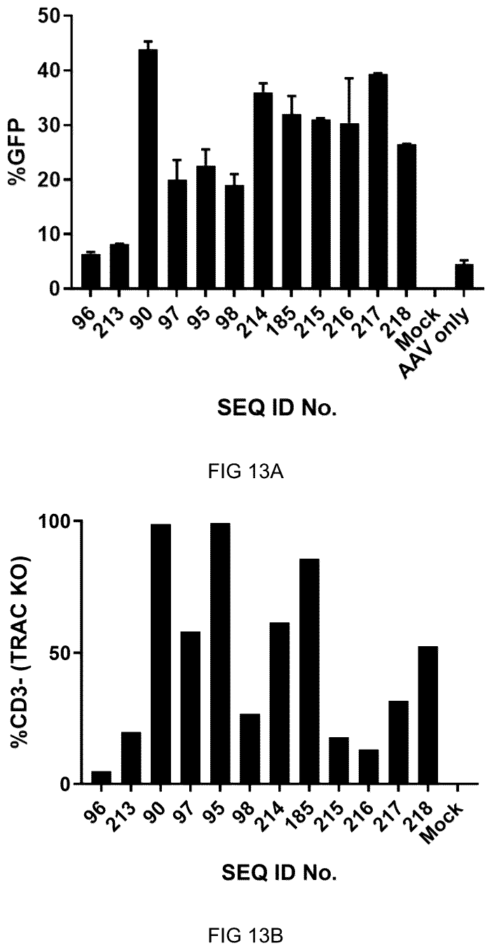

Dual guides targeting exon 1 of the TRAC locus were screened in T cells to evaluate insertion efficiency. The initial screen used an AAV-based insertion template (AV9) encoding a GFP reporter gene AV9 was designed with an 80 bp gap designed to cover the target sites of the guides listed in Table 23. AV9 is described in Table 37, below. T cells were nucleofected in duplicate with RNP containing a dual guide except for the AAV only and no RNP (mock) samples that were used as controls. Procedures for cell transduction with RNP were carried out as described in Example 10 with the exception that the guide to Cas9 ratio was 1:1. The insertion efficiency of the TRAC guides was determined by flow cytometry, as described in Example 10, to detect the fluorescence of GFP from the inserted construct as shown in Table 23 and FIG. 13A. The knockdown efficiency of TCR surface expression for editing by TRAC guides was assessed by flow cytometry targeting the presence or absence of CD3. The percentage of T cells devoid of the CD3 molecule are shown in Table 23 and FIG. 13B.

TABLE 23
Percent GFP expression of inserted guides

| | | GFP % | % CD3− |
|---|---|---|---|
| Guide | GFP % | SD | cells (n = 1) |
| 96 | 6.44 | 0.28 | 4.96 |
| 213 | 8.22 | 0.06 | 19.8 |
| 90 | 43.9 | 1.41 | 98.9 |
| 97 | 20.05 | 3.61 | 58.1 |
| 95 | 22.65 | 2.9 | 99.2 |
| 98 | 19.1 | 1.98 | 26.8 |
| 214 | 36.05 | 1.63 | 61.5 |
| 185 | 32.05 | 3.32 | 85.7 |
| 215 | 31 | 0.28 | 17.9 |
| 216 | 30.4 | 8.2 | 13.1 |
| 217 | 39.4 | 0.14 | 31.6 |
| 218 | 26.55 | 0.07 | 52.5 |
| Mock | 0 | 0 | 0.45 |
| AAV only | 4.52 | 0.72 | NA |

### Example 12. Single Guide Screen for TRAC Insertion

Single guides targeting exon 1 of the TRAC locus were then screened in T cells to evaluate the insertion efficiency using AV9. Procedures for T cell preparation and RNP transduction were carried out in duplicate as described in Example 10 with the exception that the guide:Cas9 ratio was 1:1. AV9 was designed as described in Example 11 with the guides listed in Table 24. NGS indel analysis was conducted as described in Example 1.

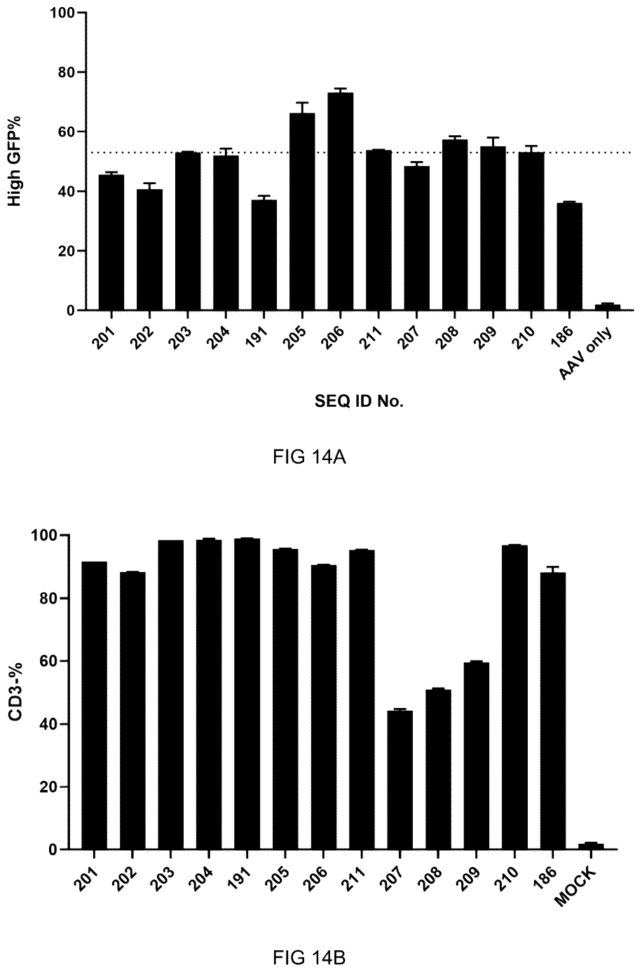

The insertion efficiency of the TRAC guides was determined by flow cytometry to detect the fluorescence of GFP from the inserted construct as shown in Table 24 and FIG. 14A. The knockout efficiency of TCR surface expression by TRAC guides was assessed by flow cytometry targeting the presence or absence of CD3. The percentage of T cells devoid of the CD3 molecule are shown in Table 24 and FIG. 14B. Editing efficiencies of chemically modified and unmodified guides were also assayed and found to have activity.

TABLE 24
Percentage of GFP positive cells

| | % GFP | % GFP | % CD3− | % CD3− |
|---|---|---|---|---|
| Guide | (Mean) | SD | cells | cells SD |
| 201 | 45.65 | 0.78 | 88.85 | 0.21 |
| 202 | 40.65 | 2.05 | 89.10 | 0.42 |
| 203 | 53.05 | 0.21 | 96.15 | 0.21 |
| 204 | 51.95 | 2.33 | 95.70 | 0.71 |
| 191 | 37.15 | 1.34 | 95.95 | 0.21 |
| 205 | 66.35 | 3.46 | 93.50 | 0.71 |
| 206 | 73.15 | 1.34 | 93.25 | 0.78 |
| 211 | 53.75 | 0.21 | 92.55 | 0.21 |
| 207 | 48.45 | 1.34 | 62.25 | 0.64 |
| 208 | 57.35 | 1.06 | 71.50 | 0.14 |
| 209 | 55.00 | 2.97 | 72.15 | 1.91 |
| 210 | 53.15 | 2.05 | 93.70 | 0.28 |
| 186 | 36.15 | 0.35 | 85.50 | 0.57 |
| Mock | 1.88 | 0.41 | 0.63 | 0.01 |
| AAV only | 0.00 | 0.00 | 1.71 | 0.78 |

### Example 13—Assessing Promoters and ITRs for Exogenous TCR Insertion and Expression

### Engineering of Gene-Editing TCR-T Cells for Functional Assays

TCR insertion and subsequent surface expression was tested with a variety of promoters and two ITR lengths. T cell isolation and RNP transfection with guide SEQ ID NO: 185 were conducted as described in Examples 10 with the exception of using a 1:1 molar ratio of gRNA to SpyCas9. RNP transduction of AAV constructs containing TCR-C into T cells was conducted as described in Example 11. The AAV templates contained 500 bp homology arms corresponding to the 500 bp flanking each side of the SEQ ID NO: 185 cut site in the TRAC locus. Each AAV construct featured different combinations of promoters and ITRs as shown in Table 25. The edited T cells were tetramer stained for flow cytometric analysis as described in Example 11.

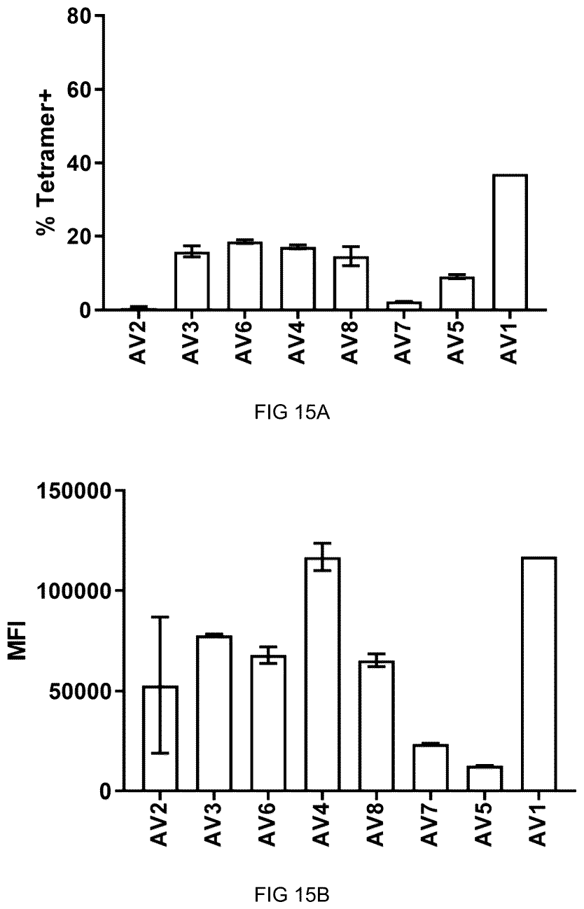

TCR-C insertion efficiency was evaluated by cytofluorimetric analysis assessing both T cell identity through the presence of CD3+ on the cell surface and the number of cells expressing the inserted TCR via to a tetramer of the TCR's specific ligand, the RMF peptide (see, e.g., US20160083449A1, the contents of which are hereby incorporated by reference). The percentage of CD3+ Tetramer+ cells is shown in Table 25 and FIG. 15A. Mean fluorescence intensity (MFI) of the for the tetramer stained T cells is shown in Table 25 and FIG. 15B.

TABLE 25
Promoters and ITRs for AAV templates
The sequence elements and sequences shown
in this table are further defined in Table 37.

| | | | % | | Fluores- | | |
|---|---|---|---|---|---|---|---|
| AAV | | | Tetra- | | cence | | |
| Tem- | | ITR | mer+, | | intensity | | |
| plate | Pro- | length | CD3+ | | CD3+ | | |
| ID | moter | (bp) | (Mean) | SD | (mean) | SD | n |
| AV2 | Ef1a - | 145 | 0.48 | 0.48 | 52752 | 34002 | 2 |
| | short | | | | | | |
| AV3 | MND-1 | 141 | 15.85 | 1.48 | 77704 | 646 | 2 |
| AV6 | MND-1 | 145 | 18.55 | 0.49 | 67845 | 4084 | 2 |
| AV4 | MND-2 | 141 | 17.10 | 0.57 | 116978 | 6798 | 2 |
| AV8 | MND-2 | 145 | 14.55 | 2.62 | 65281 | 3117 | 2 |
| AV5 | PGK | 135 | 2.35 | 0.03 | 23410 | 437 | 2 |
| AV7 | PGK | 146 | 9.01 | 0.57 | 12596 | 78 | 2 |
| AV1 | EF1a | 146 | 37 | — | 117223 | — | 1 |

### Example 14: GFP Expression in Engineered T Cells Using Endogenous TRAC Promoter

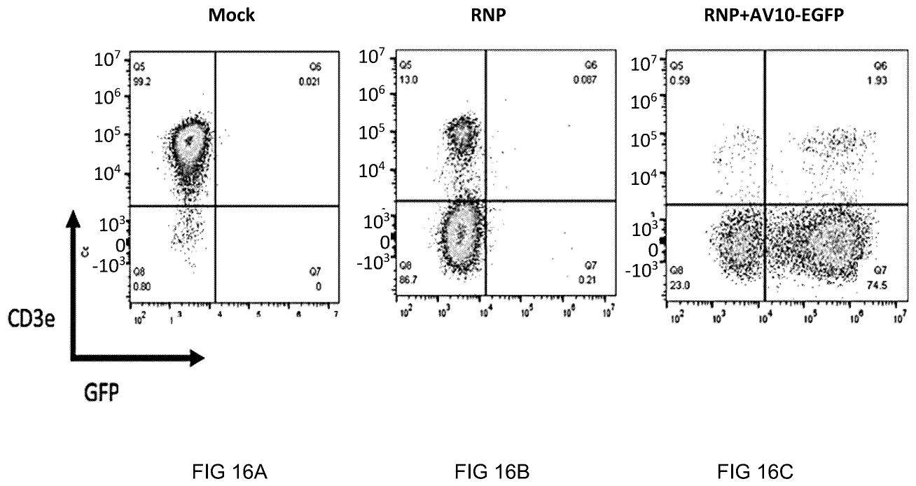

In another experiment, an AAV template (AV10) containing a P2A ribosomal skip site followed by a GFP reporter gene (ie. without an exogenous promoter) was inserted into exon 1 of the TRAC locus to determine if the endogenous TRAC promoter could drive GFP expression. T cells were nucleofected with RNP containing guide SEQ ID NO: 186 and transduced with AAV AV10 as described in Example 10. Control conditions included unedited T cells (Mock) and T cells which received RNP nucleofection but not AAV (RNP only). After 4-days of culture edited T cells were co-stained with APC-Cy7 conjugated anti-CD3e (Biolegend, 300318) for CD3 and analyzed by flow cytometry for GFP expression and TCR knockout as shown in Table 26 and FIGS. 16A-C.

TABLE 26
Percentage of edited CD3− and GFP positive cells in population.

| Sample | % CD3− | SD | % GFP+ | SD |
|---|---|---|---|---|
| AV10-GFP + RNP | 97.5 | 0.05 | 76.4 | 1.9 |
| RNP only | 86.7 | 2.05 | 1.1 | 0.0 |
| Mock | 0.8 | 0.04 | 0.0 | 0.0 |

### Example 15: TCR Expression in Engineered T Cells Using Endogenous and Exogenous TRAC Promoter

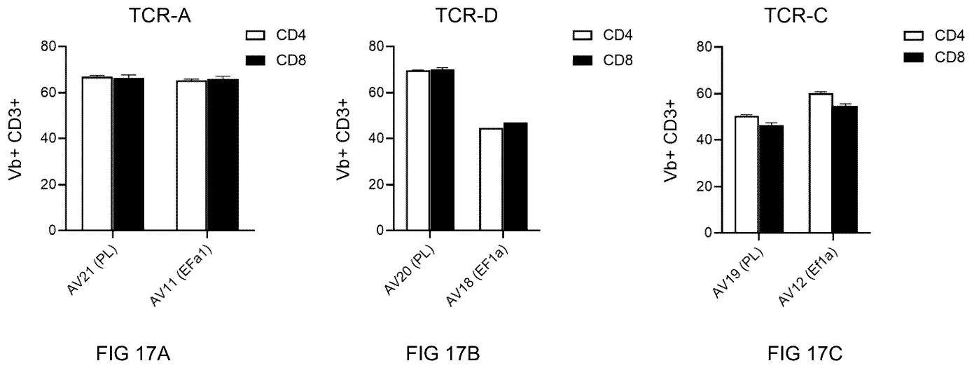

In an additional experiment, AAV templates containing TCR-A, TCR-C, or TCR-D with or without an exogenous promoter were inserted into the TRAC locus to determine if the endogenous TRAC promoter could drive expression and function of the inserted TCRs. T cell transfection and AAV insertion were conducted in duplicate as described in Examples 10 and 11 respectively. T cells were nucleofected with RNP containing TRAC (SEQ ID NO: 186) and TRBC (guide SEQ ID NO: 180) with the RNP containing guide contained various AAV templates shown in Table 27. T cells expressing the inserted TCR were detected by flow cytometry using antibodies specific to the TCR Vbeta chain of the inserted TCR in CD4+ and CD8+ cells as shown in Table 27 and FIGS. 17A-C (anti-Vbeta8 for TCR-A, [Biolegend Cat #140104], Vbeta7.2 for TCR-D in FIG. 17B [Beckman Coulter, Cat #IM3604], and Vbeta17 for TCR-C shown in FIG. 17C [Beckman Coulter, Cat #IM2048]).

TABLE 27
Surface expression of engineered TCRs
with and without exogenous promoters

| AAV | | | % CD4+ | | % CD8+ | |
|---|---|---|---|---|---|---|
| tem- | | | CD3+ Vb+ | | CD3+ Vb+ | |
| plate | TCR | Promoter | (mean) | SD | (mean) | SD |
| AV21 | TCR-A | Endog- | 67.05 | 0.49 | 66.50 | 1.27 |
| | | enous(PL) | | | | |
| AV11 | TCR-A | EF1a | 65.45 | 0.49 | 66.05 | 1.20 |
| AV20 | TCR-D | Endog- | 69.85 | 0.07 | 70.25 | 0.64 |
| | | enous(PL) | | | | |
| AV18 | TCR-D | EF1a | 44.60 | 0.00 | 47.00 | 0.00 |
| AV19 | TCR-C | Endog- | 50.45 | 0.35 | 46.40 | 0.99 |
| | | enous(PL) | | | | |
| AV12 | TCR-C | EF1a | 60.20 | 0.71 | 54.70 | 0.85 |

*PL—promoterless template construct

The sequence elements and sequences shown in this table are further defined in Table 37.

T-cell degranulation and cytokine release in response to the specific WT1 peptide antigen was performed to test the functionality of engineered effector T cells expressed from an endogenous promoter or exogenous TRAC promoter. Specifically, engineered T cells were assessed for upregulation of CD107α and expression of IL2 and IFNy after co-culture with peptide pulsed cells from the cancer cell lines OCI-AML3 (DSMZ, Cat. ACC 582). Briefly, 100,000 OCI-AML3 cells were pulsed in base media (Xvivo base media: No Cytokines+1 uL/mL GolgiPlug+0.7 uL/mL GolgiStop with 30 uL/mL CD107a APC/Cy7 (3 ul/well)) for 4-5 hours at 37 degrees with titrated amounts of the 9-mer peptides VLDFAPPGA (VLD) or RMFPNAPYL (RMF) peptide concentrations ranging from 0 to 5000 nM. Gene edited TCR+ T cells were suspended at 1×10\hat{ }6 TCR inserted cells/ml in XVIVO15 base media containing for a final concentration of 30 ul/ml CD107a APC/Cyanine7 antibody (Biolegend), 1 ul/ml Golgiplug (BD) and 0.7 ul/ml Golgistop (BD) and added to the peptide pulsed target cells at a 1:1 Effector:Target (E:T)cell ratio. Co-cultures were incubated overnight at 37° C., and the cells stained for surface markers CD3, CD4, CD8 and the specific TCR beta chain of TCR-A or TCR-D, and incubated for 30 minutes. Following surface staining, cells were fixed and permeabilized using a commercially available kit (Invitrogen) for intracellular IFN-β and TNFα staining followed by a 30 minute incubation at room temperature. Following intracellular stain, cells were washed and analyzed by flow cytometry.

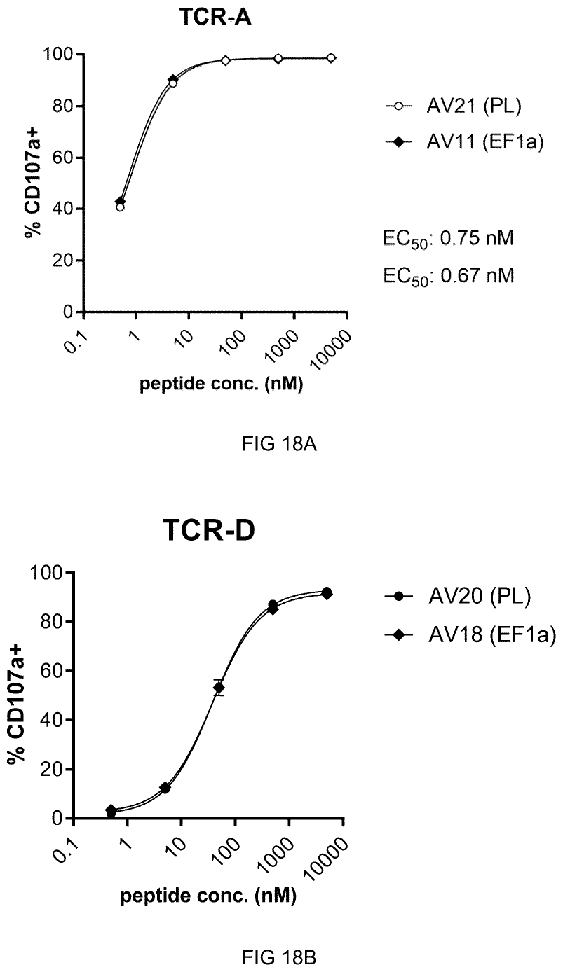

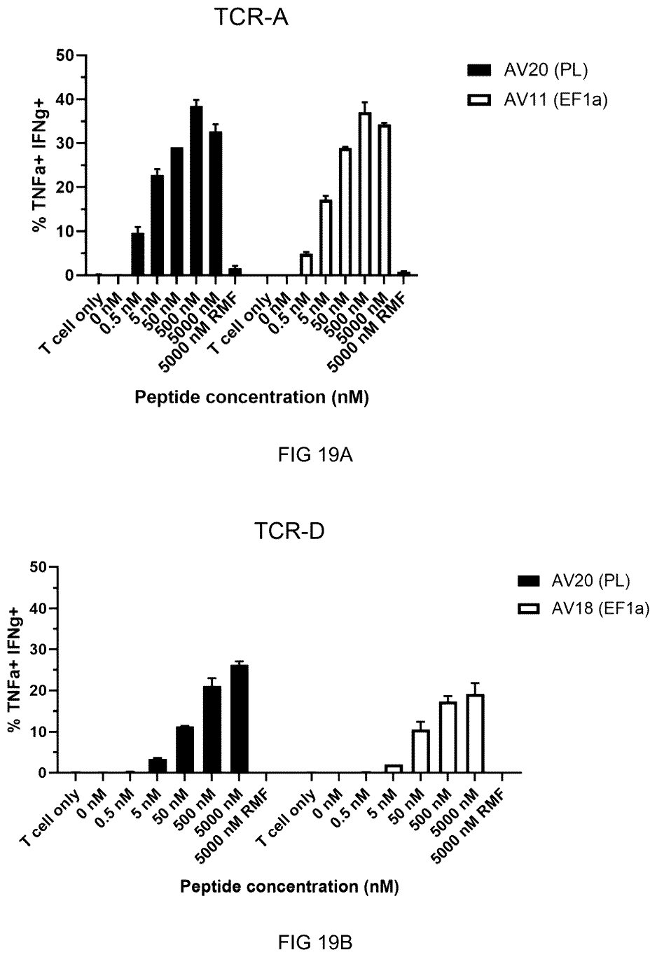

The impact of the EF1a (or EF-1α) and endogenous promoters on T cell killing and cytokine release was measured by the percentage of cells positive for CD107a, IFNy, and or TNFa present in each sample. The effector T cells with TCRs and promoter type are listed in Table 28. Flow cytometry results for the TCR+ T cells are shown in Table 28 and dose response curves for cells expressing TCR-A are shown in FIG. 18A (AV21 and AV11). The percentage of CD107a+ cells expressing TCR-D are shown in Table 28 and FIG. 18B (AV20 and AV18). The immune response elicited by the T cells was evaluated by measuring the percentage of TNF-alpha and interferon gamma as shown in Table 28 and FIGS. 19A-B.

TABLE 28
Degranulation and Interferon gamma response in engineered T
cells expressing TCRs with and without exogenous promoters

| | | | | | | | % | |
|---|---|---|---|---|---|---|---|---|
| | | | Peptide | | % | | TNFa+ | |
| Construct | TCR | Promoter | Concentration | | CD107a+ | SD | IFNg+ | SD |
| AV21 | TCR-A | Endogenous | 0 | nM | 3.79 | 0.49 | 0.08 | 0.02 |
| | | | 0.5 | nM | 40.55 | 0.35 | 9.66 | 1.34 |
| | | | 5 | nM | 88.70 | 0.42 | 22.75 | 1.34 |
| | | | 50 | nM | 97.55 | 0.07 | 29.10 | 0.00 |
| | | | 500 | nM | 98.55 | 0.07 | 38.55 | 1.34 |
| | | | 5000 | nM | 98.65 | 0.35 | 32.80 | 1.56 |
| | | | 5000 | nM RMF | 11.50 | 0.71 | 1.60 | 0.57 |
| AV11 | TCR-A | EF1a | 0 | nM | 3.83 | 0.33 | 0.02 | 0.03 |
| | | | 0.5 | nM | 42.85 | 0.07 | 4.92 | 0.40 |
| | | | 5 | nM | 90.15 | 0.78 | 17.20 | 0.85 |
| | | | 50 | nM | 97.65 | 0.21 | 28.90 | 0.28 |
| | | | 500 | nM | 98.15 | 0.07 | 37.15 | 2.19 |
| | | | 5000 | nM | 98.60 | 0.00 | 34.35 | 0.35 |
| | | | 5000 | nM RMF | 11.45 | 0.21 | 0.83 | 0.11 |
| AV20 | TCR-D | Endogenous | 0 | nM | 1.73 | 0.18 | 0.05 | 0.04 |
| | | | 0.5 | nM | 2.16 | 0.19 | 0.21 | 0.08 |
| | | | 5 | nM | 11.95 | 0.21 | 3.40 | 0.23 |
| | | | 50 | nM | 53.30 | 0.85 | 11.35 | 0.07 |
| | | | 500 | nM | 87.15 | 0.64 | 21.05 | 1.91 |
| | | | 5000 | nM | 92.30 | 0.28 | 26.20 | 0.85 |
| | | | 5000 | nM RMF | 1.82 | 0.13 | 0.01 | 0.01 |
| AV18 | TCR-D | EF1a | 0 | nM | 2.56 | 0.11 | 0.01 | 0.02 |
| | | | 0.5 | nM | 3.52 | 0.45 | 0.12 | 0.08 |
| | | | 5 | nM | 12.70 | 1.98 | 2.12 | 0.00 |
| | | | 50 | nM | 53.25 | 4.60 | 10.58 | 1.87 |
| | | | 500 | nM | 85.10 | 2.40 | 17.25 | 1.34 |
| | | | 5000 | nM | 91.35 | 1.63 | 19.15 | 2.62 |
| | | | 5000 | nM RMF | 2.86 | 0.25 | 0.05 | 0.01 |

The sequence elements and sequences shown in this table are further defined in Table 37.

### Example 16: Enhancing TCR Expression by Knockout of Both Endogenous Tcell Receptor Alpha and Beta Chains

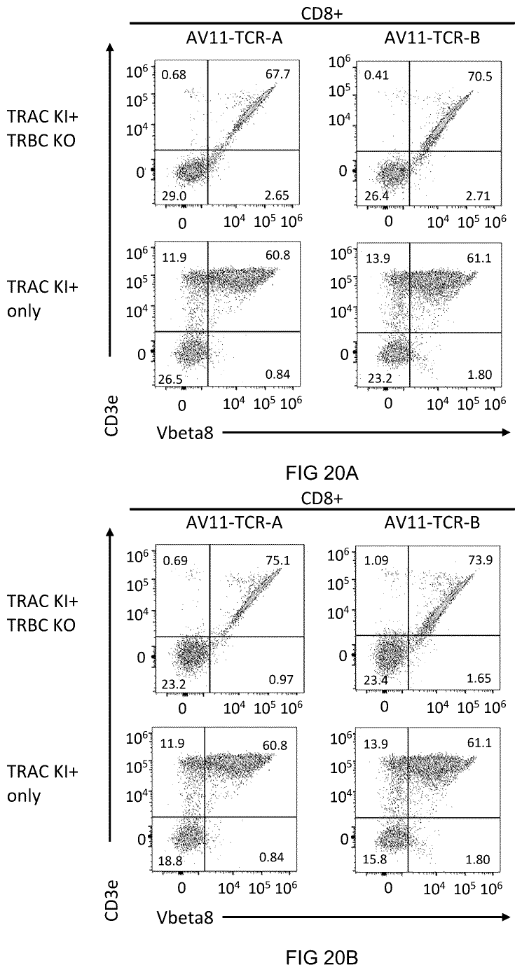

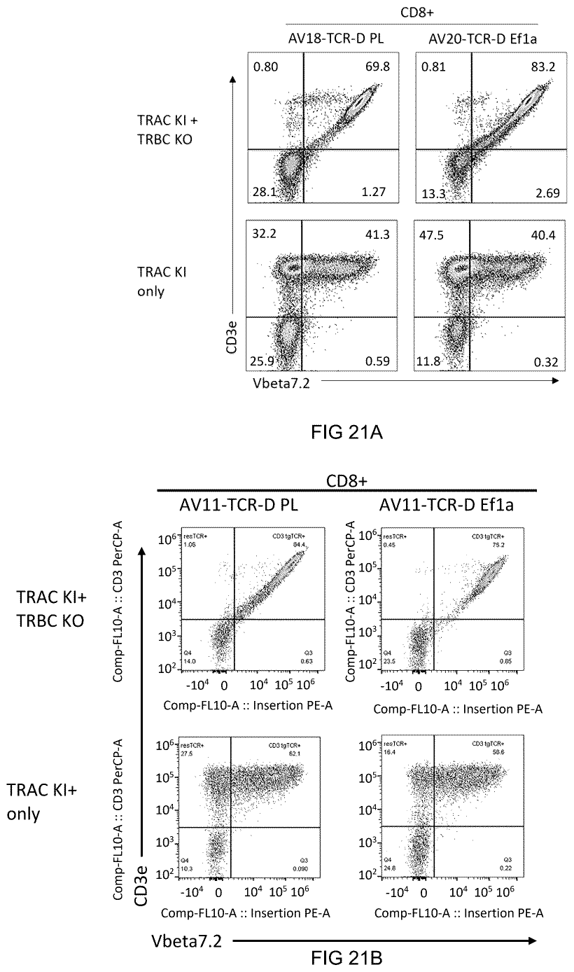

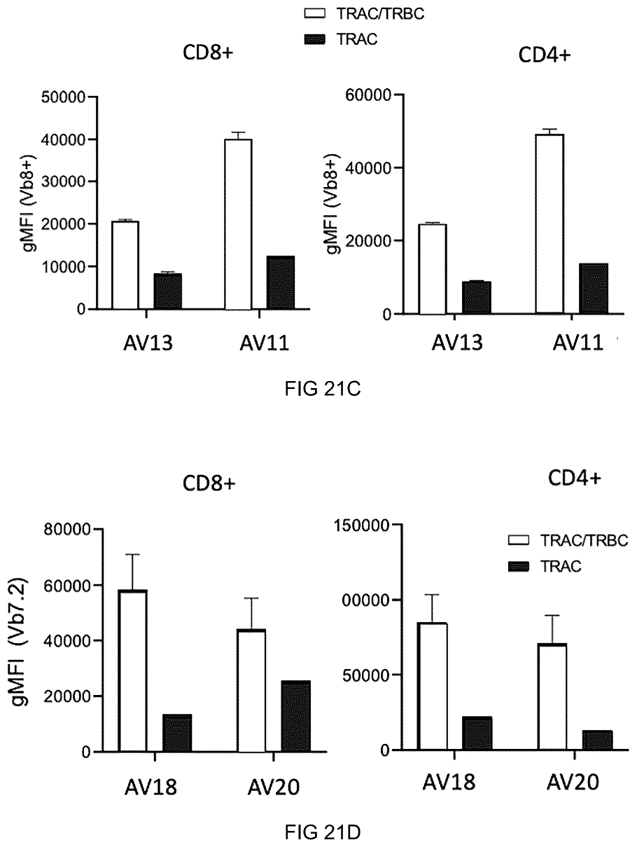

T cells were evaluated for surface expression of engineered TCR-A, TCR-B, or TCR-D comprising both the inserted alpha chain and inserted beta chain. Three conditions to increase pairing of inserted alpha and beta chains were tested: (1) the addition of cysteine residues that may form cysteine bridges to associate the inserted alpha and beta chains; (2) expression from the endogenous TRAC promoter or from and exogenous promoter and (3) the disruption of endogenous copies of TRAC, TRBC1 and TRBC2. T cell transfection and AAV transduction were conducted as described in Example 10. TCRs were inserted via AAV templates AV 11, AV13, AV18, or AV20 with either RNP targeting only the TRAC locus or RNP targeting both the TRAC, TRBC1, and TRBC2 loci, as described above. Post transfection, T cells were expanded as described in Example 10. Nine days post cell expansion, edited T cells were co-stained with anti-CD3e and an appropriate V-beta reagent (PE) to identify engineered TCRs via flow cytometry. The percentage of TCR-A and TCR-B CD3+ Vb7.2+ cells for each condition is shown in Table 29. FIGS. 20A and 20B plot a representative example of surface expression of engineered TCR-A and TCR-B CD3+ Vb7.2+ cells for the data in Table 29. Surface expression of engineered TCR-A and TCR-B in the CD8+ and CD4+ cell populations was also measured as shown in Table 29 and FIG. 21C.

TABLE 29
Assessment of expression of engineered TCR-A and TCR-B
comprising both inserted alpha and beta chains

| | | CD8+ | | | | CD4+ | | | |
|---|---|---|---|---|---|---|---|---|---|
| | | Mean | | | | Mean | | | |
| | | CD3+ | | Vb | | CD3+ | | Vb | |
| Sample | RNP | % Vb+ | SD | MFI | SD | % Vb+ | SD | MFI | SD |
| AV13- | TRAC | 62.2 | 1.56 | 8487 | 290\grave{ } | 68.85 | 0.64 | 8947 | 291 |
| TCR-B | TRAC + | 69.05 | 2.05 | 20709 | 443.36 | 75.35 | 2.05 | 24521 | 492 |
| (with cys) | TRBC | | | | | | | | |
| AV11- | TRAC | 60.8 | nd | 12520 | nd | 69.3 | nd | 13782 | nd |
| TCR-A | TRAC + | 67.55 | 0.21 | 40104 | 2203 | 74.75 | 0.50 | 49273 | 1743 |
| (no cys) | TRBC | | | | | | | | |

The percentage of AV20-TCR-D and AV18-TCR-D CD3+Vb7.2+ cells using the endogenous or exogenous promoter is shown in Table 30. Surface expression of engineered TCR-D in the CD8+ and CD4+ cell populations was measured as shown in Table 30 and FIGS. 21A-D.

TABLE 30
Assessment of expression of engineered TCR-D comprising
both inserted alpha and beta chains

| | | CD8+ | | | | CD4+ | | | |
|---|---|---|---|---|---|---|---|---|---|
| | | CD3+ | | Vb | | CD3+ | | Vb | |
| Sample | RNP | % Vb+ | SD | MFI | SD | % Vb+ | SD | MFI | SD |
| AV18- | TRAC | 41.3 | nd | 13586 | nd | 58.6 | nd | 22324 | nd |
| TCR-D | TRAC + | 71.4 | 1.6 | 58469 | 12479 | 74.05 | 1.15 | 85360 | 18034 |
| | TRBC | | | | | | | | |
| AV20- | TRAC | 40.4 | nd | 25613 | nd | 62.1 | nd | 13406 | nd |
| TCR-E | TRAC + | 83.5 | 0.3 | 44342 | 11035 | 84.1 | 0.3 | 71007 | 18790 |
| | TRBC | | | | | | | | |

### Example 17: Impact of TRBC Knockout on Pairing Alpha and Beta Chains from Additional TCRs

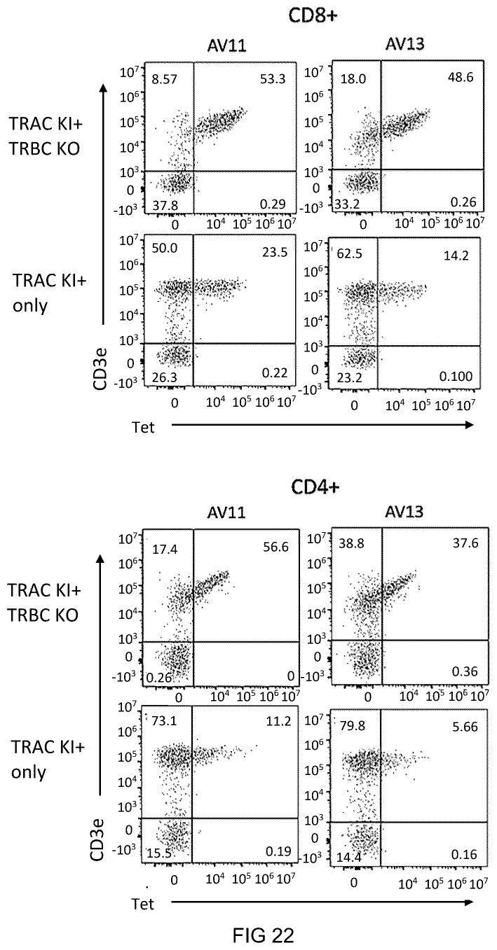

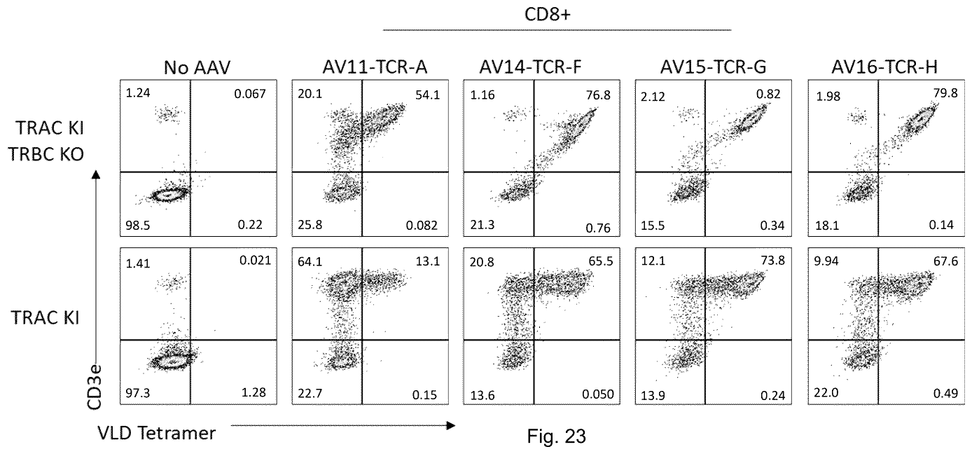

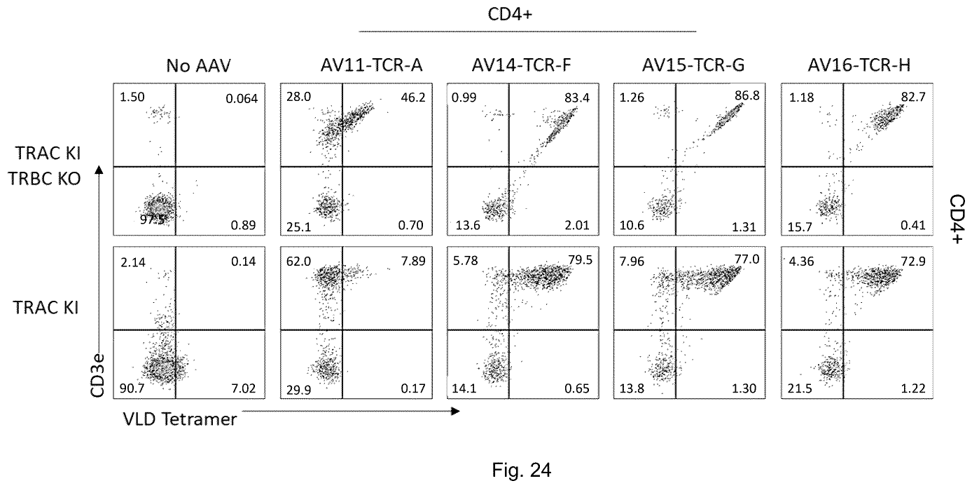

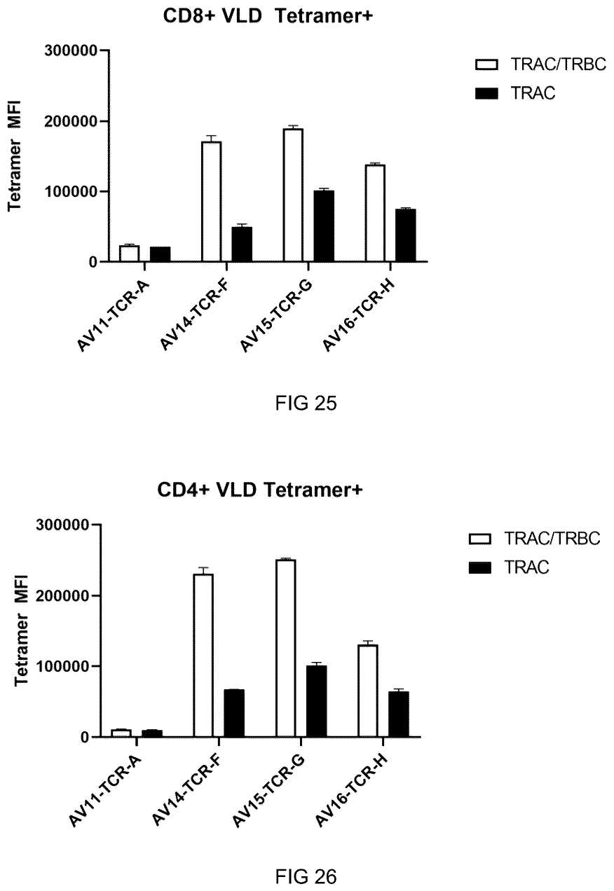

T cells were evaluated with additional engineered TCRs for the impact of endogenous beta chains on pairing of both the inserted alpha chain and inserted beta chain. The experiment was performed as described herein except tetramer staining was used to measure MFI and the AAV inserts in Table 31 were used for transduction. Nine days post cell expansion, edited T cells were co-stained with VLD-Tetramer, an orthogonal method to V-beta staining, to identify cells with TCR surface expression by flow cytometry as shown in Table 31 and data from representative experiments are shown in FIGS. 22, 23, and 24. The mean fluorescence intensity (MFI) was also determined by flow cytometry as shown in Table 31 and FIGS. 25 and 26.

TABLE 31
Surface TCR expression in engineered T cells

| | | CD8+ | | | | CD4+ | | | |
|---|---|---|---|---|---|---|---|---|---|
| | | | | Mean | | | | Mean | |
| | | Mean % | | Tetramer | | Mean % | | Tetramer | |
| Sample | RNP | Tetramer+ | SD | MFI | SD | Tetramer+ | SD | MFI | SD |
| AV11- | TRAC | 12.65 | 0.6 | 21853.5 | 12 | 7.6 | 0.4 | 10064.5 | 634 |
| TCR-A | TRAC + | 51.2 | 3.9 | 23697 | 1605 | 48 | 2.5 | 11332 | 387 |
| | TRBC | | | | | | | | |
| AV14- | TRAC | 61.05 | 6.2 | 50097 | 3828 | 78.9 | 0.8 | 67332.5 | 1379 |
| TCR-F | TRAC + | 76.6 | 0.3 | 171770 | 7974. | 81.8 | 2.3 | 231348 | 8432 |
| | TRBC | | | | | | | | |
| AV15- | TRAC | 73.6 | 0.3 | 101676 | 2594 | 77.55 | 0.77 | 101366 | 4317 |
| TCR-G | TRAC + | 81.9 | 0.1 | 190002 | 3919 | 85.35 | 2.05 | 251084 | 1569 |
| | TRBC | | | | | | | | |
| AV16- | TRAC | 68.85 | 1.8 | 75226.5 | 1704 | 71.65 | 1.8 | 64255 | 3921 |
| TCR-H | TRAC + | 77.1 | 3.8 | 138847 | 1916 | 80.25 | 3.5 | 131127 | 5313 |
| | TRBC | | | | | | | | |
| RNP | TRAC | 0.0555 | 0.05 | n.a | n.a | 0.112 | 0.04 | n.a | n.a |
| only | TRAC + | 0.052 | 0.02 | n.a | n.a | 0.107 | 0.06 | n.a | n.a |
| | TRBC | | | | | | | | |

### Example 18—Engineered T Cell Cytotoxicity and Cytokine Release

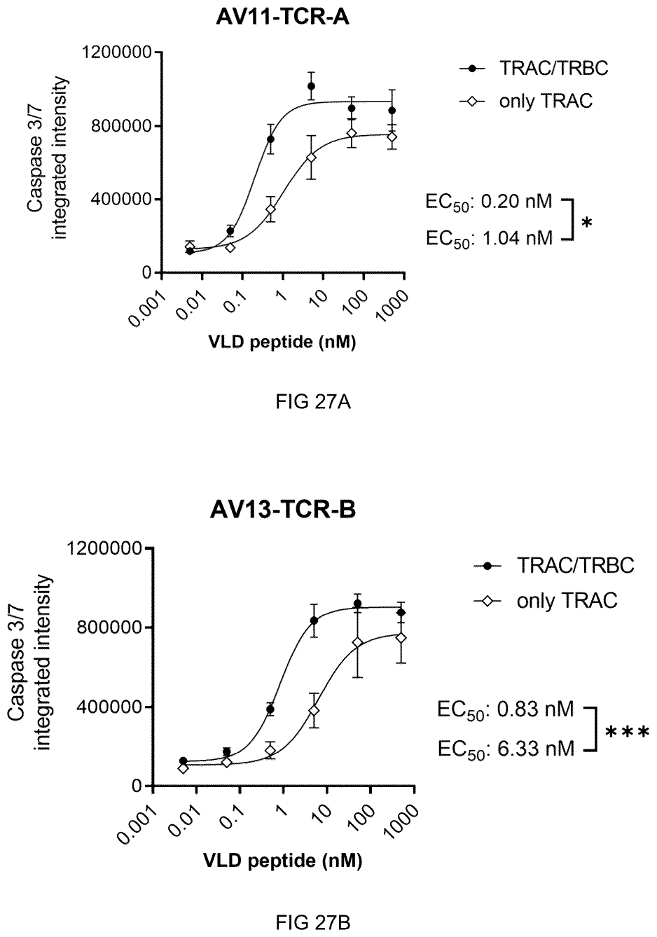

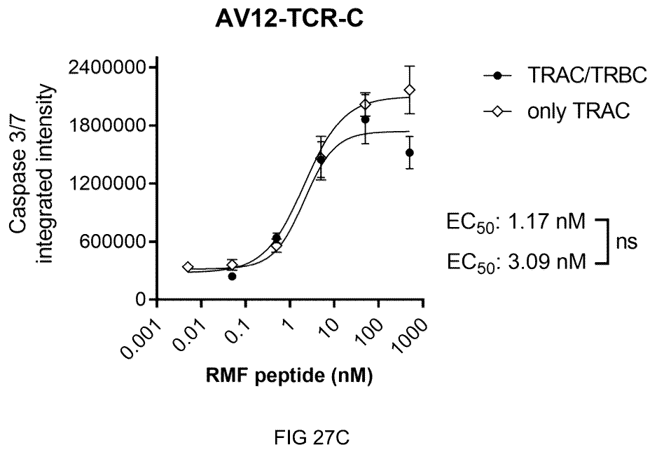

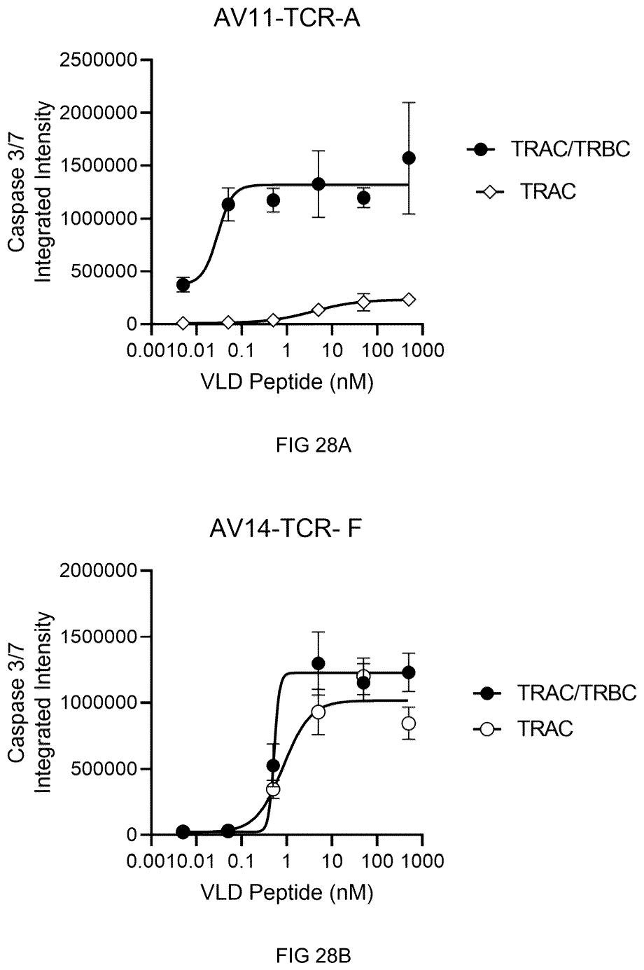

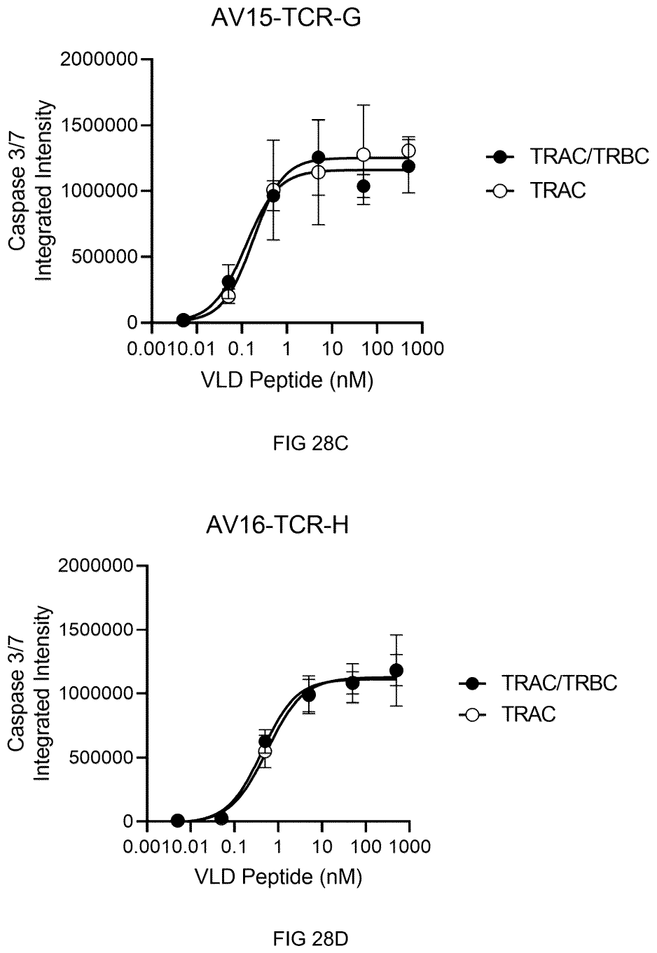

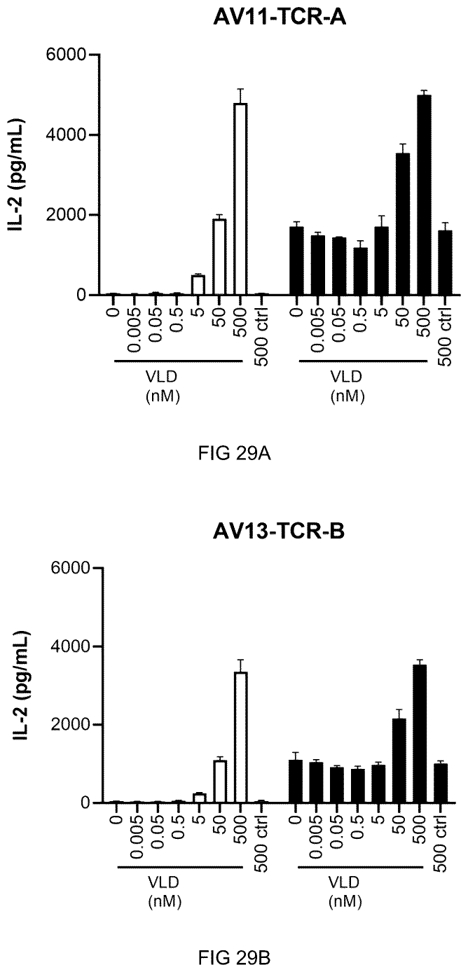

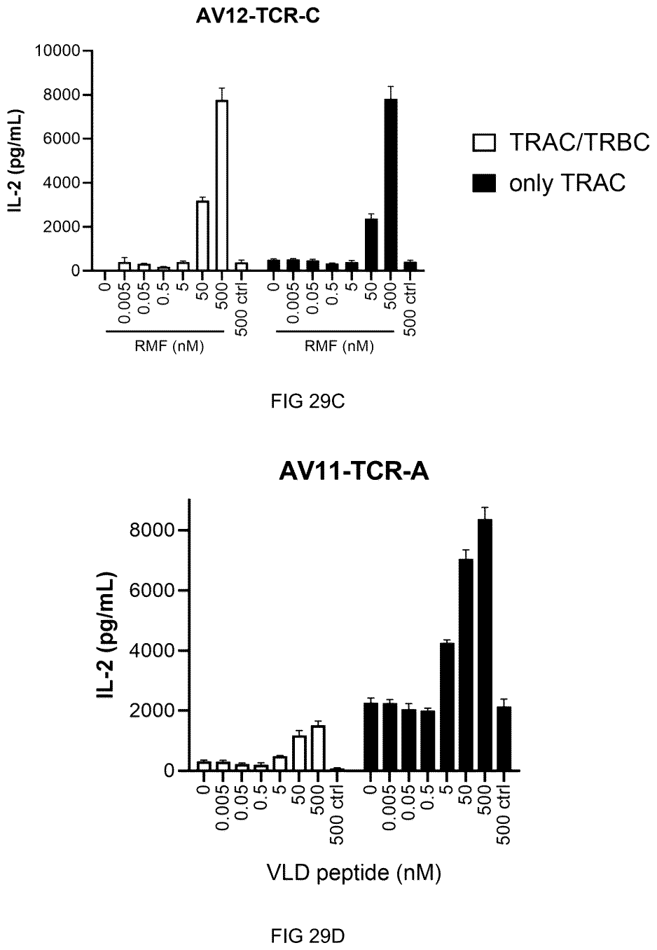

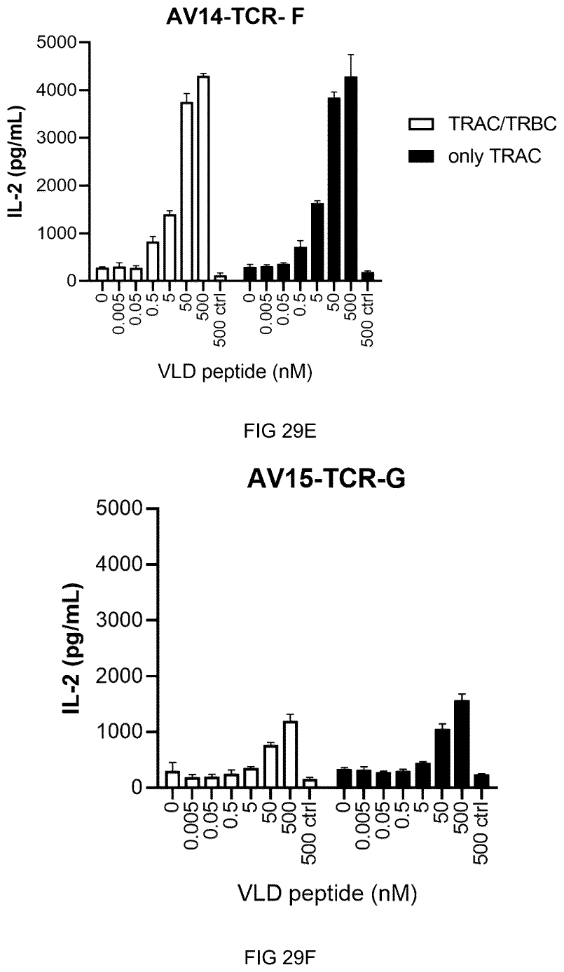

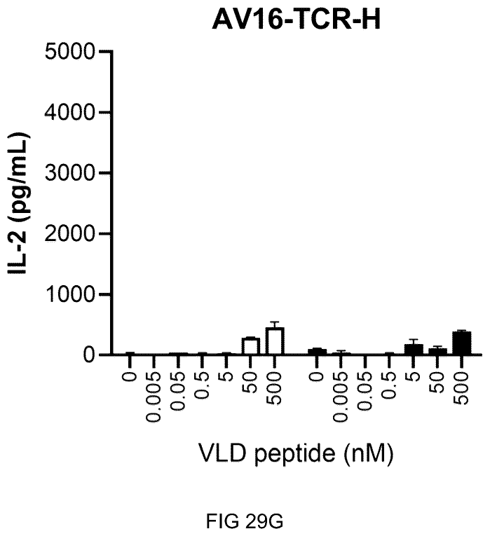

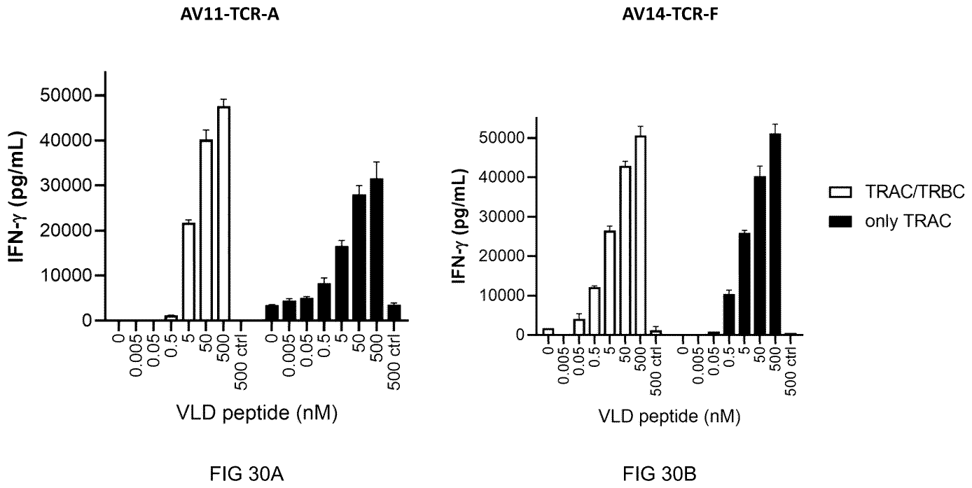

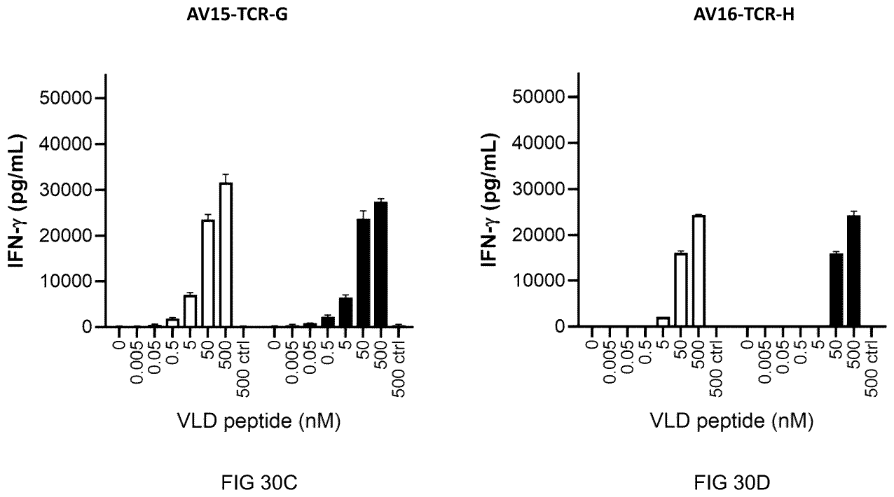

Engineered T cells were assayed for cytotoxicity and cytokine response through the measurement of apoptosis in target cells and the measurement of cytokines released into growth media, respectively. T cells were engineered as described herein using the AAV template sequences described in Table 32 with either RNP targeting only the TRAC locus or RNP targeting both the TRAC and TRBC loci. The HLA-02.01 positive T2 cell line target cells were pulsed and co-cultured with edited T cells as described in Example 15 except the cell ratio of TCR+T cells was 2.5:1 E:T. Caspase 3/7 red reagent (Essen Bioscience) was added to each well at a final concentration of 2 μM. The Incucyte Live Cell Analysis System (Essen Bioscience) and Incucyte S3 analysis software (version 2018B) was used to quantify red florescence from Caspase 3/7 apoptotic cells after 6 hours. After 24 hours supernatants from each well were harvested and IL2 and IFN-γ cytokine release was quantified by ELISA using the Duoset ELISA kit (R&D Systems) following the manufacturers protocol. Mean Caspase 3/7 integrated intensity is shown in Tables 32 and 33 and FIGS. 27A-C and 28A-D. Cytokine release is shown in Tables 34AD and FIGS. 29A-G and 30A-D.

TABLE 32
Cytotoxicity

| | | | TRAC/TRBC | | only TRAC | |
|---|---|---|---|---|---|---|
| | | | Mean | | Mean | |
| | | | Caspase | | Caspase | |
| | | | 3/7 | | 3/7 | |
| | | | integrated | | integrated | |
| Sample | peptide (nM) | | intensity | SD | intensity | SD |
| AV11- | 0 | nM VLD | 92,634 | 7,107 | 132,075 | 37,266 |
| TCR-A | 0.005 | nM VLD | 116,733 | 25,315 | 141,590 | 71,782 |
| | 0.05 | nM VLD | 227,475 | 69,325 | 135,336 | 36,246 |
| | 0.5 | nM VLD | 729,355 | 193,569 | 345,431 | 43,799 |
| | 5 | nM VLD | 1,017,766 | 174,286 | 629,094 | 151,367 |
| | 50 | nM VLD | 897,413 | 122,632 | 762,373 | 59,142 |
| | 500 | nM VLD | 885,460 | 225,388 | 741,203 | 77,370 |
| | 500 | nM RMF | 98,877 | 12,254 | 121,279 | 7,212 |
| AV13- | 0 | nM VLD | 97,684 | 21,060 | 93,536 | 19,338 |
| TCR-B | 0.005 | nM VLD | 126,983 | 7,518 | 88,855 | 42,431 |
| | 0.05 | nM VLD | 174,492 | 11,465 | 119,259 | 14,122 |
| | 0.5 | nM VLD | 388,825 | 71,913 | 180,269 | 23,378 |
| | 5 | nM VLD | 835,068 | 86,910 | 382,323 | 94,136 |
| | 50 | nM VLD | 922,968 | 49,804 | 724,521 | 280,318 |
| | 500 | nM VLD | 876,706 | 57,316 | 748,140 | 249,718 |
| | 500 | nM RMF | 78,919 | 21,138 | 81,044 | 8,512 |
| AV12- | 0 | nM RMF | 279,375 | 93,562 | 276,204 | 65,787 |
| TCR-C | 0.05 | nM RMF | 238,879 | 17,971 | 360,532 | 63,189 |
| | 0.5 | nM RMF | 639,925 | 37,882 | 560,793 | 134,482 |
| | 5 | nM RMF | 1,446,931 | 341,193 | 1,463,030 | 193,719 |
| | 50 | nM RMF | 1,864,716 | 569,777 | 2,020,037 | 65,722 |
| | 500 | nM RMF | 1,519,652 | 256,405 | 2,168,849 | 391,750 |
| | 500 | nM VLD | 218,526 | 35,777 | 330,230 | 34,681 |

TABLE 33
Cytotoxicity

| | | | TRAC/TRBC | | TRAC only | |
|---|---|---|---|---|---|---|
| | | | Mean | | Mean | |
| | | | Caspase | | Caspase | |
| | | | 3/7 | | 3/7 | |
| | | | integrated | | integrated | |
| Sample | peptide (nM) | | intensity | SD | intensity | SD |
| AV11- | 0 | nM VLD | 381,416 | 143,786 | 9,042 | 413 |
| TCR-A | 0.005 | nM VLD | 374,092 | 69,482 | 9,184 | 1,881 |
| | 0.05 | nM VLD | 1,134,226 | 154,362 | 16,907 | 5,212 |
| | 0.5 | nM VLD | 1,173,265 | 110,362 | 38,844 | 19,238 |
| | 5 | nM VLD | 1,325,404 | 313,592 | 137,374 | 35,354 |
| | 50 | nM VLD | 1,197,360 | 92,955 | 207,010 | 81,220 |
| | 500 | nM VLD | 1,571,735 | 526,514 | 233,358 | 55,713 |
| | 500 | nM RMF | 280,465 | 54,951 | 7,342 | 1,113 |
| AV14- | 0 | nM VLD | 21,702 | 8,974 | 18,238 | 3,665 |
| TCR-F | 0.005 | nM VLD | 17,809 | 2,730 | 24,480 | 7,844 |
| | 0.05 | nM VLD | 25,664 | 9,946 | 31,096 | 6,502 |
| | 0.5 | nM VLD | 528,157 | 163,446 | 346,541 | 71,382 |
| | 5 | nM VLD | 1,297,535 | 240,491 | 930,724 | 169,903 |
| | 50 | nM VLD | 1,150,859 | 143,371 | 1,198,605 | 138,751 |
| | 500 | nM VLD | 1,228,755 | 146,056 | 845,589 | 119,495 |
| | 500 | nM RMF | 8,578 | 1,615 | 9,659 | 2,534 |
| AV15- | 0 | nM VLD | 12,408 | 4,254 | 12,182 | 2,841 |
| TCR-G | 0.005 | nM VLD | 20,974 | 7,838 | 21,046 | 12,983 |
| | 0.05 | nM VLD | 310,566 | 130,784 | 199,014 | 55,935 |
| | 0.5 | nM VLD | 963,870 | 113,005 | 1,007,001 | 378,833 |
| | 5 | nM VLD | 1,255,083 | 289,237 | 1,143,162 | 399,176 |
| | 50 | nM VLD | 1,037,328 | 87,408 | 1,276,265 | 379,466 |
| | 500 | nM VLD | 1,187,161 | 202,415 | 1,305,422 | 104,837 |
| | 500 | nM RMF | 6,795 | 1,439 | 4,949 | 1,238 |
| AV16- | 0 | nM VLD | 7,388 | 1,738 | 6,560 | 1,298 |
| TCR-H | 0.005 | nM VLD | 9,815 | 1,987 | 7,651 | 4,159 |
| | 0.05 | nM VLD | 32,360 | 3,457 | 23,556 | 6,335 |
| | 0.5 | nM VLD | 627,805 | 91,413 | 549,651 | 126,913 |
| | 5 | nM VLD | 983,752 | 124,769 | 991,625 | 147,487 |
| | 50 | nM VLD | 1,080,676 | 152,098 | 1,082,346 | 86,963 |
| | 500 | nM VLD | 1,181,699 | 122,175 | 1,180,497 | 276,768 |
| | 500 | nM RMF | 3,889 | 3,101 | 4,189 | 944 |

TABLE 34A
IL2 Cytokine release

| | | IL2 TRAC/TRBC | | | IL2 only TRAC | | |
|---|---|---|---|---|---|---|---|
| | | Mean | SD | | Mean | SD | |
| Con- | | (pg/ | (pg/ | | (pg/ | (pg/ | |
| struct | peptide (nM) | ml) | ml) | N | ml) | ml) | N |
| AV11- | 0 nM VLD | 46 | 3 | 3 | 1,715 | 119 | 3 |
| TCR-A | 0.005 nM VLD | 34 | 3 | 3 | 1,490 | 79 | 3 |
| | 0.05 nM VLD | 52 | 19 | 3 | 1,436 | 18 | 3 |
| | 0.5 nM VLD | 50 | 10 | 3 | 1,186 | 170 | 3 |
| | 5 nM VLD | 506 | 29 | 3 | 1,713 | 264 | 3 |
| | 50 nM VLD | 1,902 | 104 | 3 | 3,554 | 223 | 3 |
| | 500 nM VLD | 4,804 | 345 | 3 | 5,003 | 112 | 3 |
| | 500 nM RMF | 46 | 3 | 3 | 1,615 | 194 | 3 |
| AV13- | 0 nM VLD | 46 | 3 | 3 | 1,109 | 181 | 3 |
| TCR-B | 0.005 nM VLD | 42 | 2 | 3 | 1,048 | 60 | 3 |
| | 0.05 nM VLD | 39 | 5 | 3 | 913 | 44 | 3 |
| | 0.5 nM VLD | 55 | 10 | 3 | 868 | 73 | 3 |
| | 5 nM VLD | 248 | 17 | 3 | 976 | 66 | 3 |
| | 50 nM VLD | 1,095 | 90 | 3 | 2,160 | 240 | 3 |
| | 500 nM VLD | 3,363 | 303 | 3 | 3,547 | 121 | 3 |
| | 500 nM RMF | 51 | 18 | 3 | 1,011 | 64 | 3 |
| AV12- | 0 nM RMF | n.d. | n.d. | n.d. | 498 | 49 | 3 |
| TCR-C | 0.05 nM RMF | 398 | 210 | 3 | 511 | 40 | 3 |
| | 0.5 nM RMF | 320 | 15 | 2 | 481 | 42 | 3 |
| | 5 nM RMF | 182 | 17 | 3 | 333 | 19 | 3 |
| | 50 nM RMF | 392 | 56 | 3 | 396 | 79 | 3 |
| | 500 nM RMF | 3,193 | 165 | 3 | 2,386 | 207 | 3 |
| | 500 nM VLD | 7,776 | 539 | 3 | 7,830 | 557 | 3 |
| | 0 nM VLD | 378 | 113 | 3 | 415 | 66 | 3 |

TABLE 34B
Inteferon-gamma release

| | | TRAC/TRBC | | | only TRAC | | |
|---|---|---|---|---|---|---|---|
| | | Mean | SD | | Mean | SD | |
| Construct | Peptide (nM) | (pg/ml) | (pg/ml) | N | (pg/ml) | (pg/ml) | N |
| AV11- | 0 nM VLD | <LLOD* | <LLOD | 3 | 1.313 | 446 | 3 |
| TCR-A | 0.005 nM VLD | <LLOD | <LLOD | 3 | 904 | 243 | 3 |
| | 0.05 nM VLD | <LLOD | <LLOD | 3 | 731 | 68 | 3 |
| | 0.5 nM VLD | <LLOD | <LLOD | 3 | 1,442 | 148 | 3 |
| | 5 nM VLD | 7,361 | 399 | 3 | 4,323 | 332 | 3 |
| | 50 nM VLD | 15,845 | 1,783 | 3 | 9,221 | 381 | 3 |
| | 500 nM VLD | 25,242 | 3,021 | 3 | 11,558 | 407 | 3 |
| | 500 nM RMF | <LLOD | <LLOD | 3 | 1,656 | 115 | 3 |
| AV13- | 0 nM VLD | <LLOD | <LLOD | 3 | 406 | 75 | 3 |
| TCR-B | 0.005 nM VLD | <LLOD | <LLOD | 3 | 265 | 36 | 3 |
| | 0.05 nM VLD | <LLOD | <LLOD | 3 | 981 | 896 | 3 |
| | 0.5 nM VLD | <LLOD | <LLOD | 3 | 471 | 140 | 3 |
| | 5 nM VLD | 4,643 | 154 | 3 | 1,865 | 183 | 3 |
| | 50 nM VLD | 11,273 | 643 | 3 | 3,645 | 184 | 3 |
| | 500 nM VLD | 19,868 | 1,925 | 3 | 6,476 | 434 | 3 |
| | 500 nM RMF | <LLOD | <LLOD | 3 | 648 | 199 | 3 |
| AV12- | 0 nM RMF | n.d. | n.d | n.d | 1,243 | 92 | 3 |
| TCR-C | 0.05 nM RMF | <LLOD | <LLOD | 3 | 839 | 144 | 3 |
| | 0.5 nM RMF | <LLOD | <LLOD | 2 | 1,151 | 93 | 3 |
| | 5 nM RMF | <LLOD | <LLOD | 3 | 1,181 | 96 | 3 |
| | 50 nM RMF | 4,643 | 154 | 3 | 2,434 | 26 | 3 |
| | 500 nM RMF | 11,273 | 643 | 3 | 5,302 | 723 | 3 |
| | 500 nM VLD | 19,868 | 1,925 | 3 | 16,247 | 192 | 3 |
| | 0 nM VLD | <LLOD | <LLOD | 3 | 1,058 | 93 | 3 |

*LLOD indicates that the reading is below the level of detection in the particular assay used.

TABLE 34C
IL2 Cytokine release

| | | TRAC/TRBC | | | TRAC only | | |
|---|---|---|---|---|---|---|---|
| | | Mean | SD | | Mean | SD | |
| | | (pg/ | (pg/ | | (pg/ | (pg/ | |
| TCR | peptide (nM) | ml) | ml) | N | ml) | ml) | N |
| AV11- | 0 nM VLD | 314 | 46 | 3 | 2.259 | 156 | 3 |
| TCR-A | 0.005 nM VLD | 304 | 42 | 3 | 2,247 | 122 | 3 |
| | 0.05 nM VLD | 227 | 29 | 3 | 2,046 | 188 | 3 |
| | 0.5 nM VLD | 200 | 63 | 3 | 2,007 | 73 | 3 |
| | 5 nM VLD | 494 | 15 | 3 | 4,266 | 96 | 3 |
| | 50 nM VLD | 1,182 | 157 | 3 | 7,062 | 283 | 3 |
| | 500 nM VLD | 1,511 | 141 | 3 | 8,375 | 385 | 3 |
| | 500 nM RMF | 81 | 19 | 3 | 2,135 | 245 | 3 |
| AV16- | 0 nM VLD | 17 | 21 | 2 | 102 | 9 | 2 |
| TCR-H | 0.005 nM VLD | 15 | 0 | 1 | 44 | 27 | 3 |
| | 0.05 nM VLD | 40 | 0 | 1 | 2 | 0 | 1 |
| | 0.5 nM VLD | 17 | 17 | 2 | 23 | 11 | 3 |
| | 5 nM VLD | 30 | 6 | 2 | 175 | 79 | 2 |
| | 50 nM VLD | 286 | 8 | 2 | 111 | 32 | 3 |
| | 500 nM VLD | 455 | 90 | 3 | 387 | 17 | 3 |
| | 500 nM RMF | n/a | n/a | n/a | n/a | n/a | n/a |
| AV15- | 0 nM VLD | 306 | 144 | 3 | 338 | 28 | 3 |
| TCR-G | 0.005 nM VLD | 196 | 40 | 3 | 326 | 49 | 3 |
| | 0.05 nM VLD | 198 | 42 | 3 | 284 | 14 | 3 |
| | 0.5 nM VLD | 248 | 72 | 3 | 305 | 26 | 3 |
| | 5 nM VLD | 351 | 27 | 3 | 442 | 21 | 3 |
| | 50 nM VLD | 764 | 47 | 3 | 1,053 | 89 | 3 |
| | 500 nM VLD | 1,203 | 111 | 3 | 1,566 | 112 | 3 |
| | 500 nM RMF | 157 | 29 | 3 | 242 | 10 | 3 |
| AV14- | 0 nM VLD | 283 | 16 | 3 | 295 | 55 | 3 |
| TCR-F | 0.005 nM VLD | 303 | 84 | 3 | 316 | 24 | 3 |
| | 0.05 nM VLD | 274 | 46 | 3 | 361 | 25 | 3 |
| | 0.5 nM VLD | 836 | 103 | 3 | 724 | 129 | 3 |
| | 5 nM VLD | 1,406 | 69 | 3 | 1,634 | 51 | 3 |
| | 50 nM VLD | 3,755 | 180 | 3 | 3,850 | 116 | 3 |
| | 500 nM VLD | 4,301 | 57 | 3 | 4,289 | 459 | 3 |
| | 500 nM RMF | 117 | 53 | 3 | 190 | 19 | 2 |

TABLE 34D
Inteferon-gamma release

| | | TRAC/TRBC | | | only TRAC | | |
|---|---|---|---|---|---|---|---|
| TCR | peptide (nM) | Mean | SD | N | Mean | SD | N |
| AV11- | 0 nM VLD | <LLOD | <LLOD | 3 | 3,491 | 108 | 3 |
| TCR-A | 0.005 nM VLD | <LLOD | <LLOD | 3 | 4,489 | 444 | 3 |
| | 0.05 nM VLD | <LLOD | <LLOD | 3 | 5,126 | 262 | 3 |
| | 0.5 nM VLD | 1,257 | 23 | 3 | 8,284 | 1,195 | 3 |
| | 5 nM VLD | 21,739 | 653 | 3 | 16,572 | 1,237 | 3 |
| | 50 nM VLD | 40,200 | 2,121 | 3 | 28,080 | 1,871 | 3 |
| | 500 nM VLD | 47,628 | 1,520 | 3 | 31,541 | 3,672 | 3 |
| | 500 nM RMF | 0 | 0 | 3 | 3,590 | 389 | 3 |
| AV16- | 0 nM VLD | <LLOD | <LLOD | 3 | <LLOD | <LLOD | 3 |
| TCR-H | 0.005 nM VLD | <LLOD | <LLOD | 3 | <LLOD | <LLOD | 3 |
| | 0.05 nM VLD | <LLOD | <LLOD | 3 | <LLOD | <LLOD | 3 |
| | 0.5 nM VLD | <LLOD | <LLOD | 3 | <LLOD | <LLOD | 3 |
| | 5 nM VLD | <LLOD | <LLOD | 3 | <LLOD | <LLOD | 3 |
| | 50 nM VLD | 16,058 | 422 | 3 | 15,923 | 427 | 3 |
| | 500 nM VLD | 24,426 | 61 | 3 | 24,319 | 849 | 3 |
| | 500 nM RMF | <LLOD | <LLOD | 3 | <LLOD | <LLOD | 3 |
| AV15- | 0 nM VLD | 219 | 15 | 3 | 260 | 33 | 3 |
| TCR-G | 0.005 nM VLD | 219 | 56 | 3 | 429 | 196 | 3 |
| | 0.05 nM VLD | 549 | 102 | 3 | 854 | 54 | 3 |
| | 0.5 nM VLD | 1,850 | 258 | 3 | 2,265 | 391 | 3 |
| | 5 nM VLD | 7,034 | 497 | 3 | 6,424 | 616 | 3 |
| | 50 nM VLD | 23,558 | 1,135 | 3 | 23,733 | 1,748 | 3 |
| | 500 nM VLD | 31,629 | 1,790 | 3 | 27,473 | 663 | 3 |
| | 500 nM RMF | 235 | 33 | 3 | 416 | 199 | 3 |
| AV14- | 0 nM VLD | <LLOD | <LLOD | 3 | <LLOD | <LLOD | 3 |
| TCR-F | 0.005 nM VLD | <LLOD | <LLOD | 3 | <LLOD | <LLOD | 3 |
| | 0.05 nM VLD | 4,088 | 1,334 | 3 | <LLOD | <LLOD | 3 |
| | 0.5 nM VLD | 12,141 | 321 | 3 | 10,419 | 934 | 3 |
| | 5 nM VLD | 26,510 | 1,161 | 3 | 25,934 | 611 | 3 |
| | 50 nM VLD | 42,858 | 1,197 | 3 | 40,326 | 2,516 | 3 |
| | 500 nM VLD | 50,607 | 2,350 | 3 | 51,106 | 2,385 | 3 |
| | 500 nM RMF | 1,230 | 938 | 3 | <LLOD | <LLOD | 3 |

### Example 19: Engineered T Cell Cytotoxicity as Assayed by CD107a Degranulation and Intracellular Cytokine Staining (ICS)

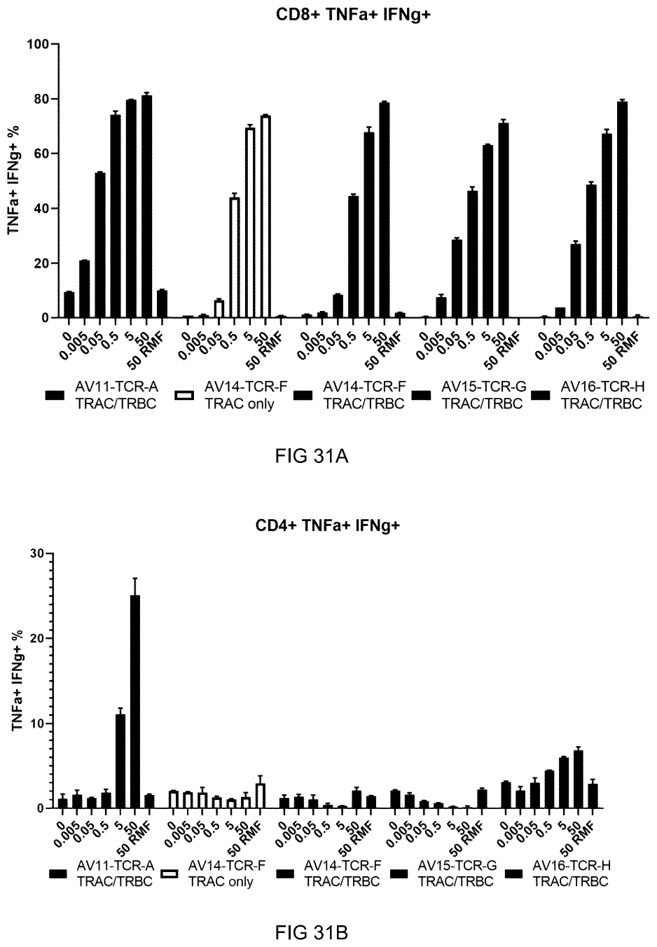

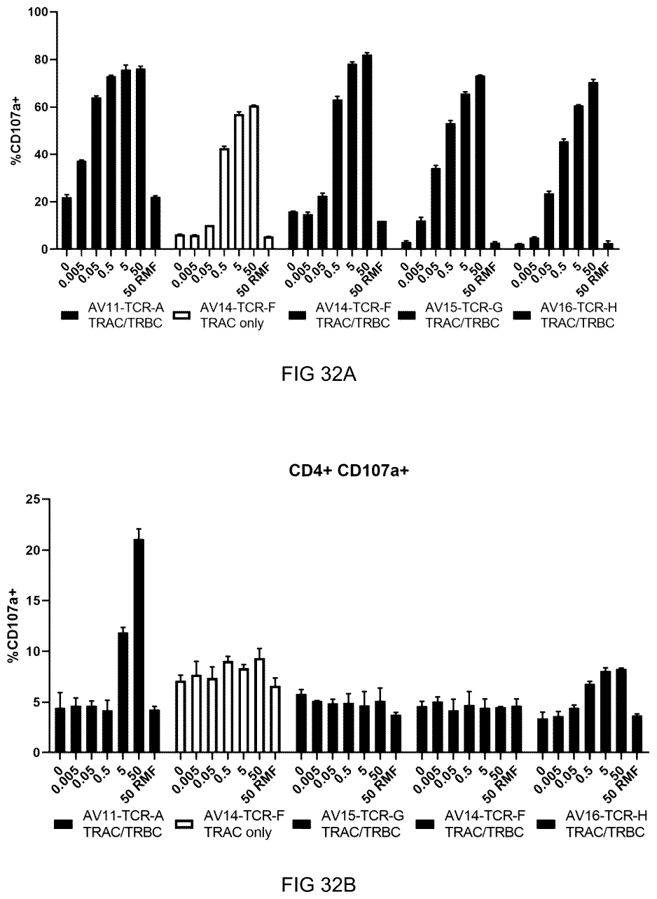

Engineered T cells were also assessed for cytotoxicity and cytokine response through the measurement of CD107α and intracellular cytokine staining, respectively. T cells were engineered as described in Example 18 using the AAV template sequences described in Table 35 with RNP targeting the TRAC locus only or both the TRAC and TRBC loci. HLA-02.01 positive T2 cell line were pulsed as described in Example 18 except edited TCR+ T cells were added to the peptide pulsed target cells at a 1:1 E:T cell ratio. Co-cultures were incubated overnight at 37 C, and the cells stained for surface markers CD3, CD4, CD8 and the specific TCR beta chain or tetramer. Following surface staining, cells were fixed and permeabilized for intracellular IFN-γ and TNFα staining using a commercially available kit (Invitrogen). The immune response elicited to TCR expression is shown in Table 35A and FIGS. 31A-B. Cytotoxicity resulting from expressed TCRs with and with TRBC knockout was evaluated by measuring CD107a in engineered T cells as shown in Table 35B and FIG. 32A-B.

TABLE 35A
Intracellular cytokine staining

| | VLD | CD8+TNFa+ IFNg+ | | | CD4+TNFa+ IFNg+ | | |
|---|---|---|---|---|---|---|---|
| Sample | peptide | Mean | | | Mean | | |
| Name | (nM) | % cells | SD | N | % cells | SD | N |
| AV11- | 0 | 9.4 | 0.2 | 2 | 1.1 | 0.5 | 2 |
| TCR-A | 0.005 | 21.0 | 0.1 | 2 | 1.6 | 0.5 | 2 |
| TRAC/ | 0.05 | 53.0 | 0.4 | 2 | 1.2 | 0.1 | 2 |
| TRBC | 0.5 | 74.3 | 1.3 | 2 | 1.8 | 0.4 | 2 |
| | 5 | 79.8 | 0.1 | 2 | 11.1 | 0.7 | 2 |
| | 50 | 81.3 | 1.0 | 2 | 25.1 | 2.0 | 2 |
| | 50 RMF | 10.0 | 0.3 | 2 | 1.6 | 0.1 | 2 |
| AV14- | 0 | 0.8 | 0.0 | 2 | 2.0 | 0.0 | 2 |
| TCR-F | 0.005 | 1.0 | 0.2 | 2 | 1.9 | 0.0 | 2 |
| TRAC | 0.05 | 6.4 | 0.6 | 2 | 1.9 | 0.6 | 2 |
| only | 0.5 | 44.1 | 1.5 | 2 | 1.3 | 0.1 | 2 |
| | 5 | 69.6 | 1.0 | 2 | 1.0 | 0.1 | 2 |
| | 50 | 74.1 | 0.2 | 2 | 1.3 | 0.5 | 2 |
| | 50 RMF | 0.7 | 0.1 | 2 | 2.9 | 0.9 | 2 |
| AV14- | 0 | 1.3 | 0.0 | 2 | 1.2 | 0.3 | 2 |
| TCR-F | 0.005 | 1.9 | 0.3 | 2 | 1.4 | 0.3 | 2 |
| TRAC/ | 0.05 | 8.5 | 0.3 | 2 | 1.1 | 0.5 | 2 |
| TRBC | 0.5 | 44.6 | 0.6 | 2 | 0.4 | 0.2 | 2 |
| | 5 | 68.0 | 1.8 | 2 | 0.3 | 0.0 | 2 |
| | 50 | 78.7 | 0.4 | 2 | 2.1 | 0.4 | 2 |
| | 50 RMF | 1.8 | 0.1 | 2 | 1.5 | 0.0 | 2 |
| AV15- | 0 | 0.4 | 0.1 | 2 | 2.1 | 0.1 | 2 |
| TCR-G | 0.005 | 7.6 | 0.9 | 2 | 1.6 | 0.2 | 2 |
| TRAC/ | 0.05 | 28.7 | 0.6 | 2 | 0.9 | 0.0 | 2 |
| TRBC | 0.5 | 46.5 | 1.3 | 2 | 0.6 | 0.0 | 2 |
| | 5 | 63.1 | 0.4 | 2 | 0.2 | 0.0 | 2 |
| | 50 | 71.4 | 1.2 | 2 | 0.1 | 0.1 | 2 |
| | 50 RMF | 0.4 | 0.0 | 2 | 2.2 | 0.1 | 2 |
| AV16- | 0 | 0.5 | 0.1 | 2 | 3.1 | 0.1 | 2 |
| TCR-H | 0.005 | 3.7 | 0.0 | 2 | 2.1 | 0.5 | 2 |
| TRAC/ | 0.05 | 27.1 | 1.0 | 2 | 3.0 | 0.6 | 2 |
| TRBC | 0.5 | 48.7 | 1.0 | 2 | 4.4 | 0.1 | 2 |
| | 5 | 67.5 | 1.5 | 2 | 6.0 | 0.1 | 2 |
| | 50 | 79.2 | 0.6 | 2 | 6.9 | 0.4 | 2 |
| | 50 RMF | 0.6 | 0.4 | 2 | 2.9 | 0.5 | 2 |

TABLE 35B
CD107a degranulation

| | VLD | CD8+ CD107a+ | | | CD4+ CD107a+ | | |
|---|---|---|---|---|---|---|---|
| Sample | peptide | Mean | | | Mean | | |
| Name | (nM) | % cells | SD | N | % cells | SD | N |
| AV11- | 0 | 21.95 | 1.06 | 2 | 4.45 | 1.51 | 2 |
| TCR-A | 0.005 | 37.30 | 0.28 | 2 | 4.64 | 0.78 | 2 |
| TRAC/ | 0.05 | 64.10 | 0.57 | 2 | 4.68 | 0.44 | 2 |
| TRBC | 0.5 | 73.05 | 0.35 | 2 | 4.23 | 0.96 | 2 |
| | 5 | 75.80 | 1.84 | 2 | 11.85 | 0.49 | 2 |
| | 50 | 76.20 | 0.99 | 2 | 21.10 | 0.99 | 2 |
| | 50 RMF | 22.05 | 0.49 | 2 | 4.30 | 0.28 | 2 |
| AV14- | 0 | 6.33 | 0.03 | 2 | 7.14 | 0.52 | 2 |
| TCR-F | 0.005 | 5.93 | 0.13 | 2 | 7.73 | 1.29 | 2 |
| TRAC | 0.05 | 10.30 | 0.00 | 2 | 7.39 | 1.10 | 2 |
| only | 0.5 | 42.75 | 0.78 | 2 | 9.08 | 0.46 | 2 |
| | 5 | 57.05 | 0.92 | 2 | 8.35 | 0.36 | 2 |
| | 50 | 60.75 | 0.07 | 2 | 9.38 | 0.88 | 2 |
| | 50 RMF | 5.42 | 0.02 | 2 | 6.63 | 0.76 | 2 |
| AV14- | 0 | 15.90 | 0.14 | 2 | 5.84 | 0.41 | 2 |
| TCR-F | 0.005 | 14.75 | 0.92 | 2 | 5.14 | 0.03 | 2 |
| TRAC/ | 0.05 | 22.55 | 1.06 | 2 | 4.89 | 0.40 | 2 |
| TRBC | 0.5 | 63.15 | 1.34 | 2 | 4.94 | 0.90 | 2 |
| | 5 | 78.25 | 0.78 | 2 | 4.71 | 1.35 | 2 |
| | 50 | 82.05 | 0.78 | 2 | 5.14 | 1.26 | 2 |
| | 50 RMF | 12.00 | 0.00 | 2 | 3.78 | 0.23 | 2 |
| AV15- | 0 | 3.15 | 0.40 | 2 | 4.62 | 0.47 | 2 |
| TCR-G | 0.005 | 12.15 | 1.34 | 2 | 5.06 | 0.47 | 2 |
| TRAC/ | 0.05 | 34.25 | 1.06 | 2 | 4.23 | 1.06 | 2 |
| TRBC | 0.5 | 53.30 | 0.99 | 2 | 4.74 | 1.32 | 2 |
| | 5 | 65.75 | 0.64 | 2 | 4.44 | 0.88 | 2 |
| | 50 | 73.25 | 0.21 | 2 | 4.52 | 0.05 | 2 |
| AV16- | 50 RMF | 2.82 | 0.37 | 2 | 4.67 | 0.65 | 2 |
| TCR-H | 0 | 2.29 | 0.07 | 2 | 3.42 | 0.60 | 2 |
| TRAC/ | 0.005 | 5.05 | 0.17 | 2 | 3.63 | 0.46 | 2 |
| TRBC | 0.05 | 23.60 | 0.85 | 2 | 4.48 | 0.24 | 2 |
| | 0.5 | 45.65 | 0.92 | 2 | 6.82 | 0.24 | 2 |
| | 5 | 60.70 | 0.28 | 2 | 8.09 | 0.30 | 2 |
| | 50 | 70.55 | 1.06 | 2 | 8.28 | 0.08 | 2 |
| | 50 RMF | 2.64 | 0.84 | 2 | 3.70 | 0.15 | 2 |

### Example 20: Alloreactivity Assay

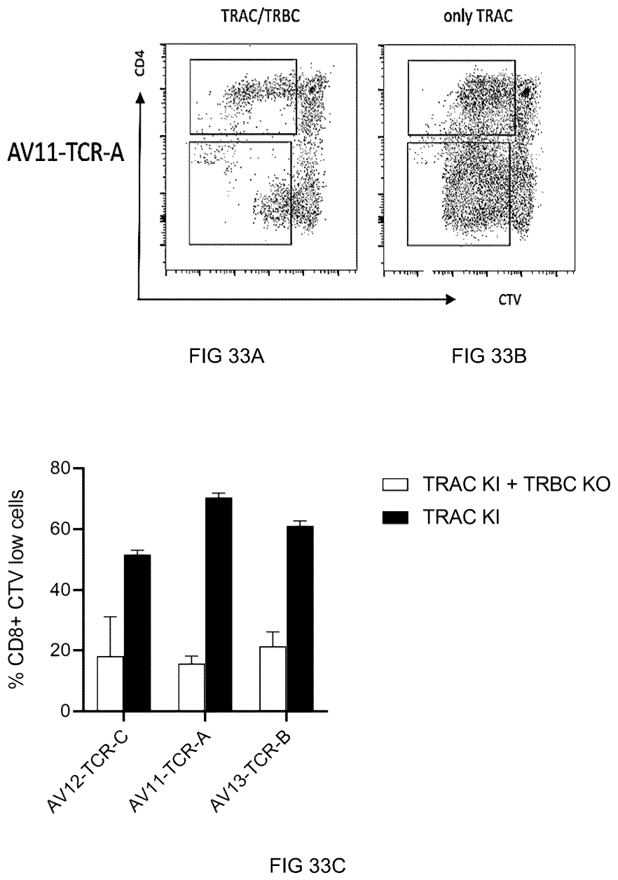

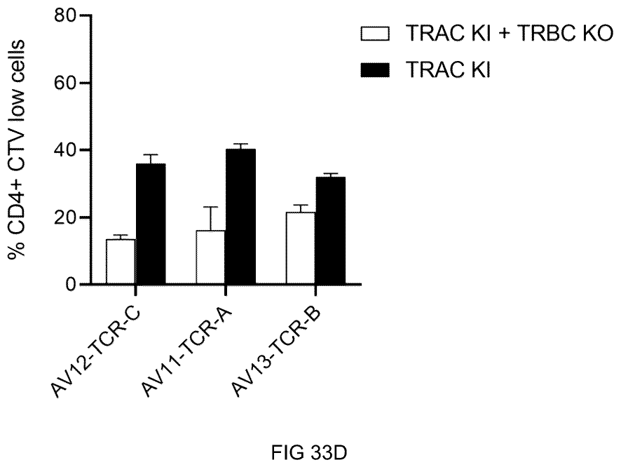

Engineered T cells prepared in Example 18 were also assayed for non-specific reactivity to antigen presenting cells in a mixed lymphocyte reaction (MVLR) suppression assay. Engineered T cells were labelled with CellTrace Violet (Invitrogen), according to manufacturer's instructions. Allogeneic PBMCs were depleted of CD3+ cells using MACS (Miltenyi) and used to stimulate the CTV-labelled T cells. Specifically, 5×10⁴ T cells were plated with 3×10⁴ CD3-depleted allogeneic PBMCs in a 96-well U-bottom plate (Corning). Five days later, the cells were harvested, stained with anti-CD4 and analyzed by flow cytometry. The degree of proliferation was quantified by dilution of the CellTrace Violet dye. Roswell Park Memorial Institute 1640 media (RPMI; Corning) with glutamine was supplemented with 100 FBS, 1× GutaMAX, 10 mM HEPES, 1× Penicillin/streptomycin, 1 mM sodium pyruvate, 50 uM 2-ME, and 1× non-essential amino acids was used throughout the MLR assay. The mean percentage of cells staining with low levels of CellTrace Violet (CTV), i.e. cells with high proliferation, is shown in Table 36 and FIGS. 33A-B. FIGS. 33A and 33B show gating used to assay CTV levels. Cells treated with TRBC RNP showed less proliferation than cells not treated with TRBC RNP in both CD4+ and CD4− (CD8+) cells for each inserted TCR tested as shown in Table 36 and FIGS. 33C and 33D.

TABLE 36
Proliferation in response to allogeneic PBMCs

| | | TRAC RNP + TRBC RNP | | | TRAC RNP | | |
|---|---|---|---|---|---|---|---|
| | | Mean % | | | Mean % | | |
| | | CTV | | | CTV | | |
| T Cell | Sample | low | | | low | | |
| Type | Name | cells | SD | n | cells | SD | n |
| CD4− | AV12-TCR-C | 18% | 13% | 3 | 52% | 2% | 3 |
| | AV11-TCR-A | 16% | 3% | 3 | 70% | 2% | 3 |
| | AV13-TCR-B | 21% | 5% | 3 | 61% | 2% | 3 |
| CD4+ | AV12-TCR-C | 14% | 1% | 3 | 36% | 3% | 3 |
| | AV11-TCR-A | 16% | 7% | 3 | 40% | 2% | 3 |
| | AV13-TCR-B | 22% | 2% | 3 | 32% | 1% | 3 |

Table 37—Nucleic Acid Templates. Note, all templates delivered as ssAAV and comprise AAV2 5′ and 3′ ITRs. Homology arms are as described above. Many additionally comprise one or more promoter, cleavage, or polyA sequences, as set forth below. An indicates a stop codon, below.

TABLE 37

| | | Promoter or | TCR ORF | |
|---|---|---|---|---|
| | Trans- | (Promoter- | Config- | |
| Template | gene | less/Peptide) | uration | PolyA |
| AV1 (SEQ ID NO: 613) | TCR-C | EF1a | β-P2A-α-* | bGH |
| AV2 (SEQ ID NO: 614) | TCR-C | Ef1a-short | β-P2A-α-* | bGH |
| AV3 (SEQ ID NO: 615) | TCR-C | MND-1 | β-P2A-α-* | bGH |
| AV4 (SEQ ID NO: 616) | TCR-C | MND-2 | β-P2A-α-* | bGH |
| AV5 (SEQ ID NO: 617) | TCR-C | PGK | β-P2A-α-* | bGH |
| AV6 (SEQ ID NO: 618) | TCR-C | MND-1 | β-P2A-α-* | bGH |
| AV7 (SEQ ID NO: 619) | TCR-C | PGK | β-P2A-α-* | bGH |
| AV8 (SEQ ID NO: 620) | TCR-C | MND-2 | β-P2A-α-* | bGH |
| AV9 (SEQ ID NO: 621) | EGFP | EF1a | n/a | SV40 |
| AV10 (SEQ ID NO: 622) | EGFP | (T2A) | n/a | bGH |
| AV11 (SEQ ID NO: 623) | TCR-A | EF1a | β-P2A-α-* | bGH |
| AV12 (SEQ ID NO: 624) | TCR-C | EF1a | β-P2A-α-* | bGH |
| AV13 (SEQ ID NO: 625) | TCR-B | EF1a | β-P2A-α-* | bGH |
| AV14 (SEQ ID NO: 626) | TCR-F | EF1a | β-P2A-α-* | bGH |
| AV15 (SEQ ID NO: 627) | TCR-G | EF1a | β-P2A-α-* | bGH |
| AV16 (SEQ ID NO: 628) | TCR-H | EF1a | β-P2A-α-* | bGH |
| AV18 (SEQ ID NO: 629) | TCR-D | EF1a | β-P2A-α-* | bGH |
| AV19 (SEQ ID NO: 630) | TCR-C | (T2A) | β-P2A-α-* | bGH |
| AV20 (SEQ ID NO: 631) | TCR-E | (T2A) | β-P2A-α-* | bGH |
| AV21 (SEQ ID NO: 632) | TCR-B | (T2A) | β-P2A-α-* | bGH |

The elements of the templates defined in Table 37 are defined according to the following sequences as follows: EF1a (SEQ ID NO: 603); EF1 (SEQ ID NO: 604); MND-1 (SEQ ID NO: 605); MND-2 (SEQ ID NO: 606); PGK (SEQ ID NO: 607); T2A (SEQ ID NO: 608); P2A (SEQ ID NO: 609); EGFP (SEQ ID NO: 610); SV40 Poly A (SEQ ID NO: 611); bGH PolyA (SEQ ID NO: 612).

1.-322. (canceled)

323. A method of reducing the expression of an endogenous T cell receptor and inserting a heterologous immunological receptor, comprising delivering to a cell: (a) a TRAC guide RNA and optionally a TRBC1/2 guide RNA; (b) an RNA-guided DNA binding agent, or a nucleic acid encoding an RNA-guided DNA binding agent; and (c) a nucleic acid encoding the heterologous immunological receptor,
wherein the TRAC guide RNA comprises:
i. a guide sequence that is SEQ ID NO: 90, or a guide sequence that is 18 or 19 contiguous nucleotides of the 3′ end of SEQ ID NO: 90; or
ii. a guide sequence that is SEQ ID NO: 95, or a guide sequence that is 18 or 19 contiguous nucleotides of the 5′ end of SEQ ID NO: 95; or
iii. a guide sequence that is SEQ ID NO: 185, or a guide sequence that is 18 or 19 contiguous nucleotides of the 5′ end of SEQ ID NO: 185; or
iv. a guide sequence that is SEQ ID NO: 214, or a guide sequence that is 18 or 19 contiguous nucleotides of the 3′ end of SEQ ID NO: 214; or
v. a guide sequence selected from SEQ ID NOs: 91-94, 96-98, 100, 102-178, 213, and 215-218, or a guide sequence that is at least 18 or 19 contiguous nucleotides of a sequence selected from SEQ ID NOs: 91-94, 96-98, 100, 102-178, 213, and 215-218; and
wherein the TRBC1/2 guide RNA, if present, comprises:
i. a guide sequence selected from SEQ ID NOs: 1-89, or a guide sequence that is at least 17, 18, 19, or 20 contiguous nucleotides of a sequence selected from SEQ ID NOs: 1-89; or
ii. a guide sequence that is at least 95% or 90% identical to a sequence selected from SEQ ID NOs: 1-89; or
iii. a guide sequence selected from SEQ ID NOs: 1-24; or
iv. a guide sequence selected from SEQ ID NOs: 1-6; or
v. a guide sequence that is SEQ ID NO: 2, or a guide sequence that is at least 17, 18, 19, or 20 contiguous nucleotides of SEQ ID NO: 2.

324. An engineered human cell, which has reduced or eliminated surface expression of TRAC relative to an unmodified cell, and which comprises a genetic modification in the TRAC locus, wherein the genetic modification comprises at least one nucleotide within the genomic coordinates of chr14:22547524-22547544, chr14:22547501-22547521, or chr14:22547556-22547576, and which comprises a nucleic acid encoding a heterologous immunological receptor inserted in the TRAC locus.

325. A population of cells, comprising one or more of the engineered human cells of claim 324.

326. The population of cells of claim 325, wherein greater than about 50%, 55%, 60%, 65%, 70%, 75%, 80%, 85%, 90%, 95%, 98%, or 99% of the cells in the population lack an endogenous T cell receptor.

327. The population of cells of claim 325, wherein greater than about 20%, 30%, 40%, 50%, 60%, 70%, 80%, 85%, or 90% of the cells in the population express the heterologous immunological receptor.

328. A method of treating cancer in a human or animal subject, comprising administering the population of cells of claim 325.

329. The engineered human cell of claim 324, wherein the genetic modification comprises at least one nucleotide within the genomic coordinates of chr14:22547524-22547544.

330. The engineered human cell of claim 324, wherein the genetic modification comprises at least one nucleotide within the genomic coordinates of chr14:22547501-22547521 or chr14:22547556-22547576.

331. The engineered human cell of claim 324, wherein the engineered human cell comprises a TRAC guide RNA comprising:
i) a guide sequence that is SEQ ID NO: 90; or
ii) a guide sequence that is SEQ ID NO: 185; or
iii) a guide sequence that is SEQ ID NO: 214.

332. The engineered human cell of claim 324, wherein the heterologous immunological receptor is a T-cell receptor.

333. The engineered human cell of claim 332, wherein the T-cell receptor recognizes a cancer antigen.

334. The engineered human cell of 324, wherein the heterologous immunological receptor is a chimeric antigen receptor.

335. The engineered human cell of 334, wherein the chimeric antigen receptor recognizes a cancer antigen.

336. A cell, modified by the method of claim 323.

337. A population of cells, comprising one or more cells of claim 336.

338. A method of treating cancer in a human or animal subject, comprising administering the population of cells of claim 337.

339. A method of reducing the expression of an endogenous T cell receptor, comprising delivering to a cell: (a) a TRAC guide RNA and a TRBC1/2 guide RNA; and (b) an RNA-guided DNA binding agent, or a nucleic acid encoding an RNA-guided DNA binding agent, wherein:
the TRAC guide RNA comprises:
i. a guide sequence selected from SEQ ID NOs: 90-178, 185, and 213-218, or a guide sequence that is at least 17, 18, 19, or 20 contiguous nucleotides of a sequence selected from SEQ ID NOs: 90-178, 185, and 213-218; or
ii. a guide sequence that is at least 95% or 90% identical to a sequence selected from SEQ ID NOs: 90-178, 185, and 213-218; or
iii. a guide sequence selected from SEQ ID NOs: 90-95, 185, and 214, or a guide sequence that is at least 17, 18, 19, or 20 contiguous nucleotides of a sequence selected from SEQ ID NOs: 90-95, 185, and 214; or
iv. a guide sequence that is SEQ ID NO: 90, and
the TRBC1/2 guide RNA comprises:
i. a guide sequence selected from SEQ ID NOs: 1 and 3-89, or a guide sequence that is at least 17, 18, 19, or 20 contiguous nucleotides of a sequence selected from SEQ ID NOs: 1 and 3-89; or
ii. a guide sequence that is at least 95% or 90% identical to a sequence selected from SEQ ID NOs: 1 and 3-89; or
iii. a guide sequence selected from SEQ ID NOs: 1 and 3-24; or
iv. a guide sequence selected from SEQ ID NOs: 1 and 3-6, or wherein:
the TRAC guide RNA comprises:
i. a guide sequence selected from SEQ ID NOs: 90-98, 100-178, 185, and 213-218, or a guide sequence that is at least 17, 18, 19, or 20 contiguous nucleotides of a sequence selected from SEQ ID NOs: 90-98, 100-178, 185, and 213-218; or
ii. a guide sequence that is at least 95% or 90% identical to a sequence selected from SEQ ID NOs: 90-98, 100-178, 185, and 213-218; or
iii. a guide sequence selected from SEQ ID NOs: 90-95, 185, and 214, or a guide sequence that is at least 17, 18, 19, or 20 contiguous nucleotides of a sequence selected from SEQ ID NOs: 90-95, 185, and 214; or
iv. a guide sequence that is SEQ ID NO: 90, and
the TRBC1/2 guide RNA comprises:
i. a guide sequence selected from SEQ ID NOs: 1-89, or a guide sequence that is at least 17, 18, 19, or 20 contiguous nucleotides of a sequence selected from SEQ ID NOs: 1-89; or
ii. a guide sequence that is at least 95% or 90% identical to a sequence selected from SEQ ID NOs: 1-89; or
iii. a guide sequence selected from SEQ ID NOs: 1-24; or
iv. a guide sequence selected from SEQ ID NOs: 1-6; or
v. a guide sequence that is SEQ ID NO: 2, or a guide sequence that is at least 17, 18, 19, or 20 contiguous nucleotides of SEQ ID NO: 2.

340. A cell, modified by the method of claim 339.

341. A population of cells, comprising one or more cells of claim 340.

342. A composition comprising a TRAC guide RNA and optionally a TRBC1/2 guide RNA,
wherein:
the TRAC guide RNA comprises:
i. a guide sequence selected from SEQ ID NOs: 90-178, 185, and 213-218, or a guide sequence that is at least 17, 18, 19, or 20 contiguous nucleotides of a sequence selected from SEQ ID NOs: 90-178, 185, and 213-218; or
ii. a guide sequence that is at least 95% or 90% identical to a sequence selected from SEQ ID NOs: 90-178, 185, and 213-218; or
iii. a guide sequence selected from SEQ ID NOs: 90-95, 185, and 214, or a guide sequence that is at least 17, 18, 19, or 20 contiguous nucleotides of a sequence selected from SEQ ID NOs: 90-95, 185, and 214; or
iv. a guide sequence that is SEQ ID NO: 90, and
the TRBC1/2 guide RNA, if present, comprises:
i. a guide sequence selected from SEQ ID NOs: 1 and 3-89, or a guide sequence that is at least 17, 18, 19, or 20 contiguous nucleotides of a sequence selected from SEQ ID NOs: 1 and 3-89; or
ii. a guide sequence that is at least 95% or 90% identical to a sequence selected from SEQ ID NOs: 1 and 3-89; or
iii. a guide sequence selected from SEQ ID NOs: 1 and 3-24; or
iv. a guide sequence selected from SEQ ID NOs: 1 and 3-6, or
wherein:
the TRAC guide RNA comprises:
i. a guide sequence selected from SEQ ID NOs: 90-98, 100-178, 185, and 213-218, or a guide sequence that is at least 17, 18, 19, or 20 contiguous nucleotides of a sequence selected from SEQ ID NOs: 90-98, 100-178, 185, and 213-218; or
ii. a guide sequence that is at least 95% or 90% identical to a sequence selected from SEQ ID NOs: 90-98, 100-178, 185, and 213-218; or
iii. a guide sequence selected from SEQ ID NOs: 90-95, 185, and 214, or a guide sequence that is at least 17, 18, 19, or 20 contiguous nucleotides of a sequence selected from SEQ ID NOs: 90-95, 185, and 214; or
iv. a guide sequence that is SEQ ID NO: 90, and
the TRBC1/2 guide RNA, if present, comprises:
i. a guide sequence selected from SEQ ID NOs: 1-89, or a guide sequence that is at least 17, 18, 19, or 20 contiguous nucleotides of a sequence selected from SEQ ID NOs: 1-89; or
ii. a guide sequence that is at least 95% or 90% identical to a sequence selected from SEQ ID NOs: 1-89; or
iii. a guide sequence selected from SEQ ID NOs: 1-24; or
iv. a guide sequence selected from SEQ ID NOs: 1-6; or
v. a guide sequence that is SEQ ID NO: 2, or a guide sequence that is at least 17, 18, 19, or 20 contiguous nucleotides of SEQ ID NO: 2.


SEQUENCE LISTING (632 sequences)

SEQ ID NO: 1 - RNA, 20 nt, synthetic construct
  misc_feature 1..20: note = synthetic construct
  source 1..20: mol_type = other RNA
ggctctcggagaatgacgag

SEQ ID NO: 2 - RNA, 20 nt, synthetic construct
  misc_feature 1..20: note = synthetic construct
  source 1..20: mol_type = other RNA
ggcctcggcgctgacgatct

SEQ ID NO: 3 - RNA, 20 nt, synthetic construct
  misc_feature 1..20: note = synthetic construct
  source 1..20: mol_type = other RNA
atgacgagtggacccaggat

SEQ ID NO: 4 - RNA, 20 nt, synthetic construct
  misc_feature 1..20: note = synthetic construct
  source 1..20: mol_type = other RNA
agaaggtggccgagaccctc

SEQ ID NO: 5 - RNA, 20 nt, synthetic construct
  misc_feature 1..20: note = synthetic construct
  source 1..20: mol_type = other RNA
tgagggtctcggccaccttc

SEQ ID NO: 6 - RNA, 20 nt, synthetic construct
  misc_feature 1..20: note = synthetic construct
  source 1..20: mol_type = other RNA
agagatctcccacacccaaa

SEQ ID NO: 7 - RNA, 20 nt, synthetic construct
  misc_feature 1..20: note = synthetic construct
  source 1..20: mol_type = other RNA
tggctcaaacacagcgacct

SEQ ID NO: 8 - RNA, 20 nt, synthetic construct
  misc_feature 1..20: note = synthetic construct
  source 1..20: mol_type = other RNA
ggcgctgacgatctgggtga

SEQ ID NO: 9 - RNA, 20 nt, synthetic construct
  misc_feature 1..20: note = synthetic construct
  source 1..20: mol_type = other RNA
tggcagacaggaccccttgc

SEQ ID NO: 10 - RNA, 20 nt, synthetic construct
  misc_feature 1..20: note = synthetic construct
  source 1..20: mol_type = other RNA
tgacgagtggacccaggata

SEQ ID NO: 11 - RNA, 20 nt, synthetic construct
  misc_feature 1..20: note = synthetic construct
  source 1..20: mol_type = other RNA
agacaggaccccttgctggt

SEQ ID NO: 12 - RNA, 20 nt, synthetic construct
  misc_feature 1..20: note = synthetic construct
  source 1..20: mol_type = other RNA
ttgacagcggaagtggttgc

SEQ ID NO: 13 - RNA, 20 nt, synthetic construct
  misc_feature 1..20: note = synthetic construct
  source 1..20: mol_type = other RNA
cgtagaactggacttgacag

SEQ ID NO: 14 - RNA, 20 nt, synthetic construct
  misc_feature 1..20: note = synthetic construct
  source 1..20: mol_type = other RNA
cgctgtcaagtccagttcta

SEQ ID NO: 15 - RNA, 20 nt, synthetic construct
  misc_feature 1..20: note = synthetic construct
  source 1..20: mol_type = other RNA
actggacttgacagcggaag

SEQ ID NO: 16 - RNA, 20 nt, synthetic construct
  misc_feature 1..20: note = synthetic construct
  source 1..20: mol_type = other RNA
gttgcgggggttctgccaga

SEQ ID NO: 17 - RNA, 20 nt, synthetic construct
  misc_feature 1..20: note = synthetic construct
  source 1..20: mol_type = other RNA
ctgcctgagcagccgcctga

SEQ ID NO: 18 - RNA, 20 nt, synthetic construct
  misc_feature 1..20: note = synthetic construct
  source 1..20: mol_type = other RNA
gacagcggaagtggttgcgg

SEQ ID NO: 19 - RNA, 20 nt, synthetic construct
  misc_feature 1..20: note = synthetic construct
  source 1..20: mol_type = other RNA
ccactcacctgctctacccc

SEQ ID NO: 20 - RNA, 20 nt, synthetic construct
  misc_feature 1..20: note = synthetic construct
  source 1..20: mol_type = other RNA
gctgtcaagtccagttctac

SEQ ID NO: 21 - RNA, 20 nt, synthetic construct
  misc_feature 1..20: note = synthetic construct
  source 1..20: mol_type = other RNA
acactggtgtgcctggccac

SEQ ID NO: 22 - RNA, 20 nt, synthetic construct
  misc_feature 1..20: note = synthetic construct
  source 1..20: mol_type = other RNA
agaccctcaggcggctgctc

SEQ ID NO: 23 - RNA, 20 nt, synthetic construct
  misc_feature 1..20: note = synthetic construct
  source 1..20: mol_type = other RNA
atgggaaggaggtgcacagt

SEQ ID NO: 24 - RNA, 20 nt, synthetic construct
  misc_feature 1..20: note = synthetic construct
  source 1..20: mol_type = other RNA
agggcgggctgctccttgag

SEQ ID NO: 25 - RNA, 20 nt, synthetic construct
  misc_feature 1..20: note = synthetic construct
  source 1..20: mol_type = other RNA
tccctagcaagatctcatag

SEQ ID NO: 26 - RNA, 20 nt, synthetic construct
  misc_feature 1..20: note = synthetic construct
  source 1..20: mol_type = other RNA
ggtgcacagtggggtcagca

SEQ ID NO: 27 - RNA, 20 nt, synthetic construct
  misc_feature 1..20: note = synthetic construct
  source 1..20: mol_type = other RNA
ccagctcagctccacgtggt

SEQ ID NO: 28 - RNA, 20 nt, synthetic construct
  misc_feature 1..20: note = synthetic construct
  source 1..20: mol_type = other RNA
ccgcaaccacttccgctgtc

SEQ ID NO: 29 - RNA, 20 nt, synthetic construct
  misc_feature 1..20: note = synthetic construct
  source 1..20: mol_type = other RNA
gtccactcgtcattctccga

SEQ ID NO: 30 - RNA, 20 nt, synthetic construct
  misc_feature 1..20: note = synthetic construct
  source 1..20: mol_type = other RNA
gcccgtagaactggacttga

SEQ ID NO: 31 - RNA, 20 nt, synthetic construct
  misc_feature 1..20: note = synthetic construct
  source 1..20: mol_type = other RNA
tcacccagatcgtcagcgcc

SEQ ID NO: 32 - RNA, 20 nt, synthetic construct
  misc_feature 1..20: note = synthetic construct
  source 1..20: mol_type = other RNA
gggtccactcgtcattctcc

SEQ ID NO: 33 - RNA, 20 nt, synthetic construct
  misc_feature 1..20: note = synthetic construct
  source 1..20: mol_type = other RNA
tccagttctacgggctctcg

SEQ ID NO: 34 - RNA, 20 nt, synthetic construct
  misc_feature 1..20: note = synthetic construct
  source 1..20: mol_type = other RNA
cggagaatgacgagtggacc

SEQ ID NO: 35 - RNA, 20 nt, synthetic construct
  misc_feature 1..20: note = synthetic construct
  source 1..20: mol_type = other RNA
accacttccgctgtcaagtc

SEQ ID NO: 36 - RNA, 20 nt, synthetic construct
  misc_feature 1..20: note = synthetic construct
  source 1..20: mol_type = other RNA
aatgacgagtggacccagga

SEQ ID NO: 37 - RNA, 20 nt, synthetic construct
  misc_feature 1..20: note = synthetic construct
  source 1..20: mol_type = other RNA
acgggctctcggagaatgac

SEQ ID NO: 38 - RNA, 20 nt, synthetic construct
  misc_feature 1..20: note = synthetic construct
  source 1..20: mol_type = other RNA
gactccagatactgcctgag

SEQ ID NO: 39 - RNA, 20 nt, synthetic construct
  misc_feature 1..20: note = synthetic construct
  source 1..20: mol_type = other RNA
cgctgtgtttgagccatcag

SEQ ID NO: 40 - RNA, 20 nt, synthetic construct
  misc_feature 1..20: note = synthetic construct
  source 1..20: mol_type = other RNA
agaactggacttgacagcgg

SEQ ID NO: 41 - RNA, 20 nt, synthetic construct
  misc_feature 1..20: note = synthetic construct
  source 1..20: mol_type = other RNA
gagaccctcaggcggctgct

SEQ ID NO: 42 - RNA, 20 nt, synthetic construct
  misc_feature 1..20: note = synthetic construct
  source 1..20: mol_type = other RNA
cgtcattctccgagagcccg

SEQ ID NO: 43 - RNA, 20 nt, synthetic construct
  misc_feature 1..20: note = synthetic construct
  source 1..20: mol_type = other RNA
cagcccgccctcaatgactc

SEQ ID NO: 44 - RNA, 20 nt, synthetic construct
  misc_feature 1..20: note = synthetic construct
  source 1..20: mol_type = other RNA
ggctgctcaggcagtatctg

SEQ ID NO: 45 - RNA, 20 nt, synthetic construct
  misc_feature 1..20: note = synthetic construct
  source 1..20: mol_type = other RNA
agtggttgcgggggttctgc

SEQ ID NO: 46 - RNA, 20 nt, synthetic construct
  misc_feature 1..20: note = synthetic construct
  source 1..20: mol_type = other RNA
ggtcgctgtgtttgagccat

SEQ ID NO: 47 - RNA, 20 nt, synthetic construct
  misc_feature 1..20: note = synthetic construct
  source 1..20: mol_type = other RNA
gatctcatagaggatggtgg

SEQ ID NO: 48 - RNA, 20 nt, synthetic construct
  misc_feature 1..20: note = synthetic construct
  source 1..20: mol_type = other RNA
tgtttgagccatcagaagca

SEQ ID NO: 49 - RNA, 20 nt, synthetic construct
  misc_feature 1..20: note = synthetic construct
  source 1..20: mol_type = other RNA
tactgcctgagcagccgcct

SEQ ID NO: 50 - RNA, 20 nt, synthetic construct
  misc_feature 1..20: note = synthetic construct
  source 1..20: mol_type = other RNA
acccgcagcccctcaaggag

SEQ ID NO: 51 - RNA, 20 nt, synthetic construct
  misc_feature 1..20: note = synthetic construct
  source 1..20: mol_type = other RNA
aggcagtatctggagtcatt

SEQ ID NO: 52 - RNA, 20 nt, synthetic construct
  misc_feature 1..20: note = synthetic construct
  source 1..20: mol_type = other RNA
tgtgtttgagccatcagaag

SEQ ID NO: 53 - RNA, 20 nt, synthetic construct
  misc_feature 1..20: note = synthetic construct
  source 1..20: mol_type = other RNA
ggttgcgggggttctgccag

SEQ ID NO: 54 - RNA, 20 nt, synthetic construct
  misc_feature 1..20: note = synthetic construct
  source 1..20: mol_type = other RNA
gggtctcggccaccttctgg

SEQ ID NO: 55 - RNA, 20 nt, synthetic construct
  misc_feature 1..20: note = synthetic construct
  source 1..20: mol_type = other RNA
cagaaggtggccgagaccct

SEQ ID NO: 56 - RNA, 20 nt, synthetic construct
  misc_feature 1..20: note = synthetic construct
  source 1..20: mol_type = other RNA
cgccgaggcctggggtagag

SEQ ID NO: 57 - RNA, 20 nt, synthetic construct
  misc_feature 1..20: note = synthetic construct
  source 1..20: mol_type = other RNA
ggttctgccagaaggtggcc

SEQ ID NO: 58 - RNA, 20 nt, synthetic construct
  misc_feature 1..20: note = synthetic construct
  source 1..20: mol_type = other RNA
cagagatctcccacacccaa

SEQ ID NO: 59 - RNA, 20 nt, synthetic construct
  misc_feature 1..20: note = synthetic construct
  source 1..20: mol_type = other RNA
ttgagggcgggctgctcctt

SEQ ID NO: 60 - RNA, 20 nt, synthetic construct
  misc_feature 1..20: note = synthetic construct
  source 1..20: mol_type = other RNA
aagcctgtggccaggcacac

SEQ ID NO: 61 - RNA, 20 nt, synthetic construct
  misc_feature 1..20: note = synthetic construct
  source 1..20: mol_type = other RNA
cagcgccgaggcctggggta

SEQ ID NO: 62 - RNA, 20 nt, synthetic construct
  misc_feature 1..20: note = synthetic construct
  source 1..20: mol_type = other RNA
cccactcacctgctctaccc

SEQ ID NO: 63 - RNA, 20 nt, synthetic construct
  misc_feature 1..20: note = synthetic construct
  source 1..20: mol_type = other RNA
tgtctgccaccatcctctat

SEQ ID NO: 64 - RNA, 20 nt, synthetic construct
  misc_feature 1..20: note = synthetic construct
  source 1..20: mol_type = other RNA
tgcttctgatggctcaaaca

SEQ ID NO: 65 - RNA, 20 nt, synthetic construct
  misc_feature 1..20: note = synthetic construct
  source 1..20: mol_type = other RNA
ttcccattcacccaccagct

SEQ ID NO: 66 - RNA, 20 nt, synthetic construct
  misc_feature 1..20: note = synthetic construct
  source 1..20: mol_type = other RNA
gtcagcgccgaggcctgggg

SEQ ID NO: 67 - RNA, 20 nt, synthetic construct
  misc_feature 1..20: note = synthetic construct
  source 1..20: mol_type = other RNA
ccctcaggcggctgctcagg

SEQ ID NO: 68 - RNA, 20 nt, synthetic construct
  misc_feature 1..20: note = synthetic construct
  source 1..20: mol_type = other RNA
aatgactccagatactgcct

SEQ ID NO: 69 - RNA, 20 nt, synthetic construct
  misc_feature 1..20: note = synthetic construct
  source 1..20: mol_type = other RNA
cacactggtgtgcctggcca

SEQ ID NO: 70 - RNA, 20 nt, synthetic construct
  misc_feature 1..20: note = synthetic construct
  source 1..20: mol_type = other RNA
tcatagaggatggtggcaga

SEQ ID NO: 71 - RNA, 20 nt, synthetic construct
  misc_feature 1..20: note = synthetic construct
  source 1..20: mol_type = other RNA
cacccagatcgtcagcgccg

SEQ ID NO: 72 - RNA, 20 nt, synthetic construct
  misc_feature 1..20: note = synthetic construct
  source 1..20: mol_type = other RNA
tgacagcggaagtggttgcg

SEQ ID NO: 73 - RNA, 20 nt, synthetic construct
  misc_feature 1..20: note = synthetic construct
  source 1..20: mol_type = other RNA
tctccgagagcccgtagaac

SEQ ID NO: 74 - RNA, 20 nt, synthetic construct
  misc_feature 1..20: note = synthetic construct
  source 1..20: mol_type = other RNA
agtccagttctacgggctct

SEQ ID NO: 75 - RNA, 20 nt, synthetic construct
  misc_feature 1..20: note = synthetic construct
  source 1..20: mol_type = other RNA
atcgtcagcgccgaggcctg

SEQ ID NO: 76 - RNA, 20 nt, synthetic construct
  misc_feature 1..20: note = synthetic construct
  source 1..20: mol_type = other RNA
aaggaggtgcacagtggggt

SEQ ID NO: 77 - RNA, 20 nt, synthetic construct
  misc_feature 1..20: note = synthetic construct
  source 1..20: mol_type = other RNA
tatctggagtcattgagggc

SEQ ID NO: 78 - RNA, 20 nt, synthetic construct
  misc_feature 1..20: note = synthetic construct
  source 1..20: mol_type = other RNA
gcgggggttctgccagaagg

SEQ ID NO: 79 - RNA, 20 nt, synthetic construct
  misc_feature 1..20: note = synthetic construct
  source 1..20: mol_type = other RNA
cttgacagcggaagtggttg

SEQ ID NO: 80 - RNA, 20 nt, synthetic construct
  misc_feature 1..20: note = synthetic construct
  source 1..20: mol_type = other RNA
gtgtggccttttgggtgtgg

SEQ ID NO: 81 - RNA, 20 nt, synthetic construct
  misc_feature 1..20: note = synthetic construct
  source 1..20: mol_type = other RNA
gatcgtcagcgccgaggcct

SEQ ID NO: 82 - RNA, 20 nt, synthetic construct
  misc_feature 1..20: note = synthetic construct
  source 1..20: mol_type = other RNA
tgtggccaggcacaccagtg

SEQ ID NO: 83 - RNA, 20 nt, synthetic construct
  misc_feature 1..20: note = synthetic construct
  source 1..20: mol_type = other RNA
aggcctcggcgctgacgatc

SEQ ID NO: 84 - RNA, 20 nt, synthetic construct
  misc_feature 1..20: note = synthetic construct
  source 1..20: mol_type = other RNA
gtgaatgggaaggaggtgca

SEQ ID NO: 85 - RNA, 20 nt, synthetic construct
  misc_feature 1..20: note = synthetic construct
  source 1..20: mol_type = other RNA
gcggctgctcaggcagtatc

SEQ ID NO: 86 - RNA, 20 nt, synthetic construct
  misc_feature 1..20: note = synthetic construct
  source 1..20: mol_type = other RNA
actgcctgagcagccgcctg

SEQ ID NO: 87 - RNA, 20 nt, synthetic construct
  misc_feature 1..20: note = synthetic construct
  source 1..20: mol_type = other RNA
aaaggccacactggtgtgcc

SEQ ID NO: 88 - RNA, 20 nt, synthetic construct
  misc_feature 1..20: note = synthetic construct
  source 1..20: mol_type = other RNA
tgagggcgggctgctccttg

SEQ ID NO: 89 - RNA, 20 nt, synthetic construct
  misc_feature 1..20: note = synthetic construct
  source 1..20: mol_type = other RNA
gagcagccgcctgagggtct

SEQ ID NO: 90 - RNA, 20 nt, synthetic construct
  misc_feature 1..20: note = synthetic construct
  source 1..20: mol_type = other RNA
ctctcagctggtacacggca

SEQ ID NO: 91 - RNA, 20 nt, synthetic construct
  misc_feature 1..20: note = synthetic construct
  source 1..20: mol_type = other RNA
ttcggaacccaatcactgac

SEQ ID NO: 92 - RNA, 20 nt, synthetic construct
  misc_feature 1..20: note = synthetic construct
  source 1..20: mol_type = other RNA
taaacccggccactttcagg

SEQ ID NO: 93 - RNA, 20 nt, synthetic construct
  misc_feature 1..20: note = synthetic construct
  source 1..20: mol_type = other RNA
gattaaacccggccactttc

SEQ ID NO: 94 - RNA, 20 nt, synthetic construct
  misc_feature 1..20: note = synthetic construct
  source 1..20: mol_type = other RNA
cgtcatgagcagattaaacc

SEQ ID NO: 95 - RNA, 20 nt, synthetic construct
  misc_feature 1..20: note = synthetic construct
  source 1..20: mol_type = other RNA
agagtctctcagctggtaca

SEQ ID NO: 96 - RNA, 20 nt, synthetic construct
  misc_feature 1..20: note = synthetic construct
  source 1..20: mol_type = other RNA
acacggcagggtcagggttc

SEQ ID NO: 97 - RNA, 20 nt, synthetic construct
  misc_feature 1..20: note = synthetic construct
  source 1..20: mol_type = other RNA
tctctcagctggtacacggc

SEQ ID NO: 98 - RNA, 20 nt, synthetic construct
  misc_feature 1..20: note = synthetic construct
  source 1..20: mol_type = other RNA
tggatttagagtctctcagc

SEQ ID NO: 99 - RNA, 20 nt, synthetic construct
  misc_feature 1..20: note = synthetic construct
  source 1..20: mol_type = other RNA
gagaatcaaaatcggtgaat

SEQ ID NO: 100 - RNA, 20 nt, synthetic construct
  misc_feature 1..20: note = synthetic construct
  source 1..20: mol_type = other RNA
acaaaactgtgctagacatg

SEQ ID NO: 101 - RNA, 20 nt, synthetic construct
  misc_feature 1..20: note = synthetic construct
  source 1..20: mol_type = other RNA
tgtgctagacatgaggtcta

SEQ ID NO: 102 - RNA, 20 nt, synthetic construct
  misc_feature 1..20: note = synthetic construct
  source 1..20: mol_type = other RNA
gcaccaaagctgcccttacc

SEQ ID NO: 103 - RNA, 20 nt, synthetic construct
  misc_feature 1..20: note = synthetic construct
  source 1..20: mol_type = other RNA
aagttcctgtgatgtcaagc

SEQ ID NO: 104 - RNA, 20 nt, synthetic construct
  misc_feature 1..20: note = synthetic construct
  source 1..20: mol_type = other RNA
ctcgaccagcttgacatcac

SEQ ID NO: 105 - RNA, 20 nt, synthetic construct
  misc_feature 1..20: note = synthetic construct
  source 1..20: mol_type = other RNA
atcctcctcctgaaagtggc

SEQ ID NO: 106 - RNA, 20 nt, synthetic construct
  misc_feature 1..20: note = synthetic construct
  source 1..20: mol_type = other RNA
acccggccactttcaggagg

SEQ ID NO: 107 - RNA, 20 nt, synthetic construct
  misc_feature 1..20: note = synthetic construct
  source 1..20: mol_type = other RNA
ttaatctgctcatgacgctg

SEQ ID NO: 108 - RNA, 20 nt, synthetic construct
  misc_feature 1..20: note = synthetic construct
  source 1..20: mol_type = other RNA
acacggatgaacaataaggc

SEQ ID NO: 109 - RNA, 20 nt, synthetic construct
  misc_feature 1..20: note = synthetic construct
  source 1..20: mol_type = other RNA
acttacacggatgaacaata

SEQ ID NO: 110 - RNA, 20 nt, synthetic construct
  misc_feature 1..20: note = synthetic construct
  source 1..20: mol_type = other RNA
gctggtacacggcagggtca

SEQ ID NO: 111 - RNA, 20 nt, synthetic construct
  misc_feature 1..20: note = synthetic construct
  source 1..20: mol_type = other RNA
ggaagctacatacctacatt

SEQ ID NO: 112 - RNA, 20 nt, synthetic construct
  misc_feature 1..20: note = synthetic construct
  source 1..20: mol_type = other RNA
tcctcactgtgtgcatcagg

SEQ ID NO: 113 - RNA, 20 nt, synthetic construct
  misc_feature 1..20: note = synthetic construct
  source 1..20: mol_type = other RNA
ctggttcctcttccaaatgt

SEQ ID NO: 114 - RNA, 20 nt, synthetic construct
  misc_feature 1..20: note = synthetic construct
  source 1..20: mol_type = other RNA
aaagtcagatttgttgctcc

SEQ ID NO: 115 - RNA, 20 nt, synthetic construct
  misc_feature 1..20: note = synthetic construct
  source 1..20: mol_type = other RNA
ttcaaaacctgtcagtgatt

SEQ ID NO: 116 - RNA, 20 nt, synthetic construct
  misc_feature 1..20: note = synthetic construct
  source 1..20: mol_type = other RNA
tgctcatgacgctgcggctg

SEQ ID NO: 117 - RNA, 20 nt, synthetic construct
  misc_feature 1..20: note = synthetic construct
  source 1..20: mol_type = other RNA
tcaaggcccctcacctcagc

SEQ ID NO: 118 - RNA, 20 nt, synthetic construct
  misc_feature 1..20: note = synthetic construct
  source 1..20: mol_type = other RNA
ggcgtttgcacatgcaaagt

SEQ ID NO: 119 - RNA, 20 nt, synthetic construct
  misc_feature 1..20: note = synthetic construct
  source 1..20: mol_type = other RNA
gaccacagccgcagcgtcat

SEQ ID NO: 120 - RNA, 20 nt, synthetic construct
  misc_feature 1..20: note = synthetic construct
  source 1..20: mol_type = other RNA
atgacgctgcggctgtggtc

SEQ ID NO: 121 - RNA, 20 nt, synthetic construct
  misc_feature 1..20: note = synthetic construct
  source 1..20: mol_type = other RNA
attcggaacccaatcactga

SEQ ID NO: 122 - RNA, 20 nt, synthetic construct
  misc_feature 1..20: note = synthetic construct
  source 1..20: mol_type = other RNA
aacccggccactttcaggag

SEQ ID NO: 123 - RNA, 20 nt, synthetic construct
  misc_feature 1..20: note = synthetic construct
  source 1..20: mol_type = other RNA
ttaaacccggccactttcag

SEQ ID NO: 124 - RNA, 20 nt, synthetic construct
  misc_feature 1..20: note = synthetic construct
  source 1..20: mol_type = other RNA
agatttgttgctccaggcca

SEQ ID NO: 125 - RNA, 20 nt, synthetic construct
  misc_feature 1..20: note = synthetic construct
  source 1..20: mol_type = other RNA
tgagaatcaaaatcggtgaa

SEQ ID NO: 126 - RNA, 20 nt, synthetic construct
  misc_feature 1..20: note = synthetic construct
  source 1..20: mol_type = other RNA
gatgtcaagctggtcgagaa

SEQ ID NO: 127 - RNA, 20 nt, synthetic construct
  misc_feature 1..20: note = synthetic construct
  source 1..20: mol_type = other RNA
gtttcaaagcttttctcgac

SEQ ID NO: 128 - RNA, 20 nt, synthetic construct
  misc_feature 1..20: note = synthetic construct
  source 1..20: mol_type = other RNA
tgaaggcgtttgcacatgca

SEQ ID NO: 129 - RNA, 20 nt, synthetic construct
  misc_feature 1..20: note = synthetic construct
  source 1..20: mol_type = other RNA
accctgaccctgccgtgtac

SEQ ID NO: 130 - RNA, 20 nt, synthetic construct
  misc_feature 1..20: note = synthetic construct
  source 1..20: mol_type = other RNA
agcttcaaggcccctcacct

SEQ ID NO: 131 - RNA, 20 nt, synthetic construct
  misc_feature 1..20: note = synthetic construct
  source 1..20: mol_type = other RNA
gttccgaatcctcctcctga

SEQ ID NO: 132 - RNA, 20 nt, synthetic construct
  misc_feature 1..20: note = synthetic construct
  source 1..20: mol_type = other RNA
agattaaacccggccacttt

SEQ ID NO: 133 - RNA, 20 nt, synthetic construct
  misc_feature 1..20: note = synthetic construct
  source 1..20: mol_type = other RNA
ccctgccgtgtaccagctga

SEQ ID NO: 134 - RNA, 20 nt, synthetic construct
  misc_feature 1..20: note = synthetic construct
  source 1..20: mol_type = other RNA
tcactggatttagagtctct

SEQ ID NO: 135 - RNA, 20 nt, synthetic construct
  misc_feature 1..20: note = synthetic construct
  source 1..20: mol_type = other RNA
tacttacacggatgaacaat

SEQ ID NO: 136 - RNA, 20 nt, synthetic construct
  misc_feature 1..20: note = synthetic construct
  source 1..20: mol_type = other RNA
tatcacagacaaaactgtgc

SEQ ID NO: 137 - RNA, 20 nt, synthetic construct
  misc_feature 1..20: note = synthetic construct
  source 1..20: mol_type = other RNA
ccacagcactgttgctcttg

SEQ ID NO: 138 - RNA, 20 nt, synthetic construct
  misc_feature 1..20: note = synthetic construct
  source 1..20: mol_type = other RNA
cctgtgatgtcaagctggtc

SEQ ID NO: 139 - RNA, 20 nt, synthetic construct
  misc_feature 1..20: note = synthetic construct
  source 1..20: mol_type = other RNA
acatgaggtctatggacttc

SEQ ID NO: 140 - RNA, 20 nt, synthetic construct
  misc_feature 1..20: note = synthetic construct
  source 1..20: mol_type = other RNA
actgttgctcttgaagtcca

SEQ ID NO: 141 - RNA, 20 nt, synthetic construct
  misc_feature 1..20: note = synthetic construct
  source 1..20: mol_type = other RNA
agctacatacctacatttgg

SEQ ID NO: 142 - RNA, 20 nt, synthetic construct
  misc_feature 1..20: note = synthetic construct
  source 1..20: mol_type = other RNA
tagaaagttcctgtgatgtc

SEQ ID NO: 143 - RNA, 20 nt, synthetic construct
  misc_feature 1..20: note = synthetic construct
  source 1..20: mol_type = other RNA
tcactgtgtgcatcaggagg

SEQ ID NO: 144 - RNA, 20 nt, synthetic construct
  misc_feature 1..20: note = synthetic construct
  source 1..20: mol_type = other RNA
gacaaaactgtgctagacat

SEQ ID NO: 145 - RNA, 20 nt, synthetic construct
  misc_feature 1..20: note = synthetic construct
  source 1..20: mol_type = other RNA
cttcaacaacagcattattc

SEQ ID NO: 146 - RNA, 20 nt, synthetic construct
  misc_feature 1..20: note = synthetic construct
  source 1..20: mol_type = other RNA
caacaacagcattattccag

SEQ ID NO: 147 - RNA, 20 nt, synthetic construct
  misc_feature 1..20: note = synthetic construct
  source 1..20: mol_type = other RNA
tctcaaacaaatgtgtcaca

SEQ ID NO: 148 - RNA, 20 nt, synthetic construct
  misc_feature 1..20: note = synthetic construct
  source 1..20: mol_type = other RNA
atgaggtctatggacttcaa

SEQ ID NO: 149 - RNA, 20 nt, synthetic construct
  misc_feature 1..20: note = synthetic construct
  source 1..20: mol_type = other RNA
gaccctgccgtgtaccagct

SEQ ID NO: 150 - RNA, 20 nt, synthetic construct
  misc_feature 1..20: note = synthetic construct
  source 1..20: mol_type = other RNA
cccctgtcttacctgtttca

SEQ ID NO: 151 - RNA, 20 nt, synthetic construct
  misc_feature 1..20: note = synthetic construct
  source 1..20: mol_type = other RNA
agcaacagtgctgtggcctg

SEQ ID NO: 152 - RNA, 20 nt, synthetic construct
  misc_feature 1..20: note = synthetic construct
  source 1..20: mol_type = other RNA
ctacatacctacatttggaa

SEQ ID NO: 153 - RNA, 20 nt, synthetic construct
  misc_feature 1..20: note = synthetic construct
  source 1..20: mol_type = other RNA
actttgtgacacatttgttt

SEQ ID NO: 154 - RNA, 20 nt, synthetic construct
  misc_feature 1..20: note = synthetic construct
  source 1..20: mol_type = other RNA
atcacagacaaaactgtgct

SEQ ID NO: 155 - RNA, 20 nt, synthetic construct
  misc_feature 1..20: note = synthetic construct
  source 1..20: mol_type = other RNA
ttcaacaacagcattattcc

SEQ ID NO: 156 - RNA, 20 nt, synthetic construct
  misc_feature 1..20: note = synthetic construct
  source 1..20: mol_type = other RNA
catgaggtctatggacttca

SEQ ID NO: 157 - RNA, 20 nt, synthetic construct
  misc_feature 1..20: note = synthetic construct
  source 1..20: mol_type = other RNA
gctacatacctacatttgga

SEQ ID NO: 158 - RNA, 20 nt, synthetic construct
  misc_feature 1..20: note = synthetic construct
  source 1..20: mol_type = other RNA
ctcttgtcccacagatatcc

SEQ ID NO: 159 - RNA, 20 nt, synthetic construct
  misc_feature 1..20: note = synthetic construct
  source 1..20: mol_type = other RNA
gattctgatgtgtatatcac

SEQ ID NO: 160 - RNA, 20 nt, synthetic construct
  misc_feature 1..20: note = synthetic construct
  source 1..20: mol_type = other RNA
ctgtgatgtcaagctggtcg

SEQ ID NO: 161 - RNA, 20 nt, synthetic construct
  misc_feature 1..20: note = synthetic construct
  source 1..20: mol_type = other RNA
cctgccgtgtaccagctgag

SEQ ID NO: 162 - RNA, 20 nt, synthetic construct
  misc_feature 1..20: note = synthetic construct
  source 1..20: mol_type = other RNA
acatacctacatttggaaga

SEQ ID NO: 163 - RNA, 20 nt, synthetic construct
  misc_feature 1..20: note = synthetic construct
  source 1..20: mol_type = other RNA
cctcactgtgtgcatcagga

SEQ ID NO: 164 - RNA, 20 nt, synthetic construct
  misc_feature 1..20: note = synthetic construct
  source 1..20: mol_type = other RNA
acaaatgtgtcacaaagtaa

SEQ ID NO: 165 - RNA, 20 nt, synthetic construct
  misc_feature 1..20: note = synthetic construct
  source 1..20: mol_type = other RNA
gagcaacagtgctgtggcct

SEQ ID NO: 166 - RNA, 20 nt, synthetic construct
  misc_feature 1..20: note = synthetic construct
  source 1..20: mol_type = other RNA
tcgaccagcttgacatcaca

SEQ ID NO: 167 - RNA, 20 nt, synthetic construct
  misc_feature 1..20: note = synthetic construct
  source 1..20: mol_type = other RNA
attaaacccggccactttca

SEQ ID NO: 168 - RNA, 20 nt, synthetic construct
  misc_feature 1..20: note = synthetic construct
  source 1..20: mol_type = other RNA
cacggcagggtcagggttct

SEQ ID NO: 169 - RNA, 20 nt, synthetic construct
  misc_feature 1..20: note = synthetic construct
  source 1..20: mol_type = other RNA
aaacccggccactttcagga

SEQ ID NO: 170 - RNA, 20 nt, synthetic construct
  misc_feature 1..20: note = synthetic construct
  source 1..20: mol_type = other RNA
caaggcccctcacctcagct

SEQ ID NO: 171 - RNA, 20 nt, synthetic construct
  misc_feature 1..20: note = synthetic construct
  source 1..20: mol_type = other RNA
cccggccactttcaggagga

SEQ ID NO: 172 - RNA, 20 nt, synthetic construct
  misc_feature 1..20: note = synthetic construct
  source 1..20: mol_type = other RNA
catttctataatacttacac

SEQ ID NO: 173 - RNA, 20 nt, synthetic construct
  misc_feature 1..20: note = synthetic construct
  source 1..20: mol_type = other RNA
acagccgcagcgtcatgagc

SEQ ID NO: 174 - RNA, 20 nt, synthetic construct
  misc_feature 1..20: note = synthetic construct
  source 1..20: mol_type = other RNA
atcaaaatcggtgaataggc

SEQ ID NO: 175 - RNA, 20 nt, synthetic construct
  misc_feature 1..20: note = synthetic construct
  source 1..20: mol_type = other RNA
gcgtttgcacatgcaaagtc

SEQ ID NO: 176 - RNA, 20 nt, synthetic construct
  misc_feature 1..20: note = synthetic construct
  source 1..20: mol_type = other RNA
ctgttgctcttgaagtccat

SEQ ID NO: 177 - RNA, 20 nt, synthetic construct
  misc_feature 1..20: note = synthetic construct
  source 1..20: mol_type = other RNA
aaatcggtgaataggcagac

SEQ ID NO: 178 - RNA, 20 nt, synthetic construct
  misc_feature 1..20: note = synthetic construct
  source 1..20: mol_type = other RNA
ttgtctgtgatatacacatc

SEQ ID NO: 179 - RNA, 100 nt, synthetic construct
  misc_feature 1..100: note = sgRNA sequence
  source 1..100: mol_type = other RNA
ggctctcggagaatgacgaggttttagagctagaaatagcaagttaaaataaggctagtccgttatcaacttgaaaaagtggcaccgagtcggtgctttt

SEQ ID NO: 180 - RNA, 100 nt, synthetic construct
  misc_feature 1..100: note = sgRNA sequence
  source 1..100: mol_type = other RNA
ggcctcggcgctgacgatctgttttagagctagaaatagcaagttaaaataaggctagtccgttatcaacttgaaaaagtggcaccgagtcggtgctttt

SEQ ID NO: 181 - RNA, 100 nt, synthetic construct
  misc_feature 1..100: note = sgRNA sequence
  source 1..100: mol_type = other RNA
atgacgagtggacccaggatgttttagagctagaaatagcaagttaaaataaggctagtccgttatcaacttgaaaaagtggcaccgagtcggtgctttt

SEQ ID NO: 182 - RNA, 100 nt, synthetic construct
  misc_feature 1..100: note = sgRNA sequence
  source 1..100: mol_type = other RNA
agaaggtggccgagaccctcgttttagagctagaaatagcaagttaaaataaggctagtccgttatcaacttgaaaaagtggcaccgagtcggtgctttt

SEQ ID NO: 183 - RNA, 100 nt, synthetic construct
  misc_feature 1..100: note = sgRNA sequence
  source 1..100: mol_type = other RNA
tgagggtctcggccaccttcgttttagagctagaaatagcaagttaaaataaggctagtccgttatcaacttgaaaaagtggcaccgagtcggtgctttt

SEQ ID NO: 184 - RNA, 100 nt, synthetic construct
  misc_feature 1..100: note = sgRNA sequence
  source 1..100: mol_type = other RNA
agagatctcccacacccaaagttttagagctagaaatagcaagttaaaataaggctagtccgttatcaacttgaaaaagtggcaccgagtcggtgctttt

SEQ ID NO: 185 - RNA, 20 nt, synthetic construct
  misc_feature 1..20: note = synthetic construct
  source 1..20: mol_type = other RNA
tcagggttctggatatctgt

SEQ ID NO: 186 - RNA, 100 nt, synthetic construct
  misc_feature 1..100: note = sgRNA sequence
  source 1..100: mol_type = other RNA
ctctcagctggtacacggcagttttagagctagaaatagcaagttaaaataaggctagtccgttatcaacttgaaaaagtggcaccgagtcggtgctttt

SEQ ID NO: 187 - RNA, 100 nt, synthetic construct
  misc_feature 1..100: note = sgRNA sequence
  source 1..100: mol_type = other RNA
ttcggaacccaatcactgacgttttagagctagaaatagcaagttaaaataaggctagtccgttatcaacttgaaaaagtggcaccgagtcggtgctttt

SEQ ID NO: 188 - RNA, 100 nt, synthetic construct
  misc_feature 1..100: note = sgRNA sequence
  source 1..100: mol_type = other RNA
taaacccggccactttcagggttttagagctagaaatagcaagttaaaataaggctagtccgttatcaacttgaaaaagtggcaccgagtcggtgctttt

SEQ ID NO: 189 - RNA, 100 nt, synthetic construct
  misc_feature 1..100: note = sgRNA sequence
  source 1..100: mol_type = other RNA
gattaaacccggccactttcgttttagagctagaaatagcaagttaaaataaggctagtccgttatcaacttgaaaaagtggcaccgagtcggtgctttt

SEQ ID NO: 190 - RNA, 100 nt, synthetic construct
  misc_feature 1..100: note = sgRNA sequence
  source 1..100: mol_type = other RNA
cgtcatgagcagattaaaccgttttagagctagaaatagcaagttaaaataaggctagtccgttatcaacttgaaaaagtggcaccgagtcggtgctttt

SEQ ID NO: 191 - RNA, 100 nt, synthetic construct
  misc_feature 1..100: note = sgRNA sequence
  source 1..100: mol_type = other RNA
agagtctctcagctggtacagttttagagctagaaatagcaagttaaaataaggctagtccgttatcaacttgaaaaagtggcaccgagtcggtgctttt

SEQ ID NO: 192 - RNA, 100 nt, synthetic construct
  misc_feature 1..100: note = sgRNA sequence
  source 1..100: mol_type = other RNA
tcagggttctggatatctgtgttttagagctagaaatagcaagttaaaataaggctagtccgttatcaacttgaaaaagtggcaccgagtcggtgctttt

SEQ ID NO: 193 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = gm
  modified_base 2: mod_base = OTHER; note = 2'-O-Methyladenosine
  modified_base 3: mod_base = gm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
gagtccgagcagaagaagaagttttagagctagaaatagcaagttaaaataaggctagtccgttatcaacttgaaaaagtggcaccgagtcggtgctttt

SEQ ID NO: 194 - RNA, 100 nt, synthetic construct; identical to SEQ ID NO: 193
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = gm
  modified_base 2: mod_base = OTHER; note = 2'-O-Methyladenosine
  modified_base 3: mod_base = cm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA

SEQ ID NO: 195 - RNA, 74 nt, synthetic construct
  misc_feature 1..74: note = sgRNA sequence
  source 1..74: mol_type = other RNA
aacagcatagcaagttaaaataaggctagtccgttatcaacttgaaaaagtggcaccgagtcggtgcttttttt

SEQ ID NO: 196 - RNA, 100 nt, synthetic construct; identical to SEQ ID NO: 179
  misc_feature 1..100: note = sgRNA sequence
  source 1..100: mol_type = other RNA

SEQ ID NO: 197 - RNA, 100 nt, synthetic construct; identical to SEQ ID NO: 180
  misc_feature 1..100: note = sgRNA sequence
  source 1..100: mol_type = other RNA

SEQ ID NO: 198 - RNA, 100 nt, synthetic construct; identical to SEQ ID NO: 181
  misc_feature 1..100: note = sgRNA sequence
  source 1..100: mol_type = other RNA

SEQ ID NO: 199 - RNA, 100 nt, synthetic construct; identical to SEQ ID NO: 183
  misc_feature 1..100: note = sgRNA sequence
  source 1..100: mol_type = other RNA

SEQ ID NO: 200 - RNA, 100 nt, synthetic construct; identical to SEQ ID NO: 184
  misc_feature 1..100: note = sgRNA sequence
  source 1..100: mol_type = other RNA

SEQ ID NO: 201 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = OTHER; note = 2'-O-Methyladenosine
  modified_base 2: mod_base = cm
  modified_base 3: mod_base = OTHER; note = 2'-O-Methyladenosine
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
acacggcagggtcagggttcgttttagagctagaaatagcaagttaaaataaggctagtccgttatcaacttgaaaaagtggcaccgagtcggtgctttt

SEQ ID NO: 202 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = OTHER; note = 2'-O-Methyladenosine
  modified_base 2: mod_base = gm
  modified_base 3: mod_base = cm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
agctggtacacggcagggtcgttttagagctagaaatagcaagttaaaataaggctagtccgttatcaacttgaaaaagtggcaccgagtcggtgctttt

SEQ ID NO: 203 - RNA, 100 nt, synthetic construct; identical to SEQ ID NO: 186
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = cm
  modified_base 2: mod_base = um
  modified_base 3: mod_base = cm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA

SEQ ID NO: 204 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = um
  modified_base 2: mod_base = cm
  modified_base 3: mod_base = um
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
tctctcagctggtacacggcgttttagagctagaaatagcaagttaaaataaggctagtccgttatcaacttgaaaaagtggcaccgagtcggtgctttt

SEQ ID NO: 205 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = um
  modified_base 2: mod_base = gm
  modified_base 3: mod_base = gm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
tggatttagagtctctcagcgttttagagctagaaatagcaagttaaaataaggctagtccgttatcaacttgaaaaagtggcaccgagtcggtgctttt

SEQ ID NO: 206 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = um
  modified_base 2: mod_base = OTHER; note = 2'-O-Methyladenosine
  modified_base 3: mod_base = gm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
taggcagacagacttgtcacgttttagagctagaaatagcaagttaaaataaggctagtccgttatcaacttgaaaaagtggcaccgagtcggtgctttt

SEQ ID NO: 207 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = um
  modified_base 2: mod_base = cm
  modified_base 3: mod_base = um
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
tctgtgggacaagaggatcagttttagagctagaaatagcaagttaaaataaggctagtccgttatcaacttgaaaaagtggcaccgagtcggtgctttt

SEQ ID NO: 208 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = OTHER; note = 2'-O-Methyladenosine
  modified_base 2: mod_base = um
  modified_base 3: mod_base = cm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
atctgtgggacaagaggatcgttttagagctagaaatagcaagttaaaataaggctagtccgttatcaacttgaaaaagtggcaccgagtcggtgctttt

SEQ ID NO: 209 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = cm
  modified_base 2: mod_base = um
  modified_base 3: mod_base = gm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
ctggatatctgtgggacaaggttttagagctagaaatagcaagttaaaataaggctagtccgttatcaacttgaaaaagtggcaccgagtcggtgctttt

SEQ ID NO: 210 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = gm
  modified_base 2: mod_base = um
  modified_base 3: mod_base = cm
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
gtcagggttctggatatctggttttagagctagaaatagcaagttaaaataaggctagtccgttatcaacttgaaaaagtggcaccgagtcggtgctttt

SEQ ID NO: 211 - RNA, 100 nt, synthetic construct; identical to SEQ ID NO: 192
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  modified_base 1: mod_base = um
  modified_base 2: mod_base = cm
  modified_base 3: mod_base = OTHER; note = 2'-O-Methyladenosine
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA

SEQ ID NO: 212 - RNA, 100 nt, synthetic construct; identical to SEQ ID NO: 206
  misc_feature 1..100: note = sgRNA sequence
  source 1..100: mol_type = other RNA

SEQ ID NO: 213 - RNA, 20 nt, synthetic construct
  misc_feature 1..20: note = synthetic construct
  source 1..20: mol_type = other RNA
agctggtacacggcagggtc

SEQ ID NO: 214 - RNA, 20 nt, synthetic construct
  misc_feature 1..20: note = synthetic construct
  source 1..20: mol_type = other RNA
taggcagacagacttgtcac

SEQ ID NO: 215 - RNA, 20 nt, synthetic construct
  misc_feature 1..20: note = synthetic construct
  source 1..20: mol_type = other RNA
tctgtgggacaagaggatca

SEQ ID NO: 216 - RNA, 20 nt, synthetic construct
  misc_feature 1..20: note = synthetic construct
  source 1..20: mol_type = other RNA
atctgtgggacaagaggatc

SEQ ID NO: 217 - RNA, 20 nt, synthetic construct
  misc_feature 1..20: note = synthetic construct
  source 1..20: mol_type = other RNA
ctggatatctgtgggacaag

SEQ ID NO: 218 - RNA, 20 nt, synthetic construct
  misc_feature 1..20: note = synthetic construct
  source 1..20: mol_type = other RNA
gtcagggttctggatatctg

SEQ ID NO: 219-249 - skipped (31 entries)

SEQ ID NO: 250 - DNA, 819 bp, Homo sapiens
  source 1..819: mol_type = genomic DNA
atggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctga

SEQ ID NO: 251 - protein, 272 aa, Homo sapiens
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTAWINDYKLSFGAGTTVTVRANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 252 - DNA, 945 bp, Homo sapiens
  source 1..945: mol_type = genomic DNA
atgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggctga

SEQ ID NO: 253 - protein, 314 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 254-299 - skipped (46 entries)

SEQ ID NO: 300 - RNA, 100 nt, synthetic construct
  misc_feature 1..100: note = Modified sgRNA
  modified_base 1..4: mod_base = OTHER; note = The 3 phosphodiester bonds between nucleotides 1 to 4 are substituted for phosphorothioate (PS) bonds
  variation 1..20: note = n is a, c, g, or u
  modified_base 29: mod_base = gm
  modified_base 30: mod_base = cm
  modified_base 31: mod_base = um
  modified_base 32: mod_base = OTHER; note = 2'-O-Methyladenosine
  modified_base 33: mod_base = gm
  modified_base 34..36: mod_base = OTHER; note = 2'-O-Methyladenosine
  modified_base 37: mod_base = um
  modified_base 38: mod_base = OTHER; note = 2'-O-Methyladenosine
  modified_base 39: mod_base = gm
  modified_base 40: mod_base = cm
  modified_base 69: mod_base = OTHER; note = 2'-O-Methyladenosine
  modified_base 70: mod_base = cm
  modified_base 71..72: mod_base = um
  modified_base 73: mod_base = gm
  modified_base 74..78: mod_base = OTHER; note = 2'-O-Methyladenosine
  modified_base 79: mod_base = gm
  modified_base 80: mod_base = um
  modified_base 81..82: mod_base = gm
  modified_base 83: mod_base = cm
  modified_base 84: mod_base = OTHER; note = 2'-O-Methyladenosine
  modified_base 85..86: mod_base = cm
  modified_base 87: mod_base = gm
  modified_base 88: mod_base = OTHER; note = 2'-O-Methyladenosine
  modified_base 89: mod_base = gm
  modified_base 90: mod_base = um
  modified_base 91: mod_base = cm
  modified_base 92..93: mod_base = gm
  modified_base 94: mod_base = um
  modified_base 95: mod_base = gm
  modified_base 96: mod_base = cm
  modified_base 97..100: mod_base = um
  modified_base 97..100: mod_base = OTHER; note = The 3 phosphodiester bonds between nucleotides 97 to 100 are substituted for phosphorothioate (PS) bonds
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 301-399 - skipped (99 entries)

SEQ ID NO: 400 - RNA, 22 nt, synthetic construct
  misc_feature 1..22: note = synthetic construct
  source 1..22: mol_type = other RNA
gttttagagctatgctgttttg

SEQ ID NO: 401 - RNA, 80 nt, synthetic construct
  misc_feature 1..80: note = synthetic construct
  source 1..80: mol_type = other RNA
gttttagagctagaaatagcaagttaaaataaggctagtccgttatcaacttgaaaaagtggcaccgagtcggtgctttt

SEQ ID NO: 402 - RNA, 76 nt, synthetic construct
  misc_feature 1..76: note = synthetic construct
  source 1..76: mol_type = other RNA
gttttagagctagaaatagcaagttaaaataaggctagtccgttatcaacttgaaaaagtggcaccgagtcggtgc

SEQ ID NO: 403 - RNA, 77 nt, synthetic construct
  misc_feature 1..77: note = synthetic construct
  variation 1: note = n is a, c, g, or u
  source 1..77: mol_type = other RNA
ngttttagagctagaaatagcaagttaaaataaggctagtccgttatcaacttgaaaaagtggcaccgagtcggtgc

SEQ ID NO: 404-499 - skipped (96 entries)

SEQ ID NO: 500 - protein, 608 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTAWINDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 501 - protein, 272 aa, Homo sapiens
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTAWINDYKLSFGAGTTVTVRANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 502 - protein, 314 aa, Homo sapiens
MGSWTLCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASRKTGGYSNQPQHFGDGTRLSILEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 503 - protein, 608 aa, Homo sapiens; identical to SEQ ID NO: 500

SEQ ID NO: 504 - protein, 272 aa, Homo sapiens; identical to SEQ ID NO: 251

SEQ ID NO: 505 - protein, 314 aa, Homo sapiens; identical to SEQ ID NO: 502

SEQ ID NO: 506 - protein, 602 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPGALYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAATEDLTLIWGAGTKLIIKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 507 - protein, 270 aa, Homo sapiens
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAATEDLTLIWGAGTKLIIKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 508 - protein, 310 aa, Homo sapiens
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPGALYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 509 - protein, 612 aa, Homo sapiens
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDYLVSNEKLFFGSGTQLSVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNLLSNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 510 - protein, 274 aa, Homo sapiens
MISLRVLLVILWLQLSWVWSQRKEVEQDPGPFNVPEGATVAFNCTYSNSASQSFFWYRQDCRKEPKLLMSVYSSGNEDGRFTAQLNRASQYISLLIRDSKLSDSATYLCVVNLLSNQGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 511 - protein, 316 aa, Homo sapiens
MGCRLLCCAVLCLLGAGELVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDYLVSNEKLFFGSGTQLSVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 512 - protein, 612 aa, Homo sapiens; identical to SEQ ID NO: 509

SEQ ID NO: 513 - protein, 274 aa, Homo sapiens; identical to SEQ ID NO: 510

SEQ ID NO: 514 - protein, 316 aa, Homo sapiens; identical to SEQ ID NO: 511

SEQ ID NO: 515 - protein, 614 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLRGGLEKLFFGSGTQLSVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAISGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 516 - protein, 273 aa, Homo sapiens
MSLSSLLKVVTASLWLGPGIAQKITQTQPGMFVQEKEAVTLDCTYDTSDPSYGLFWYKQPSSGEMIFLIYQGSYDQQNATEGRYSLNFQKARKSANLVISASQLGDSAMYFCAISGNTPLVFGKGTRLSVIANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 517 - protein, 319 aa, Homo sapiens
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLRGGLEKLFFGSGTQLSVLEDLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 518 - protein, 609 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLYRGEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARGQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 519 - protein, 277 aa, Homo sapiens
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAARGQGNLIFGKGTKLSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 520 - protein, 310 aa, Homo sapiens
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSLYRGEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 521 - protein, 641 aa, Homo sapiens
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASRGYHRLNNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMISLRVLLVILWLQLSWVWSGGGSWSHPQFEKGGGSGGGSGGSAWSHPQFEKQRKEVEQDPGPFNVPEGATVAFNCTYSNSASQSFFWYRQDCRKEPKLLMSVYSSGNEDGRFTAQLNRASQYISLLIRDSKLSDSATYLCVVKPDPGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 522 - protein, 307 aa, Homo sapiens
MISLRVLLVILWLQLSWVWSGGGSWSHPQFEKGGGSGGGSGGSAWSHPQFEKQRKEVEQDPGPFNVPEGATVAFNCTYSNSASQSFFWYRQDCRKEPKLLMSVYSSGNEDGRFTAQLNRASQYISLLIRDSKLSDSATYLCVVKPDPGAGSYQLTFGKGTKLSVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 523 - protein, 312 aa, Homo sapiens
MSLGLLCCGAFSLLWAGPVNAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASRGYHRLNNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 524-599 - skipped (76 entries)

SEQ ID NO: 600 - protein, 7 aa, Betapolyomavirus macacae
PKKKRKV

SEQ ID NO: 601 - protein, 7 aa, Betapolyomavirus macacae
PKKKRRV

SEQ ID NO: 602 - protein, 16 aa, Xenopus laevis
KRPAATKKAGQAKKKK

SEQ ID NO: 603 - DNA, 1183 bp, Homo sapiens
  source 1..1183: mol_type = genomic DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 604 - DNA, 238 bp, Homo sapiens
  source 1..238: mol_type = genomic DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggt

SEQ ID NO: 605 - DNA, 457 bp, Homo sapiens
  source 1..457: mol_type = genomic DNA
aacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcacc

SEQ ID NO: 606 - DNA, 426 bp, Homo sapiens
  source 1..426: mol_type = genomic DNA
gatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccaccggtcgccacc

SEQ ID NO: 607 - DNA, 500 bp, Homo sapiens
  source 1..500: mol_type = genomic DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 608 - DNA, 54 bp, Homo sapiens
  source 1..54: mol_type = genomic DNA
gagggccgcggcagcctgctgacctgcggcgacgtggaggagaatcccggcccc

SEQ ID NO: 609 - protein, 22 aa, synthetic construct
  REGION 1..22: note = Self-cleaving peptide derived from porcine teschovirus-1
GSGATNFSLLKQAGDVEENPGP

SEQ ID NO: 610 - protein, 478 aa, synthetic construct
  REGION 1..478: note = Enhanced Green Fluorescent Protein
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 611 - DNA, 35 bp, Betapolyomavirus macacae
  source 1..35: mol_type = genomic DNA
aataaagcaatagcatcacaaatttcacaaataaa

SEQ ID NO: 612 - DNA, 276 bp, Homo sapiens
  source 1..276: mol_type = genomic DNA
cctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccc

SEQ ID NO: 613 - DNA, 4612 bp, Homo sapiens
  source 1..4612: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacggccggccccgccaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaacagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaacgcgtggtgtaatcatggtcatag

SEQ ID NO: 614 - DNA, 3618 bp, Homo sapiens
  source 1..3618: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccaccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtcgcaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaacagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagag

SEQ ID NO: 615 - DNA, 3825 bp, Homo sapiens
  source 1..3825: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatctaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaacagatctaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 616 - DNA, 3794 bp, Homo sapiens
  source 1..3794: mol_type = genomic DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctagatctaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacgatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccaccggtcgccaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaacagatctaggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 617 - DNA, 3893 bp, Homo sapiens
  source 1..3893: mol_type = genomic DNA
cctgcaggcagctgcgcgctcgctcgctcactgaggccgcccgggcgtcgggcgacctttggtcgcccggcctcagtgagcgagcgagcgcgcagagagggagtggccaactccatcactaggggttcctagatctaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgcgaagcaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaacagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggaagcttggtgtaatca

SEQ ID NO: 618 - DNA, 3857 bp, Homo sapiens
  source 1..3857: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatcgatcccccgggctgcaggaattcaagcgagaagacaagggcagaaagcaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaacagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaacgcgtggtgtaa

SEQ ID NO: 619 - DNA, 3891 bp, Homo sapiens
  source 1..3891: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgcgaagcaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaacagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtg

SEQ ID NO: 620 - DNA, 3820 bp, Homo sapiens
  source 1..3820: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacgatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcgcctggagacgccatccacgctgttttgacctccatagaagacaccgactctagaggatccaccggtcgccaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaacagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaacgcgtg

SEQ ID NO: 621 - DNA, 3638 bp, Homo sapiens
  source 1..3638: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtccgagtaattcatacaaaaggactcgcccctgccttggggaatcccagggaccgtcgttaaactcccactaacgtagaacccagagatcgctgcgttcccgccccctcacccgcccgctctcgtcatcactgaggtggagaagagcatgcgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaacatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgttgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgcagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaaacgcgtggtgtaatca

SEQ ID NO: 622 - DNA, 2382 bp, Homo sapiens
  source 1..2382: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgagggccgcggcagcctgctgacctgcggcgacgtggaggagaatcccggccccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagaattctctggccgtcgttttac

SEQ ID NO: 623 - DNA, 4618 bp, Homo sapiens
  source 1..4618: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagactgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagaattctctgg

SEQ ID NO: 624 - DNA, 4587 bp, Homo sapiens
  source 1..4587: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacggccggccccgccaccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 625 - DNA, 4630 bp, Homo sapiens
  source 1..4630: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtctgtaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagaattctctggccgtcgttttac

SEQ ID NO: 626 - DNA, 4654 bp, Homo sapiens
  source 1..4654: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgctgagccccgatctgcctgacagcgcctggaacaccagactgctgtgccacgtgatgctgtgcctgctgggagccgtgtctgtggctgctggcgtgatccagagccccagacacctgatcaaagagaagagagagacagccaccctgaagtgctaccccatccccaggcacgacaccgtgtactggtatcagcagggcccaggccaggacccccagttcctgatcagcttctacgagaagatgcagagcgacaagggcagcatccccgacagattcagcgcccagcagttcagcgactaccacagcgagctgaacatgagcagcctggaactgggcgacagcgccctgtacttctgtgccagctctctgagaggcggcctggaaaagctgttcttcggcagcggcacccagctgagcgtgctggaagacctgaacaaggtgttccccccagaggtggccgtgttcgagccttctgaggccgaaatctcccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccacaatcctgtacgagatcctgctgggcaaggccaccctgtacgccgtgctggtgtctgccctggtgctgatggccatggtcaagcggaaggacttcggttccggagccacgaacttctctctgttaaagcaagcaggagacgtggaagaaaaccccggtcccatgagcctgagcagcctgctgaaagtcgtgaccgccagcctgtggctgggacctggaatcgcccagaagatcacccagacccagcccggcatgttcgtgcaggaaaaagaagccgtgaccctggactgcacctacgacaccagcgaccctagctacggcctgttctggtacaagcagcccagcagcggcgagatgatcttcctgatctaccagggcagctacgaccagcagaacgccaccgagggccggtacagcctgaacttccagaaggcccggaagtccgccaacctcgtgatcagcgccagccagctgggcgacagcgccatgtacttttgcgccatcagcggcaacacccccctggtgtttggcaagggcacccggctgagcgtgatcgccaacatccagaaccccgaccccgcagtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctaatcgatcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagaattctctggccgtcgttttaca

SEQ ID NO: 627 - DNA, 4638 bp, Homo sapiens
  source 1..4638: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggcaccagactgctgtgttgggccgctctgtgtctgctgggagccgaactgactgaagctggcgtggcccagagcccccggtacaagatcatcgagaagcggcagagcgtggccttctggtgcaaccctatcagcggacacgccaccctgtactggtatcagcagatcctgggccagggccccaagctgctgattcagttccagaacaacggcgtggtggacgacagccagctgcccaaggatagattcagcgccgagcggctgaagggcgtggacagcacactgaagatccagcccgccaagctggaagatagcgccgtgtacctgtgcgccagcagcctgtatagaggcgagcagtacttcggccctggcacccggctgaccgtgaccgaggatctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatggctatgctgctgggcgcctctgtgctgatcctgtggctgcagcccgactgggtcaacagccagcagaagaacgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttctgtgccgccagaggccagggcaacctgatctttggcaagggcaccaagctgagcgtgaagcccaacatccagaaccccgaccccgcagtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctaatcgatcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagaattctctggccgtcgttttac

SEQ ID NO: 628 - DNA, 4737 bp, Homo sapiens
  source 1..4737: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgtctctgggcctgctgtgctgtggcgccttctctctgctgtgggccggacctgtgaatgccggcgtgacccagacccccaagttccgggtgctgaaaaccggccagagcatgaccctgctgtgtgcccaggacatgaaccacgagtacatgtattggtacagacaggaccccggcatgggcctgcggctgatccactattctgtgggcgagggcaccaccgccaagggcgaagtgcctgatggctacaacgtgtcccggctgaagaagcagaacttcctgctgggcctggaaagcgccgctcctagccagaccagcgtgtacttctgtgccagccggggctaccaccggctgaacaacgagcagttcttcggccctggcacccggctgaccgtgctggaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggcggttccggagccacgaacttctctctgttaaagcaagcaggagacgtggaagaaaaccccggtcccatgatcagcctgcgggtgctgctcgtgatcctgtggctgcagctgagctgggtgtggtccggaggtgggtcatggtcccatccgcaattcgaaaagggaggtgggtcaggtggaggaagcggcggatccgcgtggtcacatcctcagttcgaaaagcagcggaaagaggtggaacaggaccctggccccttcaacgtgccagagggcgccaccgtggccttcaactgcacctacagcaacagcgccagccagagcttcttctggtacagacaggactgccggaaagaacccaagctgctgatgagcgtgtacagcagcggcaacgaggacggcagattcaccgcccagctgaacagagcctcccagtacatctccctgctgatccgggacagcaagctgagcgacagcgccacctacctgtgcgtcgtgaagcctgatcctggcgccggaagctaccagctgacctttggcaagggcaccaagctgtccgtgatccccaacatccagaaccccgaccccgcagtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctaatcgatcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagaattctctggccgtcgttttacaac

SEQ ID NO: 629 - DNA, 4646 bp, Homo sapiens
  source 1..4646: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgcggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatgtgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgatgcggccgccaccatgggatgtagacttctgtgttgcgccgtgctgtgtctgcttggagctggcgaactggtgcctatggaaaccggcgtgacccagacacctagacacctggtcatgggcatgacaaacaagaaaagcctgaagtgcgagcagcacctgggccacaatgccatgtactggtacaagcagagcgccaagaaacccctggaactgatgttcgtgtacagcctggaagagagggtcgagaacaacagcgtgcccagcagattcagccctgagtgccctaatagcagccacctgtttctgcatctgcacaccctgcagcctgaggactctgccctgtatctgtgtgccagcagccaggactacctggtgtccaacgagaagctgttcttcggcagcggcacacagctgagcgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggaaaagccactctgtacgctgtgctggtgtccgctctggtgctgatggccatggtcaagcggaaggatagcaggggcggctccggtgccacaaacttctccctgctcaagcaggccggagatgtggaagagaaccctggccctatgatcagcctgagagtgctgctggtcatcctgtggctgcagctgtcttgggtctggtcccagcggaaagaggtggaacaggaccccggacctttcaatgtgcctgaaggcgccaccgtggccttcaactgcacctacagcaatagcgccagccagagcttcttctggtacagacaggactgccggaaagaacccaagctgctgatgagcgtgtacagcagcggcaacgaggacggcagattcacagcccagctgaacagagccagccagtacatcagcctgctgatccgggatagcaagctgagcgatagcgccacctacctgtgcgtggtcaacctgctgtctaatcaaggcggcaagctgatcttcggccagggcacagagctgagcgtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccggatcctgctgctgaaagtggccggcttcaacctcctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaagaattctctggccgtcgttttacaacg

SEQ ID NO: 630 - DNA, 3448 bp, Homo sapiens
  source 1..3448: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgagggccgcggcagcctgctgacctgcggcgacgtggaggagaatcccggccccatgagcaatcaagtcctttgttgtgttgtcctgtgtttccttggcgcaaataccgtcgacggcgggataactcagtcccccaaatacctcttcagaaaagaaggacaaaacgtaacgctttcctgcgagcagaatcttaaccacgacgccatgtactggtacaggcaagaccctggacaaggtcttaggctcatatactactctcagattgtcaatgacttccaaaaaggggacatagctgagggctactctgtgtcccgagagaagaaggagtcatttcccctgacagtgacttctgcacaaaaaaatcccactgcattttatttgtgtgccagttcaccgggtgcattgtatgagcaatactttggccccggcactagactgacagttacggaggatctgaaaaacgtcttcccgccagaggtggcagttttcgagcccagtgaggctgaaatctctcatacccaaaaagcaacccttgtctgtctcgccactggattctatcccgaccacgtcgaattgagctggtgggtcaatgggaaagaggtacatagtggggtctgtacggatccacaaccccttaaggaacaacctgcccttaacgattcacgatactgcctgtcatcacgactcagggtatctgctaccttttggcagaacccgagaaatcactttcggtgccaggttcagttttacggacttagcgaaaatgatgaatggacacaagaccgagcaaagcccgttactcaaatagtgagcgcggaagcctgggggcgagcagactgcggcttcacctccgaaagttaccagcaaggtgttttgtcagccaccattttgtatgagattttgttggggaaggcgacactttacgcggtactggtctctgccttggttcttatggctatggtcaagaggaaagattccaggggtggctccggtgccacaaacttctccctgctcaagcaggccggagatgtggaagagaaccctggccctatgacctctatcagagctgtctttatatttctctggcttcaacttgatctggtgaatggcgaaaacgtggaacagcacccttcaacgttgagcgttcaggaaggagattcagccgtcatcaagtgtacgtattccgattccgcgtcaaactacttcccgtggtacaaacaggaacttggcaagcgcccccagctcattatcgacatcagaagcaacgtaggagagaagaaggaccaacgcatagctgtgactctcaacaaaacagctaaacatttctccctgcacattacggaaacccaaccagaggattctgccgtatacttttgtgctgctactgaggacctcactttgatctggggagcgggtacgaagctcataataaaacccgatatccaaaacccggacccagcagtttatcaattgagagatagtaagtccagtgacaaatcagtttgtttgtttacggatttcgatagccagaccaatgtcagtcagtcaaaggacagtgatgtatacattacagataaatgtgtacttgacatgcgctcaatggactttaagtctaactctgctgtagcttggtctaacaaaagtgattttgcgtgtgccaacgcatttaacaacagcatcatacctgaagacacgttctttccgagtccagaaagttcctgtgacgtgaaacttgtagaaaagagtttcgagaccgacactaaccttaactttcaaaacctttcagtgatcggatttagaatcttgctgctcaaggtggcagggttcaatctgctgatgactctgcgactgtggagttcataacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 631 - DNA, 3478 bp, Homo sapiens
  source 1..3478: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgagggccgcggcagcctgctgacctgcggcgacgtggaggagaatcccggccccatgggatgtagacttctgtgttgcgccgtgctgtgtctgcttggagctggcgaactggtgcctatggaaaccggcgtgacccagacacctagacacctggtcatgggcatgacaaacaagaaaagcctgaagtgcgagcagcacctgggccacaatgccatgtactggtacaagcagagcgccaagaaacccctggaactgatgttcgtgtacagcctggaagagagggtcgagaacaacagcgtgcccagcagattcagccctgagtgccctaatagcagccacctgtttctgcatctgcacaccctgcagcctgaggactctgccctgtatctgtgtgccagcagccaggactacctggtgtccaacgagaagctgttcttcggcagcggcacacagctgagcgtgctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggaaaagccactctgtacgctgtgctggtgtccgctctggtgctgatggccatggtcaagcggaaggatagcaggggcggctccggtgccacaaacttctccctgctcaagcaggccggagatgtggaagagaaccctggccctatgatcagcctgagagtgctgctggtcatcctgtggctgcagctgtcttgggtctggtcccagcggaaagaggtggaacaggaccccggacctttcaatgtgcctgaaggcgccaccgtggccttcaactgcacctacagcaatagcgccagccagagcttcttctggtacagacaggactgccggaaagaacccaagctgctgatgagcgtgtacagcagcggcaacgaggacggcagattcacagcccagctgaacagagccagccagtacatcagcctgctgatccgggatagcaagctgagcgatagcgccacctacctgtgcgtggtcaacctgctgtctaatcaaggcggcaagctgatcttcggccagggcacagagctgagcgtgaagcccaacattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagaccgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgtccgtgatcggcttccggatcctgctgctgaaagtggccggcttcaacctcctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 632 - DNA, 3466 bp, Homo sapiens
  source 1..3466: mol_type = genomic DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatcttgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgagggccgcggcagcctgctgacctgcggcgacgtggaggagaatcccggccccatgggatcttggacactgtgttgcgtgtccctgtgcatcctggtggccaagcacacagatgccggcgtgatccagtctcctagacacgaagtgaccgagatgggccaagaagtgaccctgcgctgcaagcctatcagcggccacgattacctgttctggtacagacagaccatgatgagaggcctggaactgctgatctacttcaacaacaacgtgcccatcgacgacagcggcatgcccgaggatagattcagcgccaagatgcccaacgccagcttcagcaccctgaagatccagcctagcgagcccagagatagcgccgtgtacttctgcgccagcagaaagacaggcggctacagcaatcagccccagcactttggagatggcacccggctgagcatcctggaagatctgaagaacgtgttcccacctgaggtggccgtgttcgagccttctgaggccgagatcagccacacacagaaagccacactcgtgtgtctggccaccggcttctatcccgatcacgtggaactgtcttggtgggtcaacggcaaagaggtgcacagcggcgtcagcaccgatcctcagcctctgaaagagcagcccgctctgaacgacagcagatactgcctgagcagcagactgagagtgtccgccaccttctggcagaaccccagaaaccacttcagatgccaggtgcagttctacggcctgagcgagaacgatgagtggacccaggatagagccaagcctgtgacacagatcgtgtctgccgaagcctggggcagagccgattgtggctttaccagcgagagctaccagcagggcgtgctgtctgccacaatcctgtacgagatcctgctgggcaaagccactctgtacgccgtgctggtgtctgccctggtgctgatggccatggtcaagcggaaggatagcaggggcggctccggtgccacaaacttctccctgctcaagcaggccggagatgtggaagagaaccctggccctatggaaaccctgctgaaggtgctgagcggcacactgctgtggcagctgacatgggtccgatctcagcagcctgtgcagtctcctcaggccgtgattctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgtggcaccgcctggatcaacgactacaagctgtctttcggagccggcaccacagtgacagtgcgggccaatattcagaaccccgatcctgccgtgtaccagctgagagacagcaagagcagcgacaagagcgtgtgcctgttcaccgacttcgacagccagaccaacgtgtcccagagcaaggacagcgacgtgtacatcaccgataagactgtgctggacatgcggagcatggacttcaagagcaacagcgccgtggcctggtccaacaagagcgatttcgcctgcgccaacgccttcaacaacagcattatccccgaggacacattcttcccaagtcctgagagcagctgcgacgtgaagctggtggaaaagagcttcgagacagacaccaacctgaacttccagaacctgagcgtgatcggcttcagaatcctgctgctcaaggtggccggcttcaacctgctgatgaccctgagactgtggtccagctaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccactagtcgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa